SEQUENCE LISTING (27 sequences)

SEQ ID NO: 1 - protein, 16 aa, Mus musculus
RSSQSIVHSNGNTYLE

SEQ ID NO: 2 - protein, 7 aa, Mus musculus
KVSNRFS

SEQ ID NO: 3 - protein, 9 aa, Mus musculus
FQGAHVPLT

SEQ ID NO: 4 - protein, 112 aa, Mus musculus
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGAHVPLTFGAGTKLELK

SEQ ID NO: 5 - protein, 5 aa, Mus musculus
TNAMN

SEQ ID NO: 6 - protein, 19 aa, Mus musculus
RIRSKRNNYATYYADSVKD

SEQ ID NO: 7 - protein, 7 aa, Mus musculus
DGLGMDY

SEQ ID NO: 8 - protein, 118 aa, Mus musculus
EVQLVETGGGLVQPKGSLKLSCAASGFTFNTNAMNWVRQAPGKGLEWVARIRSKRNNYATYYADSVKDRFTISRDESQNMLFLQMNNLKTEDTAMYYCVRDGLGMDYWGQGTSVTVSS

SEQ ID NO: 9 - protein, 19 aa, Mus musculus
RIRSKSNNYATYYADSVKD

SEQ ID NO: 10 - protein, 118 aa, Mus musculus
EVQLVETGGGLVQPKGSLKLSCAASGFTFNTNAMNWVRQAPGKGLEWVARIRSKSNNYATYYADSVKDRFTISRDESQNMLFLQMNNLKTEDTAMYYCVRDGLGMDYWGQGTSVTVSS

SEQ ID NO: 11 - protein, 16 aa, Mus musculus
RSSQSIVHSNGHTYLE

SEQ ID NO: 12 - protein, 112 aa, Mus musculus
DVLMTQTPLSLPVSLGDQASISCRSSQSIVHSNGHTYLEWYLQKPGQSPKLLIYKVSNRFSGVPDRFSGSGSGTDFTLKISRVEAEDLGVYYCFQGAHVPLTFGAGTKLELK

SEQ ID NO: 13 - protein, 15 aa, Mus musculus
RSSKSVSTSGYSYML

SEQ ID NO: 14 - protein, 7 aa, Mus musculus
LVSNLEC

SEQ ID NO: 15 - protein, 9 aa, Mus musculus
QHIRELTRS

SEQ ID NO: 16 - protein, 110 aa, Mus musculus
DIVVTQCRGSLDVSLGQRATISYRSSKSVSTSGYSYMLWNQQKSGQPPRLLMYLVSNLECGVRARFSGSGYGTEFTLNFHHVEEEDVATYSSQHIRELTRSEGGPSWKZN

SEQ ID NO: 17 - protein, 15 aa, Mus musculus
RASKSVSTSGYSYMH

SEQ ID NO: 18 - protein, 7 aa, Mus musculus
LVSNLES

SEQ ID NO: 19 - protein, 110 aa, Mus musculus
DIVLTQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGPSWKZN

SEQ ID NO: 20 - protein, 11 aa, Mus musculus
KASQNVGTNVA

SEQ ID NO: 21 - protein, 7 aa, Mus musculus
SASYRYS

SEQ ID NO: 22 - protein, 9 aa, Mus musculus
QQYNSYPYT

SEQ ID NO: 23 - protein, 107 aa, Mus musculus
DIVMTQSQKFMSTSVGDRVSVTCKASQNVGTNVAWYQQKPGQSPKALIYSASYRYSGVPDRFTGSGSGTDFTLTISNVQSEDLAEYFCQQYNSYPYTFGAGTKLELK

SEQ ID NO: 24 - protein, 16 aa, Mus musculus
  misc_feature (1)..(1): Xaa is Arg or Lys
  misc_feature (2)..(2): Xaa is Ser or Ala
  misc_feature (3)..(3): Xaa is Ser
  misc_feature (4)..(4): Xaa is Gln or Lys
  misc_feature (5)..(5): Xaa is Ser or Asn
  misc_feature (6)..(6): Xaa is Ile or Val
  misc_feature (7)..(7): Xaa is Val or absent
  misc_feature (8)..(8): Xaa is His, Ser, or absent
  misc_feature (9)..(9): Xaa is Ser, Thr, or absent
  misc_feature (10)..(10): Xaa is Asn, Ser, or absent
  misc_feature (11)..(11): Xaa is Gly or absent
  misc_feature (12)..(12): Xaa is Asn, His, Tyr, or Gly
  misc_feature (13)..(13): Xaa is Thr or Ser
  misc_feature (14)..(14): Xaa is Tyr or Asn
  misc_feature (15)..(15): Xaa is Leu, Met, or Val
  misc_feature (16)..(16): Xaa is Glu, Leu, His, or Ala
XXXXXXXXXXXXXXXX

SEQ ID NO: 25 - protein, 7 aa, Mus musculus
  misc_feature (1)..(1): Xaa is Lys, Leu, or Ser
  misc_feature (2)..(2): Xaa is Val or Ala
  misc_feature (3)..(3): Xaa is Ser
  misc_feature (4)..(4): Xaa is Asn or Tyr
  misc_feature (5)..(5): Xaa is Arg or Leu
  misc_feature (6)..(6): Xaa is Phe, Glu, or Tyr
  misc_feature (7)..(7): Xaa is Ser or Cys
XXXXXXX

SEQ ID NO: 26 - protein, 9 aa, Mus musculus
  misc_feature (1)..(1): Xaa is Phe or Gln
  misc_feature (2)..(2): Xaa is Gln or His
  misc_feature (3)..(3): Xaa is Gly, Ile, or Tyr
  misc_feature (4)..(4): Xaa is Ala, Arg, Asn
  misc_feature (5)..(5): Xaa is His, Glu, or Ser
  misc_feature (6)..(6): Xaa is Val, Leu, or Tyr
  misc_feature (7)..(7): Xaa is Pro or Thr
  misc_feature (8)..(8): Xaa is Leu, Arg, or Tyr
  misc_feature (9)..(9): Xaa is Thr or Ser
XXXXXXXXX

SEQ ID NO: 27 - protein, 19 aa, Mus musculus
  misc_feature (6)..(6): Xaa is Arg or Ser
RIRSKXNNYATYYADSVKD

(19) United States
(12) Patent Application Publication
(10) Pub. No.: US 2022/0348645 A1
(43) Pub. Date: Nov. 3, 2022

(54) CONFORMATION-SPECIFIC ANTIBODIES THAT BIND NUCLEAR FACTOR KAPPA-LIGHT-CHAIN-ENHANCER OF ACTIVATED B CELLS

(71) Applicant: Beth Israel Deaconess Medical Center, Inc., Boston, MA (US)

(72) Inventors: Kun Ping LU, Newton, MA (US); Xiao Zhen ZHOU, Newton, MA (US)

(21) Appl. No.: 17/765,514

(22) PCT Filed: Oct. 1, 2020
(86) PCT No.: PCT/US2020/053851

§ 371 Date: Mar. 31, 2022

Related U.S. Application Data
(60) Provisional application No. 62/909,123, filed on Oct. 1, 2019.

(51) Int. Cl. C07K 16/18; A61K 47/68; A61K 39/395; A61K 45/06; G01N 33/68; A61P 31/04

(52) U.S. Cl. CPC C07K 16/18; A61K 47/6803; A61K 47/6843; A61K 39/3955; A61K 45/06; G01N 33/6893; A61P 31/04; G01N 2333/4703; A61K 2039/505

(57) ABSTRACT

Described are conformation-specific antibodies or antigen-binding fragments that specifically bind to the trans conformation of phosphorylated-Threonine254-Proline (pThr254-Pro) of the p65 subunit of nuclear factor kappa-light-chain-enhancer of activated B cells (NF-κB). Also described are related pharmaceutical compositions, polynucleotides, peptides, vectors, host cells, methods of production, methods of treatment, diagnostic methods, and kits.

### STATEMENT AS TO FEDERALLY SPONSORED RESEARCH

This invention was made with government support under grant number R01CA167677 awarded by the National Institutes of Health. The government has certain rights in the invention.

### SEQUENCE LISTING

The instant application contains a Sequence Listing which has been submitted electronically in ASCII format and is hereby incorporated by reference in its entirety. The ASCII copy, created on Aug. 18, 2020, is named 01948-262WO2_Sequence_Listing_9.30.20_ST25 and is 13,443 bytes in size.

### BACKGROUND OF THE INVENTION

NF-κB (nuclear factor kappa-light-chain-enhancer of activated B cells) is a major transcription factor that plays a critical role in regulating cellular responses to various stimuli, such as stress or bacterial or viral infection. Upon activation, NF-κB is activated by the degradation of inhibitors of κB (IκBs), resulting in translocation from the cytoplasm into the nucleus, where it regulates transcription of a range of NF-κB target genes as part of the normal body defense system in response to stress, inflammation, or infection. Abnormal activation of NF-κB has been linked to cancer, inflammatory and immune diseases, viral infection, and sepsis. Abnormal activation of NF-κB has also been associated with Cytokine Release Syndrome (CRS), which is alternately referred to as Cytokine Storm Syndrome (CSS).

Sepsis is a major global health issue and a key therapeutic challenge. Sepsis affects about one million people each year in the U.S. and is the number one killer in the ICU patient, with a mortality rate of about 30 to 50%. Sepsis is one of the most expensive diseases in the U.S. medical community, accounting for ˜40% of the ICU's total expenditure. The economic burden caused by sepsis exceeds 45 billion U.S. dollars annually (Torio, C. M. & Moore, B. J. Healthcare Cost and Utilization Project (HCUP) Statistical Briefs (2006)).

Sepsis can result when pathogenic bacteria invade the bloodstream, rapidly multiply, and produce large amounts of toxins, which causes rapid and uncontrolled extreme activation of the innate immune system. Activation of the immune system can result in pro-inflammatory cytokine storm, which can cause damage to and failure of many organs and organ systems. Septic shock has a high mortality. For survivors, immunosuppressed states and slow development of organ scars become a serious long-term therapeutic challenge. Drug development for sepsis has historically emphasized suppression of extreme activation of the immune response, and many clinical trials have been conducted without success. While some of the tested drugs were observed to inhibit extreme activation of the immune system in the early stages of sepsis and to reduce the early mortality rate, they further exacerbated the immunosuppressed state of the patients, leading to immunoparalysis. Immunoparalysis results in ineffective clearance of septic foci and renders the septic patient more vulnerable to secondary infections, as well as reactivation of latent infections. Thus, the overall survival rate did not improve.

CRS is a systemic inflammatory response that can be triggered by a variety of stimuli including infections such as influenza, inflammatory diseases such as sever acute pancreatitis, and certain therapeutics, such as chimertic antigen receptor T cell (CAR-T) therapy or antibody therapy. For example, CRS is a potentially fatal side effect that has been associated with CAR-T therapy, such as CD19 CAR-T cell therap in acute lymphoblastic leukemia (B-ALL, chronic lymphoblastic leukemia (B-OLL), and B-cell non-Hodgkin lymphoma (B-NHL) (Jin, z. et al. Ann Hematol. 97:1327 (2018)). Furthermore, CRS has been described after infusion of several antibody-based therapies such as anti-thymocyte globulin (ATG), the CD28 superagonist TGN1412, rituximab, obinutuzumab, alemtuzumab, brentuximab, dacetuzumab, and nivolumab. CRS has also been observed following administration of non-protein-based cancer drugs such as oxaliplatin and lenalidomide. Moreover, CRS was reported in the setting of haploidentical donor stem cell transplantation and graft-versus-host disease. Cytokine storm due to massive T cell stimulation is also a proposed pathomechanism of severe viral infections such as influenza. CRS, alternately referred to as cytokine storm, is also associated with the hemophagocytic syndromes such as macrophage activation syndrome (MAS) and hemophagocytic lymphohistiocytosis (HLH) (Canna, S. W. and Behrens, E. M. Pediatr Clin North Am. 59(2):329-344 (2012)). The epidemiology, clinical presentation, pathophysiology, and differential diagnosis of CRS have been described, for example, in Shimabukuro-Vornhagen, A. et al. Journal for ImmunoTherapy of Cancer. 6:56 (2018); Canna, S. W. and Behrens, E. M. Pediatr Clin N Am. 59:329-344 (2012); and Chavez, J. C. et al. Hematol Oncol Stem Cell Ther. doi.org/10.1016/j.hemonc.2019.05.005, each of which is incorporated herein by reference.

There is a need for treatments for disorders associated with deregulation of NF-κB, including infection, cancer, and inflammatory and immune diseases, such as sepsis, septic shock, systemic inflammatory response syndrome (SIRS), and CRS.

### SUMMARY OF THE INVENTION

Described herein are conformation-specific antibodies or antigen-binding fragments that specifically bind to the trans conformation of phosphorylated-Threonine254-Proline (pThr254-Pro) of the p65 subunit of nuclear factor kappa-light-chain-enhancer of activated B cells (NF-κB). The present invention is based, in part, on the surprising discovery that the trans conformation of pThr254-Pro of p65 is favored in the nuclear, active form of NF-κB. Accordingly, antibodies or antigen-binding fragments are described which specifically recognize the active nuclear form, but not the inactive cytoplasmic form, of p65 NF-κB. Antibodies or antigen-binding fragments described herein may inhibit the pathogenic function of dysregulated (e.g., overexpressed) NF-κB, and may be used for the treatment of NF-κB-related diseases (e.g., infection, cancer, or immune or inflammatory disorders, such as sepsis, septic shock, systemic inflammatory response syndrome (SIRS), or CRS). The invention also provides related pharmaceutical compositions, polynucleotides, vectors, host cells, methods of production, methods of treatment, diagnostic methods, and kits.

In a first aspect, the invention features an isolated conformation-specific antibody or antigen-binding fragment thereof, wherein the antibody or antigen-binding fragment thereof specifically binds an epitope including the trans conformation of phosphorylated-Threonine254-Proline (pThr254-Pro) of the p65 subunit of nuclear factor kappa-light-chain-enhancer of activated B cells (NF-κB).

In some embodiments, the antibody or antigen-binding fragment thereof includes a complementarity-determining region (CDR) light chain 1 (CDR-L1) having the amino acid sequence of SEQ ID NO: 1, SEQ ID NO: 11, SEQ ID NO: 13, SEQ ID NO: 17, SEQ ID NO: 20, SEQ ID NO: 24, or a variant thereof. Variant CDRs for CDR-L1 are also envisioned, which may include one, two, three, four, or five amino acid substitutions, deletions, or additions relative to the recited sequence (e.g., SEQ ID NOs: 1, 11, 13, 17, 20, or 24).

In some embodiments, the antibody or antigen-binding fragment thereof includes a complementarity-determining region (CDR) light chain 2 (CDR-L2) having the amino acid sequence of SEQ ID NO: 2, SEQ ID NO: 14, SEQ ID NO: 18, SEQ ID NO: 21, SEQ ID NO: 25, or a variant thereof. Variant CDRs for CDR-L2 are also envisioned, which may include one, two, three, four, or five amino acid substitutions, deletions, or additions relative to the recited sequence (e.g., SEQ ID NOs: 2, 14, 18, 21, or 25).

In some embodiments, the antibody or antigen-binding fragment thereof includes a complementarity-determining region (CDR) light chain 3 (CDR-L3) having the amino acid sequence of SEQ ID NO: 3, SEQ ID NO: 15, SEQ ID NO: 22, SEQ ID NO: 26, or a variant thereof. Variant CDRs for CDR-L3 are also envisioned, which may include one, two, three, four, or five amino acid substitutions, deletions, or additions relative to the recited sequence (e.g., SEQ ID NOs: 3, 15, 22, or 26).

In some embodiments, the antibody or antigen-binding fragment thereof includes: a complementarity-determining region (CDR) light chain 1 (CDR-L1) having the amino acid sequence of SEQ ID NO: 1 or a variant thereof; a complementarity-determining region (CDR) light chain 2 (CDR-L2) having the amino acid sequence of SEQ ID NO: 2 or a variant thereof; and a complementarity-determining region (CDR) light chain 3 (CDR-L3) having the amino acid sequence of SEQ ID NO: 3 or a variant thereof.

In some embodiments, the antibody or antigen-binding fragment thereof includes a light chain variable domain having an amino acid sequence that is at least 70% identical (e.g., at least 75%, 80%, 85%, 90%, 95%, 96%, 97%, 98%, or 99%) identical to the amino acid sequence of SEQ ID NO: 4. In some embodiments, the antibody or antigen-binding fragment thereof includes a light chain variable domain having an amino acid sequence that is at least 85% identical to the amino acid sequence of SEQ ID NO: 4. In some embodiments, the antibody or antigen-binding fragment thereof includes a light chain variable domain having the amino acid sequence of SEQ ID NO: 4.

In some embodiments, the antibody or antigen-binding fragment thereof includes: a complementarity-determining region (CDR) light chain 1 (CDR-L1) having the amino acid sequence of SEQ ID NO: 11 or a variant thereof; a complementarity-determining region (CDR) light chain 2 (CDR-L2) having the amino acid sequence of SEQ ID NO: 2 or a variant thereof; and a complementarity-determining region (CDR) light chain 3 (CDR-L3) having the amino acid sequence of SEQ ID NO: 3 or a variant thereof.

In some embodiments, the antibody or antigen-binding fragment thereof includes a light chain variable domain having an amino acid sequence that is at least 70% identical (e.g., at least 75%, 80%, 85%, 90%, 95%, 96%, 97%, 98%, or 99%) identical to the amino acid sequence of SEQ ID NO: 12. In some embodiments, the antibody or antigen-binding fragment thereof includes a light chain variable domain having an amino acid sequence that is at least 85% identical to the amino acid sequence of SEQ ID NO: 12. In some embodiments, the antibody or antigen-binding fragment thereof includes a light chain variable domain having the amino acid sequence of SEQ ID NO: 12.

In some embodiments, the antibody or antigen-binding fragment thereof includes: a complementarity-determining region (CDR) light chain 1 (CDR-L1) having the amino acid sequence of SEQ ID NO: 13 or a variant thereof; a complementarity-determining region (CDR) light chain 2 (CDR-L2) having the amino acid sequence of SEQ ID NO: 14 or a variant thereof; and a complementarity-determining region (CDR) light chain 3 (CDR-L3) having the amino acid sequence of SEQ ID NO: 15 or a variant thereof.

In some embodiments, the antibody or antigen-binding fragment thereof includes a light chain variable domain having an amino acid sequence that is at least 70% identical (e.g., at least 75%, 80%, 85%, 90%, 95%, 96%, 97%, 98%, or 99%) identical to the amino acid sequence of SEQ ID NO: 16. In some embodiments, the antibody or antigen-binding fragment thereof includes a light chain variable domain having an amino acid sequence that is at least 85% identical to the amino acid sequence of SEQ ID NO: 16. In some embodiments, the antibody or antigen-binding fragment thereof includes a light chain variable domain having the amino acid sequence of SEQ ID NO: 16.

In some embodiments, the antibody or antigen-binding fragment thereof includes: a complementarity-determining region (CDR) light chain 1 (CDR-L1) having the amino acid sequence of SEQ ID NO: 17 or a variant thereof; a complementarity-determining region (CDR) light chain 2 (CDR-L2) having the amino acid sequence of SEQ ID NO: 18 or a variant thereof; and a complementarity-determining region (CDR) light chain 3 (CDR-L3) having the amino acid sequence of SEQ ID NO: 15 or a variant thereof.

In some embodiments, the antibody or antigen-binding fragment thereof includes a light chain variable domain having an amino acid sequence that is at least 70% identical (e.g., at least 75%, 80%, 85%, 90%, 95%, 96%, 97%, 98%, or 99%) identical to the amino acid sequence of SEQ ID NO: 19. In some embodiments, the antibody or antigen-binding fragment thereof includes a light chain variable domain having an amino acid sequence that is at least 85% identical to the amino acid sequence of SEQ ID NO: 19. In some embodiments, the antibody or antigen-binding fragment thereof includes a light chain variable domain having the amino acid sequence of SEQ ID NO: 19.

In some embodiments, the antibody or antigen-binding fragment thereof includes: a complementarity-determining region (CDR) light chain 1 (CDR-L1) having the amino acid sequence of SEQ ID NO: 20 or a variant thereof; a complementarity-determining region (CDR) light chain 2 (CDR-L2) having the amino acid sequence of SEQ ID NO: 21 or a variant thereof; and a complementarity-determining region (CDR) light chain 3 (CDR-L3) having the amino acid sequence of SEQ ID NO: 22 or a variant thereof.

In some embodiments, the antibody or antigen-binding fragment thereof includes a light chain variable domain having an amino acid sequence that is at least 70% identical (e.g., at least 75%, 80%, 85%, 90%, 95%, 96%, 97%, 98%, or 99%) identical to the amino acid sequence of SEQ ID NO: 23. In some embodiments, the antibody or antigen-binding fragment thereof includes a light chain variable domain having an amino acid sequence that is at least 85% identical to the amino acid sequence of SEQ ID NO: 23. In some embodiments, the antibody or antigen-binding fragment thereof includes a light chain variable domain having the amino acid sequence of SEQ ID NO: 23.

In some embodiments, the antibody or antigen-binding fragment thereof includes a complementarity-determining region (CDR) heavy chain 1 (CDR-H1) having the amino acid sequence of SEQ ID NO: 5 or a variant thereof. Variant CDRs for CDR-H1 are also envisioned, which may include one, two, three, four, or five amino acid substitutions, deletions, or additions relative to the recited sequence (e.g., SEQ ID NO: 5).

In some embodiments, the antibody or antigen-binding fragment thereof includes a complementarity-determining region (CDR) heavy chain 2 (CDR-H2) having the amino acid sequence of SEQ ID NO: 6, SEQ ID NO: 9, SEQ ID NO: 27, or a variant thereof. Variant CDRs for CDR-H1 are also envisioned, which may include one, two, three, four, or five amino acid substitutions, deletions, or additions relative to the recited sequence (e.g., SEQ ID NOs: 6, 9, or 27).

In some embodiments, the antibody or antigen-binding fragment thereof includes a complementarity-determining region (CDR) heavy chain 3 (CDR-H3) having the amino acid sequence of SEQ ID NO: 7, or a variant thereof. Variant CDRs for CDR-H3 are also envisioned, which may include one, two, three, four, or five amino acid substitutions, deletions, or additions relative to the recited sequence (e.g., SEQ ID NO: 7).

In some embodiments, the antibody or antigen-binding fragment thereof includes: a complementarity-determining region (CDR) heavy chain 1 (CDR-H1) having the amino acid sequence of SEQ ID NO: 5 or a variant thereof; a complementarity-determining region (CDR) heavy chain 2 (CDR-H2) having the amino acid sequence of SEQ ID NO: 6 or a variant thereof; and a complementarity-determining region (CDR) heavy chain 3 (CDR-H3) having the amino acid sequence of SEQ ID NO: 7 or a variant thereof.

In some embodiments, the antibody or antigen-binding fragment thereof includes a heavy chain variable domain having an amino acid sequence that is at least 70% identical (e.g., at least 75%, 80%, 85%, 90%, 95%, 96%, 97%, 98%, or 99%) identical to the amino acid sequence of SEQ ID NO: 8. In some embodiments, the antibody or antigen-binding fragment thereof includes a heavy chain variable domain having an amino acid sequence that is at least 85% identical to the amino acid sequence of SEQ ID NO: 8. In some embodiments, the antibody or antigen-binding fragment thereof includes a heavy chain variable domain having the amino acid sequence of SEQ ID NO: 8.

In some embodiments, the antibody or antigen-binding fragment thereof includes: a complementarity-determining region (CDR) heavy chain 1 (CDR-H1) having the amino acid sequence of SEQ ID NO: 5 or a variant thereof; a complementarity-determining region (CDR) heavy chain 2 (CDR-H2) having the amino acid sequence of SEQ ID NO: 9 or a variant thereof; and a complementarity-determining region (CDR) heavy chain 3 (CDR-H3) having the amino acid sequence of SEQ ID NO: 7 or a variant thereof.

In some embodiments, the antibody or antigen-binding fragment thereof includes a heavy chain variable domain having an amino acid sequence that is at least 70% identical (e.g., at least 75%, 80%, 85%, 90%, 95%, 96%, 97%, 98%, or 99%) identical to the amino acid sequence of SEQ ID NO: 10. In some embodiments, the antibody or antigen-binding fragment thereof includes a heavy chain variable domain having an amino acid sequence that is at least 85% identical to the amino acid sequence of SEQ ID NO: 10. In some embodiments, the antibody or antigen-binding fragment thereof includes a heavy chain variable domain having the amino acid sequence of SEQ ID NO: 10.

In some embodiments, the antibody or antigen-binding fragment thereof includes a complementarity-determining region (CDR) light chain 1 (CDR-L1) having the amino acid sequence of SEQ ID NO: 24 or a variant thereof.

In some embodiments, the antibody or antigen-binding fragment thereof includes a complementarity-determining region (CDR) light chain 2 (CDR-L2) having the amino acid sequence of SEQ ID NO: 25 or a variant thereof.

In some embodiments, the antibody or antigen-binding fragment thereof includes a complementarity-determining region (CDR) light chain 3 (CDR-L3) having the amino acid sequence of SEQ ID NO: 26 or a variant thereof.

In some embodiments, the antibody or antigen-binding fragment thereof includes: a complementarity-determining region (CDR) light chain 1 (CDR-L1) having the amino acid sequence of SEQ ID NO: 24 or a variant thereof; a complementarity-determining region (CDR) light chain 2 (CDR-L2) having the amino acid sequence of SEQ ID NO: 25 or a variant thereof; and a complementarity-determining region (CDR) light chain 3 (CDR-L3) having the amino acid sequence of SEQ ID NO: 26 or a variant thereof.

In some embodiments, the antibody or antigen-binding fragment thereof includes: a complementarity-determining region (CDR) heavy chain 2 (CDR-H2) having the amino acid sequence of SEQ ID NO: 27 or a variant thereof.

In some embodiments, the antibody or antigen-binding fragment thereof includes: a complementarity-determining region (CDR) heavy chain 1 (CDR-H1) having the amino acid sequence of SEQ ID NO: 5 or a variant thereof; a complementarity-determining region (CDR) heavy chain 2 (CDR-H2) having the amino acid sequence of SEQ ID NO: 27 or a variant thereof; and a complementarity-determining region (CDR) heavy chain 3 (CDR-H3) having the amino acid sequence of SEQ ID NO: 7 or a variant thereof.

In some embodiments, the amino acid sequence of a CDR described herein (e.g., any CDR-L1, CDR-L2, CDR-L3, CDR-H1, CDR-H2, or CDR-H3 described herein) includes between one and five amino acid mutations relative to the indicated amino acid sequence (e.g., one amino acid mutation, one to two amino acid mutations, one to three amino acid mutations, one to four amino acid mutations, or one to five amino acid mutations. In preferred embodiments, the one or more amino acid mutations are conservative mutations.

In some embodiments, the antibody or antigen-binding fragment thereof binds to the trans conformation of pThr254-Pro with at least 2-fold (e.g., at least 5-fold, 10-fold, 20-fold, 30-fold, 40-fold, 50-fold, 60-fold, 70-fold, 80-fold, 90-fold, 100-fold, 200-fold, 500-fold, or 1000-fold) greater affinity than to the cis conformation of pThr254-Pro. In some embodiments, the antibody or antigen-binding fragment thereof binds to the trans conformation of pThr254-Pro with at least 10-fold greater affinity than to the cis conformation of pThr254-Pro. In some embodiments, the antibody or antigen-binding fragment thereof binds to the trans conformation of pThr254-Pro with at least 100-fold greater affinity than to the cis conformation of pThr254-Pro.

In some embodiments, the antibody or antigen-binding fragment thereof binds specifically to the active form of NF-κB. In some embodiments, the antibody or antigen-binding fragment thereof binds to the active form of NF-κB with at least 2-fold (e.g., at least 5-fold, 10-fold, 20-fold, 30-fold, 40-fold, 50-fold, 60-fold, 70-fold, 80-fold, 90-fold, 100-fold, 200-fold, 500-fold, or 1000-fold) greater affinity than to the inactive form of NF-κB. In some embodiments, the antibody or antigen-binding fragment thereof binds to the active form of NF-κB with at least 10-fold greater affinity than to the inactive form of NF-κB. In some embodiments, the antibody or antigen-binding fragment thereof binds to the active form of NF-κB with at least 100-fold greater affinity than to the inactive form of NF-κB.

In some embodiments, the antibody or antigen-binding fragment thereof binds specifically to the nuclear form of NF-κB. In some embodiments, the antibody or antigen-binding fragment thereof binds to the nuclear form of NF-κB with at least 2-fold (e.g., at least 5-fold, 10-fold, 20-fold, 30-fold, 40-fold, 50-fold, 60-fold, 70-fold, 80-fold, 90-fold, 100-fold, 200-fold, 500-fold, or 1000-fold)greater affinity than to the cytoplasmic form of NF-κB. In some embodiments, the antibody or antigen-binding fragment thereof binds to the nuclear form of NF-κB with at least 10-fold greater affinity than to the cytoplasmic form of NF-κB. In some embodiments, the antibody or antigen-binding fragment thereof binds to the nuclear form of NF-κB with at least 100-fold greater affinity than to the cytoplasmic form of NF-κB.

In some embodiments, the antibody or antigen-binding fragment thereof inhibits NF-κB signaling in a cell (e.g., decreases NF-κB signaling by at least 5%, 10%, 20%, 30%, 40%, 50%, 60%, 70%, 80%, 90%, 95%, 98%, 99%, or 100%). In some embodiments, the cell is a mammalian cell (e.g., a human cell). In some embodiments, the cell is an immune cell (e.g., a T-cell or a B-cell) or a cancer cell.

In some embodiments, the antibody or antigen-binding fragment thereof inhibits the expression of one or more genes (e.g., decreases expression of one or more genes by at least 5%, 10%, 20%, 30%, 40%, 50%, 60%, 70%, 80%, 90%, 95%, 98%, 99%, or 100%) selected from the group consisting of IGHG4, IGHG3, APOC3, TNFRSF6, CD3G, TNFSF5, CD105, ICAM1, TPMT, IL2RA, SELE, TP53, CRP, IL1A, IL1B, IL1RN, CCR5, IL8, IL2, IL9, TAP1, TNF, LTA, IL6, CD44, NOS2A, SOD2, TNFSF6, IL11, BDKRB1, CSF1, CSF2, CSF3, GSTP1, NQO1, OPRM1, PTAFR, PTGS2, SCNN1A, VCAM1, AGER, ALOX12B, BCL2L1, TNFRSF5, TNFRSF9, IRF7, BLR1, CD48, CD69, CCR7, CR2, F3, HMOX1, TNC, IFNB1, IL13, IL15RA, IRF1, IRF2, LTB, IRF4, MYC, NFKB2, PDGFB, PLAU, LMP2, PTX3, CCL2, CCL5, CCL11, CXCL5, SELP, SLC2A5, STAT5A, VIM, IER3, NFKB1, BM2, BCL2A1, CCL15, CD83, CD74, ELF3, TGM2, DEFB4, MMP9, BCL3, CD80, VEGFC, PLCD1, TNFAIP3, RELB, TFPI2, BCL2, S100A6, TACR1, NFKBIA, CD209, CARD15, CCND1, KLK3, IL15, NR4A2, and HC3.

In some embodiments, the antibody or antigen-binding fragment thereof is selected from the group consisting of a monoclonal antibody or antigen-binding fragment thereof, a polyclonal antibody or antigen-binding fragment thereof, a human antibody or antigen-binding fragment thereof, a humanized antibody or antigen-binding fragment thereof, a primatized antibody or antigen-binding fragment thereof, a bispecific antibody or antigen-binding fragment thereof, a multi-specific antibody or antigen-binding fragment thereof, a dual-variable immunoglobulin domain, a monovalent antibody or antigen-binding fragment thereof, a chimeric antibody or antigen-binding fragment thereof, a single-chain Fv molecule (scFv), a diabody, a triabody, a nanobody, an antibody-like protein scaffold, a domain antibody, a Fv fragment, a Fab fragment, a F(ab′)2 molecule, and a tandem scFv (taFv). In some embodiments, the antibody or antigen-binding fragment thereof is a human, humanized, or chimeric antibody or antigen-binding fragment thereof.

In some embodiments, the antibody is conjugated to a therapeutic agent (e.g., a cytotoxic agent).

In another aspect, the invention features a polynucleotide encoding any one of the antibodies or antigen-binding fragments thereof described herein (e.g., an antibody or antigen-binding fragment thereof that specifically binds an epitope including the trans conformation of pThr254-Pro of the p65 subunit of NF-κB).

In another aspect, the invention features a vector including any one of the polynucleotides described herein (e.g., a polynucleotide encoding an antibody or antigen-binding fragment thereof described herein). In some embodiments, the vector is an expression vector (e.g., a eukaryotic expression vector). In some embodiments, the vector is a viral vector (e.g., a viral vector selected from the group consisting of adenovirus (Ad), retrovirus, poxvirus, adeno-associated virus, baculovirus, herpes simplex virus, and a vaccinia virus).

In another aspect, the invention features a host cell including any one of the polynucleotides or vectors described herein. In some embodiments, the host cell is a prokaryotic cell. In some embodiments, the host cell is a eukaryotic cell (e.g., a mammalian cell, such as a human cell).

In another aspect, the invention features a pharmaceutical composition including any one of the antibodies or antigen-binding fragments thereof described herein, any one of the polynucleotides described herein, any one of the vectors described herein, or any one of the host cells described herein, and a pharmaceutically acceptable carrier or excipient.

In some embodiments, the antibody or antigen-binding fragment thereof is present in the pharmaceutical composition in an amount of from about 0.001 mg/ml to about 100 mg/ml (e.g., in an amount from 0.001 mg/ml to 0.01 mg/ml, from 0.01 mg/ml to 0.1 mg/ml, from 0.1 mg/ml to 1 mg/ml, from 1 mg/ml to 10 mg/ml, or from 10 mg/ml to 100 mg/ml).

In some embodiments, the pharmaceutical composition further includes an additional therapeutic agent (e.g., an immunotherapy agent, a chimeric antigen receptor (CAR-T) agent, a chemotherapeutic agent, a small molecule anti-cancer agent, a cancer vaccine, an antibacterial agent, an antifungal agent, or an antiviral agent).

In another aspect, the invention features a method of producing any one of the antibodies or antigen-binding fragments thereof described herein, the method including expressing a polynucleotide encoding the antibody or antigen-binding fragment thereof in a host cell and recovering the antibody or antigen-binding fragment thereof from host cell medium.

In another aspect, the invention features a method of producing any one of the antibodies or antigen-binding fragments thereof described herein, the method including: (i) administering an antigenic peptide to a non-human host animal, the antigenic peptide including a phosphorylated-Threonine-Xaa (pThr-Xaa) motif, where Xaa is any natural or non-natural amino acid; (ii) isolating antisera containing the antibody or antigen-binding fragment thereof produced in the non-human host animal; and (iii) purifying the antibody or antigen-binding fragment thereof from the antisera; wherein the antibody or antigen-binding fragment thereof specifically binds to the trans conformation of phosphorylated-Threonine254-Proline (pThr254-Pro) of the p65 subunit of nuclear factor kappa-light-chain-enhancer of activated B cells (NF-κB).

In some embodiments, the method further comprises the step of (iv) assaying the purified antibody or antigen-binding fragment thereof for the ability nuclear active form of the p65 subunit of NF-κB (e.g., by quantitative immunofluorescence assay as described herein). In some embodiments, the antibody or antigen-binding fragment thereof thereof binds to the active form of NF-κB with at least 2-fold (e.g., at least 5-fold, 10-fold, 20-fold, 30-fold, 40-fold, 50-fold, 60-fold, 70-fold, 80-fold, 90-fold, 100-fold, 200-fold, 500-fold, or 1000-fold) greater affinity than to the inactive form of NF-κB. The above-described method may also be used to select a population of antibodies or antigen-binding fragments thereof which bind specifically to the nuclear active form of the p65 subunit of NF-κB.

In some embodiments, the non-human host animal is a rabbit, a cow, a horse, a dog, a cat, a goat, a sheep, a chicken, a llama, or a camel.

In another aspect, the invention features a method of treating a subject (e.g., a human subject) having or at risk of developing an immune disorder or an inflammatory disorder (e.g., sepsis, such as septic shock, SIRS, or CRS), an infection, or a cancer, wherein the method includes administering to the subject an antigenic peptide, the antigenic peptide including a phosphorylated-Threonine-Xaa (pThr-Xaa) motif, where Xaa is any natural or non-natural amino acid, wherein administration of the antigenic peptide produces an antibody or antigen-binding fragment thereof in the subject that specifically binds to the trans conformation of phosphorylated-Threonine254-Proline (pThr254-Pro) of the p65 subunit of nuclear factor kappa-light-chain-enhancer of activated B cells (NF-κB).

In some embodiments, the antigenic peptide may contain an epitope from the p65 subunit of NF-κB (GenBank Accession No. AAH33210) including a pThr-Xaa motif (e.g., pThr-254-Xaa, for example, pThr254-Pro). The antigenic peptide may further include additional residues surrounding the pThr-Xaa motif of the full-length polypeptide. For example, the antigenic peptide may include the 3-10 amino acid residues N-terminal to the pThr254 residue of a full-length polypeptide and the 3-10 amino acid residues C-terminal to the Xaa255 (e.g., Pro255) of a full-length polypeptide.

In some embodiments, the peptidyl-prolyl bond of the pThr-Xaa motif of the antigenic peptide is in a trans conformation (e.g., is preferentially in the trans conformation relative to the cis conformation, such as a ratio of trans:cis of 60%:40%, 70%:30%, 80%:20%, 90%:10%, 95%:5%, or more). In some embodiments, Xaa is Pro. In other embodiments, Xaa is any natural or non-natural amino acid, wherein the peptide bond between pThr and Xaa in the pThr-Xaa motif is preferentially in the trans conformation. Most preferably, Xaa is an amino acid that shares structural similarity to Pro, but which resides preferentially in the trans-peptide bond conformation (e.g., Xaa of the pThr-Xaa motif is selected from Ala or Gly). For example, the antigenic peptide may be a peptide containing the pThr254-Pro motif of the p65 submit of NF-κB (GenBank Accession No. AAH33210), wherein Pro255 has been replaced with a natural or non-natural amino acid that resides preferentially in the trans-peptide bond conformation, for example, Ala or Gly.

In some embodiments, the antibody of antigen-binding fragment thereof binds to the trans conformation of pThr254-Pro of the p65 subunit of NF-κB with 2-fold, 5-fold, 10-fold, 50-fold, 100-fold, 500-fold, or 1000-fold or greater specificity than to the cis conformation of pThr254-Pro of the p65 subunit. In some embodiments, the antigenic peptide is at least 8 amino acid residues in length (e.g. at least 9, 10, 11, 12, 13, 14, 15, 16, 17, 18, 19, 20, 25, 30, 35, 40, 45, or 50 amino acids or more). In some embodiments, the antigenic peptide is between 8 and 20 amino acid residues in length.

In some embodiments, the method further comprises administering an adjuvant. In other embodiments, vaccination with the antigenic peptide does not require an adjuvant in order to generate a robust response. In some embodiments, the antigenic peptide is administered only once. In other embodiments, a first dose of the antigenic peptide is administered followed by one or more booster does, e.g., a second booster dose administered 1-4 weeks, 1-2 months, 2-4 months, 4-6 months, 6-8 months, 8-10 months, or 10-12 months or more after the first dose.

In another aspect, the invention features a method of treating a subject having or at risk of developing an immune disorder or an inflammatory disorder, an infection, or a cancer, wherein the method includes administering to the subject a therapeutically effective amount of any one of the antibodies or antigen-binding fragments thereof described herein, any one of the polynucleotides described herein, any one of the vectors described herein, or any one of the host cells described herein. In some embodiments, the method includes administering to the subject a therapeutically effective amount of any one of the antibodies or antigen-binding fragments thereof described herein (e.g., an antibody or antigen-binding fragment thereof that specifically binds an epitope including the trans conformation of pThr254-Pro of the p65 subunit of NF-κB).

In some embodiments, the immune disorder or inflammatory disorder is selected from acne vulgaris; acute respiratory distress syndrome; Addison's disease; adrenocortical insufficiency; adrenogenital syndrome; allergic conjunctivitis; allergic rhinitis; allergic intraocular inflammatory diseases, ANCA-associated small-vessel vasculitis; angioedema; ankylosing spondylitis; aphthous stomatitis; arthritis, asthma; atherosclerosis; atopic dermatitis; autoimmune disease; autoimmune hemolytic anemia; autoimmune hepatitis; Behcet's disease; Bell's palsy; berylliosis; bronchial asthma; bullous herpetiformis dermatitis; bullous pemphigoid; carditis; celiac disease; cerebral ischaemia; chronic obstructive pulmonary disease; cirrhosis; Cogan's syndrome; contact dermatitis; Crohn's disease; Cushing's syndrome; Cytokine Release Syndrome (CRS); dermatomyositis; diabetes mellitus; discoid lupus erythematosus; eosinophilic fasciitis; epicondylitis; erythema nodosum; exfoliative dermatitis; fibromyalgia; focal glomerulosclerosis; giant cell arteritis; gout; gouty arthritis; graft-versus-host disease; hand eczema; Henoch-Schonlein purpura; herpes gestationis; hirsutism; hypersensitivity drug reactions; idiopathic cerato-scleritis; idiopathic pulmonary fibrosis; idiopathic thrombocytopenic purpura; inflammatory bowel or gastrointestinal disorders, inflammatory dermatoses; juvenile rheumatoid arthritis; laryngeal edema; lichen planus; Loeffler's syndrome; lupus nephritis; lupus vulgaris; lymphomatous tracheobronchitis; macular edema; multiple sclerosis; musculoskeletal and connective tissue disorder; myasthenia gravis; myositis; obstructive pulmonary disease; ocular inflammation; organ transplant rejection; osteoarthritis; pancreatitis; pemphigoid gestationis; pemphigus vulgaris; polyarteritis nodosa; polymyalgia rheumatica; primary adrenocortical insufficiency; primary billiary cirrhosis; pruritus scroti; pruritis/inflammation, psoriasis; psoriatic arthritis; Reiter's disease; relapsing polychondritis; rheumatic carditis; rheumatic fever; rheumatoid arthritis; rosacea caused by sarcoidosis; rosacea caused by scleroderma; rosacea caused by Sweet's syndrome; rosacea caused by systemic lupus erythematosus; rosacea caused by urticaria; rosacea caused by zoster-associated pain; sarcoidosis; scleroderma; segmental glomerulosclerosis; sepsis (e.g. septic shock); serum sickness; shoulder tendinitis or bursitis; Sjogren's syndrome; Still's disease; stroke-induced brain cell death; Sweet's disease; systemic dermatomyositis; systemic inflammatory response syndrome (SIRS); systemic lupus erythematosus; systemic sclerosis; Takayasu's arteritis; temporal arteritis; and thyroiditis; toxic epidermal necrolysis; tuberculosis; type-1 diabetes; ulcerative colitis; uveitis; vasculitis; and Wegener's granulomatosis. In preferred embodiments, the immune disorder or inflammatory disorder is sepsis (e.g., septic shock), SIRS or CRS.

In another aspect, the invention features a method of treating a subject having or at risk of developing sepsis (e.g., septic shock), wherein the method includes administering to the subject a therapeutically effective amount of any one of the antibodies or antigen-binding fragments thereof described herein, any one of the polynucleotides described herein, any one of the vectors described herein, or any one of the host cells described herein. In some embodiments, the method includes administering to the subject a therapeutically effective amount of any one of the antibodies or antigen-binding fragments thereof described herein (e.g., an antibody or antigen-binding fragment thereof that specifically binds an epitope including the trans conformation of pThr254-Pro of the p65 subunit of NF-κB). In some embodiments, the invention features a method of treating a subject having or at risk of developing septic shock.

In some embodiments, the sepsis or septic shock is associated with a bacterial infection, a viral infection (e.g., a betacoronavirus infection, such as SARS-CoV, MERS-CoV, or SARS-CoV-2), a fungal infection, a parasitic infection, or the sepsis is sterile sepsis. In some embodiments, the sepsis or septic shock is associated with trauma, burns, pancreatitis, or ischaemic reperfusion.

In another aspect, the invention features a method of treating a subject having or at risk of developing SIRS, wherein the method includes administering to the subject a therapeutically effective amount of any one of the antibodies or antigen-binding fragments thereof described herein, any one of the polynucleotides described herein, any one of the vectors described herein, or any one of the host cells described herein. In some embodiments, the method includes administering to the subject a therapeutically effective amount of any one of the antibodies or antigen-binding fragments thereof described herein (e.g., an antibody or antigen-binding fragment thereof that specifically binds an epitope including the trans conformation of pThr254-Pro of the p65 subunit of NF-κB).

In some embodiments, the SIRS is associated with infection (e.g., bacterial, viral, fungal, or parasitic infection), trauma, burns, pancreatitis, or ischaemic reperfusion. In some embodiments, the viral infection is a betacoronavirus infection, such as SARS-CoV, MERS-CoV, or SARS-CoV-2.

In another aspect, the invention features a method of treating a subject having or at risk of developing CRS, wherein the method includes administering to the subject a therapeutically effective amount of any one or more of the antibodies or antigen-binding fragments thereof described herein, any one or more of the polynucleotides described herein, any one or more of the vectors described herein, or any or more one of the host cells described herein. In some embodiments, the method includes administering to the subject a therapeutically effective amount of any one or more of the antibodies or antigen-binding fragments thereof described herein (e.g., an antibody or antigen-binding fragment thereof that specifically binds an epitope including the trans conformation of pThr254-Pro of the p65 subunit of NF-κB).

In some embodiments, the CRS is associated with an antibody therapy (e.g., an antibody therapy administered to treat any of the cancers described herein), a small molecule cancer therapy, stem cell transplantation, graft-versus-host disease, CAR-T (e.g., CAR-T therapy administered to treat any of the cancers described herein), an infection (e.g., a bacterial infection or a viral infection such as influenza or a betacoronavirus infection, such as SARS-CoV, MERS-CoV, or SARS-CoV-2), or a hemophagocytic syndrome (e.g., macrophage activation syndrome (MAS) or hemophagocytic lymphohistiocytosis (HLH)).

In another aspect, the invention features a subject having or at risk of developing an infection, wherein the method includes administering to the subject a therapeutically effective amount of any one of the antibodies or antigen-binding fragments thereof described herein, any one of the polynucleotides described herein, any one of the vectors described herein, or any one of the host cells described herein. In some embodiments, the method includes administering to the subject a therapeutically effective amount any one of the antibodies or antigen-binding fragments thereof described herein (e.g., an antibody or antigen-binding fragment thereof that specifically binds an epitope including the trans conformation of pThr254-Pro of the p65 subunit of NF-κB). In some embodiments, the infection is a bacterial infection, a viral infection (e.g., a betacoronavirus infection, such as SARS-CoV, MERS-CoV, or SARS-CoV-2), a fungal infection, or a parasitic infection. In some embodiments, the betacoronavirus infection is SARS-CoV-2. In some embodiments, the subject has been diagnosed with COVID-19, is suspected to have COVID-19, has been in contact with someone diagnosed with COVID-19, or has recently traveled to an area experiencing an outbreak of COVID-19.

In another aspect, the invention features a method of treating a subject having or at risk of developing a cancer, wherein the method includes administering to the subject a therapeutically effective amount of any one of the antibodies or antigen-binding fragments thereof described herein, any one of the polynucleotides described herein, any one of the vectors described herein, or any one of the host cells described herein. In some embodiments, the method includes administering to the subject a therapeutically effective amount of any one of the antibodies or antigen-binding fragments thereof described herein (e.g., an antibody or antigen-binding fragment thereof that specifically binds an epitope including the trans conformation of pThr254-Pro of the p65 subunit of NF-κB).

In some embodiments, the cancer is selected from leukemia, lymphoma, liver cancer, bone cancer, lung cancer, brain cancer, bladder cancer, gastrointestinal cancer, breast cancer, cardiac cancer, cervical cancer, uterine cancer, ovarian cancer, colon cancer, skin cancer, head and neck cancer, gallbladder cancer, laryngeal cancer, lip and oral cavity cancer, ocular cancer, melanoma, pancreatic cancer, prostate cancer, colorectal cancer, testicular cancer, and throat cancer.

In some embodiments of any of the aspects described herein, the subject in a human subject.

In some embodiments, the pharmaceutical composition (e.g., any of the pharmaceutical compositions described herein) further includes an additional therapeutic agent (e.g., an immunotherapy agent, a chimeric antigen receptor (CAR-T) agent, a chemotherapeutic agent, a small molecule anti-cancer agent, a cancer vaccine, an antibacterial agent, an antifungal agent, or an antiviral agent).

In some embodiments, the method (e.g., any of the methods of treatment described herein) further includes administering to the subject an additional therapeutic agent (e.g., an immunotherapy agent, a chimeric antigen receptor (CAR-T) agent, a chemotherapeutic agent, a small molecule anti-cancer agent, a cancer vaccine, an antibacterial agent, an antifungal agent, or an antiviral agent).

In some embodiments, the additional therapeutic agent is an immunotherapy agent. In some embodiments, the immunotherapy agent is selected from the group consisting of an anti-CTLA-4 agent, an anti-PD-1 agent, an anti-PD-L1 agent, an anti-PD-L2 agent, a TNF-α cross-linking agent, a TRAIL cross-linking agent, an anti-CD27 agent, an anti-CD30 agent, an anti-CD40 agent, an anti-4-1 BB agent, an anti-GITR agent, an anti-OX40 agent, an anti-TRAILR1 agent, an anti-TRAILR2 agent, an anti-TWEAK agent, an anti-TWEAKR agent, an anti-cell surface lymphocyte protein agent, an anti-BRAF agent, an anti-MEK agent, an anti-CD33 agent, an anti-CD20 agent, an anti-HLA-DR agent, an anti-HLA class I agent, an anti-CD52 agent, an anti-A33 agent, an anti-GD3 agent, an anti-PSMA agent, an anti-Ceacan 1 agent, an anti-Galedin 9 agent, an anti-HVEM agent, an anti-VISTA agent, an anti-B7 H4 agent, an anti-HHLA2 agent, an anti-CD155 agent, an anti-CD80 agent, an anti-BTLA agent, an anti-CD160 agent, an anti-CD28 agent, an anti-CD226 agent, an anti-CEACAM1 agent, an anti-TIM3 agent, an anti-TIGIT agent, an anti-CD96 agent, an anti-CD70 agent, an anti-CD27 agent, an anti-LIGHT agent, an anti-CD137 agent, an anti-DR4 agent, an anti-CR5 agent, an anti-TNFRS agent, an anti-TNFR1 agent, an anti-FAS agent, an anti-CD95 agent, an anti-TRAIL agent, an anti-DR6 agent, an anti-EDAR agent, an anti-NGFR agent, an anti-OPG agent, an anti-RANKL agent, an anti-LTβ receptor agent, an anti-BCMA agent, an anti-TACI agent, an anti-BAFFR agent, an anti-EDAR2 agent, an anti-TROY agent, and an anti-RELT agent, optionally wherein the immunotherapy agent is an anti-PD-1 antibody or an anti-PD-L1 antibody.

In some embodiments, the immunotherapy agent is selected from the group consisting of an anti-CTLA-4 antibody or antigen-binding fragment thereof, an anti-PD-1 antibody or antigen-binding fragment thereof, an anti-PD-L1 antibody or antigen-binding fragment thereof, an anti-PD-L2 antibody or antigen-binding fragment thereof, a TNF-α cross-linking antibody or antigen-binding fragment thereof, a TRAIL cross-linking antibody or antigen-binding fragment thereof, an anti-CD27 antibody or antigen-binding fragment thereof, an anti-CD30 antibody or antigen-binding fragment thereof, an anti-CD40 antibody or antigen-binding fragment thereof, an anti-4-1 BB antibody or antigen-binding fragment thereof, an anti-GITR antibody or antigen-binding fragment thereof, an anti-OX40 antibody or antigen-binding fragment thereof, an anti-TRAILR1 antibody or antigen-binding fragment thereof, an anti-TRAILR2 antibody or antigen-binding fragment thereof, an anti-TWEAK antibody or antigen-binding fragment thereof, an anti-TWEAKR antibody or antigen-binding fragment thereof, an anti-cell surface lymphocyte protein antibody or antigen-binding fragment thereof, an anti-BRAF antibody or antigen-binding fragment thereof, an anti-MEK antibody or antigen-binding fragment thereof, an anti-CD33 antibody or antigen-binding fragment thereof, an anti-CD20 antibody or antigen-binding fragment thereof, an anti-HLA-DR antibody or antigen-binding fragment thereof, an anti-HLA class I antibody or antigen-binding fragment thereof, an anti-CD52 antibody or antigen-binding fragment thereof, an anti-A33 antibody or antigen-binding fragment thereof, an anti-GD3 antibody or antigen-binding fragment thereof, an anti-PSMA antibody or antigen-binding fragment thereof, an anti-Ceacan 1 antibody or antigen-binding fragment thereof, an anti-Galedin 9 antibody or antigen-binding fragment thereof, an anti-HVEM antibody or antigen-binding fragment thereof, an anti-VISTA antibody or antigen-binding fragment thereof, an anti-B7 H4 antibody or antigen-binding fragment thereof, an anti-HHLA2 antibody or antigen-binding fragment thereof, an anti-CD155 antibody or antigen-binding fragment thereof, an anti-CD80 antibody or antigen-binding fragment thereof, an anti-BTLA antibody or antigen-binding fragment thereof, an anti-CD160 antibody or antigen-binding fragment thereof, an anti-CD28 antibody or antigen-binding fragment thereof, an anti-CD226 antibody or antigen-binding fragment thereof, an anti-CEACAM1 antibody or antigen-binding fragment thereof, an anti-TIM3 antibody or antigen-binding fragment thereof, an anti-TIGIT antibody or antigen-binding fragment thereof, an anti-CD96 antibody or antigen-binding fragment thereof, an anti-CD70 antibody or antigen-binding fragment thereof, an anti-CD27 antibody or antigen-binding fragment thereof, an anti-LIGHT antibody or antigen-binding fragment thereof, an anti-CD137 antibody or antigen-binding fragment thereof, an anti-DR4 antibody or antigen-binding fragment thereof, an anti-CR5 antibody or antigen-binding fragment thereof, an anti-TNFRS antibody or antigen-binding fragment thereof, an anti-TNFR1 antibody or antigen-binding fragment thereof, an anti-FAS antibody or antigen-binding fragment thereof, an anti-CD95 antibody or antigen-binding fragment thereof, an anti-TRAIL antibody or antigen-binding fragment thereof, an anti-DR6 antibody or antigen-binding fragment thereof, an anti-EDAR antibody or antigen-binding fragment thereof, an anti-NGFR antibody or antigen-binding fragment thereof, an anti-OPG antibody or antigen-binding fragment thereof, an anti-RANKL antibody or antigen-binding fragment thereof, an anti-LTβ receptor antibody or antigen-binding fragment thereof, an anti-BCMA antibody or antigen-binding fragment thereof, an anti-TACI antibody or antigen-binding fragment thereof, an anti-BAFFR antibody or antigen-binding fragment thereof, an anti-EDAR2 antibody or antigen-binding fragment thereof, an anti-TROY antibody or antigen-binding fragment thereof, and an anti-RELT antibody or antigen-binding fragment thereof.

In some embodiments, the additional therapeutic agent is a chimeric antigen receptor (CAR-T) agent, a chemotherapeutic agent, a small molecule anti-cancer agent, or a cancer vaccine.

In some embodiments, the additional therapeutic agent is an antibacterial agent. In some embodiments, the antibacterial agent is selected from the group consisting of Afenide, Amikacin, Amoxicillin, Ampicillin, Arsphenamine, Augmentin, Azithromycin, Azlocillin, Aztreonam, Bacampicillin, Bacitracin, Balofloxacin, Besifloxacin, Capreomycin, Carbacephem (loracarbef), Carbenicillin, Cefacetrile (cephacetrile), Cefaclomezine, Cefaclor, Cefadroxil (cefadroxyl), Cefalexin (cephalexin), Cefaloglycin (cephaloglycin), Cefalonium (cephalonium), Cefaloram, Cefaloridine (cephaloradine), Cefalotin (cephalothin), Cefamandole, Cefaparole, Cefapirin (cephapirin), Cefatrizine, Cefazaflur, Cefazedone, Cefazolin (cephazolin), Cefcanel, Cefcapene, Cefclidine, Cefdaloxime, Cefdinir, Cefditoren, Cefedrolor, Cefempidone, Cefepime, Cefetamet, Cefetrizole, Cefivitril, Cefixime, Cefluprenam, Cefmatilen, Cefmenoxime, Cefmepidium, Cefmetazole, Cefodizime, Cefonicid, Cefoperazone, Cefoselis, Cefotaxime, Cefotetan, Cefovecin, Cefoxazole, Cefoxitin, Cefozopran, Cefpimizole, Cefpirome, Cefpodoxime, Cefprozil (cefproxil), Cefquinome, Cefradine (cephradine), Cefrotil, Cefroxadine, Cefsumide, Ceftaroline, Ceftazidime, Ceftazidime/Avibactam, Cefteram, Ceftezole, Ceftibuten, Ceftiofur, Ceftiolene, Ceftioxide, Ceftizoxime, Ceftobiprole, Ceftriaxone, Cefuracetime, Cefuroxime, Cefuzonam, Cephalexin, Chloramphenicol, Chlorhexidine, Ciprofloxacin, Clarithromycin, Clavulanic Acid, Clinafloxacin, Clindamycin, Cloxacillin, Colimycin, Colistimethate, Colistin, Crysticillin, Cycloserine 2, Demeclocycline, Dicloxacillin, Dirithromycin, Doripenem, Doxycycline, Efprozil, Enoxacin, Ertapenem, Erythromycin, Ethambutol, Flucloxacillin, Flumequine, Fosfomycin, Furazolidone, Gatifloxacin, Geldanamycin, Gemifloxacin, Gentamicin, Glycopeptides, Grepafloxacin, Herbimycin, Imipenem, Isoniazid, Kanamycin, Levofloxacin, Lincomycin, Linezolid, Lipoglycopeptides, Lomefloxacin, Meropenem, Meticillin, Metronidazole, Mezlocillin, Minocycline, Mitomycin, Moxifloxacin, Mupirocin, Nadifloxacin, Nafcillin, Nalidixic Acid, Neomycin, Netilmicin, Nitrofurantoin, Norfloxacin, Ofloxacin, Oxacillin, Oxazolidinones, Oxolinic Acid, Oxytetracycline, Oxytetracycline, Paromomycin, Pazufloxacin, Pefloxacin, Penicillin G, Penicillin V, Pipemidic Acid, Piperacillin, Piromidic Acid, Pivampicillin, Pivmecillinam, Platensimycin, Polymyxin B, Pristinamycin, Prontosil, Prulifloxacin, Pvampicillin, Pyrazinamide, Quinupristin/dalfopristin, Rifabutin, Rifalazil, Rifampin, Rifamycin, Rifapentine, Rosoxacin, Roxithromycin, Rufloxacin, Sitafloxacin, Sparfloxacin, Spectinomycin, Spiramycin, Streptomycin, Sulbactam, Sulfacetamide, Sulfamethizole, Sulfamethoxazole, Sulfanilimide, Sulfisoxazole, Sulphonamides, Sultamicillin, Teicoplanin, Telavancin, Telithromycin, Temafloxacin, Tetracycline, Thiamphenicol, Ticarcillin, Tigecycline, Tinidazole, Tobramycin, Tosufloxacin, Trimethoprim, Trimethoprim-Sulfamethoxazole, Troleandomycin, Trovafloxacin, Tuberactinomycin, Vancomycin, and Viomycin, or a pharmaceutically acceptable salt thereof.

In some embodiments, the additional therapeutic agent is an antifungal agent. In some embodiments, the antifungal agent is selected from the group consisting of Abafungin, Albaconazole, Amorolfin, Amphotericin B, Anidulafungin, Bifonazole, Butenafine, Butoconazole, Candicidin, Caspofungin, Ciclopirox, Clotrimazole, Econazole, Fenticonazole, Filipin, Fluconazole, Flucytosine, Griseofulvin, Haloprogin, Hamycin, Isavuconazole, Isoconazole, Itraconazole, Ketoconazole, Micafungin, Miconazole, Naftifine, Natamycin, Nystatin, Omoconazole, Oxiconazole, Polygodial, Posaconazole, Ravuconazole, Rimocidin, Sertaconazole, Sulconazole, Terbinafine, Terconazole, Tioconazole, Tolnaftate, Undecylenic Acid, and Voriconazole, or a pharmaceutically acceptable salt thereof.

In some embodiments, the additional therapeutic agent is an antiviral agent. In some embodiments, the antiviral agent is selected from the group consisting of vidarabine, acyclovir, gancyclovir, valgancyclovir, AZT (zidovudine), ddl (didanosine), ddC (zalcitabine), d4T (stavudine), 3TC (lamivudine), nevirapine, delavirdine, saquinavir, ritonavir, indinavir, nelfinavir, ribavirin, and interferon, or a pharmaceutically acceptable salt thereof.

In some embodiments, the antibody or antigen-binding fragment thereof is administered to the subject in an amount of from about 0.001 mg/kg to about 100 mg/kg (e.g., in an amount from 0.001 mg/kg to 0.01 mg/kg, from 0.01 mg/kg to 0.1 mg/kg, from 0.1 mg/kg to 1 mg/kg, from 1 mg/kg to 10 mg/mkg, or from 10 mg/kg to 100 mg/kg). In another aspect, the invention features a method of determining the level of nuclear NF-κB activity in a sample from subject, the method including: (i) contacting a sample from the subject with any one of the antibodies or antigen-binding fragments thereof described herein (e.g., an antibody or antigen-binding fragment thereof that specifically binds an epitope including the trans conformation of pThr254-Pro of the p65 subunit of NF-κB); and (ii) determining the level of the nuclear NF-κB in the sample of (i) by determining the level of the antibody or antigen-binding fragment thereof bound to the nuclear NF-κB.

In some embodiments, the method further includes: (iii) comparing the level of the nuclear NF-κB determined in (ii) to a reference value of nuclear NF-κB.

In some embodiments, the subject has or is at risk of developing an immune disorder or an inflammatory disorder (e.g., sepsis, such as septic shock, SIRS, or CRS), an infection, or a cancer.

In some embodiments, the reference value of nuclear NF-κB is the average level of nuclear NF-κB in a population of subjects having an immune disorder or an inflammatory disorder (e.g., sepsis, such as septic shock, SIRS, or CRS), an infection, or a cancer.

In some embodiments, the reference value of nuclear NF-κB is the average level of nuclear NF-κB in a population of subjects not having sepsis, an infection, or a cancer.

In some embodiments, the level of nuclear NF-κB determined in (ii) is greater than the reference value of nuclear NF-κB, then the subject is treated with a therapeutically effective amount of any one of the antibodies or antigen-binding fragments thereof described herein, any one of the polynucleotides described herein, any one of the vectors described herein, or any one of the host cells described herein.

In another aspect the invention features a kit including an agent selected from the group consisting of any one of the antibodies or antigen-binding fragments thereof described herein, any one of the polynucleotides described herein, any one of the vectors, any one of the host cells described herein, or any one of the pharmaceutical compositions described herein. In some embodiments, the kit includes any one of the antibodies or antigen-binding fragments thereof described herein (e.g., an antibody or antigen-binding fragment thereof that specifically binds an epitope including the trans conformation of pThr254-Pro of the p65 subunit of NF-κB).

In some embodiments, the kit further includes an additional therapeutic agent (e.g., an immunotherapy agent, a chimeric antigen receptor (CAR-T) agent, a chemotherapeutic agent, a small molecule anti-cancer agent, a cancer vaccine, an antibacterial agent, an antifungal agent, or an antiviral agent).

In some embodiments, the kit further includes instructions for transfecting the vector into a host cell. In some embodiments, the kit further includes instructions for expressing the antibody, antigen-binding fragment thereof, or construct in the host cell. In some embodiments, the kit further includes a reagent that can be used to express the antibody, antigen-binding fragment thereof, or construct in the host cell. In some embodiments, the kit further includes instructions for administering the agent to a subject (e.g., a human subject).

Other features and advantages of the invention will be apparent from the following detailed description, figures, examples, and claims.

### Definitions

As used herein, the term “about” refers to a value that is no more than 10% above or below the value being described. For example, the term “about 5 nM” indicates a range of from 4.5 nM to 5.5 nM.

As used herein, the term “adjuvant” refers to one or more substances that cause stimulation of the immune system. In this context, an adjuvant is used to enhance an immune response to one or more vaccine antigens or antibodies. An adjuvant may be administered to a subject before, in combination with, or after administration a vaccine. Examples of chemical compounds used as adjuvants include, but are not limited to, aluminum compounds, oils, block polymers, immune stimulating complexes, vitamins and minerals (e.g., vitamin E, vitamin A, selenium, and vitamin B12), Quil A (saponins), bacterial and fungal cell wall components (e.g., lipopolysaccarides, lipoproteins, and glycoproteins), hormones, cytokines, and co-stimulatory factors.

As used herein, the term “antigen” is meant a molecule to which an antibody or fragment thereof can selectively bind. The target antigen may be a protein (e.g., an antigenic peptide), carbohydrate, nucleic acid, lipid, hapten, or other naturally occurring or synthetic compound. The target antigen may be a polypeptide (e.g., a polypeptide containing a pThr-Pro motif) or peptide mimics (e.g., a polypeptide containing a pThr-Proline analog motif). An antigen may also be administered to an animal to generate an immune response in the animal.

As used herein, the term “antibody” (Ab) refers to an immunoglobulin molecule that specifically binds to, or is immunologically reactive with, a particular antigen, and includes polyclonal, monoclonal, genetically engineered and otherwise modified forms of antibodies, including but not limited to chimeric antibodies, humanized antibodies, primatized antibodies, heteroconjugate antibodies (e.g., bi- tri- and quad-specific antibodies, diabodies, triabodies, and tetrabodies), and antigen-binding fragments of antibodies, including e.g., Fab′, F(ab′)₂, Fab, Fv, rlgG, and scFv fragments. Moreover, unless otherwise indicated, the term “monoclonal antibody” (mAb) is meant to include both intact molecules, as well as, antibody fragments (such as, for example, Fab and F(ab′)₂ fragments) that are capable of specifically binding to a target protein. Fab and F(ab′)₂ fragments lack the Fc fragment of an intact antibody, clear more rapidly from the circulation of the animal, and may have less non-specific tissue binding than an intact antibody (see Wahl et al., J. Nucl. Med. 24:316, 1983; incorporated herein by reference).

The term “antigen-binding fragment,” as used herein, refers to one or more fragments of an antibody that retain the ability to specifically bind to a target antigen. The antigen-binding function of an antibody can be performed by fragments of a full-length antibody. The antibody fragments can be a Fab, F(ab′)₂, scFv, SMIP, diabody, a triabody, an affibody, a nanobody, an aptamer, or a domain antibody. Examples of binding fragments encompassed of the term “antigen-binding fragment” of an antibody include, but are not limited to: (i) a Fab fragment, a monovalent fragment consisting of the V_{L}, V_{H}, C_{L}, and C_{H}1 domains; (ii) a F(ab′)₂ fragment, a bivalent fragment including two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb including V_{H} and V_{L} domains; (vi) a dAb fragment (Ward et al., Nature 341:544-546, 1989), which consists of a V_{H} domain; (vii) a dAb which consists of a V_{H} or a V_{L} domain; (viii) an isolated complementarity determining region (CDR); and (ix) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules (known as single-chain Fv (scFv); see, e.g., Bird et al., Science 242:423-426, 1988, and Huston et al., Proc. Natl. Acad. Sci. USA 85:5879-5883, 1988). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in some embodiments, by chemical peptide synthesis procedures known in the art.

As used herein, the terms “anti-nuclear factor kappa-light-chain-enhancer of activated B cells antibody,” “NF-κB antibody,” “anti-NF-κB antibody,” and/or “anti-NF-κB antibody fragment” and the like include any protein or peptide-containing molecule that includes at least a portion of an immunoglobulin molecule, such as, but not limited, to at least one complementarity determining region (CDR) of a heavy or light chain or a ligand-binding portion thereof, a heavy chain or light chain variable region, a heavy chain or light chain constant region, or any portion thereof, that is capable of specifically binding to NF-κB. For instance, two or more portions of an immunoglobulin molecule may be covalently bound to one another, e.g., via an amide bond, a thioether bond, a carbon-carbon bond, a disulfide bridge, or by a linker, such as a linker described herein or known in the art. NF-κB antibodies also include antibody-like protein scaffolds, such as the tenth fibronectin type III domain (¹⁰Fn3), which contains BC, DE, and FG structural loops similar in structure and solvent accessibility to antibody CDRs. The tertiary structure of the ¹⁰Fn3 domain resembles that of the variable region of the IgG heavy chain, and one of skill in the art can graft, e.g., the CDRs of a NF-κB monoclonal antibody onto the fibronectin scaffold by replacing residues of the BC, DE, and FG loops of ¹⁰Fn3 with residues from the CDR-H1, CDR-H2, or CDR-H3 regions of a NF-κB monoclonal antibody.

As used herein, the terms “antagonist NF-κB antibody” and “antagonistic NF-κB antibody” refer to NF-κB antibodies that are capable of inhibiting or reducing activation of NF-κB, attenuating one or more signal transduction pathways mediated by NF-κB, and/or reducing or inhibiting at least one activity mediated by activation of NF-κB.

As used herein, the term “binding affinity” is meant the strength of the total noncovalent interactions between a single binding site of a molecule (e.g., an antibody) and its binding partner (e.g., an antigen or antigenic peptide). Unless otherwise indicated, as used herein, “binding affinity” refers to intrinsic binding affinity, which reflects a specific interaction between members of a binding pair (e.g., antibody and antigen). The affinity of a molecule X for its partner Y can generally be represented by the dissociation constant (Kd). Affinity can be measured by standard methods known in the art, including those described herein. A low-affinity complex contains an antibody that generally tends to dissociate readily from the antigen, whereas a high-affinity complex contains an antibody that generally tends to remain bound to the antigen for a longer duration.

As used herein, the term “bispecific antibodies” refers to antibodies (e.g., monoclonal, often human or humanized antibodies) that have binding specificities for at least two different antigens. For example, one of the binding specificities can be directed towards NF-κB (e.g., an epitope including pThr254-Pro of NF-κB), the other can be for any other antigen, e.g., for a cell-surface protein, receptor, receptor subunit, tissue-specific antigen, virally derived protein, virally encoded envelope protein, bacterially derived protein, or bacterial surface protein, etc.

As used herein, the term “cancer” and “cancerous” is meant the physiological condition in mammals that is typically characterized by abnormal cell growth. Included in this definition are benign and malignant cancers, as well as dormant tumors or micro-metastases. Examples of cancer include, but are not limited to, carcinoma, lymphoma, blastoma, sarcoma, and leukemia. More particular examples of such cancers include, e.g., prostate cancer, squamous cell cancer, small-cell lung cancer, non-small-cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, vulval cancer, thyroid cancer, hepatic carcinoma, gastric cancer, melanoma, and various types of head and neck cancer.

As used herein, the phrase “chemotherapeutic agent” refers to any chemical agent with therapeutic usefulness in the treatment of cancer, such as a cancer described herein. Chemotherapeutic agents encompass both chemical and biological agents. These agents can function to inhibit a cellular activity upon which a cancer cell depends for continued survival. Categories of chemotherapeutic agents include alkylating/alkaloid agents, antimetabolites, hormones, hormone analogs, and antineoplastic drugs. Exemplary chemotherapeutic agents suitable for use in conjunction with the compositions and methods described herein include, without limitation, those set forth in Slapak and Kufe, Principles of Cancer Therapy, Chapter 86 in Harrison's Principles of Internal medicine, 14^{th} edition; Perry et al., Chemotherapeutic, Chapter 17 in Abeloff, Clinical Oncology 2^{nd} ed., 2000; Baltzer L. and Berkery R. (eds): Oncology Pocket Guide to Chemotherapeutic, 2^{nd} ed. St. Luois, mosby-Year Book, 1995; Fischer D. S., Knobf M. F., Durivage H. J. (eds): The Cancer Chemotherapeutic Handbook, 4^{th} ed. St. Luois, Mosby-Year Handbook, the disclosures of each of which are incorporated herein by reference as they pertain to chemotherapeutic agents.

As used herein, the term “chimeric” antibody refers to an antibody having variable domain sequences (e.g., CDR sequences) derived from an immunoglobulin of one source organism, such as rat or mouse, and constant regions derived from an immunoglobulin of a different organism (e.g., a human, another primate, pig, goat, rabbit, hamster, cat, dog, guinea pig, member of the bovidae family (such as cattle, bison, buffalo, elk, and yaks, among others), cow, sheep, horse, or bison, among others). Methods for producing chimeric antibodies are known in the art. See, e.g., Morrison, 1985, Science 229(4719): 1202-7; Oi et al, 1986, BioTechniques 4:214-221; Gillies et al, 1985, J. Immunol. Methods 125:191-202; U.S. Pat. Nos. 5,807,715; 4,816,567; and 4,816,397; incorporated herein by reference.

As used herein, the term “complementarity determining region” (CDR) refers to a hypervariable region found both in the light chain and the heavy chain variable domains. The more highly conserved portions of variable domains are called the framework regions (FRs). As is appreciated in the art, the amino acid positions that delineate a hypervariable region of an antibody can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. The antibodies described herein may including modifications in these hybrid hypervariable positions. The variable domains of native heavy and light chains each include four framework regions that primarily adopt a β-sheet configuration, connected by three CDRs, which form loops that connect, and in some cases form part of, the β-sheet structure. The CDRs in each chain are held together in close proximity by the FR regions in the order FR1-CDR1-FR2-CDR2-FR3-CDR3-FR4 and, with the CDRs from the other antibody chains, contribute to the formation of the target binding site of antibodies (see Kabat et al, Sequences of Proteins of Immunological Interest (National Institute of Health, Bethesda, Md. 1987; incorporated herein by reference). As used herein, numbering of immunoglobulin amino acid residues is done according to the immunoglobulin amino acid residue numbering system of Kabat et al, unless otherwise indicated.

As used herein, the term “conformation-specific antibody” is an antibody or fragment thereof that recognizes and specifically binds to a particular conformation (e.g., a conformational isomer or conformer) of its complementary antigen. For example, as described herein, the conformation-specific antibody may specifically bind to the trans conformation of a pThr-Pro motif, but will not specifically bind to the cis conformation of the pThr-Pro motif (e.g., binds specifically to an epitope including trans-pTh254-Pro of the p65 subunit of NF-κB relative to an epitope including cis-pTh254-Pro). In this case, the conformation-specific antibody will have, for example, at least 10- to 100-fold greater affinity to the trans conformation than to the cis conformation of a pThr-Pro motif. Conversely, the conformation-specific antibody may specifically bind to the cis conformation of a pThr-Pro motif, but will not specifically bind to the trans conformation of the pThr-Pro motif.

As used herein, the terms “conservative mutation,” “conservative substitution,” or “conservative amino acid substitution” refer to a substitution of one or more amino acids for one or more different amino acids that exhibit similar physicochemical properties, such as polarity, electrostatic charge, and steric volume. These properties are summarized for each of the twenty naturally-occurring amino acids in table 1 below.

TABLE 1
Representative physicochemical properties of naturally-occurring amino acids

| | | | Side- | Electrostatic | |
|---|---|---|---|---|---|
| | 3 Letter | 1 Letter | chain | character at | Steric |
| Amino Acid | Code | Code | Polarity | physiological pH (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral and | large |
| | | | | cationic forms in | |
| | | | | equilibrium at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | non- | neutral | intermediate |
| | | | polar | | |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

From this table it is appreciated that the conservative amino acid families include, e.g., (i) G, A, V, L, I, P, and M; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

Amino acid substitutions may be represented herein using the convention: (AA1)(N)(AA2), where “AA1” represents the amino acid normally present at particular site within an amino acid sequence, “N” represents the residue number within the amino acid sequence at which the substitution occurs, and “AA2” represents the amino acid present in the amino acid sequence after the substitution is effectuated. For example, the notation “C232S” in the context of an antibody hinge region, such as an IgG2 antibody hinge region, refers to a substitution of the naturally-occurring cysteine residue for a serine residue at amino acid residue 232 of the indicated hinge amino acid sequence. Likewise, the notation “C233S” in the context of an antibody hinge region, such as an IgG2 antibody hinge region, refers to a substitution of the naturally-occurring cysteine residue for a serine residue at amino acid residue 233 of the indicated hinge amino acid sequence.

As used herein, the term “conjugate” refers to a compound formed by the chemical bonding of a reactive functional group of one molecule with an appropriately reactive functional group of another molecule.

As used herein, the term “chemokine” refers to a type of small cytokine that can induce directed chemotaxis in nearby cells. Classes of chemokines include CC chemokines, CXC chemokines, C chemokines, and CX3C chemokines. Chemokines can regulate immune cell migration and homing, including the migration and homing of monocytes, macrophages, T cells, mast cells, eosinophils, and neutrophils. Chemokines responsible for immune cell migration include CCL19, CCL21, CCL14, CCL20, CCL25, CCL27, CXCL12, CXCL13, CCR9, CCR10, and CXCR5. Chemokines that can direct the migration of inflammatory leukocytes to sites of inflammation or injury include CCL2, CCL3, CCL5, CXCL1, CXCL2, and CXCL8.

As used herein, the term “cytokine” refers to a small protein involved in cell signaling. Cytokines can be produced and secreted by immune cells, such as T cells, B cells, macrophages, and mast cells, and include chemokines, interferons, interleukins, lymphokines, and tumor necrosis factors.

As used herein, the term “Cytokine Release Syndrome (CRS)” refers to a systemic inflammatory response that can be triggered by a variety of stimuli including infections and certain therapeutics. For example, CRS has been associated with antibody therapy, small molecule cancer therapeutics, stem cell transplantation, graft-versus-host disease, T cell-engaging therapies (e.g., CAR-T), infection (e.g., a bacterial or viral infection), and hemophagocytic syndromes (e.g., macrophage activation syndrome (MAS) or hemophagocytic lymphohistiocytosis (HLH)). The epidemiology, clinical presentation, pathophysiology, and differential diagnosis of CRS have been described, for example, in Shimabukuro-Vornhagen, A. et al. Journal for ImmunoTherapy of Cancer. 6:56 (2018); Canna, S. W. and Behrens, E. M. Pediatr Clin N Am. 59:329-344 (2012); and Chavez, J. C. et al. Hematol Oncol Stem Cell Ther. https://doi.org/10.1016/j.hemonc.2019.05.005, each of which is incorporated herein by reference. In brief, CRS can present with a variety of symptoms ranging from mild, flu-like symptoms to severe life-threatening manifestations corresponding to an overactive inflammatory response. Mild symptoms of CRS include fever, fatigue, headache, rash, arthralgia, and/or myalgia. More severe cases are characterized by hypotension and/or high fever and may progress to an uncontrolled systemic inflammatory response with vasopressor-requiring circulatory shock, vascular leakage, disseminated intravascular coagulation, and/or multi-organ system failure. Laboratory abnormalities that are common in patients with CRS are known to those of skill in the art and include cytopenias, elevated creatinine and liver enzymes, deranged coagulation parameters, and increased serum C-Reactive Protein (CRP).

As used herein, the term “derivatized antibodies” refers to antibodies that are modified by a chemical reaction so as to cleave residues or add chemical moieties not native to an isolated antibody. Derivatized antibodies can be obtained by glycosylation, acetylation, pegylation, phosphorylation, amidation, derivatization by addition of known chemical protecting/blocking groups, proteolytic cleavage, linkage to a cellular ligand or other protein. Any of a variety of chemical modifications can be carried out by known techniques, including, without limitation, specific chemical cleavage, acetylation, formylation, metabolic synthesis of tunicamycin, etc. using established procedures. Additionally, the derivative can contain one or more non-natural amino acids, e.g., using amber suppression technology (see, e.g., U.S. Pat. No. 6,964,859; incorporated herein by reference).

As used herein, the term “diabodies” refers to bivalent antibodies including two polypeptide chains, in which each polypeptide chain includes VH and VL domains joined by a linker that is too short (e.g., a linker composed of five amino acids) to allow for intramolecular association of VH and VL domains on the same peptide chain. This configuration forces each domain to pair with a complementary domain on another polypeptide chain so as to form a homodimeric structure. Accordingly, the term “triabodies” refers to trivalent antibodies including three peptide chains, each of which contains one VH domain and one VL domain joined by a linker that is exceedingly short (e.g., a linker composed of 1-2 amino acids) to permit intramolecular association of VH and VL domains within the same peptide chain. In order to fold into their native structure, peptides configured in this way typically trimerize so as to position the VH and VL domains of neighboring peptide chains spatially proximal to one another to permit proper folding (see Holliger et al., Proc. Natl. Acad. Sci. USA 90:6444-48, 1993; incorporated herein by reference).

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell).

As used herein, the term “epitope” refers to a portion of an antigen that is recognized and bound by a polypeptide, such as an antibody, antigen-binding fragment thereof, single-chain polypeptide, or construct as described herein. In the context of a protein antigen (such as NF-κB, e.g., the p65 subunit of NF-κB), an epitope may be a continuous epitope, which is a single, uninterrupted segment of one or more amino acids covalently linked to one another by peptide bonds in which all of the component amino acids bind the polypeptide (e.g., antibody, antigen-binding fragment thereof, single-chain polypeptide, or construct thereof). Continuous epitopes may be composed, for instance, of 1, 5, 10, 15, 20, or more amino acids within an antigen. In some embodiments, an epitope may be a discontinuous epitope, which contains two or more segments of amino acids each separated from one another in an antigen's amino acid sequence by one or more intervening amino acid residues. Discontinuous epitopes may be composed, for instance, of 1, 2, 3, 4, 5, 6, 7, 8, 9, 10, or more such segments of amino acid residues. Despite this separation by intervening amino acids, the segments that compose a discontinuous epitope may be, for instance, spatially proximal to one another in the three-dimensional conformation of the antigen. An epitope may be defined not just by its amino acid compositions, but also by the post-translation state of an amino acid of the epitope (e.g., phosphorylation) or the bond geometry of a peptide bond between two amino acids in the epitope (e.g., cis or trans).

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell). Exogenous materials include those that are provided from an external source to an organism or to cultured matter extracted there from.

As used herein, the term “framework region” or “FW region” includes amino acid residues that are adjacent to the CDRs. FW region residues may be present in, for example, human antibodies, rodent-derived antibodies (e.g., murine antibodies), humanized antibodies, primatized antibodies, chimeric antibodies, antibody fragments (e.g., Fab fragments), single-chain antibody fragments (e.g., scFv fragments), antibody domains, and bispecific antibodies, among others.

As used herein, the term “fusion protein” refers to a protein that is joined via a covalent bond to another molecule. A fusion protein can be chemically synthesized by, e.g., an amide-bond forming reaction between the N-terminus of one protein to the C-terminus of another protein. Alternatively, a fusion protein containing one protein covalently bound to another protein can be expressed recombinantly in a cell (e.g., a eukaryotic cell or prokaryotic cell) by expression of a polynucleotide encoding the fusion protein, for example, from a vector or the genome of the cell. A fusion protein may contain one protein that is covalently bound to a linker, which in turn is covalently bound to another molecule. Examples of linkers that can be used for the formation of a fusion protein include peptide-containing linkers, such as those that contain naturally occurring or non-naturally occurring amino acids. In some embodiments, it may be desirable to include D-amino acids in the linker, as these residues are not present in naturally-occurring proteins and are thus more resistant to degradation by endogenous proteases. Linkers can be prepared using a variety of strategies that are well known in the art, and depending on the reactive components of the linker, can be cleaved by enzymatic hydrolysis, photolysis, hydrolysis under acidic conditions, hydrolysis under basic conditions, oxidation, disulfide reduction, nucleophilic cleavage, or organometallic cleavage (Leriche et al., Bioorg. Med. Chem., 20:571-582, 2012).

As used herein, the term “heterospecific antibodies” refers to monoclonal, preferably human or humanized, antibodies that have binding specificities for at least two different antigens. Traditionally, the recombinant production of heterospecific antibodies is based on the co-expression of two immunoglobulin heavy chain-light chain pairs, where the two heavy chains have different specificities (Milstein et al., Nature 305:537, 1983). Similar procedures are disclosed, e.g., in WO 93/08829, U.S. Pat. Nos. 6,210,668; 6,193,967; 6,132,992; 6,106,833; 6,060,285; 6,037,453; 6,010,902; 5,989,530; 5,959,084; 5,959,083; 5,932,448; 5,833,985; 5,821,333; 5,807,706; 5,643,759, 5,601,819; 5,582,996, 5,496,549, 4,676,980, WO 91/00360, WO 92/00373, EP 03089, Traunecker et al., EMBO J. 10:3655 (1991), Suresh et al., Methods in Enzymology 121:210 (1986); incorporated herein by reference. Heterospecific antibodies can include Fc mutations that enforce correct chain association in multi-specific antibodies, as described by Klein et al, mAbs 4(6):653-663, 2012; incorporated herein by reference.

As used herein, the term “human antibody” refers to an antibody in which substantially every part of the protein (e.g., CDR, framework, C_{L}, C_{H} domains (e.g., C_{H}1, C_{H}2, C_{H}3), hinge, (V_{L}, V_{H})) is substantially non-immunogenic in humans, with only minor sequence changes or variations. A human antibody can be produced in a human cell (e.g., by recombinant expression), or by a non-human animal or a prokaryotic or eukaryotic cell that is capable of expressing functionally rearranged human immunoglobulin (e.g., heavy chain and/or light chain) genes. Further, when a human antibody is a single-chain antibody, it can include a linker peptide that is not found in native human antibodies. For example, an Fv can include a linker peptide, such as two to about eight glycine or other amino acid residues, which connects the variable region of the heavy chain and the variable region of the light chain. Such linker peptides are considered to be of human origin. Human antibodies can be made by a variety of methods known in the art including phage display methods using antibody libraries derived from human immunoglobulin sequences. See U.S. Pat. Nos. 4,444,887 and 4,716,111; and PCT publications WO 1998/46645; WO 1998/50433; WO 1998/24893; WO 1998/16654; WO 1996/34096; WO 1996/33735; and WO 1991/10741; incorporated herein by reference. Human antibodies can also be produced using transgenic mice that are incapable of expressing functional endogenous immunoglobulins, but which can express human immunoglobulin genes. See, e.g., PCT publications WO 98/24893; WO 92/01047; WO 96/34096; WO 96/33735; U.S. Pat. Nos. 5,413,923; 5,625,126; 5,633,425; 5,569,825; 5,661,016; 5,545,806; 5,814,318; 5,885,793; 5,916,771; and 5,939,598; incorporated by reference herein.

As used herein, the term “humanized” antibody refers to forms of non-human (e.g., murine) antibodies that are chimeric immunoglobulins, immunoglobulin chains or fragments thereof (such as Fv, Fab, Fab′, F(ab′)₂ or other target-binding subdomains of antibodies) which contain minimal sequences derived from non-human immunoglobulin. In general, the humanized antibody will include substantially all of at least one, and typically two, variable domains, in which all or substantially all of the CDR regions correspond to those of a non-human immunoglobulin. All or substantially all of the FR regions may also be those of a human immunoglobulin sequence. The humanized antibody can also include at least a portion of an immunoglobulin constant region (Fc), typically that of a human immunoglobulin consensus sequence. Methods of antibody humanization are known in the art. See, e.g., Riechmann et al., Nature 332:323-7, 1988; U.S. Pat. Nos. 5,530,101; 5,585,089; 5,693,761; 5,693,762; and U.S. Pat. No. 6,180,370 to Queen et al; EP239400; PCT publication WO 91/09967; U.S. Pat. No. 5,225,539; EP592106; and EP519596; incorporated herein by reference.

As used herein, the term “hydrophobic side-chain” refers to an amino acid side-chain that exhibits low solubility in water relative due to, e.g., the steric or electronic properties of the chemical moieties present within the side-chain. Examples of amino acids containing hydrophobic side-chains include those containing unsaturated aliphatic hydrocarbons, such as alanine, valine, leucine, isoleucine, proline, and methionine, as well as amino acids containing aromatic ring systems that are electrostatically neutral at physiological pH, such as tryptophan, phenylalanine, and tyrosine.

As used herein, the term “immunotherapy agent” refers to a compound, such as an antibody, antigen-binding fragment thereof, single-chain polypeptide, or construct as described herein, that specifically binds an immune checkpoint protein (e.g., immune checkpoint receptor or ligand) and exerts an antagonistic effect on the receptor or ligand, thereby reducing or inhibiting the signal transduction of the receptor or ligand that would otherwise lead to a downregulation of the immune response. Immunotherapy agents include compounds, such as antibodies, antigen-binding fragments, single-chain polypeptides, and constructs, capable of specifically binding receptors expressed on the surfaces of hematopoietic cells, such as lymphocytes (e.g., T cells), and suppressing the signaling induced by the receptor or ligand that would otherwise lead to tolerance towards an endogenous (“self”) antigen, such as a tumor-associated antigen. Immunotherapy agents may reduce the signaling induced by the receptor or ligand by, for example, 1%, 2%, 3%, 4%, 5%, 10%, 20%, 30%, 40%, 50%, 60%, 70%, 80%, 90%, 95%, 96%, 97%, 98%, 99%, 99.9%, or 100% relative to the signaling induced by the receptor or ligand exhibited in the absence of the immunotherapy agent. Exemplary assays that can be used to measure the extent of receptor or ligand signaling include, for example, enzyme-linked immunosorbant assay (ELISA) techniques to measure protein expression alterations that are associated with a particular signal transduction pathway, as well as polymerase chain reaction (PCR)-based techniques, such as quantitative PCR, reverse-transcription PCR, and real-time PCR experiments useful for determining changes in gene expression associated with a particular signal transduction pathway, among others. Exemplary methods that can be used to determine whether an agent is an “immunotherapy agent” include the assays described in Mahoney et al., Cancer Immunotherapy, 14:561-584 (2015), the disclosure of which is incorporated herein by reference in its entirety. Examples of immunotherapy agents include, e.g., antibodies or antigen-binding fragments thereof that specifically bind one or more of OX40L, TL1A, CD40L, LIGHT, BTLA, LAG3, TIM3, Singlecs, ICOS, B7-H3, B7-H4, VISTA, TMIGD2, BTNL2, CD48, KIR, LIR, LIR antibody, ILT, NKG2D, NKG2A, MICA, MICB, CD244, CSF1R, IDO, TGFβ, CD39, CD73, CXCR4, CXCL12, SIRPA, CD47, VEGF, and neuropilin. Additional example of immunotherapy agents include Targretin, Interferon-alpha, clobestasol, Peg Interferon (e.g., PEGASYS®), prednisone, Romidepsin, Bexarotene, methotrexate, Trimcinolone cream, anti-chemokines, Vorinostat, gabapentin, antibodies to lymphoid cell surface receptors and/or lymphokines, antibodies to surface cancer proteins, and/or small molecular therapies like Vorinostat. Particular examples of immunotherapy agents that may be used in conjunction with the compositions and methods described herein include anti-PD-1 antibodies and antigen-binding fragments thereof, such as nivolumab, pembrolizumab, avelumab, durvalumab, and atezolizumab, as well as anti-PD-L1 antibodies and antigen-binding fragments thereof, such as atezolizumab and avelumab, and anti-CTLA-4 antibodies and antigen-binding fragments thereof, such as ipilimumab or tremelimumab.

As used herein, the term “monoclonal antibody” refers to an antibody that is derived from a single clone, including any eukaryotic, prokaryotic, or phage clone, and not the method by which it is produced.

As used herein, the term “multi-specific antibodies” refers to antibodies that exhibit affinity for more than one target antigen. Multi-specific antibodies can have structures similar to full immunoglobulin molecules and include Fc regions, for example IgG Fc regions. Such structures can include, but not limited to, IgG-Fv, IgG-(scFv)₂, DVD-Ig, (scFv)₂-(scFv)₂-Fc and (scFv)₂-Fc-(scFv)₂. In case of IgG-(scFv)₂, the scFv can be attached to either the N-terminal or the C-terminal end of either the heavy chain or the light chain. Exemplary multi-specific molecules that include Fc regions and into which conformation-specific NF-κB antibodies or antigen-binding fragments thereof can be incorporated have been reviewed by Kontermann, 2012, mAbs 4(2):182-197, Yazaki et al, 2013, Protein Engineering, Design & Selection 26(3):187-193, and Grote et al, 2012, in Proetzel & Ebersbach (eds.), Antibody Methods and Protocols, Methods in Molecular Biology vol. 901, chapter 16:247-263; incorporated herein by reference. In some embodiments, antibody fragments can be components of multi-specific molecules without Fc regions, based on fragments of IgG or DVD or scFv. Exemplary multi-specific molecules that lack Fc regions and into which antibodies or antibody fragments can be incorporated include scFv dimers (diabodies), trimers (triabodies) and tetramers (tetrabodies), Fab dimers (conjugates by adhesive polypeptide or protein domains) and Fab trimers (chemically conjugated), are described by Hudson and Souriau, 2003, Nature Medicine 9:129-134; incorporated herein by reference.

As used herein, the term “nuclear factor kappa-light-chain-enhancer of activated B cells (NF-κB)” refers to a protein complex that controls transcription of DNA, cytokine production, and cell survival. NF-κB is found in almost all animal cell types and is involved in cellular responses to stimuli, such as stress, cytokines, free radicals, heavy metals, ultraviolet irradiation, oxidized LDL, and bacterial or viral antigens. NF-κB plays a key role in regulating the immune response to infection. Incorrect regulation of NF-κB has been linked to disorders, including but not limited to cancer, inflammatory and immune diseases (e.g., autoimmune diseases), sepsis, septic shock, infection (e.g., viral infection), and improper immune development. NF-κB regulates the expression of a large number of genes including but not limited to: IGHG4, IGHG3, APOC3, TNFRSF6, CD3G, TNFSF5, CD105, ICAM1, TPMT, IL2RA, SELE, TP53, CRP, IL1A, IL1B, IL1RN, CCR5, IL8, IL2, IL9, TAP1, TNF, LTA, IL6, CD44, NOS2A, SOD2, TNFSF6, IL11, BDKRB1, CSF1, CSF2, CSF3, GSTP1, NQO1, OPRM1, PTAFR, PTGS2, SCNN1A, VCAM1, AGER, ALOX12B, BCL2L1, TNFRSF5, TNFRSF9, IRF7, BLR1, CD48, CD69, CCR7, CR2, F3, HMOX1, TNC, IFNB1, IL13, IL15RA, IRF1, IRF2, LTB, IRF4, MYC, NFKB2, PDGFB, PLAU, LMP2, PTX3, CCL2, CCL5, CCL11, CXCL5, SELP, SLC2A5, STAT5A, VIM, IER3, NFKB1, BM2, BCL2A1, CCL15, CD83, CD74, ELF3, TGM2, DEFB4, MMP9, BCL3, CD80, VEGFC, PLCD1, TNFAIP3, RELB, TFPI2, BCL2, S100A6, TACR1, NFKBIA, CD209, CARD15, CCND1, KLK3, IL15, NR4A2, and HC3. As used herein, the the term “NF-κB signaling” and the like refer to the ability of NF-κB to regulate (e.g., increase or decrease) the gene expression of any of its target genes. An increase of decrease in NF-κB signaling may therefore be measured by an increase or decrease in one or more corresponding target genes.

As used herein, the term “non-native constant region” refers to an antibody constant region that is derived from a source that is different from the antibody variable region or that is a human-generated synthetic polypeptide having an amino sequence that is different from the native antibody constant region sequence. For instance, an antibody containing a non-native constant region may have a variable region derived from a non-human source (e.g., a mouse, rat, or rabbit) and a constant region derived from a human source (e.g., a human antibody constant region), or a constant region derived from another primate, pig, goat, rabbit, hamster, cat, dog, guinea pig, member of the bovidae family (such as cattle, bison, buffalo, elk, and yaks, among others), cow, sheep, horse, or bison, among others).

As used herein, the term “percent (%) sequence identity” refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent sequence identity (e.g., gaps can be introduced in one or both of the candidate and reference sequences for optimal alignment and non-homologous sequences can be disregarded for comparison purposes). Alignment for purposes of determining percent sequence identity can be achieved in various ways that are within the skill in the art, for instance, using publicly available computer software, such as BLAST, ALIGN, or Megalign (DNASTAR) software. Those skilled in the art can determine appropriate parameters for measuring alignment, including any algorithms needed to achieve maximal alignment over the full length of the sequences being compared. For example, a reference sequence aligned for comparison with a candidate sequence may show that the candidate sequence exhibits from 50% to 100% sequence identity across the full length of the candidate sequence or a selected portion of contiguous amino acid (or nucleic acid) residues of the candidate sequence. The length of the candidate sequence aligned for comparison purposes may be, for example, at least 30%, (e.g., 30%, 40, 50%, 60%, 70%, 80%, 90%, or 100%) of the length of the reference sequence. When a position in the candidate sequence is occupied by the same amino acid residue as the corresponding position in the reference sequence, then the molecules are identical at that position.

As used herein, the term “primatized antibody” refers to an antibody including framework regions from primate-derived antibodies and other regions, such as CDRs and/or constant regions, from antibodies of a non-primate source. Methods for producing primatized antibodies are known in the art. See e.g., U.S. Pat. Nos. 5,658,570; 5,681,722; and 5,693,780; incorporated herein by reference. For instance, a primatized antibody or antigen-binding fragment thereof described herein can be produced by inserting the CDRs of a non-primate antibody or antigen-binding fragment thereof into an antibody or antigen-binding fragment thereof that contains one or more framework regions of a primate.

As used herein, the term “pro-inflammatory cytokine” refers to a cytokine secreted from immune cells that promotes inflammation. Immune cells that produce and secrete pro-inflammatory cytokines include T cells (e.g., Th cells) macrophages, B cells, and mast cells. Pro-inflammatory cytokines include interleukin-1 (IL-1, e.g., IL-1β), IL-5, IL-6, IL-8, IL-10, IL-12, IL-13, IL-18, tumor necrosis factor (TNF, e.g., TNFα), interferon gamma (IFNγ), and granulocyte macrophage colony stimulating factor (GMCSF).

As used herein, the term “proline analog” is meant a molecule substantially similar in function to either an entire proline amino acid residue or to a fragment thereof. For example, the present invention contemplates the use of proline analogs wherein a side chain is lengthened or shortened while still providing a carboxyl, amino, or other reactive precursor functional group, as well as proline analogs having variant side chains with appropriate functional groups. Exemplary proline analogs include, without limitation, homoproline (i.e., pipecolic acid (PIP)), azetidine-2-carboxylic acid (Aze), tert-butyl-L-proline (TBP), trans-4-fluoro-L-proline (t-4F-Pro), or cis-4-fluoro-L-proline (c-4F-Pro).

As used herein, the term “operatively linked” in the context of a polynucleotide fragment is intended to mean that the two polynucleotide fragments are joined such that the amino acid sequences encoded by the two polynucleotide fragments remain in-frame.

As used herein, the term “pharmacokinetic profile” refers to the absorption, distribution, metabolism, and clearance of a drug over time following administration of the drug to a patient.

As used herein, the term “regulatory sequence” includes promoters, enhancers and other expression control elements (e.g., polyadenylation signals) that control the transcription or translation of the antibody chain genes. Such regulatory sequences are described, for example, in Goeddel, Gene Expression Technology: Methods in Enzymology 185 (Academic Press, San Diego, Calif., 1990); incorporated herein by reference.

As used herein, the term “scFv” refers to a single-chain Fv antibody in which the variable domains of the heavy chain and the light chain from an antibody have been joined to form one chain. scFv fragments contain a single polypeptide chain that includes the variable region of an antibody light chain (VL) (e.g., CDR-L1, CDR-L2, and/or CDR-L3) and the variable region of an antibody heavy chain (VH) (e.g., CDR-H1, CDR-H2, and/or CDR-H3) separated by a linker. The linker that joins the VL and VH regions of a scFv fragment can be a peptide linker composed of proteinogenic amino acids. Alternative linkers can be used to so as to increase the resistance of the scFv fragment to proteolytic degradation (e.g., linkers containing D-amino acids), in order to enhance the solubility of the scFv fragment (e.g., hydrophilic linkers such as polyethylene glycol-containing linkers or polypeptides containing repeating glycine and serine residues), to improve the biophysical stability of the molecule (e.g., a linker containing cysteine residues that form intramolecular or intermolecular disulfide bonds), or to attenuate the immunogenicity of the scFv fragment (e.g., linkers containing glycosylation sites). scFv molecules are known in the art and are described, e.g., in U.S. Pat. No. 5,892,019, Flo et al., (Gene 77:51, 1989); Bird et al., (Science 242:423, 1988); Pantoliano et al., (Biochemistry 30:10117, 1991); Milenic et al., (Cancer Research 51:6363, 1991); and Takkinen et al., (Protein Engineering 4:837, 1991). The VL and VH domains of a scFv molecule can be derived from one or more antibody molecules. It will also be understood by one of ordinary skill in the art that the variable regions of the scFv molecules described herein can be modified such that they vary in amino acid sequence from the antibody molecule from which they were derived. For example, in one embodiment, nucleotide or amino acid substitutions leading to conservative substitutions or changes at amino acid residues can be made (e.g., in CDR and/or framework residues). Alternatively, or in addition, mutations are made to CDR amino acid residues to optimize antigen binding using art recognized techniques. scFv fragments are described, for example, in WO 2011/084714; incorporated herein by reference.

As used herein, the term “sepsis” refers to a condition characterized by an inflammatory immune response, and which may arise in response to infection, trauma, or disease. Sepsis may be associated with a bacterial infection, a viral infection, a fungal infection, or a parasitic infection (e.g., as described herein). Common locations for the primary infection include the lungs, brain, urinary tract, skin, and abdominal organs. Risk factors include very young age, older age, a weakened immune system from conditions such as cancer or diabetes, major trauma, or burns. Sepsis may also arise independent from an infection and is the referred to as sterile sepsis. Sepsis therefore may also be associated with trauma, burns, pancreatitis, or ischaemic reperfusion. Common signs and symptoms include fever, increased heart rate, increased breathing rate, and confusion. There may also be symptoms related to a specific infection, such as a cough with pneumonia, or painful urination with a kidney infection. In the very young, old, and people with a weakened immune system, there may be no symptoms of a specific infection and the body temperature may be low or normal, rather than high. Severe sepsis may be characterized by poor organ function or insufficient blood flow. Insufficient blood flow may be evident by low blood pressure, high blood lactate, or low urine output. Septic shock may be characterized by low blood pressure due to sepsis that does not improve after fluid replacement. Sepsis may be characterized by an increase in pro-inflammatory cytokines in a subject, e.g., an increase in include one or more interleukin (e.g., IL-1, IL-5, IL-6, IL-8, IL-10, IL-12, IL-13, or IL-18), tumor necrosis factor (TNF, e.g., TNFα), interferon gamma (IFNγ), or granulocyte macrophage colony stimulating factor (GMCSF). Other symptoms associates with sepsis are known to those of skill in the art, and include increased white blood cell count, immature white blood cells in the circulation, elevated plasma C-reactive protein, elevated procalcitonin (PCT), low blood pressure, low central venous or mixed venous oxygen saturation, high cardiac index, low oxygen level, low urine output, high creatinine in the blood, coagulation (clotting) abnormalities, absent bowel sounds, low platelets in the blood, high bilirubin levels, high lactate in the blood, or decreased capillary filling or mottling.

As used herein, the phrase “specifically binds” refers to a binding reaction which is determinative of the presence of an antigen in a heterogeneous population of proteins and other biological molecules that is recognized, e.g., by an antibody or antigen-binding fragment thereof, with particularity. An antibody or antigen-binding fragment thereof that specifically binds to an antigen will bind to the antigen with a K_{D} of less than 100 nM. For example, an antibody or antigen-binding fragment thereof that specifically binds to an antigen will bind to the antigen with a K_{D} of up to 100 nM (e.g., between 1 μM and 100 nM). An antibody or antigen-binding fragment thereof that does not exhibit specific binding to a particular antigen or epitope thereof will exhibit a K_{D} of greater than 100 nM (e.g., greater than 500 nm, 1 μM, 100 μM, 500 μM, or 1 mM) for that particular antigen or epitope thereof. A variety of immunoassay formats may be used to select antibodies specifically immunoreactive with a particular protein or carbohydrate. For example, solid-phase ELISA immunoassays are routinely used to select antibodies specifically immunoreactive with a protein or carbohydrate. See, Harlow & Lane, Antibodies, A Laboratory Manual, Cold Spring Harbor Press, New York (1988) and Harlow & Lane, Using Antibodies, A Laboratory Manual, Cold Spring Harbor Press, New York (1999), for a description of immunoassay formats and conditions that can be used to determine specific immunoreactivity.

As used herein, the terms “subject” and “patient” refer to an organism that receives treatment for a particular disease or condition as described herein (such as cancer or an infectious disease). Examples of subjects and patients include mammals, such as humans, primates, pigs, goats, rabbits, hamsters, cats, dogs, guinea pigs, members of the bovidae family (such as cattle, bison, buffalo, elk, and yaks, among others), cows, sheep, horses, and bison, among others, receiving treatment for diseases or conditions, for example, NF-κB-related diseases (e.g., infection, cancer, or immune or inflammatory disorders such as sepsis, septic shock, SIRS, or CRS).

As used herein, the term “systemic inflammatory response syndrome (SIRS)” refers to a condition associated with systemic inflammation, organ dysfunction, and/or organ failure. It is characterized by abnormal regulation of cytokines and may include both pro- and anti-inflammatory component. SIRS may occur in response to an infectious or non-infectious insult. SIRS may be associated with, for example, infection (e.g., bacterial, viral, fungal, or parasitic infection), trauma, burns, pancreatitis, ischaemic reperfusion, hemorrhage, complications of surgery, pulmonary embolism, aortic aneurysm, cardiac tamponade, anaphylaxis, or drug overdose. Manifestations of SIRS include, but are not limited to: increased or decreased body temperature (e.g., body temperature less than 36° C. (96.8° F.) or greater than 38° C. (100.4° F.)), increased heart rate (e.g., heart rate greater than 90 beats per minute), high respiratory rate (e.g., greater than 20 breaths per minute), an arterial partial pressure of carbon dioxide less than 4.3 kPa (32 mmHg), abnormal white blood cell count (e.g., a white blood cell count less than 4000 cells/mm³ (4×109 cells/L) or greater than 12,000 cells/mm³ (12×109 cells/L)), and/or the presence of greater than 10% immature neutrophils. When two or more of these criteria are met with or without evidence of infection, patients may be diagnosed with SIRS. SIRS may also be characterized by an increase in pro-inflammatory cytokines in a subject, e.g., an increase in include one or more interleukin (e.g., IL-1, IL-5, IL-6, IL-8, IL-10, IL-12, IL-13, or IL-18), tumor necrosis factor (TNF, e.g., TNFα), interferon gamma (IFNγ), or granulocyte macrophage colony stimulating factor (GMCSF). Patients with SIRS and acute organ dysfunction may be termed severe SIRS. Additional criteria for the diagnosis of SIRS will be apparent to those of skill in the art (for example, as described in Balk RA, Systemic inflammatory response syndrome (SIRS): Where did it come from and is it still relevant today? Virulence. 5(1):20-26 (2014), which is incorporated herein by reference in its entirety).

As used herein, the term “transfection” refers to any of a wide variety of techniques commonly used for the introduction of exogenous DNA into a prokaryotic or eukaryotic host cell, e.g., electroporation, lipofection, calcium-phosphate precipitation, DEAE-dextran transfection and the like.

As used herein, the terms “treat” or “treatment” refer to therapeutic treatment, in which the object is to prevent or slow down (lessen) an undesired physiological change or disorder, such as the progression of an NF-κB-related diseases (e.g., infection, cancer, or immune or inflammatory disorders such as sepsis, septic shock, SIRS, or CRS). Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. Those in need of treatment include those already with the condition or disorder, as well as those prone to have the condition or disorder or those in which the condition or disorder is to be prevented. To treat, as used throught this application, therefore also refers to reducing likelihood of occurrence in a subject at risk of developing a disorder (e.g., relative to a subject not treated with antibody described herein and/or relative to a subject treated with an alternative therapy).

As used herein the term “variable region CDR” includes amino acids in a CDR or complementarity determining region as identified using sequence or structure-based methods. As used herein, the term “CDR” or “complementarity determining region” refers to the noncontiguous antigen-binding sites found within the variable regions of both heavy and light chain polypeptides. These particular regions have been described by Kabat et al., J. Biol. Chem. 252:6609-6616, 1977 and Kabat, et al., Sequences of Proteins of Immunological Interest, Fifth Edition, U.S. Department of Health and Human Services, NIH Publication No. 91-3242, 1991; by Chothia et al., (J. Mol. Biol. 196:901-917, 1987), and by MacCallum et al., (J. Mol. Biol. 262:732-745, 1996) where the definitions include overlapping or subsets of amino acid residues when compared against each other. The term “CDR” may be, for example, a CDR as defined by Kabat based on sequence comparisons.

As used herein, the term “vector” includes a nucleic acid vector, e.g., a DNA vector, such as a plasmid, a RNA vector, virus or other suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO 1994/11026; incorporated herein by reference. Expression vectors described herein contain a polynucleotide sequence as well as, e.g., additional sequence elements used for the expression of proteins and/or the integration of these polynucleotide sequences into the genome of a mammalian cell. Certain vectors that can be used for the expression of antibodies and antibody fragments described herein include plasmids that contain regulatory sequences, such as promoter and enhancer regions, which direct gene transcription. Other useful vectors for expression of antibodies and antibody fragments contain polynucleotide sequences that enhance the rate of translation of these genes or improve the stability or nuclear export of the mRNA that results from gene transcription. These sequence elements include, e.g., 5′ and 3′ untranslated regions, an internal ribosomal entry site (IRES), and polyadenylation signal site in order to direct efficient transcription of the gene carried on the expression vector. The expression vectors described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

As used herein, the term “VH” refers to the variable region of an immunoglobulin heavy chain of an antibody, including the heavy chain of an Fv, scFv, or Fab. References to “VL” refer to the variable region of an immunoglobulin light chain, including the light chain of an Fv, scFv, dsFv or Fab. Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150,000 Daltons, composed of two identical light (L) chains and two identical heavy (H) chains. Each heavy chain of a native antibody has at the amino terminus a variable domain (VH) followed by a number of constant domains. Each light chain of a native antibody has a variable domain at the amino terminus (VL) and a constant domain at the carboxy terminus.

### BRIEF DESCRIPTION OF THE DRAWINGS

The patent or application file contains at least one drawing in color. Copies of this patent or patent application publication with color drawings will be provided by the Office upon request and payment of the necessary fee.

FIG. 1 is a series of immunofluorescence microscopy images showing that lipopolysaccharide (LPS) stimulation of immune cells causes induction of p65 NF-κB proteins in the nucleus and cytoplasm, which are specifically detected by trans-mAb and cis-mAb, respectively.

FIGS. 2A-2B are graphs showing that trans-mAb fully reduces cytokine releases in cell cultures and mortality caused by LPS-induced septic shock in mice. FIG. 2A is a series of graphs showing that trans-mAb dose-dependently blocks LPS, TNFα, and IL-1β from inducing proinflammatory cytokines, such as IL-6 in cell cultures. FIG. 2B is a set of graphs showing that trans-mAb drastically reduces cytokine storm and mortality after injection of lethal dose of LPS in mice.

FIGS. 3A-3C show that the nuclear form of NF-κB is induced in patients with bacterial sepsis and is located in the nucleus of peripheral blood mononuclear cells (PBMCs). FIG. 3A is a series of immunofluorescence images showing strong signals for both nuclear (trans) NF-κB and cytoplasmic (cis) NF-κB in PBMCs of human sepsis patients compared to PBMCs of control healthy human subjects. FIG. 3B is a set of graphs showing the percentage of trans NF-κB positive PBMCs in septic patients. The positive ratios in the PBMC from sepsis patients were significantly higher than the PBMC from healthy control. FIG. 3C is graph showing the correlation between trans NF-kB positive ratio in staining and IL-6 in plasma. The trans NF-kB positive ratio and IL-6 value in plasma showed a positive correlation using Spearman's rank correlation coefficient.

FIG. 4 is a schematic showing the caecum ligation and puncture (CLP) model, which is widely recognized as the gold standard for sepsis studies.

FIGS. 5A-5F show that trans-mAb drastically reduces cytokine storm and mortality of septic shock in mice. FIG. 5A is a schematic showing a CLP treatment regimen. FIG. 5B is a photograph showing that trans-mAb-treated CLP mice do not develop septic shock. FIG. 5C is a series of graphs showing that trans-mAb treatment potently inhibits the cytokine storm after CLP. FIG. 5D is a set of graphs showing that trans-mAb treatment potently inhibits time-dependent releases of cytokines after CLP. FIG. 5E is a western blot showing that trans-mAb treatment potently eliminates time-dependent accumulation of nuclear trans active p65 NF-κB in the lung after CLP. FIG. 5F is a graph showing that trans-mAb treatment drastically improves survival of CLP mice.

FIGS. 6A-6B show that trans-mAb treatment after CLP allows mice to successfully fight secondary bacterial lung infections. FIG. 6A is a schematic showing a treatment regimen of the CLP two-hit model. FIG. 6B is a graph showing that trans-mAb treatment allows CLP mice to fight secondary bacterial lung infection, drastically improving survival of two-hit CLP mice.

FIGS. 7A-7C show that trans-mAb treatment after CLP effectively reduces immunosuppression in the CLP two hit model, as assayed by apoptosis of T and B cells in the spleen. FIG. 7A is a schematic showing a treatment regimen carried out using the CLP 2 hit model. FIG. 7B is a graph showing that trans-mAb treatment reduces apoptosis of splenic T cells of CLP mice before and after bacterial lung infection. FIG. 7C is a graph showing that trans-mAb treatment reduces apoptosis of splenic B cells of CLP mice before and after bacterial lung infection.

FIG. 8 is an image showing the sequence alignment of trans-mAb light chain variable domains having the amino acid sequences of SEQ ID NOs: 4, 12, 16, 19, and 23.

FIG. 9 is an image showing the sequence alignment of trans-mAb heavy chain variable domains having the amino acid sequences of SEQ ID NOs: 8 and 10.

FIG. 10A is a photographic image of a western blot showing that trans mAb eliminates LPS-induced trans p65 induction with little effects on total p65. DC2.4 cells were simulated with LPS in the presence of trans mAb (Trans) or control IgG at 1.0 μg/ml, followed by immunoblotting with trans and total p65.

FIG. 10B is a graph showing that trans mAb dose-dependently eliminates p65 in DC2.4 cells after stimulation with LPS at 1.0 μg/ml in the presence of varying concentrations of mAbs (0.01 to 1.0 μg/ml) followed by immunoblotting for trans and total p65 (n=3).

FIG. 10C is a photographic image of a western blot showing that trans mAb time-dependently eliminates p65 in DC2.4 cells after stimulation with PLS at 1.0 μg/ml in the presence of mAbs at different times followed by immunoblotting for trans and total p65 (n=3).

FIG. 11A is a photographic image of a western blot showing that trans mAb dose-dependently eliminates p65 in Raw264.7 cells after stimulation with PLS at 1.0 μg/ml in the presence of varying concentrations of mAbs (0.01 to 1.0 μg/ml) followed by immunoblotting for trans and total p65 (n=3).

FIG. 11B is a photographic image of a western blot showing that trans mAb time-dependently eliminates p65 in Raw264.7 cells after stimulation with PLS at 1.0 μg/ml in the presence of mAbs at different times followed by immunoblotting for trans and total p65 (n=3).

FIG. 12A is a photographic image of a western blot showing that trans mAb eliminates LPS-induced total p65 nuclear accumulation. Raw264.7 cells were treated simulated with LPS in the presence of control IgG or trans mAb, followed by cytosolic and nuclear fractionation before IB for total p65, with tubulin and lamin as markers.

FIG. 12B is a pair of graphs showing that trans mAb reduces LPS-induced total p65 nuclear accumulation. Raw264.7 cells were treated with LPS or vehicle in the presence of trans mAb or control IgG, followed by immunoblotting on cytosolic or nuclear fraction or immunofluorescence for total p65 and quantification (n=3).

FIG. 13 is a series of immunofluorescence images showing that trans mAb reduces LPS-induced total p65 nuclear accumulation. Raw264.7 cells were treated with LPS or vehicle in the presence of trans mAb or control IgG, followed by immunofluorescence for total p65 and quantification (n=3).

FIGS. 14A-14C are graphs showing that trans mAb (square symbols) inhibits NF-κB transcriptional activity compared to control IgG antibody (circle symbols). DC2.4 cells were treated with LPS (FIG. 14A), TNF-α (FIG. 14B), or IL-1β (FIG. 14C) in the presence of different concentrations of mAb, followed by the NF-κB luciferase reporter assay. n=2 to 3 per group for each dose. (−) indicates basal activity without LPS or cytokine stimulation.

FIGS. 15A-15B are graphs showing that trans mAb (square symbols) inhibits IL-6 (FIG. 15A) and IL-1β (FIG. 15B) transcription in response to LPS or cytokines compared to control IgG antibody (circle symbols). DC2.4 cells were treated with different concentrations of LPS in the presence of mAb (1.0 μg/ml), followed by assaying IL-6 and IL-1β mRNA using pRT-PCR (n=2 to 4 per group per dose). (−) indicates basal activity without LPS or cytokine stimulation.

FIGS. 16A-16C are graphs showing that trans (square symbols), but not cis (triangle symbols), mAb inhibits IL-6 production in response to LPS or cytokines compared to control IgG antibody (circle symbols). DC2.4 cells were simulated with LPS (FIG. 16A), TNF-α (FIG. 16B), or IL-1β (FIG. 16C) in the presence of cis or trans mAb at different concentrations, or control IgG, followed by ELISA for IL-6. (−) indicates basal IL-6 without stimulation.

FIGS. 17A-17B are graphs showing that trans mAb (square symbols) inhibits IL-6 production after stimulation by LPS (FIG. 17A) and TNF-α (FIG. 17B) compared to cis mAb (triangle symbols), IgG (circle symbols), and untreated control (diamond symbols). DC2.4 cells were treated with various doses of LPS or TNF-α in the presence of cis or trans mAb or control IgG, followed by ELISA for IL-6. n=3 per group each dose.

FIG. 18 is a series of immunofluorescence images showing that trans p65-positive PBMCs in percentage are correlated with the Sequential Organ Failure Assessment (SOFA) scores. PBMCs were divided into two groups using SOFA scores (cut-off value=2), followed by immunofluorescence staining.

FIG. 19 is a series of fluorescence images showing that cis and trans p65 are mainly observed in the cytoplasm and nucleus, respectively, in human sepsis PBMCs.

FIGS. 20A-200 are graphs showing the correlation between trans p65-positive PBMCs and various clinical parameters of sepsis patients. Septic PBMC samples were divided into two groups using MAP (mean arterial pressure) (cut-off value=70 mmHg). Lactate (cut-off value=2 mmol/l) (FIG. 20A), creatinine (cut-off value=1.1 mg/dl) (FIG. 20A), and O2 administration (with our without).

FIG. 21 is a graph showing the quantification of trans p65-positive PBMCs (%) from the immunofluorescence images in FIG. 18.

FIG. 22 is a graph showing trans p65-positive PBMCs in percentage and their correlation with SOFA scores.

FIG. 23 is a graph showing the correlation between trans p65-positive PBMCs (%) with serum lactate levels in sepsis patients.

FIGS. 24A-24B are graphs showing the correlation between trans trans p65-positive PBMCs (%) with plasma NF-κB targets IL-6 (FIG. 24A) and IL-10 (FIG. 24B).

FIG. 25A is a series of fluorescence images showing that trans mAb ablates trans p65 and attenuates the cytokine storm and death in polymicrobial sepsis. Mice underwent sham or Cecal Ligation and Puncture (CLP) operation were treated with trans mAb or IgG, as above, followed by assaying trans and total p65 in the spleen, lung, and thymus at 24 hr by immunofluorescence imaging.

FIG. 25B is a photographic image of a western blot showing that trans mAb ablates trans p65 and attenuates the cytokine storm and death in polymicrobial sepsis. Mice underwent sham or CLP operation were treated with trans mAb or IgG, as above, followed by assaying trans and total p65 in the spleen, lung, and thymus at 24 hr by immunoblotting.

FIG. 26A is a series of graphs showing ablation of trans p65 by trans mAb in CLP mice. Mice underwent sham or CLP operation and were treated with trans mAn or IgG control and 24 hours later, the spleen, the lungs, and thymus were harvested for immunofluorescence and subsequent quantification of trans p65 levels.

FIG. 26B is a set of photographic images of western blots showing ablation of trans p65 by trans mAb in CLP mice. Mice underwent sham or CLP operation and were treated with trans mAb or IgG control and 24 hours later, the spleen, and thymus were harvested for immunoblotting and measuring trans p65 levels.

FIG. 27 is a series of histological images of spleen, lung, kidney, and liver tissue of CLP mice treated with either trans mAb or IgG control. Tissue samples were treated with Gram staining to identify bacteria. Trans mAb prevented the infiltration of bacteria, macrophages, and neutrophils as well as the induction of apoptosis in the tissues surveyed.

FIG. 28 is a series of immunofluorescence images showing trans mAb prevented the infiltration of macrophages (CD68₊ cells) into spleen, lung, or thymus tissue after treatment with trans mAb.

FIG. 29 is a series of immunofluorescence images showing trans mAb prevented the infiltration of neutrophils into spleen, lung, or thymus tissue after treatment with trans mAb.

FIG. 30 is a series of immunofluorescence images showing trans mAb prevented apoptosis in spleen, lung, or thymus tissue after treatment with trans mAb.

FIG. 31 is a series of immunofluorescence images showing that trans mAb ablates trans p65 and inhibits cytokine production from human sepsis patients ex vivo. PBMCs freshly isolated from sepsis patients were cultured with trans mAb or IgG for 8 hrs, followed by IF for trans p65. n=5 per group.

FIGS. 32A-32B are graphs showing that trans mAb ablates trans p65 and inhibits cytokine production from human sepsis patients ex vivo. PBMCs freshly isolated from sepsis patients were cultured with trans mAb or IgG for 8 hrs, followed by ELISA for IL-6 (FIG. 32A) and TNF-α (FIG. 32B). n=5 per group.

FIG. 33 is a schematic drawing showing the SAP model and the treatments with trans mAb or IgG (300 μg/mouse).

FIG. 34 is a series of immunofluorescence images showing that trans mAb ablates trans p65 induction and attenuates severe acute pancreatitis in SAP mice. SAP mice were generated using caerulein and LPS and treated with trans mAb a control IgG followed by harvesting pancreatic tissue for trans p65 immunofluorescence.

FIG. 35 is a series of H&E staining images of panchreatic tissue showing that trans mAb ablates trans p65 induction and attenuates severe acute pancreatitis in SAP mice. SAP mice were generated using caerulein and LPS and treated with trans mAb a control IgG followed by harvesting pancreatic tissue for trans p65 H&E staining.

FIG. 36 is a series of H&E staining images showing that trans mAb ablates trans p65 and attenuates acute lung injury in SAP mice. Mice were injected with caerulein and LPS or vehicle, followed by the treatment with trans mAb and the subsequent labeling with H&E stain to assess acute lung injury.

FIG. 37 is a series of dot plots showing that trans mAb treatment significantly rescues expression of top differentially expressed genes (DEGs) in different immune cells. scRNA-seq was used to profile the transcriptomic changes of total ˜15,000 splenocytes from mice 6 hrs after CLP treated with trans mAb or IgG, referencing to sham littermates. Examples of top up-regulated or down-regulated DEGs in 5 major immune cell types in the spleen are shown in dot plots with average expression color coded and percentage of cells expressing the gene correlated with the size. The DEGs are ranked from most up-regulated (top) to most down-regulated (bottom).

FIG. 38 is a pair of volcano plots showing CLP-altered DEGs after trans mAb or IgG treatment with reference to sham controls for T cells. CLP up-regulated DEGs are colored in red (p<0.01) and down-regulated DEGs are in blue (p<0.01). The insignificant changes (p>=0.01), non-CLP changes (p<0.01) or CLP DEGs that have been recovered by trans mAb (p>=0.01 after trans mAb) are colored in gray.

FIG. 39 is a pair of volcano plots showing CLP-altered DEGs after trans mAb or IgG treatment with reference to sham controls for B cells. CLP up-regulated DEGs are colored in red (p<0.01) and down-regulated DEGs are in blue (p<0.01). The insignificant changes (p>=0.01), non-CLP changes (p<0.01) or CLP DEGs that have been recovered by trans mAb (p>=0.01 after trans mAb) are colored in gray.

FIG. 40 is a pair of volcano plots showing CLP-altered DEGs after trans mAb or IgG treatment with reference to sham controls for dendritic cells. CLP up-regulated DEGs are colored in red (p<0.01) and down-regulated DEGs are in blue (p<0.01). The insignificant changes (p>=0.01), non-CLP changes (p<0.01) or CLP DEGs that have been recovered by trans mAb (p>=0.01 after trans mAb) are colored in gray.

FIG. 41 is a pair of volcano plots showing CLP-altered DEGs after trans mAb or IgG treatment with reference to sham controls for erythoblasts. CLP up-regulated DEGs are colored in red (p<0.01) and down-regulated DEGs are in blue (p<0.01). The insignificant changes (p>=0.01), non-CLP changes (p<0.01) or CLP DEGs that have been recovered by trans mAb (p>=0.01 after trans mAb) are colored in gray.

FIG. 42 is a pair of volcano plots showing CLP-altered DEGs after trans mAb or IgG treatment with reference to sham controls for granulocytes. CLP up-regulated DEGs are colored in red (p<0.01) and down-regulated DEGs are in blue (p<0.01). The insignificant changes (p>=0.01), non-CLP changes (p<0.01) or CLP DEGs that have been recovered by trans mAb (p>=0.01 after trans mAb) are colored in gray.

FIG. 43 is a pair of volcano plots showing CLP-altered DEGs after trans mAb or IgG treatment with reference to sham controls for NK cells. CLP up-regulated DEGs are colored in red (p<0.01) and down-regulated DEGs are in blue (p<0.01). The insignificant changes (p>=0.01), non-CLP changes (p<0.01) or CLP DEGs that have been recovered by trans mAb (p>=0.01 after trans mAb) are colored in gray.

FIG. 44 is a pair of volcano plots showing CLP-altered DEGs after trans mAb or IgG treatment with reference to sham controls for macrophages. CLP up-regulated DEGs are colored in red (p<0.01) and down-regulated DEGs are in blue (p<0.01). The insignificant changes (p>=0.01), non-CLP changes (p<0.01) or CLP DEGs that have been recovered by trans mAb (p>=0.01 after trans mAb) are colored in gray.

FIG. 45 is a pair of volcano plots showing CLP-altered DEGs after trans mAb or IgG treatment with reference to sham controls for endothelial cells. CLP up-regulated DEGs are colored in red (p<0.01) and down-regulated DEGs are in blue (p<0.01). The insignificant changes (p>=0.01), non-CLP changes (p<0.01) or CLP DEGs that have been recovered by trans mAb (p>=0.01 after trans mAb) are colored in gray.

FIG. 46 is a graph showing the that trans mAb treatment reverts expression of ˜200 CLP-induced upregulated or downregulated known NF-κB target genes towards to the homeostatic levels. Red, upregulated DEGs in IgG-treated CLP mice; Blue, downregulated DEG in IgG-treated CLP mice; Green, the same set of upregulated or downregulated DEGs in trans mAb-treated CLP mice.

FIG. 47 is a diagram showing the rescue of expression of genes converged onto NF-κB-centered pathways at different signaling steps in multiple positive and negative feedback loops by trans mAb treatment. DEGs that were significantly upregulated in CLP mice, but downregulated after trans mAb treatment are highlighted in red, whereas DEGs that were significantly downregulated in CLP mice, but upregulated after trans mAb treatment are highlighted in blue. Positive and negative feedback loops are indicated by red arrow and blue blunt arrow, respectively. DEGs that have been independently validated by IF and/or ELISA are highlighted with yellow background.

FIG. 48 is a series of immunofluorescence images and their corresponding quantification graphs showing the up-regulated expression of CIRBP, THBS1, and CD80 in CLO spleen tissue, n=3.

FIG. 49 is a is a series of immunofluorescence images and their corresponding quantification graphs showing the down-regulated expression of PFN1 and IL-4i1 in CLO spleen tissue, n=3.

FIG. 50 is a diagram showing the rescue of hyperactivation of pro- and anti-inflammatory responses that are conserved between polymicrobial sepsis mice and human COVID-19 patients by trans mAb treatment. Average expression of selected examples, out of 439 common DEGs between sepsis mice and COVID-19 patients, in Sham, CLP₊IgG or CLP₊trans mice are computed using Seurat3 and shown in heatmaps after being normalized to those in CLP₊IgG mice, with the associated simplified gene-ontology terms color coded on the heatmap. Up-regulated pathways are labeled with red arrows and down-regulated pathways are labeled with blue blunt arrows with selected DEGs as examples.

FIG. 51 is a graph showing that trans mAb reverts common DEGs between sepsis mice and COVID-19 patients towards immune homeostasis. Gene ontology analysis of the shared and trans mAb rescued up-regulated DEGs (p=0.05) is shown.

FIG. 52 is a graph showing that trans mAb reverts common DEGs between sepsis mice and COVID-19 patients towards immune homeostasis. Gene ontology analysis of the shared and trans mAb rescued down-regulated DEGs (p=0.05) is shown.

FIG. 53 is a series of fluorescence images showing the induction of trans p65 and THBS1 in the lung of human COVID-19 patients. Autopsy lung tissues from five COVID-19 and healthy controls were assayed by IF for expression of trans p65, and THBS1.

FIGS. 54A-54B are fluorescence images showing the induction of trans p65 in the lung of three human COVID-19 patients. Lung tissues from human normal healthy and COVID-19 patients were subjected to immunofluorescence imaging (FIG. 55A) and quantification (FIG. 55B) for trans p65.

FIGS. 55A-55B are fluorescence images showing the induction of THBS1 in the lung of three human COVID-19 patients. Lung tissues from human normal healthy and COVID-19 patients were subjected to immunofluorescence imaging (FIG. 56A) and quantification (FIG. 56B) for THBS1.

FIG. 56 is a graph showing the reduction of IL-6 release over time in CLP mice after trans mAb treatment. Serum IL-6 levels after CLP mice treated with trans mAb or IgG were detected by ELISA, n=2 per group each time point.

FIG. 57 is a schematic drawing showing the two-hit model of sepsis mice treated with trans mAb or IgG controls followed by intranasal Pseudomonas aeruginosa (PA) administration.

FIG. 58 is a series of graphs showing that trans mAb treatment maintains the number and inhibits the apoptosis of T cells in the thymus in a mouse model of sepsis followed by pneumonia. After non-lethal CLP, mice were treated with trans mAb or control IgG (400 μg/mouse, IV after the surgery and 200 μg/mouse, IP 4 hrs later), followed by intranasal PA administration (50 μL, OD₆₀₀=0.5) 3 days after CLP to induce pneumonia. Apoptotic T cells in the thymus were assayed by the T cell marker CD3 and apoptosis marker Annexin V using FACS at 3 days after CLP surgery was performed (CLP) and 1 day after CLP mice were challenged with PA (CLP₊PA).

FIG. 59 is a series of graphs showing that trans mAb treatment maintains the number and inhibits the apoptosis of T cells in the spleen in a mouse model of sepsis followed by pneumonia. After non-lethal CLP, mice were treated with trans mAb or control IgG (400 μg/mouse, IV after the surgery and 200 μg/mouse, IP 4 hrs later), followed by intranasal PA administration (50 μL, OD₆₀₀=0.5) 3 days after CLP to induce pneumonia. Apoptotic T cells in the spleen were assayed by the T cell marker CD3 and apoptosis marker Annexin V using FACS at 3 days after CLP surgery was performed (CLP) and 1 day after CLP mice were challenged with PA (CLP₊PA).

FIG. 60 is a series of graphs showing that trans mAb treatment maintains the number and inhibits the apoptosis of B cells in the spleen in a mouse model of sepsis followed by pneumonia. After non-lethal CLP, mice were treated with trans mAb or control IgG (400 μg/mouse, IV after the surgery and 200 μg/mouse, IP 4 hrs later), followed by intranasal PA administration (50 μL, OD₆₀₀=0.5) 3 days after CLP to induce pneumonia. Apoptotic B cells in the spleen were assayed by the B cell marker B220 and apoptosis marker Annexin V using FACS at 3 days after CLP surgery was performed (CLP) and 1 day after CLP mice were challenged with PA (CLP₊PA).

FIG. 61 is a series of graphs showing that trans mAb treatment maintains the number and inhibits the apoptosis of CD3₊T cells in the thymus in a mouse model of sepsis followed by pneumonia. After non-lethal CLP, mice were treated with trans mAb or control IgG (400 μg/mouse, IV after the surgery and 200 μg/mouse, IP 4 hrs later), followed by intranasal PA administration (50 μL, OD₆₀₀=0.5) 3 days after CLP to induce pneumonia. Apoptotic CD3₊ T cells in the thymus were assayed by the T cell marker CD3 and apoptosis marker Annexin V using FACS at 3 days after CLP surgery was performed (CLP) and 1 day after CLP mice were challenged with PA (CLP₊PA).

FIG. 62 is a series of graphs showing that trans mAb treatment maintains the number and inhibits the apoptosis of CD3₊ T cells in the spleen in a mouse model of sepsis followed by pneumonia. After non-lethal CLP, mice were treated with trans mAb or control IgG (400 μg/mouse, IV after the surgery and 200 μg/mouse, IP 4 hrs later), followed by intranasal PA administration (50 μL, OD₆₀₀=0.5) 3 days after CLP to induce pneumonia. Apoptotic CD3₊ T cells in the spleen were assayed by the T cell marker CD3 and apoptosis marker Annexin V using FACS at 3 days after CLP surgery was performed (CLP) and 1 day after CLP mice were challenged with PA (CLP₊PA).

FIG. 63 is a series of graphs showing that trans mAb treatment maintains the number and inhibits the apoptosis of B220₊ B cells in the thymus in a mouse model of sepsis followed by pneumonia. After non-lethal CLP, mice were treated with trans mAb or control IgG (400 μg/mouse, IV after the surgery and 200 μg/mouse, IP 4 hrs later), followed by intranasal PA administration (50 μL, OD₆₀₀=0.5) 3 days after CLP to induce pneumonia. Apoptotic B220₊ B cells in the thymus were assayed by the B cell marker B220 and apoptosis marker Annexin V using FACS at 3 days after CLP surgery was performed (CLP) and 1 day after CLP mice were challenged with PA (CLP₊PA).

FIG. 64 is a series of graphs showing the attenuation of the cytokine storm after CLP. Serum IL-6, TNF-α, IL-1β, and IL-10 levels 3 days after CLP were measured using ELISA. The relative values for each cytokine were calculated as a cytokine response to bacterial challenge.

FIG. 65 is a series of graphs showing the attenuation of the cytokine storm after CLP and maintains the ability to produce proinflammatory cytokines after the secondary PA infection. Serum IL-6, TNF-α, IL-1β, and IL-10 levels were measured 1 day after the PA infection following CLP using ELISA. The relative values for each cytokine were calculated as a cytokine response to bacterial challenge.

FIG. 66 is a series of graphs showing the values for each cytokine calculated as a cytokine response to a bacterial challenge (PA).

FIG. 67 is a series of immunofluorescence images showing that trans mAb-treated, but not IgG- or DEX-treated, CLP mice induces nuclear trans p65 NF-κB in response to and recover fully from the secondary PA pneumonia. Mice underwent sham or CLP surgery were treated with trans p65 mAb, DEX or their control IgG or vehicle, and, 3 days later, challenged with intranasal PA, followed by assaying trans p65 in the spleen 20 hr after the PA challenge by immunofluorescence imaging.

FIG. 68 is a graph showing the quantified fluorescence intensity of FIG. 68. The graph shows that trans mAb-treated, but not IgG- or DEX-treated, CLP mice induces nuclear trans p65 NF-κB in response to and recover fully from the secondary PA pneumonia.

FIG. 69 is a series of immunofluorescence images showing that trans mAb-treated, but not IgG- or DEX-treated, CLP mice induces nuclear trans p65 NF-κB in response to and recover fully from the secondary PA pneumonia. Mice underwent sham or CLP surgery were treated with trans p65 mAb, DEX, or their control IgG or vehicle, and, 3 days later, challenged with intranasal PA, followed by immunofluorescence for trans p65 in the lung at 20 hr after the PA challenge.

FIG. 70 is a graph showing that trans mAb-treated, but not IgG- or DEX-treated, CLP mice induces nuclear trans p65 NF-κB in response to and recover fully from the secondary PA pneumonia. Mice underwent sham or CLP surgery were treated with trans p65 mAb, DEX or their control IgG or vehicle, and, 3 days later, challenged with intranasal PA, followed by assaying trans p65 in the spleen 20 hr after the PA challenge by Kaplan Meier survival analysis.

FIG. 71 is a schematic drawing showing a summary for the clinical course of sepsis and therapeutic effects of targeting trans p65 NF-κB.

### DETAILED DESCRIPTION

Described herein are conformation-specific antibodies and antigen-binding fragments thereof that specifically bind to the trans conformation of phosphorylated-Threonine254-Proline (pThr254-Pro) of the p65 subunit of nuclear factor kappa-light-chain-enhancer of activated B cells (NF-κB). References herein to “conformation-specific” NF-kB antibodies and antigen-binding fragments thereof of the invention, unless otherwise indicated by the context, mean antibodies and antigen-binding fragments thereof that bind the trans conformation of phosphorylated-Threonine254-Proline (pThr254-Pro) of the p65 subunit of NF-kB.

The present invention is based, in part, on the surprising discovery that the trans conformation of pThr254-Pro of p65 is favored in the nuclear, active form of NF-κB. Accordingly, antibodies or antigen-binding fragments are described which specifically recognize the active nuclear form, but not the inactive cytoplasmic form, of p65 NF-κB. Antibodies or antigen-binding fragments described herein may inhibit the pathogenic function of dysregulated (e.g., overexpressed) NF-κB, and may be used for the treatment of NF-κB-related diseases (e.g., infection, cancer, or immune or inflammatory disorders, such as sepsis, septic shock, SIRS, or CRS). The invention also provides related pharmaceutical compositions, polynucleotides, vectors, host cells, methods of production, methods of treatment, diagnostic methods, and kits.

### I. Conformation-Specific NF-kB Antibodies or Fragments Thereof

Proline is an amino acid residue unique in its ability to adopt either the cis or trans conformation. Due to the relatively large energy barrier of its isomerization (εu=14 to 24 kcal mol⁻¹), uncatalyzed isomerization is a slow process, but may be accelerated by enzymes, such as isomerases. Cis-trans isomerization of the peptidyl-prolyl bond can regulate the folding and therefore biological activity of a protein or polypeptides (e.g., NF-kB, for example the p65 subunit of NF-kB), and therefore cis-trans isomerization may affect, for example, growth-signal responses, cell-cycle progression, cellular stress responses, neuronal function, and immune responses.

NF-κB is a protein complex that controls transcription of DNA, cytokine production, and cell survival. NF-κB is found in almost all animal cell types and is involved in cellular responses to stimuli such as stress, cytokines, free radicals, heavy metals, ultraviolet irradiation, oxidized LDL, and bacterial or viral antigens. NF-κB plays a key role in regulating the immune response to infection. Incorrect regulation of NF-κB has been linked to disorders, including, but not limited to cancer, inflammatory and immune diseases (e.g., autoimmune diseases), sepsis, septic shock, infection (e.g., viral infection), and improper immune development. NF-κB regulates the expression of a large number of genes including but not limited to: IGHG4, IGHG3, APOC3, TNFRSF6, CD3G, TNFSF5, CD105, ICAM1, TPMT, IL2RA, SELE, TP53, CRP, IL1A, IL1B, IL1RN, CCR5, IL8, IL2, IL9, TAP1, TNF, LTA, IL6, CD44, NOS2A, SOD2, TNFSF6, IL11, BDKRB1, CSF1, CSF2, CSF3, GSTP1, NQO1, OPRM1, PTAFR, PTGS2, SCNN1A, VCAM1, AGER, ALOX12B, BCL2L1, TNFRSF5, TNFRSF9, IRF7, BLR1, CD48, CD69, CCR7, CR2, F3, HMOX1, TNC, IFNB1, IL13, IL15RA, IRF1, IRF2, LTB, IRF4, MYC, NFKB2, PDGFB, PLAU, LMP2, PTX3, CCL2, CCL5, CCL11, CXCL5, SELP, SLC2A5, STAT5A, VIM, IER3, NFKB1, BM2, BCL2A1, CCL15, CD83, CD74, ELF3, TGM2, DEFB4, MMP9, BCL3, CD80, VEGFC, PLCD1, TNFAIP3, RELB, TFPI2, BCL2, S100A6, TACR1, NFKBIA, CD209, CARD15, CCND1, KLK3, IL15, NR4A2, and HC3.

NF-κB is a homo- or heterodimeric complex formed by the Rel-like domain-containing proteins RELA/p65, RELB, NFKB1/p105, NFKB1/p50, REL and NFKB2/p52. The dimers bind at NF-κB sites in the DNA of their target genes and the individual dimers have distinct preferences for different kappa-B sites that they can bind with distinguishable affinity and specificity. Different dimer combinations act as transcriptional activators or repressors, respectively. NF-κB is controlled by various mechanisms of post-translational modification and subcellular compartmentalization as well as by interactions with other cofactors or corepressors. NF-κB complexes are held in the cytoplasm in an inactive state complexed with members of the NF-κB inhibitor (IκB) family. In a conventional activation pathway, IκB is phosphorylated by IκB kinases (IKKs) in response to different activators, subsequently degraded thus liberating the active NF-κB complex which translocates to the nucleus.

The present invention is based, in part, on the surprising discovery that the trans conformation of pThr254-Pro of p65 is favored in the nuclear, active form of NF-κB. Therefore, specifically inhibiting NF-κB having trans-pThr254-Pro of p65 is useful in the treatment of disorders associated with abherant NF-κB activity.

### Conformation-Specific NF-κB Antibodies or Fragments Thereof

Described herein are methods and compositions for the generation and use of conformation-specific NF-κB antibodies or fragments thereof. Conformation-specific antibodies or fragments thereof recognize and specifically bind to a particular conformation (e.g., a conformational isomer or conformer) of its complementary antigen. For example, as described herein, conformation-specific antibodies may specifically bind to the trans conformation of a pThr-Pro motif (e.g., binds preferentially to the trans conformation as compared to the cis conformation of the pThr-Pro motif). In particular, antibodies described herein bind specifically to an epitope including trans-pTh254-Pro of the p65 subunit of NF-κB (e.g., relative to an epitope including cis-pTh254-Pro of the p65 subunit of NF-κB). A conformation specific antibody may have at least 2-fold (e.g., at least 5-fold, 10-fold, 20-fold, 30-fold, 40-fold, 50-fold, 60-fold, 70-fold, 80-fold, 90-fold, 100-fold, 200-fold, 500-fold, or 1000-fold) greater affinity for the trans conformation of pThr254-Pro relative to the cis conformation of pThr254-Pro. The conformation-specific antibody may have at least 10- to 100-fold greater affinity to the the trans conformation of pThr254-Pro relative to the cis conformation of pThr254-Pro.

Particularly, the disclosure features a conformation-specific NF-κB antibody or antigen-binding fragment thereof that contains one or more, or all, of the CDR sequences of a trans-mAb, such as a human, humanized, or chimeric variant of a trans-mAb, to a human or a non-human mammal in order to treat a cancer. The following trans-mAbs were produced according to the methods described herein.

### Trans-mAb1

### Trans-mAb1 Light Chain

Trans-mAb1 includes a complementarity-determining region (CDR) light chain 1 (CDR-L1) having the amino acid sequence of RSSQSIVHSNGNTYLE (SEQ ID NO: 1); a CDR light chain 2 (CDR-L2) having the amino acid sequence of KVSNRFS (SEQ ID NO: 2); and a CDR light chain 3 (CDR-L3) having the amino acid sequence of FQGAHVPLT (SEQ ID NO: 3).

Trans-mAb1 includes a light chain variable domain having an amino acid sequence of

(SEQ ID NO: 4; CDRs underlined)
DVLMTQTPLSLPVSLGDQASISCRSSQSIVHSNGNTYLEWYLQKPGQSPK
LLIYKVSNRFSGVPDRFSGSGSGTDFTLKISRVEAEDLGVYYCFQGAHVP
LTFGAGTKLELK.

### Trans-mAb 1 Heavy Chain

Trans-mAb1 includes a complementarity-determining region (CDR) heavy chain 1 (CDR-H1) having the amino acid sequence of TNAMN (SEQ ID NO: 5); a CDR heavy chain 2 (CDR-H2) having the amino acid sequence of RIRSKRNNYATYYADSVKD (SEQ ID NO: 6); and a CDR heavy chain 3 (CDR-H3) having the amino acid sequence of DGLGMDY (SEQ ID NO: 7).

Trans-mAb1 includes a heavy chain variable domain having an amino acid sequence of

(SEQ ID NO: 8; CDRs underlined)
EVQLVETGGGLVQPKGSLKLSCAASGFTFNTNAMNWVRQAPGKGLEWVAR
IRSKRNNYATYYADSVKDRFTISRDESQNMLFLQMNNLKTEDTAMYYCVR
DGLGMDYWGQGTSVTVSS.

### Trans-mAb2

### Trans-mAb2 Light Chain

Trans-mAb2 includes a complementarity-determining region (CDR) light chain 1 (CDR-L1) having the amino acid sequence of SEQ ID NO: 1; a CDR light chain 2 (CDR-L2) having the amino acid sequence of SEQ ID NO: 2; and a CDR light chain 3 (CDR-L3) having the amino acid sequence of SEQ ID NO: 3.

Trans-mAb2 includes a light chain variable domain having an amino acid sequence of SEQ ID NO: 4.

### Trans-mAb2 Heavy Chain

Trans-mAb2 includes a complementarity-determining region (CDR) heavy chain 1 (CDR-H1) having the amino acid sequence of SEQ ID NO: 5; a CDR heavy chain 2 (CDR-H2) having the amino acid sequence of RIRSKSNNYATYYADSVKD (SEQ ID NO: 9); and a CDR heavy chain 3 (CDR-H3) having the amino acid sequence of SEQ ID NO: 7.

Trans-mAb2 includes a heavy chain variable domain having an amino acid sequence of

(SEQ ID NO: 10; CDRs underlined)
EVQLVETGGGLVQPKGSLKLSCAASGFTFNTNAMNWVRQAPGKGLEWVAR
IRSKSNNYATYYADSVKDRFTISRDESQNMLFLQMNNLKTEDTAMYYCVR
DGLGMDYWGQGTSVTVSS.

### Trans-mAb3

### Trans-mAb3 Light Chain

Trans-mAb3 includes a complementarity-determining region (CDR) light chain 1 (CDR-L1) having the amino acid sequence of RSSQSIVHSNGHTYLE (SEQ ID NO: 11); a CDR light chain 2 (CDR-L2) having the amino acid sequence of SEQ ID NO: 2; and a CDR light chain 3 (CDR-L3) having the amino acid sequence of SEQ ID NO: 3.

Trans-mAb3 includes a light chain variable domain having an amino acid sequence of

(SEQ ID NO: 12; CDRs underlined)
DVLMTQTPLSLPVSLGDQASISCRSSQSIVHSNGHTYLEWYLQKPGQSPK
LLIYKVSNRFSGVPDRFSGSGSGTDFTLKISRVEAEDLGVYYCFQGAHVP
LTFGAGTKLELK.

### Trans-mAb3 Heavy Chain

Trans-mAb3 includes a complementarity-determining region (CDR) heavy chain 1 (CDR-H1) having the amino acid sequence of SEQ ID NO: 5; a CDR heavy chain 2 (CDR-H2) having the amino acid sequence of SEQ ID NO: 6; and a CDR heavy chain 3 (CDR-H3) having the amino acid sequence of SEQ ID NO: 7.

Trans-mAb3 includes a heavy chain variable domain having an amino acid sequence of SEQ ID NO: 8.

### Trans-mAb4

### Trans-mAb4 Light Chain

Trans-mAb4 includes a complementarity-determining region (CDR) light chain 1 (CDR-L1) having the amino acid sequence of SEQ ID NO: 11; a CDR light chain 2 (CDR-L2) having the amino acid sequence of SEQ ID NO: 2; and a CDR light chain 3 (CDR-L3) having the amino acid sequence of SEQ ID NO: 3.

Trans-mAb4 includes a light chain variable domain having an amino acid sequence of SEQ ID NO: 12.

### Trans-mAb4 Heavy Chain

Trans-mAb4 includes a complementarity-determining region (CDR) heavy chain 1 (CDR-H1) having the amino acid sequence of SEQ ID NO: 5; a CDR heavy chain 2 (CDR-H2) having the amino acid sequence of SEQ ID NO: 9; and a CDR heavy chain 3 (CDR-H3) having the amino acid sequence of SEQ ID NO: 7.

Trans-mAb4 includes a heavy chain variable domain having an amino acid sequence of SEQ ID NO: 10.

### Trans-mAb5

### Trans-mAb5 Light Chain

Trans-mAb5 includes a complementarity-determining region (CDR) light chain 1 (CDR-L1) having the amino acid sequence of RSSKSVSTSGYSYML (SEQ ID NO: 13); a CDR light chain 2 (CDR-L2) having the amino acid sequence of LVSNLEC (SEQ ID NO: 14); and a CDR light chain 3 (CDR-L3) having the amino acid sequence of QHIRELTRS (SEQ ID NO: 15).

Trans-mAb5 includes a light chain variable domain having an amino acid sequence of

(SEQ ID NO: 16; CDRs underlined)
DIVVTQCRGSLDVSLGQRATISYRSSKSVSTSGYSYMLWNQQKSGQPPRL
LMYLVSNLECGVRARFSGSGYGTEFTLNFHHVEEEDVATYSSQHIRELTR
SEGGPSWKZN.

### Trans-mAb5 Heavy Chain

Trans-mAb5 includes a complementarity-determining region (CDR) heavy chain 1 (CDR-H1) having the amino acid sequence of SEQ ID NO: 5; a CDR heavy chain 2 (CDR-H2) having the amino acid sequence of SEQ ID NO: 6; and a CDR heavy chain 3 (CDR-H3) having the amino acid sequence of SEQ ID NO: 7.

Trans-mAb5 includes a heavy chain variable domain having an amino acid sequence of SEQ ID NO: 8.

### Trans-mAb6

### Trans-mAb6 Light Chain

Trans-mAb6 includes a complementarity-determining region (CDR) light chain 1 (CDR-L1) having the amino acid sequence of SEQ ID NO: 13; a CDR light chain 2 (CDR-L2) having the amino acid sequence of SEQ ID NO: 14; and a CDR light chain 3 (CDR-L3) having the amino acid sequence of SEQ ID NO: 15.

Trans-mAb6 includes a light chain variable domain having an amino acid sequence of SEQ ID NO: 16.

### Trans-mAb6 Heavy Chain

Trans-mAb6 includes a complementarity-determining region (CDR) heavy chain 1 (CDR-H1) having the amino acid sequence of SEQ ID NO: 5; a CDR heavy chain 2 (CDR-H2) having the amino acid sequence of SEQ ID NO: 9; and a CDR heavy chain 3 (CDR-H3) having the amino acid sequence of SEQ ID NO: 7.

Trans-mAb6 includes a heavy chain variable domain having an amino acid sequence of SEQ ID NO: 10.

### Trans-mAb7

### Trans-mAb7 Light Chain

Trans-mAb7 includes a complementarity-determining region (CDR) light chain 1 (CDR-L1) having the amino acid sequence of RASKSVSTSGYSYMH (SEQ ID NO: 17); a CDR light chain 2 (CDR-L2) having the amino acid sequence of LVSNLES (SEQ ID NO: 18); and a CDR light chain 3 (CDR-L3) having the amino acid sequence of SEQ ID NO: 15.

Trans-mAb7 includes a light chain variable domain having an amino acid sequence of

(SEQ ID NO: 19; CDRs underlined)
DIVLTQSPASLAVSLGQRATISYRASKSVSTSGYSYMHWNQQKPGQPPRL
LIYLVSNLESGVPARFSGSGSGTDFTLNIHPVEEEDAATYYCQHIRELTR
SEGGPSWKZN.

### Trans-mAb7 Heavy Chain

Trans-mAb7 includes a complementarity-determining region (CDR) heavy chain 1 (CDR-H1) having the amino acid sequence of SEQ ID NO: 5; a CDR heavy chain 2 (CDR-H2) having the amino acid sequence of SEQ ID NO: 6; and a CDR heavy chain 3 (CDR-H3) having the amino acid sequence of SEQ ID NO: 7.

Trans-mAb7 includes a heavy chain variable domain having an amino acid sequence of SEQ ID NO: 8.

### Trans-mAb8

### Trans-mAb8 Light Chain

Trans-mAb8 includes a complementarity-determining region (CDR) light chain 1 (CDR-L1) having the amino acid sequence of SEQ ID NO: 17; a CDR light chain 2 (CDR-L2) having the amino acid sequence of SEQ ID NO: 18; and a CDR light chain 3 (CDR-L3) having the amino acid sequence of SEQ ID NO: 15.

Trans-mAb8 includes a light chain variable domain having an amino acid sequence of SEQ ID NO: 19.

### Trans-mAb8 Heavy Chain

Trans-mAb8 includes a complementarity-determining region (CDR) heavy chain 1 (CDR-H1) having the amino acid sequence of SEQ ID NO: 5; a CDR heavy chain 2 (CDR-H2) having the amino acid sequence of SEQ ID NO: 9; and a CDR heavy chain 3 (CDR-H3) having the amino acid sequence of SEQ ID NO: 7.

Trans-mAb8 includes a heavy chain variable domain having an amino acid sequence of SEQ ID NO: 10.

### Trans-mAb9

### Trans-mAb9 Light Chain

Trans-mAb9 includes a complementarity-determining region (CDR) light chain 1 (CDR-L1) having the amino acid sequence of KASQNVGTNVA (SEQ ID NO: 20); a CDR light chain 2 (CDR-L2) having the amino acid sequence of SASYRYS (SEQ ID NO: 21); and a CDR light chain 3 (CDR-L3) having the amino acid sequence of QQYNSYPYT (SEQ ID NO: 22).

Trans-mAb9 includes a light chain variable domain having an amino acid sequence of

(SEQ ID NO: 23; CDRs underlined)
DIVMTQSQKFMSTSVGDRVSVTCKASQNVGTNVAWYQQKPGQSPKALIYS
ASYRYSGVPDRFTGSGSGTDFTLTISNVQSEDLAEYFCQQYNSYPYTFGA
GTKLELK.

### Trans-mAb9 Heavy Chain

Trans-mAb9 includes a complementarity-determining region (CDR) heavy chain 1 (CDR-H1) having the amino acid sequence of SEQ ID NO: 5; a CDR heavy chain 2 (CDR-H2) having the amino acid sequence of SEQ ID NO: 6; and a CDR heavy chain 3 (CDR-H3) having the amino acid sequence of SEQ ID NO: 7.

Trans-mAb9 includes a heavy chain variable domain having an amino acid sequence of SEQ ID NO: 8.

### Trans-mAb10

### Trans-mAb 10 Light Chain

Trans-mAb10 includes a complementarity-determining region (CDR) light chain 1 (CDR-L1) having the amino acid sequence of SEQ ID NO: 20; a CDR light chain 2 (CDR-L2) having the amino acid sequence of SEQ ID NO: 21; and a CDR light chain 3 (CDR-L3) having the amino acid sequence of SEQ ID NO: 22.

Trans-mAb10 includes a light chain variable domain having an amino acid sequence of SEQ ID NO: 23.

### Trans-mAb 10 Heavy Chain

Trans-mAb10 includes a complementarity-determining region (CDR) heavy chain 1 (CDR-H1) having the amino acid sequence of SEQ ID NO: 5; a CDR heavy chain 2 (CDR-H2) having the amino acid sequence of SEQ ID NO: 9; and a CDR heavy chain 3 (CDR-H3) having the amino acid sequence of SEQ ID NO: 7.

Trans-mAb10 includes a heavy chain variable domain having an amino acid sequence of SEQ ID NO: 10.

### Trans-mAb CDR Consensus Sequences

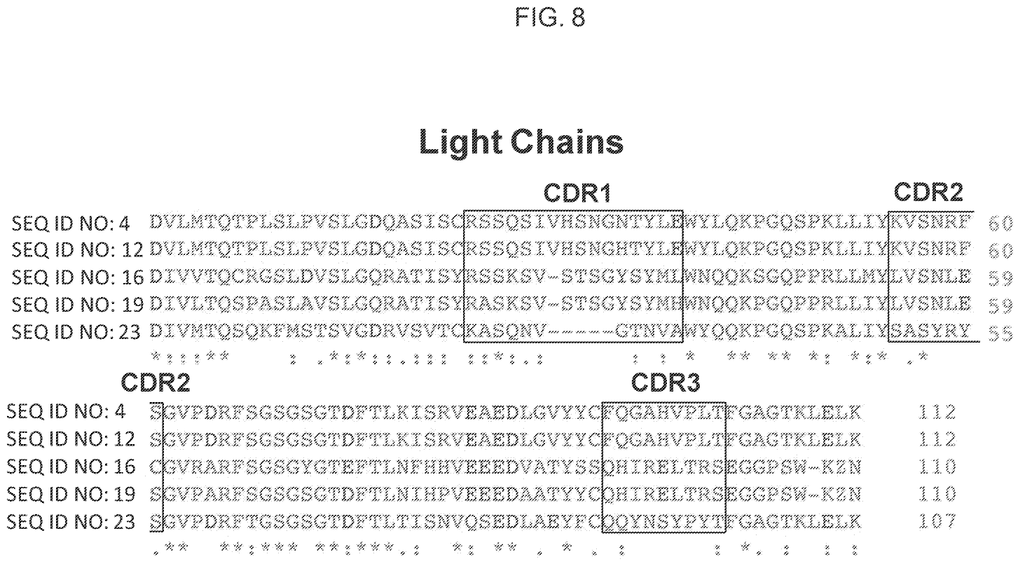

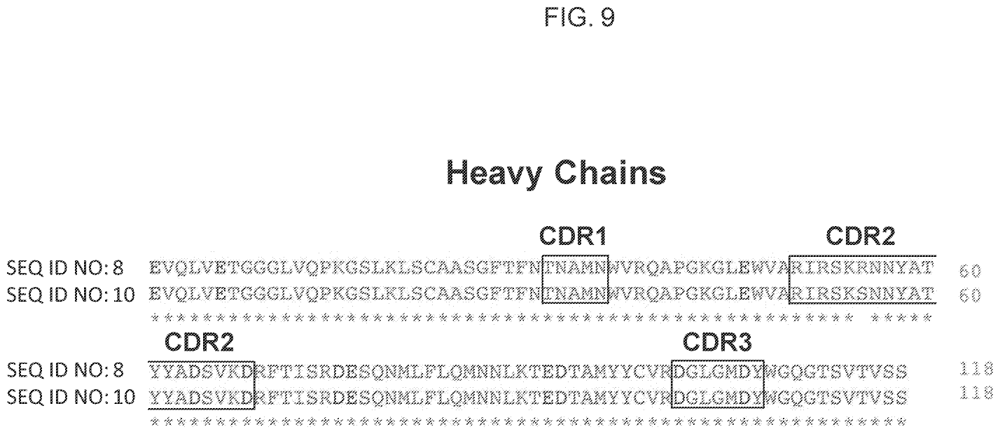

A concensus sequence for each of CDR-L1, CDR-L2, CDR-L3, CDR-H1, CDR-H2, and CDR-H3 may be generated in consideration of the above sequences for trans-mAb1 through trans-mAb10 (see also the sequence alignments for trans-mAb light chains (FIG. 8) and trans-mAb heavy chains (FIG. 9)). The invention provides an antibody or antigen binding fragment thereof comprising one or more of the following concensus CDR sequences.

CDR-L1 concensus sequence
(SEQ ID NO: 24)
X₁X₂X₃X₄X₅X₆X₇X₈X₉X₁₀X₁₁X₁₂X₁₃X₁₄X₁₅X₁₆

where X₁ is R or K; X₂ is S or A; X₃ is S; X₄ is Q or K; X₅ is S or N; X₆ is I or V; X₇ is V or absent; X₈ is H, S, or absent; X₉ is S, T, or absent; X₁₀ is N, S, or absent; X₁₁ is G or absent; X₁₂ is N, H, Y, or G; X₁₃ is T or S; X₁₄ is Y or N, X₁₅ is L, M, or V, and X₁₆ is E, L, H, or A.

CDR-L2 concensus sequence
(SEQ ID NO: 25)
X₁X₂X₃X₄X₅X₆X₇

where X₁ is K, L, or S; X₂ is V or A; X₃ is S; X₄ is N or Y; X₅ is R or L; X₆ is F, E, or Y; X₇ is S or C.

CDR-L3 concensus sequence
(SEQ ID NO: 26)
X₁X₂X₃X₄X₅X₆X₇X₈X₉

where X₁ is F or Q; X₂ is Q or H; X₃ is G, I, or Y; X₄ is A, R, or N; X₅ is H, E, or S; X₆ is V, L, or Y; X₇ is P or T; X₈ is L, R, or Y; and X₉ is T or S.

### CDR-H1 Concensus Sequence (SEQ ID NO: 5)

The concensus sequence for CDR-H1 corresponds to SEQ ID NO: 5, which is present in both heavy chain variable domains corresponding to SEQ ID NO: 8 and SEQ ID NO: 10.

CDR-H2 concensus sequence
(SEQ ID NO: 27)
RIRSKXNNYATYYADSVKD

where X is R or S.

### CDR-H3 Concensus Sequence (SEQ ID NO: 7)

The concensus sequence for CDR-H3 corresponds to SEQ ID NO: 7, which is present in both heavy chain variable domains corresponding to SEQ ID NO: 8 and SEQ ID NO: 10.

### Fully Human, Humanized, Primatized, and Chimeric Antibodies

Antibodies described herein include fully human, humanized, primatized, and chimeric antibodies. Additionally, antibodies described herein include fully human, humanized, primatized, and chimeric antibodies that contain one or more, or all, of the CDR-H1, CDR-H2, CDR-H3, CDR-L1, CDR-L2, and CDR-L3 sequences described herein in which one or more, or all, of the CDR sequences exhibits at least 70% sequence identity (e.g., 75%, 80%, 85%, 90%, 95%, 96%, 97%, 98%, 99%, or 100% sequence identity) to the corresponding CDR sequence of a trans-mAb described herein (e.g., any one of trans-mAb1, trans-mAb2, trans-mAb3, trans-mAb4, trans-mAb5, trans-mAb6, trans-mAb7, trans-mAb8, trans-mAb9, trans-mAb10, or a concensus sequence thereof). Conformation-specific NF-κB antibodies described herein further include fully human, humanized, primatized, and chimeric antibodies that contain one or more, or all, of the CDR-H1, CDR-H2, CDR-H3, CDR-L1, CDR-L2, and CDR-L3 sequences in which one or more, or all, of the CDR sequences contains one or more (for instance, up to 3) amino acid substitutions (e.g., one or more conservative amino acid substitutions) relative to the corresponding CDR sequence of a trans-mAb described herein. For example, conformation-specific NF-κB antibodies described herein can be generated by incorporating any one or more of the CDR sequences of a trans-mAb described herein into the framework regions (e.g., FW1, FW2, FW3, and FW4) of a human antibody. Exemplary framework regions that can be used for the development of a humanized anti-NF-κB antibody containing one or more of the CDRs of a trans-mAb described herein include, without limitation, those described in U.S. Pat. Nos. 7,732,578, 8,093,068, and WO 2003/105782; the disclosures of each of which are incorporated herein by reference.

As an example, one strategy that can be used to design humanized antibodies described herein is to align the sequences of the heavy chain variable region and light chain variable region of a trans-mAb described herein with the heavy chain variable region and light chain variable region of a consensus human antibody. Consensus human antibody heavy chain and light chain sequences are known in the art (see e.g., the “VBASE” human germline sequence database; see also Kabat, et al., Sequences of Proteins of Immunological Interest, Fifth Edition, U.S. Department of Health and Human Services, NIH Publication No. 91-3242, 1991; Tomlinson et al., J. Mol. Biol. 227:776-98, 1992; and Cox et al, Eur. J. Immunol. 24:827-836, 1994; the disclosure of which is incorporated herein by reference). In this way, the variable domain framework residues and CDRs can be identified by sequence alignment (see Kabat, supra). One can substitute, for example, one or more of the CDRs of the consensus human antibody with the corresponding CDR(s) of a trans-mAb described herein, in order to produce a humanized NF-κB antagonist antibody. Exemplary variable domains of a consensus human antibody include the heavy chain variable domain are identified in U.S. Pat. No. 6,054,297; the disclosure of which is incorporated herein by reference. These amino acid substitutions can be made, for example, by recombinant expression of polynucleotides encoding the heavy and light chains of a humanized antibody in a host cell using methods known in the art or described herein.

Similarly, this strategy can also be used to produce primatized conformation-specific NF-κB antibodies, as one can substitute, for example, one or more, or all, of the CDRs of a primate antibody consensus sequence with, for example, one or more, or all, of the CDRs of a trans-mAb described herein. Consensus primate antibody sequences known in the art (see e.g., U.S. Pat. Nos. 5,658,570; 5,681,722; and 5,693,780; the disclosures of each of which are incorporated herein by reference).

In some embodiments, it may be desirable to import particular framework residues in addition to CDR sequences from a conformation-specific NF-κB antibody, such as a trans-mAb described herein, into the heavy and/or light chain variable domains of a human antibody. For instance, U.S. Pat. No. 6,054,297 identifies several instances when it may be advantageous to retain certain framework residues from a particular antibody heavy chain or light chain variable region in the resulting humanized antibody. In some embodiments, framework residues may engage in non-covalent interactions with the antigen and thus contribute to the affinity of the antibody for the target antigen. In some embodiments, individual framework residues may modulate the conformation of a CDR, and thus indirectly influence the interaction of the antibody with the antigen. Certain framework residues may form the interface between VH and VL domains and may therefore contribute to the global antibody structure. In some cases, framework residues may constitute functional glycosylation sites (e.g., Asn-X-Ser/Thr) which may dictate antibody structure and antigen affinity upon attachment to carbohydrate moieties. In cases such as those described above, it may be beneficial to retain certain framework residues of a conformation-specific NF-κB antibody (e.g., a trans-mAb described herein in, e.g., a humanized or primatized antagonistic antibody or antigen-binding fragment thereof, as various framework residues may promote high epitope affinity and improved biochemical activity of the antibody or antigen-binding fragment thereof.

Antibodies described herein also include antibody fragments, Fab domains, F(ab′) molecules, F(ab′)₂ molecules, single-chain variable fragments (scFvs), tandem scFv fragments, diabodies, triabodies, dual variable domain immunoglobulins, multi-specific antibodies, bispecific antibodies, and heterospecific antibodies that contain one or more, or all, of the CDRs of a trans-mAb described herein, or one or more, or all, of the CDR-H1, CDR-H2, CDR-H3, CDR-L1, CDR-L2, and CDR-L3 sequences in which one or more, or all, of the CDR sequences exhibits at least 70% sequence identity (e.g., 75%, 80%, 85%, 90%, 95%, 96%, 97%, 98%, 99%, or 100% sequence identity) to the corresponding CDR sequence of a trans-mAb described herein. Conformation-specific NF-κB antibodies described herein further include fully human, humanized, primatized, and chimeric antibodies that contain one or more, or all, of the CDR-H1, CDR-H2, CDR-H3, CDR-L1, CDR-L2, and CDR-L3 sequences in which one or more, or all, of the CDR sequences contains one or more (for instance, up to 3) amino acid substitutions (e.g., one or more conservative amino acid substitutions) relative to the corresponding CDR sequence of a trans-mAb described herein. These molecules can be expressed recombinantly, e.g., by incorporating polynucleotides encoding these proteins into expression vectors for transfection in a eukaryotic or prokaryotic cell using techniques described herein or known in the art, or synthesized chemically, e.g., by solid phase peptide synthesis methods described herein or known in the art.

Polypeptides described herein additionally include antibody-like scaffolds that contain, for example, one or more, or all, of the CDRs of a trans-mAb described herein, or one or more, or all, of the CDR-H1, CDR-H2, CDR-H3, CDR-L1, CDR-L2, and CDR-L3 sequences in which one or more, or all, of the CDR sequences exhibits at least 70% sequence identity (e.g., 75%, 80%, 85%, 90%, 95%, 96%, 97%, 98%, 99%, or 100% sequence identity) to the corresponding CDR sequence of a trans-mAb described herein or contains one or more (for instance, up to 3) amino acid substitutions (e.g., one or more conservative amino acid substitutions) relative to the corresponding CDR sequence of a trans-mAb described herein. Examples of antibody-like scaffolds include proteins that contain a tenth fibronectin type III domain (¹⁰Fn3), which contains BC, DE, and FG structural loops analogous to canonical antibodies. The tertiary structure of the ¹⁰Fn3 domain resembles that of the variable region of the IgG heavy chain, and one of skill in the art can graft, e.g., one or more, or all, of the CDR sequences of a trans-mAb described herein or sequences having at least 70% sequence identity (e.g., 75%, 80%, 85%, 90%, 95%, 97%, 99%, or 100% sequence identity) to any one or more of these CDR sequences or sequences containing amino acid substitutions, such as conservative or nonconservative amino acid substitutions (e.g., up to 3 amino acid substitutions) relative to one or more of these CDR sequences onto the fibronectin scaffold by replacing residues of the BC, DE, and FG loops of ¹⁰Fn3 with residues of the corresponding CDR sequence of a trans-mAb described herein. This can be achieved by recombinant expression of a modified ¹⁰Fn3 domain in a prokaryotic or eukaryotic cell (e.g., using the vectors and techniques described herein). Examples of using the ¹⁰Fn3 domain as an antibody-like scaffold for the grafting of CDRs from antibodies onto the BC, DE, and FG structural loops are reported in WO 2000/034784, WO 2009/142773, WO 2012/088006, and U.S. Pat. No. 8,278,419; the disclosures of each of which are incorporated herein by reference.

### II. Nucleic Acids and Expression Systems

Conformation-specific NF-κB antibodies or antigen-binding fragments thereof described herein can be prepared by any of a variety of established techniques. For instance, a conformation-specific NF-κB antibody or antigen-binding fragment thereof described herein can be prepared by recombinant expression of immunoglobulin light and heavy chain genes in a host cell. To express an antibody recombinantly, a host cell can be transfected with one or more recombinant expression vectors carrying DNA fragments encoding the immunoglobulin light and heavy chains of the antibody such that the light and heavy chains are expressed in the host cell and, optionally, secreted into the medium in which the host cells are cultured, from which medium the antibodies can be recovered. Standard recombinant DNA methodologies are used to obtain antibody heavy and light chain genes, incorporate these genes into recombinant expression vectors and introduce the vectors into host cells, such as those described in Molecular Cloning; A Laboratory Manual, Second Edition (Sambrook, Fritsch and Maniatis (eds), Cold Spring Harbor, N. Y., 1989), Current Protocols in Molecular Biology (Ausubel et al., eds., Greene Publishing Associates, 1989), and in U.S. Pat. No. 4,816,397; the disclosures of each of which are incorporated herein by reference.

### Vectors for Expression of Conformation-Specific NF-κB Antibodies

Viral genomes provide a rich source of vectors that can be used for the efficient delivery of exogenous genes into the genome of a cell (e.g., a eukaryotic or prokaryotic cell). Viral genomes are particularly useful vectors for gene delivery because the polynucleotides contained within such genomes are typically incorporated into the genome of a target cell by generalized or specialized transduction. These processes occur as part of the natural viral replication cycle, and do not require added proteins or reagents in order to induce gene integration. Examples of viral vectors include a retrovirus, adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses useful for delivering polynucleotides encoding antibody light and heavy chains or antibody fragments described herein include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D-type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in McVey et al., (U.S. Pat. No. 5,801,030); the disclosures of each of which are incorporated herein by reference.

### Genome Editing Techniques

In addition to viral vectors, a variety of additional methods have been developed for the incorporation of genes, e.g., those encoding antibody light and heavy chains, single-chain polypeptides, single-chain variable fragments (scFvs), tandem scFvs, Fab domains, F(ab′)₂ domains, diabodies, and triabodies, among others, into the genomes of target cells for polypeptide expression. One such method that can be used for incorporating polynucleotides encoding conformation-specific NF-κB antibodies or fragments thereof into prokaryotic or eukaryotic cells includes transposons. Transposons are polynucleotides that encode transposase enzymes and contain a polynucleotide sequence or gene of interest flanked by excision sites at the 5′ and 3′ positions. Once a transposon has been delivered into a cell, expression of the transposase gene commences and results in active enzymes that cleave the gene of interest from the transposon. This activity is mediated by the site-specific recognition of transposon excision sites by the transposase. In some embodiments, these excision sites may be terminal repeats or inverted terminal repeats. Once excised from the transposon, the gene of interest can be integrated into the genome of a prokaryotic or eukaryotic cell by transposase-catalyzed cleavage of similar excision sites that exist within nuclear genome of the cell. This allows the gene encoding a conformation-specific NF-κB antibody or fragment or domain thereof to be inserted into the cleaved nuclear DNA at the excision sites, and subsequent ligation of the phosphodiester bonds that join the gene of interest to the DNA of the prokaryotic or eukaryotic cell genome completes the incorporation process. In some embodiments, the transposon may be a retrotransposon, such that the gene encoding the antibody is first transcribed to an RNA product and then reverse-transcribed to DNA before incorporation in the prokaryotic or eukaryotic cell genome. Exemplary transposon systems include the piggyback transposon (described in detail in WO 2010/085699) and the sleeping beauty transposon (described in detail in US20050112764); the disclosures of each of which are incorporated herein by reference.

Another useful method for the integration of nucleic acid molecules encoding conformation-specific NF-κB antibodies or fragments thereof into the genome of a prokaryotic or eukaryotic cell is the clustered regularly interspaced short palindromic repeats (CRISPR)/Cas system, which is a system that originally evolved as an adaptive defense mechanism in bacteria and archaea against infection by viruses. The CRISPR/Cas system consists of palindromic repeat sequences within plasmid DNA and an associated Cas9 nuclease. This ensemble of DNA and protein directs site specific DNA cleavage of a target sequence by first incorporating foreign DNA into CRISPR loci. Polynucleotides containing these foreign sequences and the repeat-spacer elements of the CRISPR locus are in turn transcribed in a host cell to create a guide RNA, which can subsequently anneal to a target sequence and localize the Cas9 nuclease to this site. In this manner, highly site-specific cas9-mediated DNA cleavage can be engendered in a foreign polynucleotide because the interaction that brings cas9 within close proximity of the target DNA molecule is governed by RNA:DNA hybridization. As a result, one can theoretically design a CRISPR/Cas system to cleave any target DNA molecule of interest. This technique has been exploited in order to edit eukaryotic genomes (Hwang et al., Nat. Biotech., 31:227-229, 2013) and can be used as an efficient means of site-specifically editing eukaryotic or prokaryotic genomes in order to cleave DNA prior to the incorporation of a polynucleotide encoding a conformation-specific NF-κB antibody or fragment thereof described herein. The use of CRISPR/Cas to modulate gene expression has been described in U.S. Pat. No. 8,697,359, the disclosure of which is incorporated herein by reference.

Alternative methods for site-specifically cleaving genomic DNA prior to the incorporation of a polynucleotide encoding a conformation-specific NF-κB antibody or fragment thereof described herein include the use of zinc finger nucleases and transcription activator-like effector nucleases (TALENs). Unlike the CRISPR/Cas system, these enzymes do not contain a guiding polynucleotide to localize to a specific target sequence. Target specificity is instead controlled by DNA binding domains within these enzymes. Zinc finger nucleases and TALENs for use in genome editing applications are described in Urnov et al. (Nat. Rev. Genet., 11:636-646, 2010); and in Joung et al., (Nat. Rev. Mol. Cell. Bio. 14:49-55, 2013); incorporated herein by reference. Additional genome editing techniques that can be used to incorporate polynucleotides encoding antibodies described herein into the genome of a prokaryotic or eukaryotic cell include the use of ARCUS™ meganucleases that can be rationally designed so as to site-specifically cleave genomic DNA. The use of these enzymes for the incorporation of polynucleotides encoding conformation-specific NF-κB antibodies or fragments thereof described herein into the genome of a prokaryotic or eukaryotic cell is particularly advantageous in view of the structure-activity relationships that have been established for such enzymes. Single-chain meganucleases can thus be modified at certain amino acid positions in order to create nucleases that selectively cleave DNA at desired locations. These single-chain nucleases have been described extensively, e.g., in U.S. Pat. Nos. 8,021,867 and 8,445,251; the disclosures of each of which are incorporated herein by reference.

### Polynucleotide Sequence Elements

To express conformation-specific NF-κB antibodies or fragments thereof described herein, polynucleotides encoding partial or full-length light and heavy chains, e.g., polynucleotides that encode a one or more, or all, of the CDR sequences of an antibody or antigen-binding fragment thereof described herein, can be inserted into expression vectors such that the genes are operatively linked to transcriptional and translational control sequences. The expression vector and expression control sequences are chosen to be compatible with the expression host cell used. Polynucleotides encoding the light chain gene and the heavy chain of a conformation-specific NF-κB antibody or fragment thereof can be inserted into separate vectors, or, optionally, both polynucleotides can be incorporated into the same expression vector using established techniques described herein or known in the art.

In addition to polynucleotides encoding the heavy and light chains of an antibody (or a polynucleotide encoding a single-chain polypeptide, an antibody fragment, such as a scFv molecule, or a construct described herein), the recombinant expression vectors described herein may carry regulatory sequences that control the expression of the antibody chain genes in a host cell. The design of the expression vector, including the selection of regulatory sequences, may depend on such factors as the choice of the host cell to be transformed or the level of expression of protein desired. For instance, suitable regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. Viral regulatory elements, and sequences thereof, are described in detail, for instance, in U.S. Pat. Nos. 5,168,062, 4,510,245, and 4,968,615, the disclosures of each of which are incorporated herein by reference.

In addition to the antibody chain genes and regulatory sequences, the recombinant expression vectors described herein can carry additional sequences, such as sequences that regulate replication of the vector in host cells (e.g., origins of replication) and selectable marker genes. A selectable marker gene facilitates selection of host cells into which the vector has been introduced (see e.g., U.S. Pat. Nos. 4,399,216, 4,634,665 and 5,179,017). For example, typically the selectable marker gene confers resistance to cytotoxic drugs, such as G418, puromycin, blasticidin, hygromycin or methotrexate, to a host cell into which the vector has been introduced. Suitable selectable marker genes include the dihydrofolate reductase (DHFR) gene (for use in DHFR″ host cells with methotrexate selection/amplification) and the neo gene (for G418 selection). In order to express the light and heavy chains of a conformation-specific NF-κB antibody or fragment thereof, the expression vector(s) containing polynucleotides encoding the heavy and light chains can be transfected into a host cell by standard techniques.

### Polynucleotides Encoding Modified Conformation-Specific NF-κB Antibodies

Conformation-specific NF-κB antibodies or fragments thereof described herein may contain one or more, or all, of the CDRs of a trans-mAb described herein, or one or more, or all, of the CDR-H1, CDR-H2, CDR-H3, CDR-L1, CDR-L2, and CDR-L3 sequences in which one or more, or all, of the CDR sequences exhibits at least 70% sequence identity (e.g., 75%, 80%, 85%, 90%, 95%, 96%, 97%, 98%, 99%, or 100% sequence identity) to the corresponding CDR sequence of a trans-mAb described herein or contains one or more (for instance, up to 3) amino acid substitutions (e.g., one or more conservative amino acid substitutions) relative to the corresponding CDR sequence of a trans-mAb described herein, but may feature differences in one or more framework regions of a trans-mAb described herein. For instance, one or more framework regions of a trans-mAb described herein may be substituted with the framework region of a human antibody. Exemplary framework regions include, for example, human framework regions described in U.S. Pat. No. 7,829,086, and primate framework regions as described in EP 1945668; the disclosures of each of which are incorporated herein by reference. To generate nucleic acids encoding such conformation-specific NF-κB antibodies or fragments thereof, DNA fragments encoding, e.g., at least one, or both, of the light chain variable regions and the heavy chain variable regions can be produced by chemical synthesis (e.g., by solid phase polynucleotide synthesis techniques), in vitro gene amplification (e.g., by polymerase chain reaction techniques), or by replication of the polynucleotide in a host organism. For instance, nucleic acids encoding conformation-specific NF-κB antibodies or fragments thereof described herein may be obtained by amplification and modification of germline DNA or cDNA encoding light and heavy chain variable sequences so as to incorporate one or more, or all, of the CDRs of a trans-mAb described herein into the framework residues of a consensus antibody.

In some embodiments, a humanized conformation-specific NF-κB antibody or fragment thereof may include one or more, or all, of the CDR-H1, CDR-H2, CDR-H3, CDR-L1, CDR-L2, and CDR-L3 sequences in which one or more, or all, of the CDR sequences exhibits at least 70% sequence identity (e.g., 75%, 80%, 85%, 90%, 95%, 96%, 97%, 98%, 99%, or 100% sequence identity) to the corresponding CDR sequence of a trans-mAb described herein or contains one or more (for instance, up to 3) amino acid substitutions (e.g., one or more conservative amino acid substitutions) relative to the corresponding CDR sequence of a trans-mAb described herein. This can be achieved, for example, by performing site-directed mutagenesis of germline DNA or cDNA and amplifying the resulting polynucleotides using the polymerase chain reaction (PCR) according to established procedures. Germline DNA sequences for human heavy and light chain variable region genes are known in the art (see, e.g., the “VBASE” human germline sequence database; see also Kabat et al., Sequences of Proteins of Immunological Interest, Fifth Edition, U.S. Department of Health and Human Services, NIH Publication No. 91-3242, 1991; Tomlinson et al., J. Mol. Biol. 227:776-798, 1992; and Cox et al., Eur. J. Immunol. 24:827-836, 1994; incorporated herein by reference). Chimeric nucleic acid constructs encoding human heavy and light chain variable regions containing one or more, or all, of the CDRs of a trans-mAb described herein, or a similar sequence as described above, can be produced, e.g., using established cloning techniques known in the art. Additionally, a polynucleotide encoding a heavy chain variable region containing the one or more of the CDRs of a trans-mAb described herein, or a similar sequence as described above, can be synthesized and used as a template for mutagenesis to generate a variant as described herein using routine mutagenesis techniques. Alternatively, a DNA fragment encoding the variant can be directly synthesized (e.g., by established solid phase nucleic acid chemical synthesis procedures).

Once DNA fragments encoding VH segments containing one or more, or all, of the CDR-H1, CDR-H2, and CDR-H3 sequences of a trans-mAb described herein are obtained, these DNA fragments can be further manipulated by standard recombinant DNA techniques, e.g., to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker.

The isolated DNA encoding the VH region of a conformation-specific NF-κB antibody described herein can be converted to a full-length heavy chain gene (as well as a Fab heavy chain gene), e.g., by operatively linking the VH-encoding DNA to another DNA molecule encoding heavy chain constant region domains (CH1, CH2, CH3, and, optionally, CH4). The sequences of human heavy chain constant region genes are known in the art (see e.g., Kabat et al., Sequences of Proteins of Immunological Interest, Fifth Edition, U.S. Department of Health and Human Services, NIH Publication No. 91-3242, 1991) and DNA fragments encompassing these regions can be obtained by standard PCR amplification. The heavy chain constant region can be an IgG1, IgG2, IgG3, IgG4, IgA, IgE, IgM or IgD constant region, and in certain embodiments is an IgG1 constant region. For a Fab fragment heavy chain gene, the VH-encoding DNA can be operatively linked to another DNA molecule encoding only the heavy chain CH1 domain.

Isolated DNA encoding the VL region of a conformation-specific NF-κB antibody can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat et al., Sequences of Proteins of Immunological Interest, Fifth Edition (U.S. Department of Health and Human Services, NIH Publication No. 91-3242, 1991)) and DNA fragments encompassing these regions can be obtained, e.g., by amplification in a prokaryotic or eukaryotic cell of a polynucleotide encoding these regions, by PCR amplification, or by chemical polynucleotide synthesis. The light chain constant region can be a kappa (κ) or lambda (λ) constant region, but in certain embodiments is a kappa constant region. To create a scFv gene, the VH and VL-encoding DNA fragments are operatively linked to another fragment encoding a flexible linker, e.g., a polynucleotide encoding a flexible, hydrophilic amino acid sequence, such as the amino acid sequence (Gly₄Ser)₃, such that the V_{H} and V_{L} sequences can be expressed as a contiguous single-chain protein, with the V_{L} and V_{H} regions joined by the linker (see e.g., Bird et al., Science 242:423-426, 1988; Huston et al., Proc. Natl. Acad. Sci. USA 85:5879-5883, 1988; McCafferty et al., Nature 348:552-554, 1990).

Recombinant DNA technology can also be used to remove some or all of the DNA encoding either or both of the light and heavy chains that is not necessary for binding to to a particular epitope of NF-κB. The molecules expressed from such truncated DNA molecules are also encompassed by the antibodies described herein. In addition, bifunctional antibodies can be produced in which one heavy contains one or more, or all, of the CDRs of a trans-mAb described herein, or a similar CDR sequence as described above, and the other heavy chain and/or the light chains are specific for an antigen other than NF-κB. Such antibodies can be generated, e.g., by crosslinking a heavy chain and light chain containing one or more, or all, of the CDRs of a trans-mAb described herein, or a similar CDR sequence as described above, to a heavy chain and light chain of a second antibody specific for a different antigen, for instance, using standard chemical crosslinking methods (e.g., by disulfide bond formation). Bifunctional antibodies can also be made by expressing a nucleic acid molecule engineered to encode a bifunctional antibody in a prokaryotic or eukaryotic cell.

Dual specific antibodies, i.e., antibodies that bind a particular epitope of NF-κB and a different antigen using the same binding site, can be produced by mutating amino acid residues in the light chain and/or heavy chain CDRs. In some embodiments, dual specific antibodies that bind two antigens, such as NF-κB and a second cell-surface receptor, can be produced by mutating amino acid residues in the periphery of the antigen binding site (Bostrom et al., Science 323: 1610-1614, 2009). Dual functional antibodies can be made by expressing a polynucleotide engineered to encode a dual specific antibody.

Modified conformation-specific NF-κB antibodies or fragments thereof described herein can also be produced by chemical synthesis (e.g., by the methods described in Solid Phase Peptide Synthesis, 2nd ed., 1984 The Pierce Chemical Co., Rockford, 111; incorporated herein by reference). Variant antibodies can also be generated using a cell-free synthetic platform (see, e.g., Chu et al., Biochemia No. 2, 2001 (Roche Molecular Biologicals); incorporated herein by reference).

### Host Cells for Expression of Conformation-Specific NF-κB Antibody or Fragment Thereof

It is possible to express the antibodies of fragments thereof described herein in either prokaryotic or eukaryotic host cells. In certain embodiments, expression of antibodies or fragments thereof is performed in eukaryotic cells, e.g., mammalian host cells, for optimal secretion of a properly folded and immunologically active antibody. Exemplary mammalian host cells for expressing the recombinant antibodies or antigen-binding fragments thereof described herein include Chinese Hamster Ovary (CHO cells) (including DHFR CHO cells, described in Urlaub and Chasin (1980, Proc. Natl. Acad. Sci. USA 77:4216-4220), used with a DHFR selectable marker, e.g., as described in Kaufman and Sharp (1982, Mol. Biol. 159:601-621), NSO myeloma cells, COS cells, 293 cells, and SP2/0 cells. Additional cell types that may be useful for the expression of antibodies and fragments thereof include bacterial cells, such as BL-21(DE3) E. coli cells, which can be transformed with vectors containing foreign DNA according to established protocols. Additional eukaryotic cells that may be useful for expression of antibodies include yeast cells, such as auxotrophic strains of S. cerevisiae, which can be transformed and selectively grown in incomplete media according to established procedures known in the art. When recombinant expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown.

Antibodies or antigen-binding fragments thereof can be recovered from the culture medium using standard protein purification methods. Host cells can also be used to produce portions of intact antibodies, such as Fab fragments or scFv molecules. Also included herein are methods in which the above procedure is varied according to established protocols known in the art. For example, it can be desirable to transfect a host cell with DNA encoding either the light chain or the heavy chain (but not both) of a conformation-specific NF-κB antibody or fragment thereof described herein in order to produce an antigen-binding fragment of the antibody.

Once a conformation-specific NF-κB antibody or fragment thereof described herein has been produced by recombinant expression, it can be purified by any method known in the art, such as a method useful for purification of an immunoglobulin molecule, for example, by chromatography (e.g., ion exchange, affinity, and sizing column chromatography), centrifugation, differential solubility, or by any other standard technique for the purification of proteins. Further, the conformation-specific NF-κB antibody or fragment thereof described herein or fragments thereof can be fused to heterologous polypeptide sequences described herein or otherwise known in the art to facilitate purification or to produce therapeutic conjugates.

Once isolated, a conformation-specific NF-κB antibody or fragment thereof can, if desired, be further purified, e.g., by high performance liquid chromatography (see, e.g., Fisher, Laboratory Techniques in Biochemistry and Molecular Biology (Work and Burdon, eds., Elsevier, 1980); incorporated herein by reference), or by gel filtration chromatography, such as on a Superdex™ 75 column (Pharmacia Biotech AB, Uppsala, Sweden).

### III. Platforms for Generating Conformation-Specific NF-κB Antibodies or Fragments Thereof

### Antigenic Peptides

Conformation-specific antibodies may be generated using immunogenic antigens (e.g., antigenic peptides) containing, for example, a phosphorylated or non-phosphorylated Thr-Pro motif (e.g., a phosphorylated Thr-Pro motif), where the peptidyl prolyl bond is fixed in a particular conformation (e.g., the cis or trans conformation) or is mixed cis and trans conformations or any other motif or amino acid sequence that is capable of cis/trans isomerization. For example, the cis or trans content of a phosphorylated or non-phosphorylated Thr-Pro-containing antigenic peptide may be fixed by stereoselective synthesis of (Z)- and (E)-alkene mimics by Still-Wittig and Ireland-Claisen rearrangements (J. Org. Chem., 68: 2343-2349, 2003; hereby incorporated by reference). Alternatively, the cis or trans content of phosphorylated or nonphosphorylated Thr-Pro-containing antigenic peptides of the invention may be increased or fixed by substituting a proline amino acid residue with a proline analog. Proline analogs include, without limitation, homoproline, azetidine-2-carboxylic acid (Aze), tert-butyl-L-proline (TBP), trans-4-fluoro-L-proline (t-4F-Pro), and cis-4-fluoro-L-proline (c-4F-Pro). The cis or trans content of a given antigen may be analyzed by, for example, nuclear magnetic resonance (NMR) analysis.

Antigenic peptides of the invention may contain a phosphorylated or nonphosphorylated Thr-Pro motif (e.g., a pThr-Pro motif) which is capable of cis/trans isomerization. The antigenic peptide may contain an epitope from the p65 subunit of NF-κB (GenBank Accession No. AAH33210) including a pThr-Pro motif (e.g., pThr254-Pro). The antigenic peptide may further include additional residues surrounding the Thr-Pro motif of the full-length polypeptide. For example, the antigenic peptide may include the 3-10 amino acid residues N-terminal to the S residue of a full-length polypeptide and the 3-10 amino acid residues C-terminal to the proline of a full-length polypeptide.

An antigenic peptide may contain an epitope from the p65 subunit of NF-κB (GenBank Accession No. AAH33210) including a pThr-Xaa motif (e.g., pThr254-Xaa). Xaa may be selected from Pro, a proline analog, or any natural or non-natural amino acid. Preferably, Xaa is any proline analog, or natural or non-natural amino acid wherein the peptide bond between pThr and Xaa in the pThr-Xaa motif is preferentially in the trans conformation. Most preferably, Xaa is an amino acid that share structural similarity to Pro, but which resides preferentially in the trans-peptide bond conformation (e.g., Xaa of the Thr-Xaa motif is selected from Ala or Gly). For example, the antigenic peptide may be a peptide containing the pThr254-Pro motif of the p65 submit of NF-κB (GenBank Accession No. AAH33210), wherein Pro255 has been replaced with a natural or non-natural amino acid that resides preferentially in the trans-peptide bond conformation, for example, Ala or Gly.

The antigenic peptide of the invention may be, for example, at least 4, 5, 6, 7, or 8 amino acid residues in length. The antigenic peptide may be between 8 and 20 amino acid residues in length (e.g., 8, 9, 10, 11, 12, 13, 14, 15, 16, 17, 18, 19, or 20 amino acids residues in length) or may be over 20 amino acid residues in length.

Such antigens may be produced and purified by any of a variety of methods known to one of skill in the art. Antigenic peptides may be produced and purified by, e.g., solid-phase chemical synthesis, in vitro transcription/translation, or by recombinant technology. The antigenic peptides may optionally be chemically coupled to a carrier protein or the peptides may be generated as fusion proteins to increase antigenicity. Antigenic peptides may be screened based upon their ability to induce the production of conformation-specific antibodies. In this respect, such screening techniques may include, but are not limited to, enzyme-linked immunosorbant assays (ELISA), immunoprecipitation, or other immunoassays.

Exemplary antigens useful in the production of conformation-specific antibodies include antigens containing a phosphorylated or nonphosphorylated Ser/Thr-homoproline, Ser/Thr-Aze, Ser/Thr-TBP, Ser/Thr-t-4F-Pro, Ser/Thr-c-4F-Pro motif. Such peptides may be used as antigens for generating, e.g., polyclonal or monoclonal antibodies (e.g., rabbit or mouse monoclonal antibodies).

### Generation and Purification of Conformation-Specific Antibodies

The antigens of the present invention may be used to generate, for example, monoclonal, polyclonal, chimeric, humanized, or recombinant conformation-specific antibodies by any method known in the art. These methods include the immunological methods described by Kohler and Milstein (Nature 256: 495-497, 1975 and Eur. J. Immunol. 6: 511-519, 1976) and Campbell (“Monoclonal Antibody Technology, The Production and Characterization of Rodent and Human Hybridomas,” in Burdon et al., Eds., Laboratory Techniques in Biochemistry and Molecular Biology, Volume 13, Elsevier Science Publishers, Amsterdam, 1985), as well as by the recombinant DNA method described by Huse et al. (Science 246: 1275-1281, 1989).

Briefly, the antigens of the present invention may, in combination with an adjuvant, be administered to a host animal (e.g., rabbits, mice, rats, goats, guinea pigs, hamsters, horses, and sheep, as well as non-human primates). The administration of such antigens may be accomplished by any of a variety of methods, including, but not limited to, subcutaneous or intramuscular injection. Once administered, the results of antibody titers produced in the host animal are monitored, which may be conducted by any of a variety of techniques well-known in the art (e.g., routine bleeds), with the antisera being isolated (e.g., via centrifugation) and thereafter screened for the presence of antibodies having a binding affinity for, e.g., the cis or trans conformation of a polypeptide or polypeptide fragment. Screening for the desired antibody may be accomplished by techniques including, e.g., radioimmunoassays, ELISA, sandwich immunoassays, immunoradiometric assays, gel diffusion precipitation reactions, in situ immunoassays (e.g., using colloidal gold, enzymatic, or radioisotope labels), Western blots, precipitation reactions, agglutination assays (e.g., gel agglutination assays or hemagglutination assays), complement fixation assays, immunofluorescence assays, protein A assays, and immunoelectrophoresis assays.

The resultant antisera derived from the host animal may be affinity purified to derive the antibodies for the present invention. The antisera may be purified via conventional techniques, such as the introduction of the antisera onto a separation column. The antigens of the present invention may be immobilized on the column to isolate and purify conformation-specific antibodies. For example, an antigenic peptide containing a Ser/Thr-Proline (e.g., pThr-Pro) motif that is used to generate a conformation-specific antibody (e.g., a trans-specific) may be immobilized on a column and used to purify the resulting conformation-specific antibody. The column may then be washed to remove antibodies not having specificity for the antigen immobilized on the column, with the remaining conformation-specific antibody ultimately being eluted from the column. The isolated conformation-specific antibody may then be stored per conventional practices known to those skilled in the art.

Established procedures for immunizing primates are known in the art (see, e.g., WO 1986/6004782; incorporated herein by reference). Immunization represents a robust method of producing monoclonal antibodies by exploiting the antigen specificity of B lymphocytes. For example, monoclonal antibodies can be prepared by the Kohler-Millstein procedure (described, e.g., in EP 0110716; incorporated herein by reference), wherein spleen cells from a non-human animal (e.g., a primate) administer peptide with an antigenic peptide. A clonally-expanded B lymphocyte produced by immunization can be isolated from the serum of the animal and subsequently fused with a myeloma cell in order to form a hybridoma. Hybridomas are particularly useful agents for antibody production, as these immortalized cells can provide a lasting supply of an antigen-specific antibody. Antibodies from such hybridomas can subsequently be isolated using techniques known in the art, e.g., by purifying the antibodies from the cell culture medium by affinity chromatography.

Alternatively, antibody libraries (e.g., naive antibody libraries, synthetic antibody libraries, semi-synthetic antibody libraries, or combinatorial libraries) may be screened for the identification of conformation-specific antibodies. Such libraries are commercially available from a number of sources (e.g., Cambridge Antibody, Cambridge, United Kingdom, Genetastix Corporation, Pacific Northwest Laboratory, Richland, Wash., and MorphoSys AG, Munich, Germany (e.g., HuCal GOLD)). See, e.g., U.S. Pat. Nos. 6,696,248; 6,706,484; 6,828,422; and 7,264,963, hereby incorporated by reference.

Screening of an antibody library may be performed by using one of the methods known to one of skill in the art including, e.g., phage-display, selectively infective phage, polysome technology, and assay systems for enzymatic activity or protein stability. Antibodies having the desired property can be identified, for example, by sequencing of the corresponding nucleic acid sequence, by amino acid sequencing, or by mass spectrometry. Optimization is performed by replacing sub-sequences with different sequences (e.g., random sequences) and then repeating the screening step one or more times. The antibodies may be screened for, e.g., optimized affinity or specificity for a target molecule (e.g., the cis or trans conformation of a target molecule), optimized expression yields, optimized stability, or optimized solubility.

Conformation-specific antibodies of the present invention recognize and specifically bind to, for example, a particular conformation (e.g., the cis or trans conformation) of its complementary antigen. For example, as described herein, the conformation-specific antibody may specifically bind to the trans conformation of a phosphorylated Thr-Pro motif of a polypeptide (e.g., pThr254-Pro of the p65 subunit of NF-κB), relative to the cis conformation. In this case, the Kd between the conformation-specific antibody and its antigen is, for example, at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M or greater. In addition to the binding specificity, the conformation-specific antibody will have, for example, at least 10- to 100-fold greater affinity to one conformation (e.g., the trans conformation) than to another conformation (e.g., the cis conformation) of the pThr-Pro motif.

### IV. Conformation-Specific NF-κB Antibody Conjugates

Prior to administration of a conformation-specific NF-κB antibody or antigen-binding fragment thereof described herein to a mammalian subject (e.g., a human), it may be desirable to conjugate the antibody or fragment thereof to a second molecule, e g., to modulate the activity of the antibody in vivo. Conformation-specific NF-κB antibodies or antigen-binding fragments thereof can be conjugated to other molecules at either the N-terminus or C-terminus of a light or heavy chain of the antibody using any one of a variety of established conjugation strategies that are well-known in the art. Examples of pairs of reactive functional groups that can be used to covalently tether a conformation-specific NF-κB antibody or antigen-binding fragment thereof to another molecule include, without limitation, thiol pairs, carboxylic acids and amino groups, ketones and amino groups, aldehydes and amino groups, thiols and alpha,beta-unsaturated moieties (such as maleimides or dehydroalanine), thiols and alpha-halo amides, carboxylic acids and hydrazides, aldehydes and hydrazides, and ketones and hydrazides.

Conformation-specific NF-κB antibodies or antigen-binding fragments thereof can be covalently appended directly to another molecule by chemical conjugation as described. Alternatively, fusion proteins containing a conformation-specific NF-κB antibody or antigen-binding fragment thereof can be expressed recombinantly from a cell (e.g., a eukaryotic cell or prokaryotic cell). This can be accomplished, for example, by incorporating a polynucleotide encoding the fusion protein into the nuclear genome of a cell (e.g., using techniques described herein or known in the art). Optionally, antibodies and fragments thereof described herein can be joined to a second molecule by forming a covalent bond between the antibody and a linker. This linker can then be subsequently conjugated to another molecule, or the linker can be conjugated to another molecule prior to ligation to the a conformation-specific NF-κB antibody or antigen-binding fragment thereof. Examples of linkers that can be used for the formation of a conjugate include polypeptide linkers, such as those that contain naturally occurring or non-naturally occurring amino acids. In some embodiments, it may be desirable to include D-amino acids in the linker, as these residues are not present in naturally-occurring proteins and are thus more resistant to degradation by endogenous proteases. Fusion proteins containing polypeptide linkers can be made using chemical synthesis techniques, such as those described herein, or through recombinant expression of a polynucleotide encoding the fusion protein in a cell (e.g., a prokaryotic or eukaryotic cell). Linkers can be prepared using a variety of strategies that are well known in the art, and depending on the reactive components of the linker, can be cleaved by enzymatic hydrolysis, photolysis, hydrolysis under acidic conditions, hydrolysis under basic conditions, oxidation, disulfide reduction, nucleophilic cleavage, or organometallic cleavage (Leriche et al., Bioorg. Med. Chem., 20:571-582, 2012).

### Drug-Polypeptide Conjugates

A conformation-specific NF-κB antibody or antigen-binding fragment thereof described herein can additionally be conjugated to, admixed with, or administered separately from a therapeutic agent, such as a cytotoxic molecule. Conjugates described herein may be applicable to the treatment or reduction of an NF-κB-related diseases (e.g., infection, cancer, or immune or inflammatory disorders such as sepsis, septic shock, SIRS, or CRS). Exemplary cytotoxic agents that can be conjugated to, admixed with, or administered separately from a conformation-specific NF-κB antibody or antigen-binding fragment thereof include, without limitation, antineoplastic agents such as: acivicin; aclarubicin; acodazole hydrochloride; acronine; adozelesin; adriamycin; aldesleukin; altretamine; ambomycin; a metantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; camptothecin; caracemide; carbetimer; carboplatin; carmustine; carubicin hydrochloride; carzelesin; cedefingol; chlorambucil; cirolemycin; cisplatin; cladribine; combretestatin a-4; crisnatol mesylate; cyclophosphamide; cytarabine; dacarbazine; daca (n-[2-(dimethyl-amino) ethyl]acridine-4-carboxamide); dactinomycin; daunorubicin hydrochloride; daunomycin; decitabine; dexormaplatin; dezaguanine; dezaguanine mesylate; diaziquone; docetaxel; dolasatins; doxorubicin; doxorubicin hydrochloride; droloxifene; droloxifene citrate; dromostanolone propionate; duazomycin; edatrexate; eflornithine hydrochloride; ellipticine; elsamitrucin; enloplatin; enpromate; epipropidine; epirubicin hydrochloride; erbulozole; esorubicin hydrochloride; estramustine; estramustine phosphate sodium; etanidazole; ethiodized oil i 131; etoposide; etoposide phosphate; etoprine; fadrozole hydrochloride; fazarabine; fenretinide; floxuridine; fludarabine phosphate; fluorouracil; 5-fdump; flurocitabine; fosquidone; fostriecin sodium; gemcitabine; gemcitabine hydrochloride; gold au 198; homocamptothecin; hydroxyurea; idarubicin hydrochloride; ifosfamide; ilmofosine; interferon alfa-2a; interferon alfa-2b; interferon alfa-n1; interferon alfa-n3; interferon beta-i a; interferon gamma-i b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peploycinsulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; rhizoxin; rhizoxin d; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; strontium chloride sr 89; sulofenur; talisomycin; taxane; taxoid; tecogalan sodium; tegafur; teloxantrone hydrochloride; temoporfin; teniposide; teroxirone; testolactone; thiamiprine; thioguanine; thiotepa; thymitaq; tiazofurin; tirapazamine; tomudex; top53; topotecan hydrochloride; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine; vinblastine sulfate; vincristine; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride; 2-chlorodeoxyadenosine; 2′ deoxyformycin; 9-aminocamptothecin; raltitrexed; N-propargyl-5,8-dideazafolic acid; 2chloro-2′-arabino-fluoro-2′-deoxyadenosine; 2-chloro-2′-deoxyadenosine; anisomycin; trichostatin A; hPRL-G129R; CEP-751; linomide; sulfur mustard; nitrogen mustard (mechlor ethamine); cyclophosphamide; melphalan; chlorambucil; ifosfamide; busulfan; N-methyl-Nnitrosourea (MNU); N,N′-Bis (2-chloroethyl)-N-nitrosourea (BCNU); N-(2-chloroethyl)-N′ cyclohexyl-N-nitrosourea (CCNU); N-(2-chloroethyl)-N′-(trans-4-methylcyclohexyl-N-nitrosourea (MeCCNU); N-(2-chloroethyl)-N′-(diethyl) ethylphosphonate-N-nitrosourea (fotemustine); streptozotocin; diacarbazine (DTIC); mitozolomide; temozolomide; thiotepa; mitomycin C; AZQ; adozelesin; cisplatin; carboplatin; ormaplatin; oxaliplatin; C1-973; DWA 2114R; JM216; JM335; Bis (platinum); tomudex; azacitidine; cytarabine; gemcitabine; 6-mercaptopurine; 6-thioguanine; hypoxanthine; teniposide 9-amino camptothecin; topotecan; CPT-11; Doxorubicin; Daunomycin; Epirubicin; darubicin; mitoxantrone; losoxantrone; Dactinomycin (Actinomycin D); amsacrine; pyrazoloacridine; all-trans retinol; 14-hydroxy-retro-retinol; all-trans retinoic acid; N-(4-hydroxyphenyl) retinamide; 13-cis retinoic acid; 3-methyl TTNEB; 9-cis retinoic acid; fludarabine (2-F-ara-AMP); or 2-chlorodeoxyadenosine (2-Cda).

Other therapeutic compounds that can be conjugated to, admixed with, or administered separately from a conformation-specific NF-κβ antibody or antigen-binding fragment thereof described herein in order to treat, reduce, or study the progression of a disease associated with NF-κB dysregulation (e.g., an immune or inflammatory disorder, such as sepsis, septic shock, SIRS, or CRS; a cancer; or an infection) include, but are not limited to, cytotoxic agents such as 20-pi-1,25 dihydroxyvitamin D3; 5-ethynyluracil; abiraterone; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; argininedeaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bleomycin A2; bleomycin B2; bropirimine; budotitane; buthionine sulfoximine; calcipotriol; calphostin C; camptothecin derivatives (e.g., 10-hydroxy-camptothecin); canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; 2′deoxycoformycin (DCF); deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; discodermolide; docosanol; dolasetron; doxifluridine; droloxifene; dronabinol; duocarmycin SA; ebselen; ecomustine; edelfosine; edrecolomab; eflornithine; elemene; emitefur; epirubicin; epothilones (A, R=H; B, R=Me); epithilones; epristeride; estramustine analogue; estrogen agonists; estrogen antagonists; etanidazole; etoposide; etoposide 4′-phosphate (etopofos); exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; homoharringtonine (HHT); hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maytansine; mannostatin A; marimastat; masoprocol; maspin; matrilysin inhibitors; matrix metalloproteinase inhibitors; menogaril; rnerbarone; meterelin; methioninase; metoclopramide; MIF inhibitor; ifepristone; miltefosine; mirimostim; mismatched double stranded RNA; mithracin; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; podophyllotoxin; porfimer sodium; porfiromycin; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B 1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single-chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

Labeled conformation-specific NF-κB antibodies or antigen-binding fragments thereof

In some embodiments, conformation-specific NF-κB antibodies or antigen-binding fragments thereof described herein are conjugated to another molecule (e.g., an epitope tag) for the purpose of purification or detection. Examples of such molecules that are useful in protein purification include those that present structural epitopes capable of being recognized by a second molecule. This is a common strategy that is employed in protein purification by affinity chromatography, in which a molecule is immobilized on a solid support and exposed to a heterogeneous mixture containing a target protein conjugated to a molecule capable of binding the immobilized compound. Examples of epitope tag molecules that can be conjugated to conformation-specific NF-κB antibodies or antigen-binding fragments thereof for the purposes of molecular recognition include, without limitation, maltose-binding protein, glutathione-S-transferase, a poly-histidine tag, a FLAG-tag, a myc-tag, human influenza hemagglutinin (HA) tag, biotin, streptavidin. Conjugates containing the epitopes presented by these molecules are capable of being recognized by such complementary molecules as maltose, glutathione, a nickel-containing complex, an anti-FLAG antibody, an anti-myc antibody, an anti-HA antibody, streptavidin, or biotin, respectively. For example, one can purify an antibody or fragment thereof described herein that has been conjugated to an epitope tag from a complex mixture of other proteins and biomolecules (e.g., DNA, RNA, carbohydrates, phospholipids, etc) by treating the mixture with a solid phase resin containing an complementary molecule that can selectively recognize and bind the epitope tag of the antibody or fragment thereof. Examples of solid phase resins include agarose beads, which are compatible with purifications in aqueous solution.

A conformation-specific NF-κB antibody or antigen-binding fragment thereof described herein can also be covalently appended to a fluorescent molecule, e.g., to detect the antibody or antigen-binding fragment thereof by fluorimetry and/or by direct visualization using fluorescence microscopy. Exemplary fluorescent molecules that can be conjugated to antibodies described herein include green fluorescent protein, cyan fluorescent protein, yellow fluorescent protein, red fluorescent protein, phycoerythrin, allophycocyanin, hoescht, 4′,6-diamidino-2-phenylindole (DAPI), propidium iodide, fluorescein, coumarin, rhodamine, tetramethylrhoadmine, and cyanine. Additional examples of fluorescent molecules suitable for conjugation to antibodies described herein are well-known in the art and have been described in detail in, e.g., U.S. Pat. Nos. 7,417,131 and 7,413,874, each of which is incorporated by reference herein.

Conformation-specific NF-κB antibodies or antigen-binding fragments thereof containing a fluorescent molecule are particularly useful for monitoring the cell-surface localization properties of antibodies and fragments thereof described herein. For instance, one can expose cultured mammalian cells to conformation-specific NF-κB antibodies or antigen-binding fragments thereof described herein that have been covalently conjugated to a fluorescent molecule and subsequently analyze these cells using conventional fluorescent microscopy techniques known in the art. Confocal fluorescent microscopy is a particularly powerful method for determining cell-surface localization of tagged antibodies, as individual planes of a cell can be analyzed in order to distinguish antibodies or fragments thereof that have been internalized into a cell's interior, e.g., by receptor-mediated endocytosis, from those that are bound to the external face of the cell membrane. Additionally, cells can be treated with an antibody conjugated to a fluorescent molecule that emits visible light of a particular wavelength (e.g., fluorescein, which fluoresces at about 535 nm) and an additional fluorescent molecule that is known to localize to a particular site on the cell surface and that fluoresces at a different wavelength (e.g., a molecule that localizes to CD25 and that fluoresces at about 599 nm). The resulting emission patterns can be visualized by confocal fluorescence microscopy and the images from these two wavelengths can be merged in order to reveal information regarding the location of the antibody or antigen-binding fragment thereof on the cell surface with respect to other receptors.

Bioluminescent proteins can also be incorporated into a fusion protein for the purposes of detection and visualization of antibodies or fragments thereof. Bioluminescent proteins, such as Luciferase and aequorin, emit light as part of a chemical reaction with a substrate (e.g., luciferin and coelenterazine). Exemplary bioluminescent proteins suitable for use as a diagnostic sequence and methods for their use are described in, e.g., U.S. Pat. Nos. 5,292,658, 5,670,356, 6,171,809, and 7,183,092, each of which is herein incorporated by reference. Conformation-specific NF-κB antibodies or antigen-binding fragments thereof labeled with bioluminescent proteins are a useful tool for the detection of antibodies described herein following an in vitro assay. For instance, the presence of an antibody that has been conjugated to a bioluminescent protein can be detected among a complex mixture of additional proteins by separating the components of the mixture using gel electrophoresis methods known in the art (e.g., native gel analysis) and subsequently transferring the separated proteins to a membrane in order to perform a Western blot. Detection of the antibody among the mixture of other proteins can be achieved by treating the membrane with an appropriate Luciferase substrate and subsequently visualizing the mixture of proteins on film using established protocols.

A conformation-specific NF-κB antibody or antigen-binding fragment thereof described herein can also be conjugated to a molecule including a radioactive nucleus, such that an antibody or fragment thereof described herein can be detected by analyzing the radioactive emission pattern of the nucleus. Alternatively, an antibody or fragment thereof can be modified directly by incorporating a radioactive nucleus within the antibody during the preparation of the protein. Radioactive isotopes of methionine (³⁵S), nitrogen (¹⁵N), or carbon (¹³C) can be incorporated into antibodies or fragments thereof described herein by, e.g., culturing bacteria in media that has been supplemented with nutrients containing these isotopes. Optionally, tyrosine derivatives containing a radioactive halogen can be incorporated into an antibody by, e.g., culturing bacterial cells in media supplemented with radiolabeled tyrosine. It has been shown that tyrosine functionalized with a radioactive halogen at the C2 position of the phenol system are rapidly incorporated into elongating polypeptide chains using the endogenous translation enzymes in vivo (U.S. Pat. No. 4,925,651; incorporated herein by reference). The halogens include fluorine, chlorine, bromine, iodine, and astatine. Additionally, an antibody can be modified following isolation and purification from cell culture by functionalizing polypeptides described herein with a radioactive isotope. The halogens represent a class of isotopes that can be readily incorporated into a purified protein by aromatic substitution at tyrosine or tryptophan, e.g., via reaction of one or more of these residues with an electrophilic halogen species. Examples of radioactive halogen isotopes include ¹⁸F, ⁷⁵Br, ⁷⁷Br, ¹²²I, ¹²³I, ¹²⁴I, ¹²⁵I, ¹²⁹I, ¹³¹I, or ²¹¹At.

Another alternative strategy for the incorporation of a radioactive isotope is the covalent attachment of a chelating group to the antibody or fragment thereof, or construct. Chelating groups can be covalently appended to an antibody or fragment thereof by attachment to a reactive functional group, such as a thiol, amino group, alcohol, or carboxylic acid. The chelating groups can then be modified to contain any of a variety of metallic radioisotopes, including, without limitation, such radioactive nuclides as ¹²⁵I, ⁶⁷Ga, ⁹⁹Tc, ¹⁶⁹Yb, ¹⁸⁶Re, ¹²³I, ¹²⁴I, ¹²⁵I, ¹³¹I, ^{99m}Tc, ¹¹¹In, ⁶⁴cu, ⁶⁷Cu, ¹⁸⁶Re, ¹⁸⁸Re, ¹⁷⁷Lu, ⁹⁰Y, ⁷⁷As, ⁷²AS, ⁸⁶Y, ⁸⁹Zr, ²¹¹At, ²¹²At, ²¹³Bi, or ²²⁵Ac.

In some embodiments, it may be desirable to covalently conjugate the antibodies or fragments thereof described herein with a chelating group capable of binding a metal ion from heavy elements or rare earth ions, such as Gd³⁺, Fe³⁺, Mn³⁺, or Cr²⁺. Conjugates containing chelating groups that are coordinated to such paramagnetic metals are useful as in MRI imaging applications. Paramagnetic metals include, but are not limited to, chromium (III), manganese (II), iron (II), iron (III), cobalt (II), nickel (II), copper (II), praseodymium (III), neodymium (III), samarium (III), gadolinium (III), terbium (III), dysprosium (III), holmium (III), erbium (III), and ytterbium (III). In this way, antibodies can be detected by MRI spectroscopy. For instance, one can administer antibodies or fragments thereof conjugated to chelating groups bound to paramagnetic ions to a mammalian subject (e.g., a human patient) in order to monitor the distribution of the antibody following administration. This can be achieved by administration of the antibody to a patient by any of the administration routes described herein, such as intravenously, and subsequently analyzing the location of the administered antibody by recording an MRI of the patient according to established protocols.

A conformation-specific NF-κB antibody or antigen-binding fragment thereof can additionally be conjugated to other molecules for the purpose of improving the solubility and stability of the protein in aqueous solution. Examples of such molecules include PEG, PSA, bovine serum albumin (BSA), and human serum albumin (HSA), among others. For instance, one can conjugate an antibody to carbohydrate moieties in order to evade detection of the antibody or fragment thereof by the immune system of the patient receiving treatment. This process of hyperglycosylation reduces the immunogenicity of therapeutic proteins by sterically inhibiting the interaction of the protein with B cell receptors in circulation. Alternatively, antibodies or fragments thereof can be conjugated to molecules that prevent clearance from human serum and improve the pharmacokinetic profile of antibodies described herein. Exemplary molecules that can be conjugated to or inserted within conformation-specific NF-κB antibody or antigen-binding fragment thereof described herein so as to attenuate clearance and improve the pharmacokinetic profile of these antibodies and fragments include salvage receptor binding epitopes. These epitopes are found within the Fc region of an IgG immunoglobulin and have been shown to bind Fc receptors and prolong antibody half-life in human serum. The insertion of salvage receptor binding epitopes into antibodies or fragments thereof can be achieved, e.g., as described in U.S. Pat. No. 5,739,277; incorporated herein by reference.

### V. Methods of Treatment

Conformation-specific NF-κB antibodies or antigen-binding fragments thereof described herein, can be used to treat a patient suffering from an NF-κB-related diseases (e.g., infection, cancer, or immune or inflammatory disorders such as sepsis, septic shock, SIRS, or CRS).

### Methods of Treating Cancer

Conformation-specific NF-κB antibodies or antigen-binding fragments thereof described herein are useful therapeutics for the treatment of a wide array of cancers. Conformation-specific NF-κB antibodies or antigen-binding fragments thereof can be administered to a mammalian subject, such as a human, suffering from a cancer, e.g., to enhance the effectiveness of the adaptive immune response against the target cancer cells.

Exemplary compositions of the disclosure that can be used for these purposes include conformation-specific NF-κB antibodies or antigen-binding fragments thereof that bind specifically to an epitope including the pThr254-Pro motif of the p65 subunit of NF-κB (e.g., antibodies that bind specifically to the trans conformation of pThr254-Pro motif of the p65 subunit of NF-κB). Particularly, methods described herein include administering a conformation-specific NF-κB antibody or antigen-binding fragment thereof that contains one or more, or all, of the CDR sequences of a trans-mAb described herein, such as a human, humanized, or chimeric variant of a trans-mAb described herein, to a human or a non-human mammal in order to treat a cancer.

Conformation-specific NF-κB antibodies or antigen-binding fragments thereof described herein can be administered to a mammalian subject (e.g., a human) suffering from cancer in order to improve the condition of the patient by promoting the immune response against cancer cells and tumorogenic material. Polypeptides described herein can be administered to a subject, e.g., via any of the routes of administration described herein. Polypeptides described herein can also be formulated with excipients, biologically acceptable carriers, and may be optionally conjugated to, admixed with, or co-administered separately (e.g., sequentially) with additional therapeutic agents, such as anti-cancer agents. Cancers that can be treated by administration of antibodies or antigen-binding fragments thereof described herein include such cancers as leukemia, lymphoma, liver cancer, bone cancer, lung cancer, brain cancer, bladder cancer, gastrointestinal cancer, breast cancer, cardiac cancer, cervical cancer, uterine cancer, head and neck cancer, gallbladder cancer, laryngeal cancer, lip and oral cavity cancer, ocular cancer, melanoma, pancreatic cancer, prostate cancer, colorectal cancer, testicular cancer, and throat cancer. Particular cancers that can be treated by administration of antibodies or antigen-binding fragments thereof described herein include, without limitation, acute lymphoblastic leukemia (ALL), acute myeloid leukemia (AML), chronic lymphocytic leukemia (CLL), chronic myelogenous leukemia (CML), adrenocortical carcinoma, AIDS-related lymphoma, primary CNS lymphoma, anal cancer, appendix cancer, astrocytoma, atypical teratoid/rhabdoid tumor, basal cell carcinoma, bile duct cancer, extrahepatic cancer, ewing sarcoma family, osteosarcoma and malignant fibrous histiocytoma, central nervous system embryonal tumors, central nervous system germ cell tumors, craniopharyngioma, ependymoma, bronchial tumors, burkitt lymphoma, carcinoid tumor, primary lymphoma, chordoma, chronic myeloproliferative neoplasms, colon cancer, extrahepatic bile duct cancer, ductal carcinoma in situ (DCIS), endometrial cancer, ependymoma, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors (GIST), testicular germ cell tumor, gestational trophoblastic disease, glioma, childhood brain stem glioma, hairy cell leukemia, hepatocellular cancer, langerhans cell histiocytosis, hodgkin lymphoma, hypopharyngeal cancer, islet cell tumors, pancreatic neuroendocrine tumors, wilms tumor and other childhood kidney tumors, langerhans cell histiocytosis, small cell lung cancer, cutaneous T cell lymphoma, intraocular melanoma, merkel cell carcinoma, mesothelioma, metastatic squamous neck cancer, midline tract carcinoma, multiple endocrine neoplasia syndromes, multiple myeloma/plasma cell neoplasm, myelodysplastic syndromes, nasal cavity and paranasal sinus cancer, nasopharyngeal cancer, neuroblastoma, non-hodgkin lymphoma (NHL), non-small cell lung cancer (NSCLC), epithelial ovarian cancer, germ cell ovarian cancer, low malignant potential ovarian cancer, pancreatic neuroendocrine tumors, papillomatosis, paraganglioma, paranasal sinus and nasal cavity cancer, parathyroid cancer, penile cancer, pharyngeal cancer, pheochromocytoma, pituitary tumor, pleuropulmonary blastoma, primary peritoneal cancer, rectal cancer, retinoblastoma, rhabdomyosarcoma, salivary gland cancer, kaposi sarcoma, rhabdomyosarcoma, sézary syndrome, small intestine cancer, soft tissue sarcoma, throat cancer, thymoma and thymic carcinoma, thyroid cancer, transitional cell cancer of the renal pelvis and ureter, urethral cancer, endometrial uterine cancer, uterine sarcoma, vaginal cancer, vulvar cancer, and Waldenström macroglobulinemia.

For example, conformation-specific NF-κB antibodies or antigen-binding fragments thereof described herein can be administered to a patient (e.g., a mammalian patient, such as a human patient) in order to treat a cancer characterized dyregulation (e.g., increased activity or expression) or NF-κB (e.g., NF-κB signaling).

Conformation-specific NF-κB antibodies or antigen-binding fragments thereof can also be co-administered with a therapeutic antibody that exhibits reactivity towards a cancer cell. Conformation-specific NF-κB antibodies or antigen-binding fragments thereof described herein may synergize not only with the adaptive immune response, e.g., by prolonging T lymphocyte tumor reactivity, but also with other inhibitors of tumor cell growth. Examples of additional therapeutic antibodies that can be used to treat cancer and other cell proliferation disorders include those that exhibit reactivity with a tumor antigen or a cell-surface protein that is overexpressed on the surface of a cancer cell. Exemplary antibodies that can be admixed, co-administered, or sequentially administered with conformation-specific NF-κB antibodies or antigen-binding fragments thereof described herein include, without limitation, Trastuzamb (HERCEPTIN®), Bevacizumab (AVASTIN®), Cetuximab (ERBITUX®), Panitumumab (VECTIBIX®), Ipilimumab (YERVOY®), Rituximab (RITUXAN® and MABTHERA®), Alemtuzumab (CAMPATH®), Ofatumumab (ARZERRA®), Gemtuzumab ozogamicin (MYLOTARG®), Brentuximab vedotin (ADCETRIS®), ⁹⁰Y-Ibritumomab Tiuxetan (ZEVALIN®), and ¹³¹I-Tositumomab (BEXXAR®), which are described in detail in Scott et al. (Cancer Immun., 12:14-21, 2012); incorporated herein by reference.

Antibodies or antigen-binding fragments thereof described herein can be monitored for their ability to attenuate the progression of a cancer, by any of a variety of methods known in the art. For instance, a physician may monitor the response of a mammalian subject (e.g., a human) to treatment by analyzing the volume of one or more tumors in the patient. For example, polypeptides (e.g., single-chain polypeptides, antibodies, antigen-binding fragments thereof, and constructs thereof) described herein may be capable of reducing tumor volume by between 1% and 100% (e.g., 1%, 5%, 10%, 20%, 30%, 40%, 50%, 60%, 70%, 80%, 90%, 95%, 99%, or 100%). Alternatively, a physician may monitor the responsiveness of a subject (e.g., a human) to treatment by analyzing the T-reg cell population in the lymph of a particular subject. For instance, a physician may withdraw a sample of blood from a mammalian subject (e.g., a human) and determine the quantity or density of T-reg cells (e.g., CD4₊CD25₃₀ FOXP3₊ T-reg cells or CD17₊ T-reg cells) using established procedures, such as fluorescence activated cell sorting.

### Methods of Treating Infectious Diseases

Conformation-specific NF-κB antibodies or antigen-binding fragments thereof described herein can also be used for treating infectious diseases, such as those caused by any one or more of a virus, a bacterium, a fungus, or a parasite (e.g., a eukaryotic parasite). For instance, conformation-specific NF-κB antibodies or antigen-binding fragments thereof can be administered to a mammalian subject (e.g., a human) suffering from an infectious disease in order to treat the disease, as well as to alleviate one or more symptoms of the disease.

In some embodiments, a conformation-specific NF-κB antibody or antigen-binding fragment thereof can be used to treat a betacoronavirus infection (e.g., SARS-CoV, MERS-CoV, or SARS-CoV-2) or symptoms associated with a betacoronavirus infection. In some embodiments, a conformation-specific NF-κB antibody or antigen-binding fragment thereof can be used to treat or prevent an immune or inflammatory response associated with a betacoronavirus infection. For example, a conformation-specific NF-κB antibody or antigen-binding fragment thereof can be used to treat or prevent sepsis, SIRS, or CRS associated with a betacoronavirus infection, as described herein. In some embodiments, the subject has been diagnosed with COVID-19, is suspected to have COVID-19, has been in contact with someone diagnosed with COVID-19, or has recently traveled to an area experiencing an outbreak of COVID-19.

Exemplary compositions of the disclosure that can be used for these purposes include conformation-specific NF-κB antibodies or antigen-binding fragments thereof that bind specifically to an epitope including the pThr254-Pro motif of the p65 subunit of NF-κB (e.g., antibodies that bind specifically to the trans conformation of pThr254-Pro motif of the p65 subunit of NF-κB). Particularly, methods described herein include administering a conformation-specific NF-κB antibody or antigen-binding fragment thereof that contains one or more, or all, of the CDR sequences of a trans-mAb described herein, such as a human, humanized, or chimeric variant of a trans-mAb described herein, to a human or a non-human mammal in order to treat an infection.

For example, conformation-specific NF-κB antibodies or antigen-binding fragments thereof described herein can be used for treating, or alleviating one or more symptoms of, viral infections in a mammalian subject, such as a human, that are caused by, e.g., a member of the Flaviviridae family (e.g., a member of the Flavivirus, Pestivirus, and Hepacivirus genera), which includes the hepatitis C virus, Yellow fever virus; Tick-borne viruses, such as the Gadgets Gully virus, Kadam virus, Kyasanur Forest disease virus, Langat virus, Omsk hemorrhagic fever virus, Powassan virus, Royal Farm virus, Karshi virus, tick-borne encephalitis virus, Neudoerfl virus, Sofjin virus, Louping ill virus and the Negishi virus; seabird tick-borne viruses, such as the Meaban virus, Saumarez Reef virus, and the Tyuleniy virus; mosquito-borne viruses, such as the Aroa virus, dengue virus, Kedougou virus, Cacipacore virus, Koutango virus, Japanese encephalitis virus, Murray Valley encephalitis virus, St. Louis encephalitis virus, Usutu virus, West Nile virus, Yaounde virus, Kokobera virus, Bagaza virus, Ilheus virus, Israel turkey meningoencephalo-myelitis virus, Ntaya virus, Tembusu virus, Zika virus, Banzi virus, Bouboui virus, Edge Hill virus, Jugra virus, Saboya virus, Sepik virus, Uganda S virus, Wesselsbron virus, yellow fever virus; and viruses with no known arthropod vector, such as the Entebbe bat virus, Yokose virus, Apoi virus, Cowbone Ridge virus, Jutiapa virus, Modoc virus, Sal Vieja virus, San Perlita virus, Bukalasa bat virus, Carey Island virus, Dakar bat virus, Montana myotis leukoencephalitis virus, Phnom Penh bat virus, Rio Bravo virus, Tamana bat virus, and the Cell fusing agent virus; a member of the Arenaviridae family, which includes the Ippy virus, Lassa virus (e.g., the Josiah, LP, or GA391 strain), lymphocytic choriomeningitis virus (LCMV), Mobala virus, Mopeia virus, Amapari virus, Flexal virus, Guanarito virus, Junin virus, Latino virus, Machupo virus, Oliveros virus, Parana virus, Pichinde virus, Pirital virus, Sabia virus, Tacaribe virus, Tamiami virus, Whitewater Arroyo virus, Chapare virus, and Lujo virus; a member of the Bunyaviridae family (e.g., a member of the Hantavirus, Nairovirus, Orthobunyavirus, and Phlebovirus genera), which includes the Hantaan virus, Sin Nombre virus, Dugbe virus, Bunyamwera virus, Rift Valley fever virus, La Crosse virus, California encephalitis virus, and Crimean-Congo hemorrhagic fever (CCHF) virus; a member of the Filoviridae family, which includes the Ebola virus (e.g., the Zaire, Sudan, Ivory Coast, Reston, and Uganda strains) and the Marburg virus (e.g., the Angola, Ci67, Musoke, Popp, Ravn and Lake Victoria strains); a member of the Togaviridae family (e.g., a member of the Alphavirus genus), which includes the Venezuelan equine encephalitis virus (VEE), Eastern equine encephalitis virus (EEE), Western equine encephalitis virus (WEE), Sindbis virus, rubella virus, Semliki Forest virus, Ross River virus, Barmah Forest virus, O'nyong'nyong virus, and the chikungunya virus; a member of the Poxviridae family (e.g., a member of the Orthopoxvirus genus), which includes the smallpox virus, monkeypox virus, and vaccinia virus; a member of the Herpesviridae family, which includes the herpes simplex virus (HSV; types 1, 2, and 6), human herpes virus (e.g., types 7 and 8), cytomegalovirus (CMV), Epstein-Barr virus (EBV), Varicella-Zoster virus, and Kaposi's sarcoma associated-herpesvirus (KSHV); a member of the Orthomyxoviridae family, which includes the influenza virus (A, B, and C), such as the H5N1 avian influenza virus or H1 N1 swine flu; a member of the Coronaviridae family, which includes a betacoronavirus infection, such as Severe Acute Respiratory Syndrome coronavirus (SARS-CoV), Middle Eastern Respiratory Syndrome coronavirus (MERS-CoV), or Coronavirus Disease 2019 (COVID-19 associated with SARS-CoV-2); a member of the Rhabdoviridae family, which includes the rabies virus and vesicular stomatitis virus (VSV); a member of the Paramyxoviridae family, which includes the human respiratory syncytial virus (RSV), Newcastle disease virus, hendravirus, nipahvirus, measles virus, rinderpest virus, canine distemper virus, Sendai virus, human parainfluenza virus (e.g., 1, 2, 3, and 4), rhinovirus, and mumps virus; a member of the Picornaviridae family, which includes the poliovirus, human enterovirus (A, B, C, and D), hepatitis A virus, and the coxsackievirus; a member of the Hepadnaviridae family, which includes the hepatitis B virus; a member of the Papillamoviridae family, which includes the human papilloma virus; a member of the Parvoviridae family, which includes the adeno-associated virus; a member of the Astroviridae family, which includes the astrovirus; a member of the Polyomaviridae family, which includes the JC virus, BK virus, and SV40 virus; a member of the Calciviridae family, which includes the Norwalk virus; a member of the Reoviridae family, which includes the rotavirus; and a member of the Retroviridae family, which includes the human immunodeficiency virus (HIV; e.g., types 1 and 2), and human T lymphotropic virus Types I and II (HTLV-1 and HTLV-2, respectively); Friend Leukemia Virus; and transmissible spongiform encephalopathy, such as chronic wasting disease. Particularly, methods described herein include administering a conformation-specific NF-κB antibody or antigen-binding fragment thereof described herein to a human in order to treat an HIV infection (such as a human suffering from AIDS).

Conformation-specific NF-κB antibodies or antigen-binding fragments thereof described herein can also be used for treating, or alleviating one or more symptoms of, bacterial infections in a mammalian subject (e.g., a human). Examples of bacterial infections that may be treated by administration of a conformation-specific NF-κB antibody or antigen-binding fragment thereof described herein include, without limitation, those caused by bacteria within the genera Streptococcus, Bacillus, Listeria, Corynebacterium, Nocardia, Neisseria, Actinobacter, Moraxella, Enterobacteriacece (e.g., E. coli, such as O157:H7), Pseudomonas (such as Pseudomonas aeruginosa), Escherichia, Klebsiella, Serratia, Enterobacter, Proteus, Salmonella, Shigella, Yersinia, Haemophilus, Bordetella (such as Bordetella pertussis), Legionella, Pasteurella, Francisella, Brucella, Bartonella, Clostridium, Vibrio, Campylobacter, Staphylococcus, Mycobacterium (such as Mycobacterium tuberculosis and Mycobacterium avium paratuberculosis, and Helicobacter (such as Helicobacter pylori and Helicobacter hepaticus).

Conformation-specific NF-κB antibodies or antigen-binding fragments thereof described herein can also be administered to a mammalian subject (e.g., a human) for treating, or alleviating one or more symptoms of, parasitic infections caused by a protozoan parasite (e.g., an intestinal protozoa, a tissue protozoa, or a blood protozoa) or a helminthic parasite (e.g., a nematode, a helminth, an adenophorea, a secementea, a trematode, a fluke (blood flukes, liver flukes, intestinal flukes, and lung flukes), or a cestode). Exemplary protozoan parasites that can be treated according to the methods described herein include, without limitation, Entamoeba hystolytica, Giardia lamblia, Cryptosporidium muris, Trypanosomatida gambiense, Trypanosomatida rhodesiense, Trypanosomatida crusi, Leishmania mexicana, Leishmania braziliensis, Leishmania tropica, Leishmania donovani, Leishmania major, Toxoplasma gondii, Plasmodium vivax, Plasmodium ovale, Plasmodium malariae, Plasmodium falciparum, Plasmodium yoelli, Trichomonas vaginalis, and Histomonas meleagridis. Exemplary helminthic parasites include richuris trichiura, Ascaris lumbricoides, Enterobius vermicularis, Ancylostoma duodenale, Necator americanus, Strongyloides stercoralis, Wuchereria bancrofti, and Dracunculus medinensis, Schistosoma mansoni, Schistosoma haematobium, Schistosoma japonicum, Fasciola hepatica, Fasciola gigantica, Heterophyes, Paragonimus westermani, Taenia solium, Taenia saginata, Hymenolepis nana, and Echinococcus granulosus. Additional parasitic infections that can be treated according to the methods described herein include Onchocercas volvulus.

Conformation-specific NF-κB antibodies or antigen-binding fragments thereof can also be administered to a mammalian subject (e.g., a human) in order to treat, or to alleviate one or more symptoms of, fungal infections. Examples of fungal infections that may be treated according to the methods described herein include, without limitation, those caused by, e.g., Aspergillus, Candida, Malassezia, Trichosporon, Fusarium, Acremonium, Rhizopus, Mucor, Pneumocystis, and Absidia. Exemplary fungal infections that can be treated according to the methods described herein also include Pneumocystis carinii, Paracoccidioides brasiliensis and Histoplasma capsulatum.

### Methods of Treating Immune or Inflammatory Disorders

Conformation-specific NF-κB antibodies or antigen-binding fragments thereof described herein are useful therapeutics for the treatment of immune or inflammatory disorders, including autoimmune disorders, sepsis, septic shock, SIRS, and CRS. Conformation-specific NF-κB antibodies or antigen-binding fragments thereof can be administered to a mammalian subject, such as a human, suffering from an immune or inflammatory disorder, e.g., to modulate an immune response or an inflammatory response.

Exemplary compositions of the disclosure that can be used for these purposes include conformation-specific NF-κB antibodies or antigen-binding fragments thereof that bind specifically to an epitope including the pThr254-Pro motif of the p65 subunit of NF-κB (e.g., antibodies that bind specifically to the trans conformation of pThr254-Pro motif of the p65 subunit of NF-κB). Particularly, methods described herein include administering a conformation-specific NF-κB antibody or antigen-binding fragment thereof that contains one or more, or all, of the CDR sequences of a trans-mAb described herein, such as a human, humanized, or chimeric variant of a trans-mAb described herein, to a human or a non-human mammal in order to treat an immune or inflammatory disorder.

The methods described herein can be used to inhibit an immune response in a subject in need thereof, e.g., the subject has an autoimmune condition and is in need of inhibiting an immune response against self- or auto-antibodies (e.g., the subject has Graves' disease, systemic lupus erythematosus (SLE or lupus), type 1 diabetes, multiple sclerosis (MS), plaque psoriasis, rheumatoid arthritis (RA) or another autoimmune condition described herein). The methods described herein can also include a step of selecting a subject in need of inhibiting an immune response, e.g., selecting a subject who has or who has been identified to have an inflammatory or autoimmune condition.

The methods described herein can be used to modulate an immune response in a subject or cell by administering to a subject or cell a conformation-specific NF-κB antibody or antigen-binding fragment thereof in a dose (e.g., an effective amount) and for a time sufficient to modulate the immune response. These methods can be used to treat a subject in need of modulating an immune response, e.g., a subject with an inflammatory condition, an autoimmune disease or condition, or a chronic infection. One way to modulate an immune response is to modulate an immune cell activity. This modulation can occur in vivo (e.g., in a human subject or animal model) or in vitro (e.g., in acutely isolated or cultured cells, such as human cells from a patient, repository, or cell line, or rodent cells). The types of cells that can be modulated include T cells (e.g., peripheral T cells, cytotoxic T cells/CD8₊ T cells, T helper cells/CD4₊ T cells, memory T cells, regulatory T cells/Tregs, natural killer T cells/NKTs, mucosal associated invariant T cells, and gamma delta T cells), B cells (e.g., memory B cells, plasmablasts, plasma cells, follicular B cells/B-2 cells, marginal zone B cells, B-1 cells, regulatory B cells/Bregs), dendritic cells (e.g., myeloid DCs/conventional DCs, plasmacytoid DCs, or follicular DCs), granulocytes (e.g., eosinophils, mast cells, neutrophils, and basophils), monocytes, macrophages (e.g., peripheral macrophages or tissue resident macrophages), myeloid-derived suppressor cells, natural killer (NK) cells, innate lymphoid cells, thymocytes, and megakaryocytes.

The immune cell activities that can be modulated by administering to a subject or contacting a cell with an effective amount of a conformation-specific NF-κB antibody or antigen-binding fragment thereof described herein include activation (e.g., macrophage, T cell, NK cell, B cell, dendritic cell, neutrophil, eosinophil, or basophil activation), phagocytosis (e.g., macrophage, neutrophil, monocyte, mast cell, B cell, eosinophil, or dendritic cell phagocytosis), antibody-dependent cellular phagocytosis (e.g., ADCP by monocytes, macrophages, neutrophils, or dendritic cells), antibody-dependent cellular cytotoxicity (e.g., ADCC by NK cells, monocytes, macrophages, neutrophils, eosinophils, dendritic cells, or T cells), polarization (e.g., macrophage polarization toward an M1 or M2 phenotype or T cell polarization), proliferation (e.g., proliferation of B cells, T cells, monocytes, macrophages, dendritic cells, NK cells, mast cells, neutrophils, eosinophils, or basophils), lymph node homing (e.g., lymph node homing of T cells, B cells, dendritic cells, or macrophages), lymph node egress (e.g., lymph node egress of T cells, B cells, dendritic cells, or macrophages), recruitment (e.g., recruitment of B cells, T cells, monocytes, macrophages, dendritic cells, NK cells, mast cells, neutrophils, eosinophils, or basophils), migration (e.g., migration of B cells, T cells, monocytes, macrophages, dendritic cells, NK cells, mast cells, neutrophils, eosinophils, or basophils), differentiation (e.g., regulatory T cell differentiation), immune cell cytokine production, antigen presentation (e.g., dendritic cell, macrophage, and B cell antigen presentation), maturation (e.g., dendritic cell maturation), and degranulation (e.g., mast cell, NK cell, cytotoxic T cell, neutrophil, eosinophil, or basophil degranulation). Innervation of lymph nodes or lymphoid organs, development of high endothelial venules (HEVs), and development of ectopic or tertiary lymphoid organs (TLOs) can also be modulated using the methods described herein. Modulation can increase or decrease these activities.

In some embodiments, an effective amount a conformation-specific NF-κB antibody or antigen-binding fragment thereof is an amount sufficient to modulate (e.g., increase or decrease) one or more (e.g., 2 or more, 3 or more, 4 or more) of the following immune cell activities in the subject or cell: T cell polarization; T cell activation; dendritic cell activation; neutrophil activation; eosinophil activation; basophil activation; T cell proliferation; B cell proliferation; T cell proliferation; monocyte proliferation; macrophage proliferation; dendritic cell proliferation; NK cell proliferation; mast cell proliferation; neutrophil proliferation; eosinophil proliferation; basophil proliferation; cytotoxic T cell activation; circulating monocytes; peripheral blood hematopoietic stem cells; macrophage polarization; macrophage phagocytosis; macrophage ADCP, neutrophil phagocytosis; monocyte phagocytosis; mast cell phagocytosis; B cell phagocytosis; eosinophil phagocytosis; dendritic cell phagocytosis; macrophage activation; antigen presentation (e.g., dendritic cell, macrophage, and B cell antigen presentation); antigen presenting cell migration (e.g., dendritic cell, macrophage, and B cell migration); lymph node immune cell homing and cell egress (e.g., lymph node homing and egress of T cells, B cells, dendritic cells, or macrophages); NK cell activation; NK cell ADCC, mast cell degranulation; NK cell degranulation; cytotoxic T cell degranulation; neutrophil degranulation; eosinophil degranulation; basophil degranulation; neutrophil recruitment; eosinophil recruitment; NKT cell activation; B cell activation; regulatory T cell differentiation; dendritic cell maturation; development of high endothelial venules (HEVs); development of ectopic or tertiary lymphoid organs (TLOs); or lymph node or secondary lymphoid organ innervation. In certain embodiments, the immune response (e.g., an immune cell activity listed herein) is increased or decreased in the subject or cell at least 1%, 2%, 5%, 10%, 15%, 20%, 25%, 30%, 35%, 40%, 50%, 60%, 70%, 80%, 100%, 150%, 200%, 300%, 400%, 500% or more, compared to before the administration. In certain embodiments, the immune response is increased or decreased in the subject or cell between 5-20%, between 5-50%, between 10-50%, between 20-80%, between 20-70%, between 50-200%, between 100%-500%.

After a conformation-specific NF-κB antibody or antigen-binding fragment thereof is administered to treat a patient or contact a cell, a readout can be used to assess the effect on immune cell activity. Immune cell activity can be assessed by measuring a cytokine (e.g., IFN-γ, IL-2, IL-6, IL-12, IL-18, IL-27, TNFα, or TNFβ/LTα) a chemokine, or marker associated with a particular immune cell type. In certain embodiments, the parameter is increased or decreased in the subject at least 1%, 2%, 5%, 10%, 15%, 20%, 25%, 30%, 35%, 40%, 50%, 60%, 70%, 80%, 100%, 150%, 200%, 300%, 400%, 500% or more, compared to before the administration. In certain embodiments, the parameter is increased or decreased in the subject between 5-20%, between 5-50%, between 10-50%, between 20-80%, between 20-70%, between 50-200%, between 100%-500%. A conformation-specific NF-κB antibody or antigen-binding fragment thereof can be administered at a dose (e.g., an effective amount) and for a time sufficient to modulate an immune cell activity described herein below. When treating inflammatory conditions, administration of the conformation-specific NF-κB antibody or antigen-binding fragment thereof, such as a conformation-specific NF-κB antibody or antigen-binding fragment thereof that contains one or more, or all, of the CDR sequences of a trans-mAb described herein, such as a human, humanized, or chimeric variant of a trans-mAb described herein, achieves a reduction in one or more inflammatory cytokines, such as IL-6.

After a conformation-specific NF-κB antibody or antigen-binding fragment thereof is administered to treat a patient or contact a cell, a readout can be used to assess the effect on immune cell migration. Immune cell migration can be assessed by measuring the number of immune cells in a location of interest (e.g., a lymph node or secondary lymphoid organ, or a site of inflammation). Immune cell migration can also be assessed by measuring a marker associated with immune cell migration (e.g., P-selectin, E-selectin, PNAd, MAdCAM, VCAM-1, Chemokines, ICAM-1, ICAM-2, PECAM1 (CD31), JAM-A/-B/-C, ESAM, CD99, CD99L2, VE-cadherin, PVR, or S1P. In certain embodiments, the parameter is increased or decreased in the subject at least 1%, 2%, 5%, 10%, 15%, 20%, 25%, 30%, 35%, 40%, 50%, 60%, 70%, 80%, 100%, 150%, 200%, 300%, 400%, 500% or more, compared to before the administration. In certain embodiments, the parameter is increased or decreased in the subject between 5-20%, between 5-50%, between 10-50%, between 20-80%, between 20-70%, between 50-200%, between 100%-500%. A conformation-specific NF-κB antibody or antigen-binding fragment thereof can be administered at a dose (e.g., an effective amount) and for a time sufficient to modulate an immune cell migration.

A conformation-specific NF-κB antibody or antigen-binding fragment thereof described herein can affect immune cell migration. Immune cell migration between peripheral tissues, the blood, and the lymphatic system as well as lymphoid organs is essential for the orchestration of productive innate and adaptive immune responses. Immune cell migration is largely regulated by trafficking molecules including integrins, immunoglobulin cell-adhesion molecules (IgSF CAMs), cadherins, selectins, and a family of small cytokines called chemokines. Cell adhesion molecules and chemokines regulate immune cell migration by both inducing extravasation from the circulation into peripheral tissues and acting as guidance cues within peripheral tissues themselves. For extravasation to occur, chemokines must act in concert with multiple trafficking molecules including C-type lectins (L-, P-, and E-selectin), multiple integrins, and cell adhesion molecules (ICAM-1, VCAM-1 and MAdCAM-1) to enable a multi-step cascade of immune cell capturing, rolling, arrest, and transmigration via the blood endothelial barrier. Some trafficking molecules are constitutively expressed and manage the migration of immune cells during homeostasis, while others are specifically upregulated by inflammatory processes such as infection and autoimmunity.

Aberrant immune cell migration is observed in multiple immune-related pathologies. Immune cell adhesion deficiencies, caused by molecular defects in integrin expression, fucosylation of selectin ligands, or inside-out activation of integrins on leukocytes and platelets, lead to impaired immune cell migration into peripheral tissues. This results in leukocytosis and in increased susceptibility to recurrent bacterial and fungal infections, which can be difficult to treat and potentially life-threatening. Alternatively, exaggerated migration of specific immune cell subsets into specific peripheral tissues is associated with a multitude of pathologies. For example, excessive neutrophil accumulation in peripheral tissues contributes to the development of ischemia-reperfusion injury, such as that observed during acute myocardial infarction, stroke, shock and acute respiratory distress syndrome. Excessive Th1 inflammation characterized by tissue infiltration of interferon-gamma secreting effector T cells and activated macrophages is associated with atherosclerosis, allograft rejection, hepatitis, and multiple autoimmune diseases including multiple sclerosis, rheumatoid arthritis, psoriasis, Crohn's disease, type 1 diabetes and lupus erythematodes. Excessive Th2 inflammation characterized by tissue infiltration of IL-4, IL-5, and IL-13 secreting Th2 cells, eosinophils and mast cells is associated with asthma, food allergies and atopic dermatitis.

Conformation-specific NF-κB antibodies or antigen-binding fragments thereof described herein can be administered to a mammalian subject (e.g., a human) suffering from an immune or inflammatory disorder to improve the condition of the patient by modulating (e.g., increasing or decreasing) an immune response. Antibodies described herein can be administered to a subject, e.g., via any of the routes of administration described herein. Antibodies described herein can also be formulated with excipients, biologically acceptable carriers, and may be optionally conjugated to, admixed with, or co-administered separately (e.g., sequentially) with additional therapeutic agents. Immune or inflammatory disorders that can be treated by administration of antibodies or antigen-binding fragments thereof described herein include such disorders as acne vulgaris; acute respiratory distress syndrome; Addison's disease; adrenocortical insufficiency; adrenogenital syndrome; allergic conjunctivitis; allergic rhinitis; allergic intraocular inflammatory diseases, ANCA-associated small-vessel vasculitis; angioedema; ankylosing spondylitis; aphthous stomatitis; arthritis, asthma; atherosclerosis; atopic dermatitis; autoimmune disease; autoimmune hemolytic anemia; autoimmune hepatitis; Behcet's disease; Bell's palsy; berylliosis; bronchial asthma; bullous herpetiformis dermatitis; bullous pemphigoid; carditis; celiac disease; cerebral ischaemia; chronic obstructive pulmonary disease; cirrhosis; Cogan's syndrome; contact dermatitis; Crohn's disease; Cushing's syndrome; Cytokine Release Syndrome (CRS); dermatomyositis; diabetes mellitus; discoid lupus erythematosus; eosinophilic fasciitis; epicondylitis; erythema nodosum; exfoliative dermatitis; fibromyalgia; focal glomerulosclerosis; giant cell arteritis; gout; gouty arthritis; graft-versus-host disease; hand eczema; Henoch-Schonlein purpura; herpes gestationis; hirsutism; hypersensitivity drug reactions; idiopathic cerato-scleritis; idiopathic pulmonary fibrosis; idiopathic thrombocytopenic purpura; inflammatory bowel or gastrointestinal disorders, inflammatory dermatoses; juvenile rheumatoid arthritis; laryngeal edema; lichen planus; Loeffler's syndrome; lupus nephritis; lupus vulgaris; lymphomatous tracheobronchitis; macular edema; multiple sclerosis; musculoskeletal and connective tissue disorder; myasthenia gravis; myositis; obstructive pulmonary disease; ocular inflammation; organ transplant rejection; osteoarthritis; pancreatitis; pemphigoid gestationis; pemphigus vulgaris; polyarteritis nodosa; polymyalgia rheumatica; primary adrenocortical insufficiency; primary billiary cirrhosis; pruritus scroti; pruritis/inflammation, psoriasis; psoriatic arthritis; Reiter's disease; relapsing polychondritis; rheumatic carditis; rheumatic fever; rheumatoid arthritis; rosacea caused by sarcoidosis; rosacea caused by scleroderma; rosacea caused by Sweet's syndrome; rosacea caused by systemic lupus erythematosus; rosacea caused by urticaria; rosacea caused by zoster-associated pain; sarcoidosis; scleroderma; segmental glomerulosclerosis; sepsis; serum sickness; shoulder tendinitis or bursitis; Sjogren's syndrome; Still's disease; stroke-induced brain cell death; Sweet's disease; systemic dermatomyositis; SIRS; systemic lupus erythematosus; systemic sclerosis; Takayasu's arteritis; temporal arteritis; and thyroiditis; toxic epidermal necrolysis; tuberculosis; type-1 diabetes; ulcerative colitis; uveitis; vasculitis; and Wegener's granulomatosis. In particular, antibodies described herein may be used to treat sepsis, septic shock, SIRS, CRS, or autoimmune disorders.

Conformation-specific NF-κB antibodies or antigen-binding fragments thereof described herein can be administered to a patient (e.g., a mammalian patient, such as a human patient) in order to treat an immune or inflammatory disorder characterized dyregulation (e.g., increased activity or expression) or NF-κB (e.g., NF-κB signaling).

Conformation-specific NF-κB antibodies or antigen-binding fragments thereof can also be co-administered with an additional therapeutic agent (e.g., an immunotherapy agent), as described herein.

### Methods of Treating Sepsis and Septic Shock

Conformation-specific NF-κB antibodies or antigen-binding fragments thereof described herein are useful therapeutics for the treatment of sepsis, including, the treatment of septic shock (e.g., the prevention or reduction of septic shock). Conformation-specific NF-κB antibodies or antigen-binding fragments thereof can be administered to a mammalian subject, such as a human, suffering from or at risk of developing sepsis or septic shock. Subjects at higher risk of developing sepsis include subject at higher risk of contracting an infection. These include the very young, the very old, those with chronic illnesses, and those with a weakened or impaired immune system.

Exemplary compositions of the disclosure that can be used for these purposes include conformation-specific NF-κB antibodies or antigen-binding fragments thereof that bind specifically to an epitope including the pThr254-Pro motif of the p65 subunit of NF-κB (e.g., antibodies that bind specifically to the trans conformation of pThr254-Pro motif of the p65 subunit of NF-κB). Particularly, methods described herein include administering a conformation-specific NF-κB antibody or antigen-binding fragment thereof that contains one or more, or all, of the CDR sequences of a trans-mAb described herein, such as a human, humanized, or chimeric variant of a trans-mAb described herein, to a human or a non-human mammal in order to treat (e.g., ameliorate, reduce, or prevent) sepsis or septic shock. Sepsis is a potentially life-threatening condition characterized by an inflammatory immune response, and which may arise in response to infection, trauma, or disease. Sepsis may be associated with a bacterial infection, a viral infection (e.g., a betacoronavirus infection), a fungal infection, or a parasitic infection (e.g., as described herein). Common locations for the primary infection include the lungs, brain, urinary tract, skin, and abdominal organs. Risk factors include very young age, older age, a weakened immune system from conditions such as cancer or diabetes, major trauma, or burns. Sepsis may also arise independent from an infection and is the referred to as sterile sepsis. Sepsis therefore may also be associated with trauma, burns, pancreatitis, or ischaemic reperfusion.

Common signs and symptoms include fever, increased heart rate, increased breathing rate, and confusion. There may also be symptoms related to a specific infection, such as a cough with pneumonia, or painful urination with a kidney infection. In the very young, old, and people with a weakened immune system, there may be no symptoms of a specific infection and the body temperature may be low or normal, rather than high. Severe sepsis may be characterized by poor organ function or insufficient blood flow. Insufficient blood flow may be evident by low blood pressure, high blood lactate, or low urine output. Septic shock may be characterized by low blood pressure due to sepsis that does not improve after fluid replacement.

Conformation-specific NF-κB antibodies or antigen-binding fragments thereof described herein can be administered to a mammalian subject (e.g., a human) suffering from or at risk of developing sepsis or septic shock to prevent or improve the condition of the patient by modulating (e.g., increasing or decreasing) an immune response, as described above. For example, treatment with an antibody described herein may decrease the levels of pro-inflammatory cytokines in the subject. Immune cells that produce and secrete pro-inflammatory cytokines include T cells (e.g., Th cells) macrophages, B cells, and mast cells. Pro-inflammatory cytokines include interleukin-1 (IL-1, e.g., IL-1β), IL-5, IL-6, IL-8, IL-10, IL-12, IL-13, IL-18, tumor necrosis factor (TNF, e.g., TNFα), interferon gamma (IFNγ), and granulocyte macrophage colony stimulating factor (GMCSF). Treatment may reduce the level of one or more pro-inflamatory cytokines by 5%, 10%, 15%, 20%, 30%, 40%, 50%, 60%, 70%, 80%, 90%, 100%, 150%, 200%, 500%, or 1000% or more.

Administration of a conformation-specific NF-κB antibody or antigen-binding fragment thereof described herein to a mammalian subject (e.g., a human) suffering from or at risk of developing sepsis or septic shock may prevent, reduce, or ameliorate one or more symptoms associated with sepsis or septic shock. Symptoms associate with sepsis are known to those of skill in the art, and include increased white blood cell count, immature white blood cells in the circulation, elevated plasma C-reactive protein, elevated procalcitonin (PCT), low blood pressure, low central venous or mixed venous oxygen saturation, high cardiac index, low oxygen level, low urine output, high creatinine in the blood, coagulation (clotting) abnormalities, absent bowel sounds, low platelets in the blood, high bilirubin levels, high lactate in the blood, or decreased capillary filling or mottling. Administration of a conformation-specific NF-κB antibody or antigen-binding fragment thereof to a subject having or at risk of developing sepsis (including septic shock) may reduce any of the above-described symptoms by at least 1%, 2%, 5%, 10%, 15%, 20%, 25%, 30%, 35%, 40%, 50%, 60%, 70%, 80%, 100%, 150%, 200%, 300%, 400%, 500% or more, compared to before the administration.

### Methods of Treating Systemic Inflammatory Response Syndrome (SIRS)

Conformation-specific NF-κB antibodies or antigen-binding fragments thereof described herein are useful therapeutics for the treatment of SIRS, including the prevention of SIRS. Conformation-specific NF-κB antibodies or antigen-binding fragments thereof can be administered to a mammalian subject, such as a human, suffering from or at risk of developing SIRS.

Exemplary compositions of the disclosure that can be used for these purposes include conformation-specific NF-κB antibodies or antigen-binding fragments thereof that bind specifically to an epitope including the pThr254-Pro motif of the p65 subunit of NF-κB (e.g., antibodies that bind specifically to the trans conformation of pThr254-Pro motif of the p65 subunit of NF-κB). Particularly, methods described herein include administering a conformation-specific NF-κB antibody or antigen-binding fragment thereof that contains one or more, or all, of the CDR sequences of a trans-mAb described herein, such as a human, humanized, or chimeric variant of a trans-mAb described herein, to a human or a non-human mammal in order to treat (e.g., ameliorate, reduce, or prevent) SIRS.

SIRS is a serious condition related to systemic inflammation, organ dysfunction, and/or organ failure. It is characterized by abnormal regulation of cytokines, and may include both pro- and anti-inflammatory component. SIRS may occur in response to an infectious or non-infectious insult. SIRS may be associated with, for example, infection (e.g., bacterial, viral, fungal, or parasitic infection), trauma, burns, pancreatitis, ischaemic reperfusion, hemorrhage, complications of surgery, pulmonary embolism, aortic aneurysm, cardiac tamponade, anaphylaxis, or drug overdose. Manifestations of SIRS include, but are not limited to: increased or decreased body temperature (e.g., body temperature less than 36° C. (96.8° F.) or greater than 38° C. (100.4° F.)), increased heart rate (e.g., heart rate greater than 90 beats per minute), high respiratory rate (e.g., greater than 20 breaths per minute), an arterial partial pressure of carbon dioxide less than 4.3 kPa (32 mmHg), abnormal white blood cell count (e.g., a white blood cell count less than 4000 cells/mm³ (4×109 cells/L) or greater than 12,000 cells/mm³ (12×109 cells/L)), and/or the presence of greater than 10% immature neutrophils. When two or more of these criteria are met with or without evidence of infection, patients may be diagnosed with SIRS. Patients with SIRS and acute organ dysfunction may be termed severe SIRS. Additional criteria for the diagnosis of SIRS will be apparent to those of skill in the art (for example, as described in Balk RA, Systemic inflammatory response syndrome (SIRS): Where did it come from and is it still relevant today? Virulence. 5(1):20-26 (2014), which is incorporated herein by reference in its entirety). Administration of a conformation-specific NF-κB antibody or antigen-binding fragment thereof described herein to a mammalian subject (e.g., a human) suffering from or at risk of developing SIRS may prevent, reduce, or ameliorate one or more symptoms associated with SIRS, such as symptoms described herein or known to those of skill in the art. Administration of a conformation-specific NF-κB antibody or antigen-binding fragment thereof to a subject having or at risk of developing SIRS may reduce any of the above-described symptoms by at least 1%, 2%, 5%, 10%, 15%, 20%, 25%, 30%, 35%, 40%, 50%, 60%, 70%, 80%, 100%, 150%, 200%, 300%, 400%, 500% or more, compared to before the administration.

Conformation-specific NF-κB antibodies or antigen-binding fragments thereof described herein can be administered to a mammalian subject (e.g., a human) suffering from or at risk of developing SIRS to prevent or improve the condition of the patient by modulating (e.g., increasing or decreasing) an immune response, as described above. Treatment with an antibody described herein may decrease the levels of pro-inflammatory cytokines in the subject. Immune cells that produce and secrete pro-inflammatory cytokines include T cells (e.g., Th cells) macrophages, B cells, and mast cells. Pro-inflammatory cytokines include interleukin-1 (IL-1, e.g., IL-1β), IL-5, IL-6, IL-8, IL-10, IL-12, IL-13, IL-18, tumor necrosis factor (TNF, e.g., TNFα), interferon gamma (IFNγ), and granulocyte macrophage colony stimulating factor (GMCSF). Treatment may reduce the level of one or more pro-inflamatory cytokines by 5%, 10%, 15%, 20%, 30%, 40%, 50%, 60%, 70%, 80%, 90%, 100%, 150%, 200%, 500%, or 1000% or more.

### Methods of Treating Cytokine Release Syndrome (CRS)

Conformation-specific NF-κB antibodies or antigen-binding fragments thereof described herein are useful therapeutics for the treatment of CRS, including the prevention of CRS. Conformation-specific NF-κB antibodies or antigen-binding fragments thereof can be administered to a mammalian subject, such as a human, suffering from or at risk of developing CRS.

Exemplary compositions of the disclosure that can be used for these purposes include conformation-specific NF-κB antibodies or antigen-binding fragments thereof that bind specifically to an epitope including the pThr254-Pro motif of the p65 subunit of NF-κB (e.g., antibodies that bind specifically to the trans conformation of pThr254-Pro motif of the p65 subunit of NF-κB). Particularly, methods described herein include administering a conformation-specific NF-κB antibody or antigen-binding fragment thereof that contains one or more, or all, of the CDR sequences of a trans-mAb described herein, such as a human, humanized, or chimeric variant of a trans-mAb described herein, to a human or a non-human mammal in order to treat (e.g., ameliorate, reduce, or prevent) CRS.

CRS is a systemic inflammatory response that can be triggered by a variety of stimuli including infections and certain therapeutics. For example, CRS has been associated with antibody therapy, for example, following treatment with anti-thymocyte globulin, TGN1412, rituximab, obinutuzumab, alemtuzumab, brentuximab, dacetuzumab, nivolumab, or blinatumomab. CRS has also been associated with small molecule cancer therapeutics, such as xaliplatin or lenalidomide. CRS has also been observed in connection with stem cell transplantation, graft-versus-host disease, T cell-engaging therapies (e.g., CAR-T or T cell-engaging antibodies), infection (e.g., a bacterial or viral infection), or hemophagocytic syndromes (e.g., macrophage activation syndrome (MAS) or hemophagocytic lymphohistiocytosis (HLH)). The epidemiology, clinical presentation, pathophysiology, and differential diagnosis of CRS are known to those of skill in the art and have been described, for example, in Shimabukuro-Vornhagen, A. et al. Journal for ImmunoTherapy of Cancer. 6:56 (2018); Canna, S. W. and Behrens, E. M. Pediatr Clin N Am. 59:329-344 (2012); and Chavez, J. C. et al. Hematol Oncol Stem Cell Ther. https://doi.org/10.1016/j.hemonc.2019.05.005, each of which is incorporated herein by reference.

CRS can present with a variety of symptoms ranging from mild, flu-like symptoms to severe life-threatening manifestations corresponding to an overactive inflammatory response. Mild symptoms of CRS include fever, fatigue, headache, rash, arthralgia, and/or myalgia. More severe cases are characterized by hypotension and/or high fever and may progress to an uncontrolled systemic inflammatory response with vasopressor-requiring circulatory shock, vascular leakage, disseminated intravascular coagulation, and/or multi-organ system failure. Laboratory abnormalities that are common in patients with CRS are known to those of skill in the art and include cytopenias, elevated creatinine and liver enzymes, deranged coagulation parameters, and increased serum C-Reactive Protein (CRP).

Respiratory symptoms are common in patients with CRS. Mild cases may display cough and tachypnea but can progress to acute respiratory distress syndrome (ARDS) with dyspnea, hypoxemia, and bilateral opacities on chest X-ray. ARDS may sometimes require mechanical ventilation. Of note, in patients with CRS the need for mechanical ventilation is oftentimes not due to respiratory distress but instead a consequence of the inability to protect the airway secondary to neurotoxicity. Patients with severe CRS can also develop renal failure or signs of cardiac dysfunction with reduced ejection fraction on ultrasound. In addition, patients with severe CRS frequently display vascular leakage with peripheral and pulmonary edema.

In severe cases CRS can be accompanied by clinical signs and laboratory abnormalities that resemble hemophagocytic lymphohistiocytosis (HLH) or macrophage activation syndrome (MAS). Patients with CRS-associated HLH display the typical clinical and laboratory findings of HLH/MAS such as high fevers, highly elevated ferritin levels, and hypertriglyeridemia. In a phase III study of blinatumomab in B-ALL four out of 13 CRS patients showed signs of HLH.

The American Society for Transplantation and Cellular Therapy (ASTCT), has recently developed consensus guidelines for grading CAR-T toxicities including CRS. This further emphasized that the key hallmarks of CRS are fever, hypotension, and/or hypoxia. Fever is required for the diagnosis of CRS, although it may not persist for the entire duration of the CRS toxicity. The grading of CRS severity is then based on clinical criteria for hypotension and/or hypoxia. Although CRS may cause other organs to be affected (e.g., causing transaminitis or arrhythmias or other organ specific manifestations), these toxicities generally occur in concert with hypotension and/or hypoxia.

Administration of a conformation-specific NF-κB antibody or antigen-binding fragment thereof described herein to a mammalian subject (e.g., a human) suffering from or at risk of developing CRS may prevent, reduce, or ameliorate one or more symptoms associated with CRS, such as symptoms described herein or known to those of skill in the art. Administration of a conformation-specific NF-κB antibody or antigen-binding fragment thereof to a subject having or at risk of developing CRS may reduce any of the above-described symptoms by at least 1%, 2%, 5%, 10%, 15%, 20%, 25%, 30%, 35%, 40%, 50%, 60%, 70%, 80%, 100%, 150%, 200%, 300%, 400%, 500% or more, compared to before the administration.

Conformation-specific NF-κB antibodies or antigen-binding fragments thereof described herein can be administered to a mammalian subject (e.g., a human) suffering from or at risk of developing CRS to prevent or improve the condition of the patient by modulating (e.g., increasing or decreasing) an immune response, as described above. Treatment with an antibody described herein may decrease the levels of pro-inflammatory cytokines in the subject. Immune cells that produce and secrete pro-inflammatory cytokines include T cells (e.g., Th cells) macrophages, B cells, and mast cells. Pro-inflammatory cytokines include interleukin-1 (IL-1, e.g., IL-1β), IL-5, IL-6, IL-8, IL-10, IL-12, IL-13, IL-18, tumor necrosis factor (TNF, e.g., TNFα), interferon gamma (IFNγ), and granulocyte macrophage colony stimulating factor (GMCSF). Treatment may reduce the level of one or more pro-inflamatory cytokines by 5%, 10%, 15%, 20%, 30%, 40%, 50%, 60%, 70%, 80%, 90%, 100%, 150%, 200%, 500%, or 1000% or more.

### VI. Pharmaceutical Compositions

Pharmaceutical compositions containing a conformation-specific NF-κB antibody or antigen-binding fragment thereof described herein can be prepared using methods known in the art. Exemplary conformation-specific NF-κB antibodies or antigen-binding fragments thereof that can be incorporated into pharmaceutical compositions of the disclosure include conformation-specific NF-κB antibodies or antigen-binding fragments thereof that bind specifically to an epitope including the pThr254-Pro motif of the p65 subunit of NF-κB (e.g., antibodies that bind specifically to the trans conformation of pThr254-Pro motif of the p65 subunit of NF-κB). Particularly, conformation-specific NF-κB antibodies or antigen-binding fragments thereof that can be incorporated into pharmaceutical compositions of the disclosure include a conformation-specific NF-κB antibody or antigen-binding fragment thereof that contains one or more, or all, of the CDR sequences of a trans-mAb described herein, such as a human, humanized, or chimeric variant of a trans-mAb described herein.

Pharmaceutical compositions described herein may contain a conformation-specific NF-κB antibody or antigen-binding fragment thereof described herein in combination with one or more pharmaceutically acceptable excipients. For instance, pharmaceutical compositions described herein can be prepared using, e.g., physiologically acceptable carriers, excipients or stabilizers (Remington's Pharmaceutical Sciences 16th edition, Osol, A. Ed. (1980); incorporated herein by reference), and in a desired form, e.g., in the form of lyophilized formulations or aqueous solutions. The compositions can also be prepared so as to contain the active agent at a desired concentration. For example, a pharmaceutical composition described herein may contain at least 10% (e.g., 10%, 20%, 30%, 40%, 50%, 60%, 70%, 80%, 90%, 95%, 97%, 98%, 99%, 99.5%, 99.9%, or 100%) active agent by weight (w/w).

Additionally, an active agent that can be incorporated into a pharmaceutical formulation can itself have a desired level of purity. For example, a conformation-specific NF-κB antibody or antigen-binding fragment thereof described herein may be characterized by a certain degree of purity after isolating the antibody from cell culture media or after chemical synthesis, e.g., of a single-chain antibody fragment (e.g., scFv) by established solid phase peptide synthesis methods or native chemical ligation as described herein. A conformation-specific NF-κB antibody or antigen-binding fragment thereof described herein may be at least 10% pure prior to incorporating the antibody into a pharmaceutical composition (e.g., 10%, 20%, 30%, 40%, 50%, 60%, 70%, 80%, 90%, 95%, 97%, 98%, 99%, 99.5%, 99.9%, 99.99%, or 100% pure).

Pharmaceutical compositions of conformation-specific NF-κB antibodies or antigen-binding fragments thereof described herein can be prepared for storage as lyophilized formulations or aqueous solutions by mixing the antibody having the desired degree of purity with optional pharmaceutically acceptable carriers, excipients or stabilizers typically employed in the art, e.g., buffering agents, stabilizing agents, preservatives, isotonifiers, non-ionic detergents, antioxidants, and other miscellaneous additives. See, e.g., Remington's Pharmaceutical Sciences, 16th edition (Osol, ed. 1980; incorporated herein by reference). Such additives must be nontoxic to the recipients at the dosages and concentrations employed.

### Buffering Agents

Buffering agents help to maintain the pH in the range which approximates physiological conditions. They can be present at concentration ranging from about 2 mM to about 50 mM. Suitable buffering agents for use with conformation-specific NF-κB antibodies or antigen-binding fragments thereof described herein include both organic and inorganic acids and salts thereof such as citrate buffers {e.g., monosodium citrate-disodium citrate mixture, citric acid-trisodium citrate mixture, citric acid-monosodium citrate mixture, etc.), succinate buffers {e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers {e.g., fumaric acid-monosodium fumarate mixture, fumaric acid-disodium fumarate mixture, monosodium fumarate-disodium fumarate mixture, etc.), gluconate buffers (e.g., gluconic acid-sodium gluconate mixture, gluconic acid-sodium hydroxide mixture, gluconic acid-potassium gluconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers {e.g., acetic acid-sodium acetate mixture, acetic acid-sodium hydroxide mixture, etc.). Additionally, phosphate buffers, histidine buffers and trimethylamine salts such as Tris can be used.

### Preservatives

Preservatives can be added to a composition described herein to retard microbial growth, and can be added in amounts ranging from 0.2%-1% (w/v). Suitable preservatives for use with conformation-specific NF-κB antibodies or antigen-binding fragments thereof described herein include phenol, benzyl alcohol, meta-cresol, methyl paraben, propyl paraben, octadecyldimethylbenzyl ammonium chloride, benzalconium halides {e.g., chloride, bromide, and iodide), hexamethonium chloride, and alkyl parabens such as methyl or propyl paraben, catechol, resorcinol, cyclohexanol, and 3-pentanol. Isotonifiers sometimes known as “stabilizers” can be added to ensure isotonicity of liquid compositions described herein and include polhydric sugar alcohols, for example trihydric or higher sugar alcohols, such as glycerin, arabitol, xylitol, sorbitol and mannitol. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, a-monothioglycerol and sodium thio sulfate; low molecular weight polypeptides (e.g., peptides of 10 residues or fewer); proteins such as human serum albumin, bovine serum albumin, gelatin or immunoglobulins; hydrophilic polymers, such as polyvinylpyrrolidone monosaccharides, such as xylose, mannose, fructose, glucose; disaccharides such as lactose, maltose, sucrose and trisaccharides such as raffinose; and polysaccharides such as dextran. Stabilizers can be present in the range from 0.1 to 10,000 weights per part of weight active protein.

### Detergents

Non-ionic surfactants or detergents (also known as “wetting agents”) can be added to help solubilize the therapeutic agent as well as to protect the therapeutic protein against agitation-induced aggregation, which also permits the formulation to be exposed to shear surface stressed without causing denaturation of the protein. Suitable non-ionic surfactants include polysorbates (20, 80, etc.), polyoxamers (184, 188 etc.), Pluronic polyols, polyoxyethylene sorbitan monoethers (TWEEN®-20, TWEEN®-80, etc.). Non-ionic surfactants can be present in a range of about 0.05 mg/mL to about 1.0 mg/mL, for example about 0.07 mg/mL to about 0.2 mg/mL.

Additional miscellaneous excipients include bulking agents (e.g., starch), chelating agents (e.g., EDTA), antioxidants (e.g., ascorbic acid, methionine, vitamin E), and cosolvents.

### Other Pharmaceutical Carriers

Alternative pharmaceutically acceptable carriers that can be incorporated into a pharmaceutical composition described herein may include dextrose, sucrose, sorbitol, mannitol, starch, rubber arable, potassium phosphate, arginate, gelatin, potassium silicate, microcrystalline cellulose, polyvinylpyrrolidone, cellulose, water, syrups, methyl cellulose, methylhydroxy benzoate, propylhydroxy benzoate, talc, magnesium stearate, and mineral oils, but not limited to. A composition containing antibody described herein may further include a lubricant, a humectant, a sweetener, a flavoring agent, an emulsifier, a suspending agent, and a preservative. Details of suitable pharmaceutically acceptable carriers and formulations can be found in Remington's Pharmaceutical Sciences (19th ed., 1995), which is incorporated herein by reference.

### VII. Compositions and Methods for Combination Therapy

Pharmaceutical compositions described herein may optionally include more than one active agents. For instance, compositions described herein may contain a conformation-specific NF-κB antibody or antigen-binding fragment thereof conjugated to, admixed with, or administered separately from another pharmaceutically active molecule, (e.g., an immunotherapy agent, a chimeric antigen receptor (CAR-T) agent, a chemotherapeutic agent, a small molecule anti-cancer agent, a cancer vaccine, an antibacterial agent, an antifungal agent, or an antiviral agent). For instance, a conformation-specific NF-κB antibody or antigen-binding fragment thereof or therapeutic conjugate thereof (e.g., a drug-antibody conjugate described herein), may be admixed with one or more additional active agents that can be used to treat a disorder described herein, such as a cancer, an infection, or an immune disorder or an inflammatory disorder (e.g., sepsis, septic shock, SIRS, or CRS). Alternatively, pharmaceutical compositions described herein may be formulated for co-administration or sequential administration with one or more additional active agents (e.g., an immunotherapy agent, a chimeric antigen receptor (CAR-T) agent, a chemotherapeutic agent, a small molecule anti-cancer agent, a cancer vaccine, an antibacterial agent, an antifungal agent, or an antiviral agent) that can be used to treat a cancer, an infection, or an immune disorder or an inflammatory disorder (e.g., sepsis, septic shock, SIRS, or CRS).

Using the methods described herein, a conformation-specific NF-κB antibody or antigen-binding fragment thereof described herein may be co-administered with (e.g., admixed with) or administered separately from an additional therapeutic agent (e.g., an immunotherapy agent, a chimeric antigen receptor (CAR-T) agent, a chemotherapeutic agent, a small molecule anti-cancer agent, a cancer vaccine, an antibacterial agent, an antifungal agent, or an antiviral agent). For example, a conformation-specific NF-κB antibody or antigen-binding fragment thereof may be administered to a patient, such as a human patient suffering from a disorder described herein, either simultaneously or at different times. In some embodiments, the conformation-specific NF-κB antibody or antigen-binding fragment thereof is administered to the patient prior to administration of an additional therapeutic agent (e.g., an immunotherapy agent, a chimeric antigen receptor (CAR-T) agent, a chemotherapeutic agent, a small molecule anti-cancer agent, a cancer vaccine, an antibacterial agent, an antifungal agent, or an antiviral agent). Alternatively, the conformation-specific NF-κB antibody or antigen-binding fragment thereof may be administered to the patient after an additional therapeutic agent (e.g., an immunotherapy agent, a chimeric antigen receptor (CAR-T) agent, a chemotherapeutic agent, a small molecule anti-cancer agent, a cancer vaccine, an antibacterial agent, an antifungal agent, or an antiviral agent). For example, conformation-specific NF-κB antibody or antigen-binding fragment thereof may be administered to the patient after a failed treatment, such as a failed immunotherapy, chemotherapy, antibacterial, antiviral, or antifungal treatment. A physician of skill in the art can monitor the efficacy of treatment to determine whether the therapy has successfully ameliorated the pathology being treated using methods described herein and known in the art.

For instance, a physician of skill in the art may monitor the quantity of cancer cells in a sample isolated from a patient (e.g., a blood sample or biopsy sample), such as a human patient, for instance, using flow cytometry or FACS analysis. Additionally, or alternatively, a physician of skill in the art can monitor the progression of a cancerous disease in a patient, for instance, by monitoring the size of one or more tumors in the patient, for example, by CT scan, MRI, or X-ray analysis. A physician of skill in the art may monitor the progression of a cancer, such as a cancer described herein, by evaluating the quantity and/or concentration of tumor biomarkers in the patient, such as the quantity and/or concentration of cell surface-bound tumor associated antigens or secreted tumor antigens present in the blood of the patient as an indicator of tumor presence. A finding that the quantity of cancer cells, the size of a tumor, and/or the quantity or concentration of one or more tumor antigens present in the patient or in a sample isolated from the patient has not decreased, for instance, by a statistically significant amount following administration of the immunotherapy agent within a specified time period (e.g., from 1 day to 6 months, such as 1 day, 2 days, 3 days, 4 days, 5 days, 6 days, 7 days, 2 weeks, 3 weeks, 4 weeks, 2 months, 3 months, 4 months, 5 months, or 6 months) can indicate that the immunotherapy treatment has failed to ameliorate the cancer. Based on this indication, a physician of skill in the art may administer a conformation-specific NF-κB antibody or antigen-binding fragment thereof described herein.

Additionally, or alternatively, a physician of skill in the art may monitor the progression of an infectious disease by evaluating the symptoms of a patient suffering from such a pathology. For instance, a physician may monitor the patient by determining whether the frequency and/or severity of one or more symptoms of the infectious disease have stabilized (e.g., remained the same) or decreased following treatment with therapeutic agent. A finding that the quantity of bacterial, fungal, or parasitic cells or viral particles in a sample isolated from the patient and/or a finding that the frequency or severity of one or more symptoms of the infectious disease have not decreased, for instance, by a statistically significant amount following administration of the therapeutic agent within a specified time period (e.g., from 1 day to 6 months, such as 1 day, 2 days, 3 days, 4 days, 5 days, 6 days, 7 days, 2 weeks, 3 weeks, 4 weeks, 2 months, 3 months, 4 months, 5 months, or 6 months) can indicate that the therapeutic treatment has failed to ameliorate the infectious disease. Based on this indication, a physician of skill in the art may administer a conformation-specific NF-κB antibody or antigen-binding fragment thereof described herein.

### Immunotherapy Agents

A conformation-specific NF-κB antibody or antigen-binding fragment thereof described herein may be admixed, conjugated, administered with, or administered separately from an immunotherapy agent, for instance, for the treatment of a disorder described herein, such as a cancer, an infection, or an immune disorder or an inflammatory disorder (e.g., sepsis, septic shock, SIRS, or CRS). Exemplary immunotherapy agents useful in conjunction with the compositions and methods described herein include, without limitation, an anti-CTLA-4 agent, an anti-PD-1 agent, an anti-PD-L1 agent, an anti-PD-L2 agent, an anti-TNF-α cross-linking agent, an anti-TRAIL cross-linking agent, an anti-CD27 agent, an anti-CD30 agent, an anti-CD40 agent, an anti-4-1 BB agent, an anti-GITR agent, an anti-OX40 agent, an anti-TRAILR1 agent, an anti-TRAILR2 agent, and an anti-TWEAKR agent, as well as, for example, agents directed toward the immunological targets described in Table 1 of Mahoney et al., Cancer Immunotherapy, 14:561-584 (2015), the disclosure of which is incorporated herein by reference in its entirety. For example, the immunotherapy agent may be an anti-CTLA-4 antibody or antigen-binding fragment thereof, such as ipilimumab and tremelimumab. The immunotherapy agent may be an anti-PD-1 antibody or antigen-binding fragment thereof, such as nivolumab, pembrolizumab, avelumab, durvalumab, and atezolizumab. The immunotherapy agent may be an anti-PD-L1 antibody or antigen-binding fragment thereof, such as atezolizumab or avelumab. As other examples, immunological target 4-1 BB ligand may be targeted with an anti-4-1 BB ligand antibody; immunological target OX40L may be targeted with an anti-OX40L antibody; immunological target GITR may be targeted with an anti-GITR antibody; immunological target CD27 may be targeted with an anti-CD27 antibody; immunological target TL1A may be targeted with an anti-TL1A antibody; immunological target CD40L or CD40 may be targeted with an anti-CD40L antibody; immunological target LIGHT may be targeted with an anti-LIGHT antibody; immunological target BTLA may be targeted with an anti-BTLA antibody; immunological target LAG3 may be targeted with an anti-LAG3 antibody; immunological target TIM3 may be targeted with an anti-TIM3 antibody; immunological target Singlecs may be targeted with an anti-Singlecs antibody; immunological target ICOS ligand may be targeted with an anti-ICOS ligand antibody; immunological target B7-H3 may be targeted with an anti-B7-H3 antibody; immunological target B7-H4 may be targeted with an anti-B7-H4 antibody; immunological target VISTA may be targeted with an anti-VISTA antibody; immunological target TMIGD2 may be targeted with an anti-TMIGD2 antibody; immunological target BTNL2 may be targeted with an anti-BTNL2 antibody; immunological target CD48 may be targeted with an anti-CD48 antibody; immunological target KIR may be targeted with an anti-KIR antibody; immunological target LIR may be targeted with an anti-LIR antibody; immunological target ILT may be targeted with an anti-ILT antibody; immunological target NKG2D may be targeted with an anti-NKG2D antibody; immunological target NKG2A may be targeted with an anti-NKG2A antibody; immunological target MICA may be targeted with an anti-MICA antibody; immunological target MICB may be targeted with an anti-MICB antibody; immunological target CD244 may be targeted with an anti-CD244 antibody; immunological target CSF1R may be targeted with an anti-CSF1R antibody; immunological target IDO may be targeted with an anti-IDO antibody; immunological target TGFβ may be targeted with an anti-TGFβ antibody; immunological target CD39 may be targeted with an anti-CD39 antibody; immunological target CD73 may be targeted with an anti-CD73 antibody; immunological target CXCR4 may be targeted with an anti-CXCR4 antibody; immunological target CXCL12 may be targeted with an anti-CXCL12 antibody; immunological target SIRPA may be targeted with an anti-SIRPA antibody; immunological target CD47 may be targeted with an anti-CD47 antibody; immunological target VEGF may be targeted with an anti-VEGF antibody; and immunological target neuropilin may be targeted with an anti-neuropilin antibody (see, e.g., Table 1 of Mahoney et al.).

Immunotherapy agents that may be used in conjunction with the compositions and methods described herein include, for instance, an anti-TWEAK agent, an anti-cell surface lymphocyte protein agent, an anti-BRAF agent, an anti-MEK agent, an anti-CD33 agent, an anti-CD20 agent, an anti-HLA-DR agent, an anti-HLA class I agent, an anti-CD52 agent, an anti-A33 agent, an anti-GD3 agent, an anti-PSMA agent, an anti-Ceacan 1 agent, an anti-Galedin 9 agent, an anti-HVEM agent, an anti-VISTA agent, an anti-B7 H4 agent, an anti-HHLA2 agent, an anti-CD155 agent, an anti-CD80 agent, an anti-BTLA agent, an anti-CD160 agent, an anti-CD28 agent, an anti-CD226 agent, an anti-CEACAM1 agent, an anti-TIM3 agent, an anti-TIGIT agent, an anti-CD96 agent, an anti-CD70 agent, an anti-CD27 agent, an anti-LIGHT agent, an anti-CD137 agent, an anti-DR4 agent, an anti-CR5 agent, an anti-TNFRS agent, an anti-TNFR1 agent, an anti-FAS agent, an anti-CD95 agent, an anti-TRAIL agent, an anti-DR6 agent, an anti-EDAR agent, an anti-NGFR agent, an anti-OPG agent, an anti-RANKL agent, an anti-LTβ receptor agent, an anti-BCMA agent, an anti-TACI agent, an anti-BAFFR agent, an anti-EDAR2 agent, an anti-TROY agent, and an anti-RELT agent. For instance, the immunotherapy agent may be an anti-TWEAK antibody or antigen-binding fragment thereof, an anti-cell surface lymphocyte protein antibody or antigen-binding fragment thereof, an anti-BRAF antibody or antigen-binding fragment thereof, an anti-MEK antibody or antigen-binding fragment thereof, an anti-CD33 antibody or antigen-binding fragment thereof, an anti-CD20 antibody or antigen-binding fragment thereof, an anti-HLA-DR antibody or antigen-binding fragment thereof, an anti-HLA class I antibody or antigen-binding fragment thereof, an anti-CD52 antibody or antigen-binding fragment thereof, an anti-A33 antibody or antigen-binding fragment thereof, an anti-GD3 antibody or antigen-binding fragment thereof, an anti-PSMA antibody or antigen-binding fragment thereof, an anti-Ceacan 1 antibody or antigen-binding fragment thereof, an anti-Galedin 9 antibody or antigen-binding fragment thereof, an anti-HVEM antibody or antigen-binding fragment thereof, an anti-VISTA antibody or antigen-binding fragment thereof, an anti-B7 H4 antibody or antigen-binding fragment thereof, an anti-HHLA2 antibody or antigen-binding fragment thereof, an anti-CD155 antibody or antigen-binding fragment thereof, an anti-CD80 antibody or antigen-binding fragment thereof, an anti-BTLA antibody or antigen-binding fragment thereof, an anti-CD160 antibody or antigen-binding fragment thereof, an anti-CD28 antibody or antigen-binding fragment thereof, an anti-CD226 antibody or antigen-binding fragment thereof, an anti-CEACAM1 antibody or antigen-binding fragment thereof, an anti-TIM3 antibody or antigen-binding fragment thereof, an anti-TIGIT antibody or antigen-binding fragment thereof, an anti-CD96 antibody or antigen-binding fragment thereof, an anti-CD70 antibody or antigen-binding fragment thereof, an anti-CD27 antibody or antigen-binding fragment thereof, an anti-LIGHT antibody or antigen-binding fragment thereof, an anti-CD137 antibody or antigen-binding fragment thereof, an anti-DR4 antibody or antigen-binding fragment thereof, an anti-CR5 antibody or antigen-binding fragment thereof, an anti-TNFRS antibody or antigen-binding fragment thereof, an anti-TNFR1 antibody or antigen-binding fragment thereof, an anti-FAS antibody or antigen-binding fragment thereof, an anti-CD95 antibody or antigen-binding fragment thereof, an anti-TRAIL antibody or antigen-binding fragment thereof, an anti-DR6 antibody or antigen-binding fragment thereof, an anti-EDAR antibody or antigen-binding fragment thereof, an anti-NGFR antibody or antigen-binding fragment thereof, an anti-OPG antibody or antigen-binding fragment thereof, an anti-RANKL antibody or antigen-binding fragment thereof, an anti-LTβ receptor antibody or antigen-binding fragment thereof, an anti-BCMA antibody or antigen-binding fragment thereof, an anti-TACI antibody or antigen-binding fragment thereof, an anti-BAFFR antibody or antigen-binding fragment thereof, an anti-EDAR2 antibody or antigen-binding fragment thereof, an anti-TROY antibody or antigen-binding fragment thereof, or an anti-RELT antibody or antigen-binding fragment thereof.

In some embodiments, the immunotherapy agent is an anti-cell surface lymphocyte protein antibody or antigen-binding fragment thereof, such as an antibody or antigen-binding fragment thereof that binds one or more of CD1, CD2, CD3, CD4, CD5, CD6, CD7, CD8, CD9, CD10, CD11, CD12, CD13, CD14, CD15, CD16, CD17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD43, CD44, CD45, CD46, CD47, CD48, CD49, CD50, CD51, CD52, CD53, CD54, CD55, CD56, CD57, CD58, CD59, CD60, CD61, CD62, CD63, CD64, CD65, CD66, CD67, CD68, CD69, CD70, CD71, CD72, CD73, CD74, CD75, CD76, CD77, CD78, CD79, CD80, CD81, CD82, CD83, CD84, CD85, CD86, CD87, CD88, CD89, CD90, CD91, CD92, CD93, CD94, CD95, CD96, CD97, CD98, CD99, CD100, CD101, CD102, CD103, CD104, CD105, CD106, CD107, CD108, CD109, CD110, CD111, CD112, CD113, CD114, CD115, CD116, CD117, CD118, CD119, CD120, CD121, CD122, CD123, CD124, CD125, CD126, CD127, CD128, CD129, CD130, CD131, CD132, CD133, CD134, CD135, CD136, CD137, CD138, CD139, CD140, CD141, CD142, CD143, CD144, CD145, CD146, CD147, CD148, CD149, CD150, CD151, CD152, CD153, CD154, CD155, CD156, CD157, CD158, CD159, CD160, CD161, CD162, CD163, CD164, CD165, CD166, CD167, CD168, CD169, CD170, CD171, CD172, CD173, CD174, CD175, CD176, CD177, CD178, CD179, CD180, CD181, CD182, CD183, CD184, CD185, CD186, CD187, CD188, CD189, CD190, CD191, CD192, CD193, CD194, CD195, CD196, CD197, CD198, CD199, CD200, CD201, CD202, CD203, CD204, CD205, CD206, CD207, CD208, CD209, CD210, CD211, CD212, CD213, CD214, CD215, CD216, CD217, CD218, CD219, CD220, CD221, CD222, CD223, CD224, CD225, CD226, CD227, CD228, CD229, CD230, CD231, CD232, CD233, CD234, CD235, CD236, CD237, CD238, CD239, CD240, CD241, CD242, CD243, CD244, CD245, CD246, CD247, CD248, CD249, CD250, CD251, CD252, CD253, CD254, CD255, CD256, CD257, CD258, CD259, CD260, CD261, CD262, CD263, CD264, CD265, CD266, CD267, CD268, CD269, CD270, CD271, CD272, CD273, CD274, CD275, CD276, CD277, CD278, CD279, CD280, CD281, CD282, CD283, CD284, CD285, CD286, CD287, CD288, CD289, CD290, CD291, CD292, CD293, CD294, CD295, CD296, CD297, CD298, CD299, CD300, CD301, CD302, CD303, CD304, CD305, CD306, CD307, CD308, CD309, CD310, CD311, CD312, CD313, CD314, CD315, CD316, CD317, CD318, CD319, and/or CD320.

In some embodiments, the immunotherapy agent is an agent (e.g., a polypeptide, antibody, antigen-binding fragment thereof, a single-chain polypeptide, or construct thereof) that binds a chemokine or lymphokine, such as a chemokine or lymphokine involved in tumor growth. For instance, exemplary immunotherapy agents that may be used in conjunction with the compositions and methods described herein include agents (e.g., polypeptides, antibodies, antigen-binding fragments thereof, single-chain polypeptides, and constructs thereof) that bind and inhibit the activity of one or more, or all, of CXCL1, CXCL2, CXCL3, CXCL8, CCL2 and CCL5. Exemplary chemokines involved in tumor growth and that may be targeted using an immunotherapy agent as described herein include those described, for instance, in Chow et al., Cancer Immunol. Res., 2:1125-1131, 2014, the disclosure of which is incorporated herein by reference. Exemplary immunotherapy agents that may be used in conjunction with the compositions and methods described herein additionally include agents (e.g., polypeptides, antibodies, antigen-binding fragments thereof, single-chain polypeptides, and constructs thereof) that bind and inhibit the activity of one or more, or all, of CCL3, CCL4, CCL8, and CCL22, which are described, for instance, in Balkwill, Nat. Rev. Cancer, 4:540-550, 2004, the disclosure of which is incorporated herein by reference.

Additional examples of immunotherapy agents that can be used in conjunction with the compositions and methods described herein include Targretin, Interferon-alpha, clobestasol, Peg Interferon (e.g., PEGASYS®), prednisone, Romidepsin, Bexarotene, methotrexate, Trimcinolone cream, anti-chemokines, Vorinostat, gabapentin, antibodies to lymphoid cell surface receptors and/or lymphokines, antibodies to surface cancer proteins, and/or small molecular therapies like Vorinostat.

### Chemotherapy Agents and Radiation Therapy

A conformation-specific NF-κB antibody or antigen-binding fragment thereof described herein may be admixed, conjugated, administered with, or administered separately from a chemotherapy agent, for instance, for the treatment of a disorder described herein, such as a cancer, an infection, or an immune disorder or an inflammatory disorder (e.g., sepsis, septic shock, SIRS, or CRS). Exemplary chemotherapy agents useful in conjunction with the compositions and methods described herein include, without limitation, Abiraterone Acetate, ABITREXATE® (Methotrexate), ABRAXANE® (Paclitaxel Albumin), ADRIAMYCIN®, bleomycin, vinblastine, and dacarbazine (ABVD), ADRIAMYCIN®, bleomycin, vincristine sulfate, and etoposide phosphate (ABVE), ADRIAMYCIN®, bleomycin, vincristine sulfate, etoposide phosphate, prednisone, and cyclophosphamide (ABVE-PC), doxorubicin and cyclophosphamide (AC), doxorubicin, cyclophosphamide, and paclitaxel or docetaxel (AC-T), ADCETRIS® (Brentuximab Vedotin), cytarabine, daunorubicin, and etoposide (ADE), ado-trastuzumab emtansine, ADRIAMYCIN® (doxorubicin hydrochloride), afatinib dimaleate, AFINITOR® (Everolimus), AKYNZEO® (netupitant and palonosetron hydrochloride), ALDARA® (imiquimod), aldesleukin, ALECENSA® (alectinib), alectinib, alemtuzumab, ALKERAN® for Injection (Melphalan Hydrochloride), ALKERAN® tablets (melphalan), ALIMTA® (pemetrexed disodium), ALOXI® (palonosetron hydrochloride), AMBOCHLORIN® (chlorambucil), AMBOCLORIN® (Chlorambucil), aminolevulinic acid, anastrozole, aprepitant, AREDIA® (pamidronate disodium), ARIMIDEX® (anastrozole), AROMASIN® (exemestane), ARRANON® (nelarabine), arsenic trioxide, ARZERRA® (ofatumumab), asparaginase Erwinia chrysanthemi, AVASTIN® (bevacizumab), axitinib, azacitidine, BEACOPP Becenum (carmustine), BELEODAQ® (Belinostat), belinostat, bendamustine hydrochloride, bleomycin, etoposide, and cisplatin (BEP), bevacizumab, bexarotene, BEXXAR® (tositumomab and iodine ¹³¹I tositumomab), bicalutamide, BiCNU (carmustine), bleomycin, blinatumomab, BLINCYTO® (blinatumomab), bortezomib, BOSULIF® (bosutinib), bosutinib, brentuximab vedotin, busulfan, BUSULFEX® (busulfan), cabazitaxel, cabozantinib-S-malate, CAF, CAM PATH® (alemtuzumab), CAMPTOSAR® (irinotecan hydrochloride), capecitabine, CAPDX, CARAC® (fluorouracil), carboplatin, CARBOPLATIN-TAXOL®, carfilzomib, CARMUBRIS® (carmustine), carmustine, carmustine implant, CASODEX® (bicalutamide), CEENU (lomustine), cisplatin, etoposide, and methotrexate (CEM), ceritinib, CERUBIDINE® (daunorubicin hydrochloride), CERVARIX® (recombinant HPV bivalent vaccine), cetuximab, chlorambucil, chlorambucil-prednisone, CHOP, cisplatin, CLAFEN® (cyclophosphamide), clofarabine, CLOFAREX® (clofarabine), CLOLAR® (Clofarabine), CMF, cobimetinib, cometriq (cabozantinib-S-malate), COPDAC, COPP, COPP-ABV, COSMEGEN® (dactinomycin), COTELLIC® (cobimetinib), crizotinib, CVP, cyclophosphamide, CYFOS® (ifosfamide), CYRAMZA® (ramucirumab), cytarabine, cytarabine liposome, CYTOSAR-U® (cytarabine), CYTOXAN® (cyclophosphamide), dabrafenib, dacarbazine, DACOGEN® (decitabine), dactinomycin, daratumumab, DARZALEX® (daratumumab), dasatinib, daunorubicin hydrochloride, decitabine, degarelix, denileukin diftitox, denosumab, DEPOCYT® (cytarabine liposome), dexamethasone, dexrazoxane hydrochloride, dinutuximab, docetaxel, DOXIL® (doxorubicin hydrochloride), doxorubicin hydrochloride, DOX-SL® (doxorubicin hydrochloride), DTIC-DOME® (dacarbazine), EFUDEX (fluorouracil), ELITEK® (rasburicase), ELLENCE® (epirubicin hydrochloride), elotuzumab, ELOXATIN® (oxaliplatin), eltrombopag olamine, EMEND® (aprepitant), EMPLICITI® (elotuzumab), enzalutamide, epirubicin hydrochloride, EPOCH, ERBITUX® (cetuximab), eribulin mesylate, ERIVEDGE® (vismodegib), erlotinib hydrochloride, ERWINAZE® (asparaginase Erwinia chrysanthemi), ETOPOPHOS® (etoposide phosphate), etoposide, etoposide phosphate, EVACET® (doxorubicin hydrochloride liposome), everolimus, EVISTA® (raloxifene hydrochloride), EVOMELA® (melphalan hydrochloride), exemestane, 5-FU (5-fluorouracil), FARESTON® (toremifene), FARYDAK® (panobinostat), FASLODEX® (fulvestrant), FEC, FEMARA® (letrozole), filgrastim, FLUDARA® (fludarabine phosphate), fludarabine phosphate, FLUOROPLEX® (fluorouracil), fluorouracil injection, flutamide, FOLEX® (methotrexate), FOLEX® PFS (methotrexate), FOLFIRI, FOLFIRI-bevacizumab, FOLFIRI-cetuximab, FOLFIRINOX, FOLFOX, FOLOTYN® (pralatrexate), FU-LV, fulvestrant, GARDASIL® (recombinant HPV quadrivalent vaccine), GARDASIL 9® (recombinant HPV nonavalent vaccine), GAZYVA® (obinutuzumab), gefitinib, gemcitabine hydrochloride, gemcitabine-cisplatin, gemcitabine-oxaliplatin, gemtuzumab ozogamicin, GEMZAR® (gemcitabine hydrochloride), GILOTRIF® (afatinib dimaleate), GLEEVEC® (imatinib mesylate), GLIADEL® (carmustine implant), GLIADEL® wafer (carmustine implant), glucarpidase, goserelin acetate, HALAVEN® (eribulin mesylate), HERCEPTIN® (trastuzumab), HPV bivalent vaccine, HYCAMTIN® (topotecan hydrochloride), Hyper-CVAD, IBRANCE (palbociclib), IBRITUMOMAB® tiuxetan, ibrutinib, ICE, ICLUSIG® (ponatinib hydrochloride), IDAMYCIN® (idarubicin hydrochloride), idarubicin hydrochloride, idelalisib, IFEX® (ifosfamide), ifosfamide, ifosfamidum, IL-2 (aldesleukin), imatinib mesylate, IMBRUVICA® (ibrutinib), ilmiquimod, IMLYGIC® (talimogene laherparepvec), INLYTA (axitinib), recombinant interferon alpha-2b, intron A, tositumomab, such as ¹³¹I tositumomab, ipilimumab, IRESSA® (gefitinib), irinotecan hydrochloride, ISTODAX® (romidepsin), ixabepilone, ixazomib citrate, IXEMPRA® (ixabepilone), JAKAFI® (ruxolitinib phosphate), JEVTANA® (cabazitaxel), KADCYLA® (ado-trastuzumab emtansine), KEOXIFENE® (raloxifene hydrochloride), KEPIVANCE® (palifermin), KEYTRUDA® (pembrolizumab), KYPROLIS® (carfilzomib), lanreotide acetate, lapatinib ditosylate, lenalidomide, lenvatinib mesylate, LENVIMA® (lenvatinib mesylate), letrozole, leucovorin calcium, leukeran (chlorambucil), leuprolide acetate, levulan (aminolevulinic acid), LINFOLIZIN® (chlorambucil), LIPODOX® (doxorubicin hydrochloride liposome), lomustine, LONSURF® (trifluridine and tipiracil hydrochloride), LUPRON® (leuprolide acetate), LYNPARZA® (olaparib), MARQIBO® (vincristine sulfate liposome), MATULANE® (procarbazine hydrochloride), mechlorethamine hydrochloride, megestrol acetate, MEKINIST® (trametinib), melphalan, melphalan hydrochloride, mercaptopurine, MESNEX® (mesna), METHAZOLASTONE® (temozolomide), methotrexate, methotrexate LPF, MEXATE® (methotrexate), MEXATE-AQ® (methotrexate), mitomycin C, mitoxantrone hydrochloride, MITOZYTREX® (mitomycin C), MOPP, MOZOBIL® (plerixafor), MUSTARGEN® (mechlorethamine hydrochloride), MUTAMYCIN® (mitomycin C), MYLERAN® (busulfan), MYLOSAR® (azacitidine), MYLOTARG® (gemtuzumab ozogamicin), nanoparticle paclitaxel, NAVELBINE® (vinorelbine tartrate), NECITUMUMAB, nelarabine, NEOSAR® (cyclophosphamide), netupitant and palonosetron hydrochloride, NEUPOGEN® (filgrastim), NEXAVAR® (sorafenib tosylate), NILOTINIB, NINLARO® (ixazomib citrate), nivolumab, NOLVADEX® (tamoxifen citrate), NPLATE® (romiplostim), obinutuzumab, ODOMZO® (sonidegib), OEPA, ofatumumab, OFF, olaparib, omacetaxine mepesuccinate, ONCASPAR® (pegaspargase), ondansetron hydrochloride, ONIVYDE® (irinotecan hydrochloride liposome), ONTAK® (denileukin diftitox), OPDIVO® (nivolumab), OPPA, osimertinib, oxaliplatin, paclitaxel, paclitaxel albumin-stabilized nanoparticle formulation, PAD, palbociclib, palifermin, palonosetron hydrochloride, palonosetron hydrochloride and netupitant, pamidronate disodium, panitumumab, panobinostat, PARAPLAT® (carboplatin), PARPLATIN® (carboplatin), pazopanib hydrochloride, PCV, pegaspargase, peginterferon alpha-2b, PEG-INTRON® (peginterferon alpha-2b), pembrolizumab, pemetrexed disodium, PERJETA® (pertuzumab), pertuzumab, PLATINOL® (cisplatin), PLATINOL-AQ® (cisplatin), plerixafor, pomalidomide, POMALYST® (pomalidomide), ponatinib hydrochloride, PORTRAZZA® (necitumumab), pralatrexate, prednisone, procarbazine hydrochloride, PROLEUKIN® (aldesleukin), PROLIA® (denosumab), PROMACTA (eltrombopag olamine), PROVENGE® (sipuleucel-T), PURINETHOL® (mercaptopurine), PURIXAN® (mercaptopurine), ²²³Ra dichloride, raloxifene hydrochloride, ramucirumab, rasburicase, R-CHOP, R-CVP, recombinant human papillomavirus (HPV), recombinant interferon alpha-2b, regorafenib, R-EPOCH, REVLIMID® (lenalidomide), RHEUMATREX® (methotrexate), RITUXAN® (rituximab), rolapitant hydrochloride, romidepsin, romiplostim, rubidomycin (daunorubicin hydrochloride), ruxolitinib phosphate, SCLEROSOL® intrapleural aerosol (talc), siltuximab, sipuleucel-T, somatuline depot (lanreotide acetate), sonidegib, sorafenib tosylate, SPRYCEL® (dasatinib), STANFORD V, sterile talc powder (talc), STERITALC® (talc), STIVARGA® (regorafenib), sunitinib malate, SUTENT® (sunitinib malate), SYLATRON® (peginterferon alpha-2b), SYLVANT® (siltuximab), SYNOVIR® (thalidomide), SYNRIBO® (omacetaxine mepesuccinate), thioguanine, TAC, TAFINLAR® (dabrafenib), TAGRISSO® (osimertinib), talimogene laherparepvec, tamoxifen citrate, tarabine PFS (cytarabine), TARCEVA (erlotinib hydrochloride), TARGRETIN® (bexarotene), TASIGNA® (nilotinib), TAXOL® (paclitaxel), TAXOTERE® (docetaxel), TEMODAR® (temozolomide), temsirolimus, thalidomide, THALOMID® (thalidomide), thioguanine, thiotepa, TOLAK® (topical fluorouracil), topotecan hydrochloride, toremifene, TORISEL® (temsirolimus), TOTECT® (dexrazoxane hydrochloride), TPF, trabectedin, trametinib, TREANDA® (bendamustine hydrochloride), trifluridine and tipiracil hydrochloride, TRISENOX® (arsenic trioxide), TYKERB® (lapatinib ditosylate), UNITUXIN® (dinutuximab), uridine triacetate, VAC, vandetanib, VAMP, VARUBI® (rolapitant hydrochloride), vectibix (panitumumab), VeIP, VELBAN® (vinblastine sulfate), VELCADE® (bortezomib), VELSAR (vinblastine sulfate), VEMURAFENIB, VIADUR (leuprolide acetate), VIDAZA (azacitidine), vinblastine sulfate, VINCASAR® PFS (vincristine sulfate), vincristine sulfate, vinorelbine tartrate, VIP, vismodegib, VISTOGARD® (uridine triacetate), VORAXAZE® (glucarpidase), vorinostat, VOTRIENT® (pazopanib hydrochloride), WELLCOVORIN® (leucovorin calcium), XALKORI® (crizotinib), XELODA® (capecitabine), XELIRI, XELOX, XGEVA® (denosumab), XOFIGO® (²²³Ra dichloride), XTANDI® (enzalutamide), YERVOY® (ipilimumab), YONDELIS® (trabectedin), ZALTRAP® (ziv-aflibercept), ZARXIO® (filgrastim), ZELBORAF® (vemurafenib), ZEVALIN® (ibritumomab tiuxetan), ZINECARD® (dexrazoxane hydrochloride), ziv-aflibercept, ZOFRAN® (ondansetron hydrochloride), ZOLADEX® (gGoserelin acetate), zoledronic acid, ZOLINZA® (vorinostat), ZOMETA® (zoledronic acid), ZYDELIG® (idelalisib), ZYKADIA® (ceritinib), and ZYTIGA (abiraterone acetate).

Additionally, or alternatively, a conformation-specific NF-κB antibody or antigen-binding fragment thereof described herein may be administered simultaneously with, or administered separately from, radiation therapy. For instance, a physician of skill in the art may administer radiation therapy to a patient, such as a human patient suffering from a cancer described herein, by treating the patient with external and/or internal electromagnetic radiation. The energy delivered by such radiation, which is typically in the form of X-rays, gamma rays, and similar forms of low-wavelength energy, can cause oxidative damage to the DNA of cancer cells, thereby leading to cell death, for instance, by apoptosis. External radiation therapy can be administered, for instance, using machinery such as a radiation beam to expose the patient to a controlled pulse of electromagnetic radiation. Additionally, or alternatively, the patient may be administered internal radiation, for instance, by administering to the patient a therapeutic agent that contains a radioactive substituent, such as agents that contain ²²³Ra or ¹³¹I, which emit high-energy alpha and beta particles, respectively. Exemplary therapeutic agents that may be conjugated to a radiolabel include, for example, small molecule chemotherapeutics, antibodies, and antigen-binding fragments thereof, among others. For instance, a conformation-specific NF-κB antibody or antigen-binding fragment thereof described herein may be conjugated to a radioactive substituent or a moiety that ligate such a substituent, for example, using bond-forming techniques known in the art or described herein. Such conjugates can be administered to the subject in order to deliver a therapeutic dosage of radiation therapy and a conformation-specific NF-κB antibody or antigen-binding fragment thereof described herein in a simultaneous administration.

### Chimeric Antigen Receptor (CAR-T) Agents

Additional agents that can be conjugated to, admixed with, or administered separately from conformation-specific NF-κB antibodies or antigen-binding fragments thereof described herein include T lymphocytes that exhibit reactivity with a specific antigen associated with a particular pathology. For instance, conformation-specific NF-κB antibodies or antigen-binding fragments thereof described herein can be formulated for administration with a T cell that expresses a chimeric antigen receptor (CAR-T) in order to treat a disorder described herein, such as a cancer, an infection, or an immune disorder or an inflammatory disorder (e.g., sepsis, septic shock, SIRS, or CRS). Conformation-specific NF-κB antibodies or antigen-binding fragments thereof can synergize with CAR-T therapy. CAR-T cells can be administered to a patient prior to, concurrently with, or after administration of a conformation-specific NF-κB antibody or antigen-binding fragment thereof in order to treat a mammalian subject (e.g., a human) suffering from a cancer, an infection, or an immune disorder or an inflammatory disorder (e.g., sepsis, septic shock, SIRS, or CRS).

CAR-T therapy is a particularly robust platform for targeting cancer cells in view of the ability to genetically engineer T lymphocytes to express an antigen receptor specific to a tumor-associated antigen. For instance, identification of antigens overexpressed on the surfaces of tumors and other cancer cells can inform the design and discovery of chimeric T cell receptors, which are often composed of cytoplasmic and transmembrane domains derived from a naturally-occurring T cell receptor operatively linked to an extracellular scFv fragment that specifically binds to a particular antigenic peptide. T cells can be genetically modified in order to express an antigen receptor that specifically binds to a particular tumor antigen by any of a variety of genome editing techniques described herein or known in the art. Exemplary techniques for modifying a T cell genome so as to incorporate a gene encoding a chimeric antigen receptor include the CRISPER/Cas, zinc finger nuclease, TALEN, ARCUS™ platforms described herein. Methods for the genetic engineering of CAR-T lymphocytes have been described, e.g., in WO 2014/127261, WO 2014/039523, WO 2014/099671, and WO 20120790000; the disclosures of each of which are incorporated by reference herein.

CAR-T cells useful in the compositions and methods described herein include those that have been genetically modified such that the cell does not express the endogenous T cell receptor. For instance, a CAR-T cell may be modified by genome-editing techniques, such as those described herein, so as to suppress expression of the endogenous T cell receptor in order to prevent graft-versus-host reactions in a patient receiving a CAR-T infusion. Additionally, or alternatively, CAR-T cells can be genetically modified so as to reduce the expression of one or more endogenous MHC proteins. This is a particularly useful technique for the infusion of allogeneic T lymphocytes, as recognition of foreign MHC proteins represents one mechanism that promotes allograft rejection. One of skill in the art can also modify a T lymphocyte so as to suppress the expression of immune suppressor proteins, such as programmed cell death protein 1 (PD-1) and cytotoxic T lymphocyte-associated protein 4 (CTLA-4). These proteins are cell surface receptors that, when activated, attenuate T cell activation. Infusion of CAR-T cells that have been genetically modified so as to diminish the expression of one or more immunosupressor proteins represents one strategy that can be used to prolong the T lymphocyte-mediated cytotoxicity in vivo.

In addition to deleting specific genes, one can also modify CAR-T cells in order to express a T cell receptor with a desired antigen specificity. For instance, one can genetically modify a T lymphocyte in order to express a T cell receptor that specifically binds to a tumor-associated antigen in order to target infused T cells to cancer cells. An exemplary T cell receptor that may be expressed by a CAR-T cell is one that binds PD-L1, a cell surface protein that is often overexpressed on various tumor cells. As PD-L1 activates PD-1 on the surface of T lymphocytes, targeting this tumor antigen with CAR-T therapy can synergize with conformation-specific NF-κB antibodies or antigen-binding fragments thereof described herein in order to increase the duration of an immune response mediated by a T lymphocyte in vivo. CAR-T cells can also be modified so as to express a T cell receptor that specifically binds an antigen associated with one or more infectious disease, such as an antigen derived from a viral protein, a bacterial cell, a fungus, or other parasitic organism.

### Antibacterial Agents

A conformation-specific NF-κB antibody or antigen-binding fragment thereof described herein may be admixed, conjugated, administered with, or administered separately from an antibacterial agent, for instance, for the treatment of a disorder described herein, such as a cancer, an infection, or an immune disorder or an inflammatory disorder (e.g., sepsis, septic shock, SIRS, or CRS). Exemplary antibacterial agents useful in conjunction with the compositions and methods described herein include, without limitation, Afenide, Amikacin, Amoxicillin, Ampicillin, Arsphenamine, Augmentin, Azithromycin, Azlocillin, Aztreonam, Bacampicillin, Bacitracin, Balofloxacin, Besifloxacin, Capreomycin, Carbacephem (loracarbef), Carbenicillin, Cefacetrile (cephacetrile), Cefaclomezine, Cefaclor, Cefadroxil (cefadroxyl), Cefalexin (cephalexin), Cefaloglycin (cephaloglycin), Cefalonium (cephalonium), Cefaloram, Cefaloridine (cephaloradine), Cefalotin (cephalothin), Cefamandole, Cefaparole, Cefapirin (cephapirin), Cefatrizine, Cefazaflur, Cefazedone, Cefazolin (cephazolin), Cefcanel, Cefcapene, Cefclidine, Cefdaloxime, Cefdinir, Cefditoren, Cefedrolor, Cefempidone, Cefepime, Cefetamet, Cefetrizole, Cefivitril, Cefixime, Cefluprenam, Cefmatilen, Cefmenoxime, Cefmepidium, Cefmetazole, Cefodizime, Cefonicid, Cefoperazone, Cefoselis, Cefotaxime, Cefotetan, Cefovecin, Cefoxazole, Cefoxitin, Cefozopran, Cefpimizole, Cefpirome, Cefpodoxime, Cefprozil (cefproxil), Cefquinome, Cefradine (cephradine), Cefrotil, Cefroxadine, Cefsumide, Ceftaroline, Ceftazidime, Ceftazidime/Avibactam, Cefteram, Ceftezole, Ceftibuten, Ceftiofur, Ceftiolene, Ceftioxide, Ceftizoxime, Ceftobiprole, Ceftriaxone, Cefuracetime, Cefuroxime, Cefuzonam, Cephalexin, Chloramphenicol, Chlorhexidine, Ciprofloxacin, Clarithromycin, Clavulanic Acid, Clinafloxacin, Clindamycin, Cloxacillin, Colimycin, Colistimethate, Colistin, Crysticillin, Cycloserine 2, Demeclocycline, Dicloxacillin, Dirithromycin, Doripenem, Doxycycline, Efprozil, Enoxacin, Ertapenem, Erythromycin, Ethambutol, Flucloxacillin, Flumequine, Fosfomycin, Furazolidone, Gatifloxacin, Geldanamycin, Gemifloxacin, Gentamicin, Glycopeptides, Grepafloxacin, Herbimycin, Imipenem, Isoniazid, Kanamycin, Levofloxacin, Lincomycin, Linezolid, Lipoglycopeptides, Lomefloxacin, Meropenem, Meticillin, Metronidazole, Mezlocillin, Minocycline, Mitomycin, Moxifloxacin, Mupirocin, Nadifloxacin, Nafcillin, Nalidixic Acid, Neomycin, Netilmicin, Nitrofurantoin, Norfloxacin, Ofloxacin, Oxacillin, Oxazolidinones, Oxolinic Acid, Oxytetracycline, Oxytetracycline, Paromomycin, Pazufloxacin, Pefloxacin, Penicillin G, Penicillin V, Pipemidic Acid, Piperacillin, Piromidic Acid, Pivampicillin, Pivmecillinam, Platensimycin, Polymyxin B, Pristinamycin, Prontosil, Prulifloxacin, Pvampicillin, Pyrazinamide, Quinupristin/dalfopristin, Rifabutin, Rifalazil, Rifampin, Rifamycin, Rifapentine, Rosoxacin, Roxithromycin, Rufloxacin, Sitafloxacin, Sparfloxacin, Spectinomycin, Spiramycin, Streptomycin, Sulbactam, Sulfacetamide, Sulfamethizole, Sulfamethoxazole, Sulfanilimide, Sulfisoxazole, Sulphonamides, Sultamicillin, Teicoplanin, Telavancin, Telithromycin, Temafloxacin, Tetracycline, Thiamphenicol, Ticarcillin, Tigecycline, Tinidazole, Tobramycin, Tosufloxacin, Trimethoprim, Trimethoprim-Sulfamethoxazole, Troleandomycin, Trovafloxacin, Tuberactinomycin, Vancomycin, and Viomycin, or a pharmaceutically acceptable salt thereof.

### Antiviral Agents

A conformation-specific NF-κB antibody or antigen-binding fragment thereof described herein may be admixed, conjugated, administered with, or administered separately from an antiviral agent, for instance, for the treatment of a disorder described herein, such as a cancer, an infection, or an immune disorder or an inflammatory disorder (e.g., sepsis, septic shock, SIRS, or CRS). Exemplary antiviral agents useful in conjunction with the compositions and methods described herein include, without limitation, vidarabine, acyclovir, gancyclovir, valgancyclovir, AZT (zidovudine), ddl (didanosine), ddC (zalcitabine), d4T (stavudine), 3TC (lamivudine), nevirapine, delavirdine, saquinavir, ritonavir, indinavir, nelfinavir, ribavirin, and interferon, or a pharmaceutically acceptable salt thereof.

### Antifungal Agents

A conformation-specific NF-κB antibody or antigen-binding fragment thereof described herein may be admixed, conjugated, administered with, or administered separately from an antifungal agent, for instance, for the treatment of a disorder described herein, such as a cancer, an infection, or an immune disorder or an inflammatory disorder (e.g., sepsis, septic shock, SIRS, or CRS). Exemplary antifiungal agents useful in conjunction with the compositions and methods described herein include, without limitation, Abafungin, Albaconazole, Amorolfin, Amphotericin B, Anidulafungin, Bifonazole, Butenafine, Butoconazole, Candicidin, Caspofungin, Ciclopirox, Clotrimazole, Econazole, Fenticonazole, Filipin, Fluconazole, Flucytosine, Griseofulvin, Haloprogin, Hamycin, Isavuconazole, Isoconazole, Itraconazole, Ketoconazole, Micafungin, Miconazole, Naftifine, Natamycin, Nystatin, Omoconazole, Oxiconazole, Polygodial, Posaconazole, Ravuconazole, Rimocidin, Sertaconazole, Sulconazole, Terbinafine, Terconazole, Tioconazole, Tolnaftate, Undecylenic Acid, and Voriconazole, or a pharmaceutically acceptable salt thereof.

### VIII. Routes of Administration and Dosing

A conformation-specific NF-κB antibody or antigen-binding fragment thereof described herein can be administered to a mammalian subject (e.g., a human) by a variety of routes, such as orally, transdermally, subcutaneously, intranasally, intravenously, intramuscularly, intraocularly, intratumorally, parenterally, topically, intrathecally and intracerebroventricularly, for the treatment of, e.g., the diseases and conditions described herein. The most suitable route for administration in any given case will depend on the particular polypeptide administered, the patient, pharmaceutical formulation methods, administration methods (e.g., administration time and administration route), the patients age, body weight, sex, severity of the diseases being treated, the patient's diet, and the patient's excretion rate. A physician having ordinary skill in the art can readily determine an effective amount of a conformation-specific NF-κB antibody or antigen-binding fragment thereof for administration to a mammalian subject (e.g., a human) in need thereof. For example, a physician could start prescribing doses of an antibody described herein at levels lower than that required in order to achieve the desired therapeutic effect and gradually increase the dosage until the desired effect is achieved. Alternatively, a physician may begin a treatment regimen by administering a conformation-specific NF-κB antibody or antigen-binding fragment thereof at a high dose and subsequently administering progressively lower doses until a therapeutic effect is achieved. In general, a suitable daily dose of an antibody or antigen-binding fragment thereof will be an amount of the compound which is the lowest dose effective to produce a therapeutic effect. A conformation-specific NF-κB antibody or antigen-binding fragment thereof described herein may be administered, e.g., by injection, such as by intravenous, intramuscular, intraperitoneal, or subcutaneous injection, optionally proximal to the site of the target tissue. A daily dose of a therapeutic composition of an antibody described herein may be administered as a single dose or as two, three, four, five, six or more doses administered separately at appropriate intervals throughout the day, week, month, or year, or as needed, optionally, in unit dosage forms. While it is possible for an antibody described herein to be administered alone, it may also be administered as a pharmaceutical formulation in combination with excipients, carriers, and optionally, additional therapeutic agents.

The effective dose of a conformation-specific NF-κB antibody or antigen-binding fragment thereof described herein can range, for instance, from about 0.0001 to about 100 mg/kg of body weight per single (e.g., bolus) administration, multiple administrations or continuous administration (e.g., a continuous infusion), or to achieve a serum concentration of 0.0001-5000 μg/mL serum concentration per single (e.g., bolus) administration, multiple administrations or continuous administration (e.g., continuous infusion), or any effective range or value therein depending on the condition being treated, the route of administration and the age, weight, and condition of the subject. In certain embodiments, each dose can range from about 0.0001 mg to about 500 mg/kg of body weight. For instance, a pharmaceutical composition described herein may be administered in a daily dose in the range of 0.001-100 mg/kg (body weight). The dose may be administered one or more times (e.g., 2-10 times) per day, week, month, or year to a mammalian subject (e.g., a human) in need thereof.

Conformation-specific NF-κB antibodies or antigen-binding fragments thereof can be administered to a patient by way of a continuous intravenous infusion or as a single bolus administration. The conformation-specific NF-κB antibodies or antigen-binding fragments thereof may be administered to a patient in an amount of, for example, from 0.01 μg to about 5 g in a volume of, for example, from 10 μL to 10 mL. The conformation-specific NF-κB antibodies or antigen-binding fragments thereof may be administered to a patient over the course of several minutes to several hours. For example, the conformation-specific NF-κB antibodies or antigen-binding fragments thereof described herein may be administered to a patient over the course of from 5 minutes to 5 hours, such as over the course of 5 minutes, 10 minutes, 15 minutes, 20 minutes, 25 minutes, 30 minutes, 35 minutes, 40 minutes, 45 minutes, 50 minutes, 55 minutes, 60 minutes, 65 minutes, 70 minutes, 80 minutes, 90 minutes, 95 minutes, 100 minutes, 105 minutes, 110 minutes, 115 minutes, 120 minutes, 125 minutes, 130 minutes, 135 minutes, 140 minutes, 145 minutes, 150 minutes, 155 minutes, 160 minutes, 165 minutes, 170 minutes, 175 minutes, 180 minutes, 185 minutes, 190 minutes, 195 minutes, 200 minutes, 205 minutes, 210 minutes, 215 minutes, 220 minutes, 225 minutes, 230 minutes, 235 minutes, 240 minutes, 245 minutes, 250 minutes, 255 minutes, 260 minutes, 265 minutes, 270 minutes, 275 minutes, 280 minutes, 285 minutes, 290 minutes, 295 minutes, or 300 minutes, or more.

Antagonistic conformation-specific NF-κB antibodies or antigen-binding fragments thereof described herein may be administered in combination with one or more additional active agents (e.g., an immunotherapy agent, a chimeric antigen receptor (CAR-T) agent, a chemotherapeutic agent, a small molecule anti-cancer agent, a cancer vaccine, an antibacterial agent, an antifungal agent, or an antiviral agent)

When an additional therapeutic agent is administered to a patient in combination with a conformation-specific NF-κB antibody or antigen-binding fragment thereof, the additional therapeutic agent may be administered to the patient by way of a single bolus administration or continuous intravenous infusion.

When conformation-specific NF-κB antibodies or antigen-binding fragments thereof are administered to a patient in combination with an additional therapeutic agent, the conformation-specific NF-κB antibody or antigen-binding fragment thereof and the additional therapeutic agent may be co-administered to the patient, for example, by way of a continuous intravenous infusion or bolus administration of the first agent, followed by a continuous intravenous infusion or bolus administration of the second agent. The administration of the two agents may occur concurrently. Alternatively, the administration of the conformation-specific NF-κB antibody or antigen-binding fragment thereof may precede or follow the administration of the additional therapeutic agent. In some embodiments, administration of the second agent (e.g., the conformation-specific NF-κB antibody or antigen-binding fragment thereof) commences within from about 5 minutes to about 4 weeks, or more, of the end of the administration of the first agent (e.g., the additional therapeutic agent). For example, administration of the second agent may commence within about 5 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 2 days, 3 days, 4 days, 5 days, 6 days, 7 days, 2 weeks, 3 weeks, 4 weeks, or more, of the end of the administration of the first agent.

Therapeutic compositions can be administered with medical devices known in the art. For example, in an embodiment, a therapeutic composition described herein can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in conjunction with the compositions and methods described herein include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

### IX. Diagnostic Methods

The present invention features methods and compositions to treat, diagnose, and monitor the progression of a disorder described herein (e.g., a cancer, an infection, or an immune disorder or inflammatory disorder (e.g., sepsis, septic shock, SIRS, or CRS)). The methods and compositions can include the detection and measurement of, for example, NF-κB (e.g., the p65 subunit of NF-κB), or any fragments or derivatives thereof, containing a phosphorylated Thr-Pro motif in a cis or trans conformation (e.g., pThr254-Pro, specifically the trans conformation of pThr254-Pro or the ratio of trans:trans of pThr254-Pro). The methods can include measurement of absolute levels of NF-κB (e.g., the p65 subunit of NF-κB), or any fragments or derivatives thereof in a cis or trans conformation as compared to a normal reference.

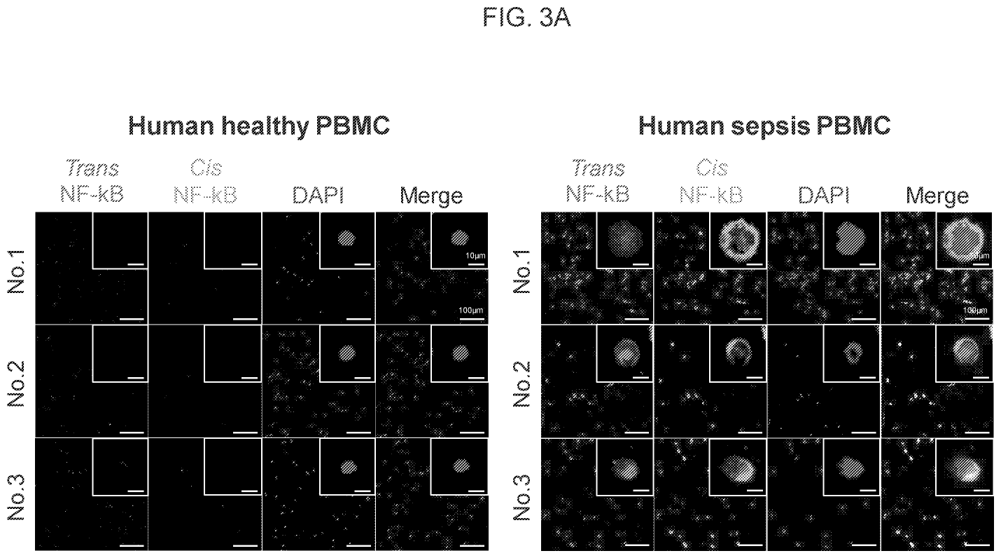

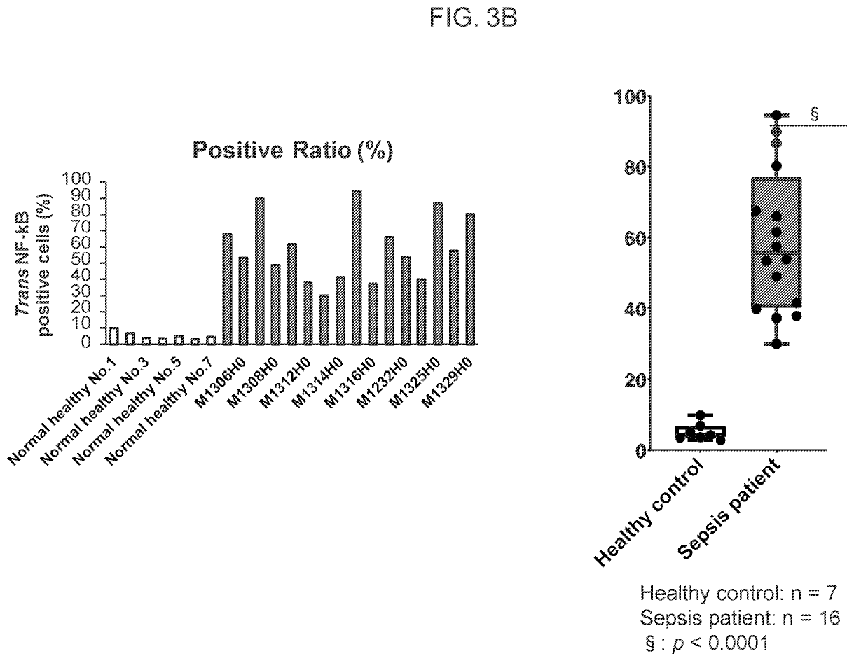

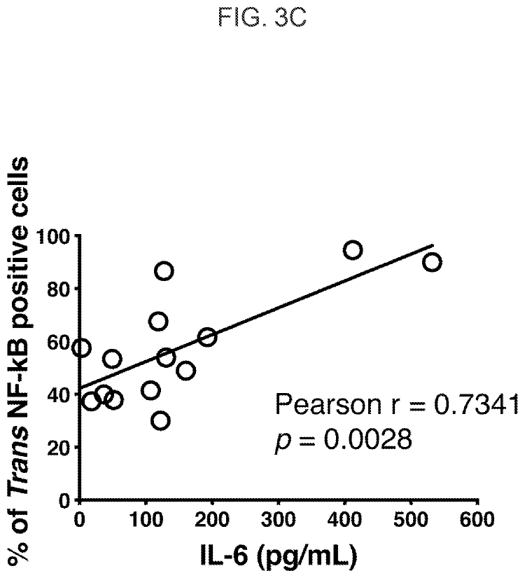

In particular, the inventors have observed that human sepsis patients have an increased level of the active nuclear form of NF-kB in their peripheral blood mononuclear cells (PBMCs) as compared to healthy control (see, e.g., Example 3, FIGS. 3A-3C). Accordingly, an antibody or antigen-binding fragment thereof described herein (e.g., an antibody or antigen-binding fragment thereof that specifically binds an epitope including the trans conformation of pThr254-Pro of the p65 subunit of NF-κB) may be used (1) to diagnose a patient as having a particular disorder characterized by inflammation (e.g., a cancer, an infection, or an immune disorder or inflammatory disorder such as sepsis, septic shock, SIRS, or CRS); (2) to determine whether a subject is likely to be responsive to treatment, and/or (3) to monitor the therapeutic response to treatment.

A serum level of a NF-κB (e.g., the p65 subunit of NF-κB), or any fragments or derivatives thereof in the cis or trans conformation that is less than 5 ng/ml, 4 ng/ml, 3 ng/ml, 2 ng/ml, or less than 1 ng/ml serum is considered to be predictive of a good outcome in a patient diagnosed with a disorder (e.g., a disorder associated with a deregulation of NF-κB activity). A serum level of the substrate in the cis or trans conformation that is greater than 5 ng/ml, 10 ng/ml, 20 ng/ml, 30 ng/ml, 40 ng/ml, or 50 ng/ml is considered diagnostic of a poor outcome in a subject already diagnosed with a disorder, e.g., associated with a deregulation of NF-κB activity.

For diagnoses based on relative levels of substrate in a particular conformation (e.g., a NF-κB substrate in the trans conformation), a subject with a disorder (e.g., a disorder associated with a deregulation of NF-κB activity) will show an alteration (e.g., an increase of 10%, 20%, 30%, 40%, 50%, 60%, 70%, 80%, 90%, or more) in the amount of the substrate in, for example, the trans conformation. A subject with a disorder may be diagnosed on the basis of an increased ratio of trans:cis of pThr254-Pro, for example as measured in PBMCs (e.g., an increase of 10%, 20%, 30%, 40%, 50%, 60%, 70%, 80%, 90%, or more). A normal reference sample can be, for example, a prior sample taken from the same subject prior to the development of the disorder or of symptoms suggestive of the disorder, a sample from a subject not having the disorder, a sample from a subject not having symptoms of the disorder, or a sample of a purified reference polypeptide in a given conformation at a known normal concentration (i.e., not indicative of the disorder).

Standard methods may be used to measure levels of the substrate in any bodily fluid, including, but not limited to, urine, blood, serum, plasma, saliva, amniotic fluid, or cerebrospinal fluid. Such methods include immunoassay, ELISA, Western blotting, and quantitative enzyme immunoassay techniques.

The disclosure specifically contemplates method of determining the level of nuclear NF-κB activity (e.g., determining the level of trans conformation of pThr254-Pro or the ratio of trans:cis of pThr254-Pro) in a sample from subject, wherein the method includes the steps of (i) contacting a sample from the subject with any one of the antibodies or antigen-binding fragments thereof described herein (e.g., an antibody or antigen-binding fragment thereof that specifically binds an epitope including the trans conformation of pThr254-Pro of the p65 subunit of NF-κB); and (ii) determining the level of the nuclear NF-κB in the sample of (i) by determining the level of the antibody or antigen-binding fragment thereof bound to the nuclear NF-κB. The method may further include the step of (iii) comparing the level of the nuclear NF-κB determined in (ii) to a reference value of nuclear NF-κB. The reference value of nuclear NF-κB may be the average level of nuclear NF-κB in a population of subjects having an immune disorder or an inflammatory disorder (e.g., sepsis, such as septic shock, SIRS, or CRS), an infection, or a cancer. The reference value of nuclear NF-κB may be the average level of nuclear NF-κB in a population of subjects not having sepsis, an infection, or a cancer. Wherein the level of nuclear NF-κB determined in (ii) is greater than the reference value of nuclear NF-κB, then the subject is treated with a therapeutically effective amount of any one of the antibodies or antigen-binding fragments thereof described herein, any one of the polynucleotides described herein, any one of the vectors described herein, or any one of the host cells described herein.

For diagnostic purposes, the conformation-specific antibodies may be labeled. Labeling of the antibody is intended to encompass direct labeling of the antibody by coupling (e.g., physically linking) a detectable substance to the antibody, as well as indirect labeling the antibody by reacting the antibody with another reagent that is directly labeled. For example, the antibody can be labeled with a radioactive or fluorescent marker whose presence and location in a subject can be detected by standard imaging techniques.

The diagnostic methods described herein can be used individually or in combination with any other diagnostic method described herein for a more accurate diagnosis of the presence or severity of a disorder (e.g., a cancer, an infection, or an immune disorder or an inflammatory disorder). Examples of additional methods for diagnosing such disorders include, e.g., examining a subject's health history, immunohistochemical staining of tissues, computed tomography (CT) scans, or culture growths.

### Subject Monitoring

The diagnostic methods described herein can also be used to monitor the progression of a disorder (e.g., a cancer, an infection, or an immune disorder or an inflammatory disorder (e.g., sepsis, septic shock, SIRS, or CRS)) during therapy or to determine the dosages of therapeutic compounds. In one embodiment, the levels of NF-κB p65 trans-pThr254-Pro and/or NF-κB activity are measured repeatedly as a method of diagnosing the disorder and monitoring the treatment or management of the disorder. In order to monitor the progression of the disorder in a subject, subject samples can be obtained at several time points and may then be compared. For example, the diagnostic methods can be used to monitor subjects during therapy. In this example, serum samples from a subject can be obtained before treatment with a therapeutic agent, again during treatment with a therapeutic agent, and again after treatment with a therapeutic agent. In this example, the level of NF-κB p65 trans-pThr254-Pro, the ratio of trans:cis of pThr254-Pro, and/or NF-κB activity in a subject is closely monitored using the conformation-specific antibodies of the invention and, if the level of NF-κB p65 trans-pThr254-Pro and/or NF-κB activity begins to increase during therapy, the therapeutic regimen for treatment of the disorder can be modified as determined by the clinician (e.g., the dosage of the therapy may be changed or a different therapeutic may be administered). The monitoring methods of the invention may also be used, for example, in assessing the efficacy of a particular drug or therapy in a subject, determining dosages, or in assessing progression, status, or stage of a cancer, infection, sepsis, SIRS, or CRS.

### X. Kits Containing Conformation-Specific NF-κB Antibodies or Antigen-Binding Fragments Thereof

Also included herein are kits that contain conformation-specific NF-κB antibodies or antigen-binding fragments thereof. The kits provided herein may contain any of the conformation-specific NF-κB antibodies or antigen-binding fragments thereof described above, as well as any of the polynucleotides encoding these polypeptides, vectors containing these polynucleotides, or cells engineered to express and secrete antibodies described herein (e.g., prokaryotic or eukaryotic cells).

Exemplary compositions of the disclosure that can be incorporated into a kit described herein include conformation-specific NF-κB antibodies or antigen-binding fragments thereof that bind specifically to an epitope including the pThr254-Pro motif of the p65 subunit of NF-κB (e.g., antibodies that bind specifically to the trans conformation of pThr254-Pro motif of the p65 subunit of NF-κB). Particularly, methods described herein include administering a conformation-specific NF-κB antibody or antigen-binding fragment thereof that contains one or more, or all, of the CDR sequences of a trans-mAb described herein, such as a human, humanized, or chimeric variant of a trans-mAb described herein, to a human or a non-human mammal in order to treat a cancer.

A kit described herein may include reagents that can be used to produce the compositions described herein (e.g., a conformation-specific NF-κB antibody or antigen-binding fragment thereof). Optionally, kits described herein may include reagents that can induce the expression of a conformation-specific NF-κB antibody or antigen-binding fragment thereof within cells (e.g., mammalian cells), such as doxycycline or tetracycline. In other cases, a kit described herein may contain a compound capable of binding and detecting a fusion protein that contains a conformation-specific NF-κB antibody or antigen-binding fragment thereof and an epitope tag. For instance, in such cases a kit described herein may contain maltose, glutathione, a nickel-containing complex, an anti-FLAG antibody, an anti-myc antibody, an anti-HA antibody, biotin, or streptavidin.

Kits described herein may also include reagents that are capable of detecting a conformation-specific NF-κB antibody or antigen-binding fragment thereof directly. Examples of such reagents include secondary antibodies that selectively recognize and bind particular structural features within the Fc region of a conformation-specific NF-κB antibody or antigen-binding fragment thereof described herein. Kits described herein may contain secondary antibodies that recognize the Fc region of a conformation-specific NF-κB antibody or antigen-binding fragment thereof and that are conjugated to a fluorescent molecule. These antibody-fluorophore conjugates provide a tool for analyzing the localization of conformation-specific NF-κB antibodies or antigen-binding fragments thereof, e.g., in a particular tissue or cultured mammalian cell using established immunofluorescence techniques. In some embodiments, kits described herein may include additional fluorescent compounds that exhibit known sub-cellular localization patterns. These reagents can be used in combination with another antibody-fluorophore conjugate, e.g., one that specifically recognizes a different receptor on the cell surface in order to analyze the localization of a conformation-specific NF-κB antibody or antigen-binding fragment thereof relative to other cell-surface proteins.

Kits described herein may also contain a reagent that can be used for the analysis of a patient's response to treatment by administration of conformation-specific NF-κB antibodies or antigen-binding fragments thereof described herein. For instance, kits described herein may include a conformation-specific NF-κB antibody or antigen-binding fragment thereof and one or more reagents that can be used to determine the quantity of T-reg cells in a blood sample withdrawn from a subject (e.g., a human) that is undergoing treatment with an antibody described herein. Kits may contain, e.g., antibodies that selectively bind cell-surface antigens presented by T-reg cells, such as CD4 and CD25. Optionally, these antibodies may be labeled with a fluorescent dye, such as fluorescein or tetramethylrhodamine, in order to facilitate analysis of T-reg cells by fluorescence-activated cell sorting (FACS) methods known in the art. Kits described herein may optionally contain one or more reagents that can be used to quantify tumor-reactive T lymphocytes in order to determine the effectiveness of an antagonistic conformation-specific NF-κB antibodies or antigen-binding fragments thereof in restoring tumor-infiltrating lymphocyte proliferation. For instance, kits described herein may contain an antibody that selectively binds cell-surface markers on the surface of a cytotoxic T cell, such as CD8 or CD3. Optionally, these antibodies may be labeled with fluorescent molecules so as to enable quantitation by FACS analysis.

A kit described herein may also contain one or more reagents useful for determining the affinity and selectivity of a conformation-specific NF-κB antibody or antigen-binding fragment thereof described herein for one or more peptides derived from NF-κB. For instance, a kit may contain a conformation-specific NF-κB antibody or antigen-binding fragment thereof and one or more reagents that can be used in an ELISA assay to determine the K_{D} of an antibody described herein for one or more peptides that present a NF-κB epitope in a conformation similar to that of the epitope in the native protein. A kit may contain, e.g., a microtiter plate containing wells that have been previously conjugated to avidin, and may contain a library of NF-κB-derived peptides, each of which conjugated to a biotin moiety. Such a kit may optionally contain a secondary antibody that specifically binds to the Fc region of a conformation-specific NF-κB antibody or antigen-binding fragment thereof described herein, and the secondary antibody may be conjugated to an enzyme (e.g., horseradish peroxidase) that catalyzes a chemical reaction that results in the emission of luminescent light.

Kits described herein may also contain a conformation-specific NF-κB antibody or antigen-binding fragment thereof described herein and a reagent that can be conjugated to such an antibody, including those previously described (e.g., a cytotoxic agent, a fluorescent molecule, a bioluminescent molecule, a molecule containing a radioactive isotope, a molecule containing a chelating group bound to a paramagnetic ion, etc). These kits may additionally contain instructions for how the conjugation of a conformation-specific NF-κB antibody or antigen-binding fragment thereof described herein to a second molecule, such as those described above, can be achieved.

A kit described herein may also contain a vector containing a polynucleotide that encodes a conformation-specific NF-κB antibody or antigen-binding fragment thereof, such as any of the vectors described herein. Alternatively, a kit may include mammalian cells (e.g., CHO cells) that have been genetically altered to express and secrete conformation-specific NF-κB antibodies or antigen-binding fragments thereof or fragments thereof from the nuclear genome of the cell. Such a kit may also contain instructions describing how expression of the conformation-specific NF-κB antibody or antigen-binding fragment thereof from a polynucleotide can be induced, and may additionally include reagents (such as, e.g., doxycycline or tetracycline) that can be used to promote the transcription of these polynucleotides. Such kits may be useful for the manufacture of conformation-specific NF-κB antibodies or antigen-binding fragments thereof described herein.

Other kits described herein may include tools for engineering a prokaryotic or eukaryotic cell (e.g., a CHO cell or a BL21(DE3) E. coli cell) so as to express and secrete a conformation-specific NF-κB antibody or antigen-binding fragment thereof described herein from the nuclear genome of the cell. For example, a kit may contain CHO cells stored in an appropriate media and optionally frozen according to methods known in the art. The kit may also provide a vector containing a polynucleotide that encodes a nuclease (e.g., such as the CRISPER/Cas, zinc finger nuclease, TALEN, ARCUS™ nucleases described herein) as well as reagents for expressing the nuclease in the cell. The kit can additionally provide tools for modifying the polynucleotide that encodes the nuclease so as to enable one to alter the DNA sequence of the nuclease in order to direct the cleavage of a specific target DNA sequence of interest. Examples of such tools include primers for the amplification and site-directed mutagenesis of the polynucleotide encoding the nuclease of interest. The kit may also include restriction enzymes that can be used to selectively excise the nuclease-encoding polynucleotide from the vector and subsequently re-introduce the modified polynucleotide back into the vector once the user has modified the gene. Such a kit may also include a DNA ligase that can be used to catalyze the formation of covalent phosphodiester linkages between the modified nuclease-encoding polynucleotide and the target vector. A kit described herein may also provide a polynucleotide encoding a conformation-specific NF-κB antibody or antigen-binding fragment thereof, as well as a package insert describing the methods one can use to selectively cleave a particular DNA sequence in the genome of the cell in order to incorporate the polynucleotide encoding a conformation-specific NF-κB antibody or antigen-binding fragment thereof into the genome at this site. Optionally, the kit may provide a polynucleotide encoding a fusion protein that contains a conformation-specific NF-κB antibody or antigen-binding fragment thereof or fragment thereof and an additional polypeptide, such as, e.g., those described herein.

### EXAMPLES

The following examples are put forth so as to provide those of ordinary skill in the art with a description of how the compositions and methods claimed herein are performed, made, and evaluated, and are intended to be purely exemplary described herein and are not intended to limit the scope of what the inventor regards as her invention.

### Example 1. Production and Sequencing of Antibodies Selective for Cis and Trans pThr254-Pro Motif of the p65 Subunit of NF-κB

We developed novel antibodies selective against the cytoplasmic (cis) or nuclear (trans) form of NF-κB by raising mouse monoclonal antibodies against the p65 subunit of NF-κB that is phosphorylated specifically on the Thr254 residue. Trans-mAbs 1 through 10 were generated by immunizing mice with a pThr254-Pro p65 peptide, followed by generating antibody-producing hybridoma clones using the mouse spleen. A corresponding cis-specific antibody was also generated. The DNA sequences of the light chains (FIG. 8) and heavy chains (FIG. 9) of the trans-mAbs against of the p65 subunit of NF-κB were determined using 5′ RACE RT-PCR techniques. The predicted protein sequences are highly conserved in framework regions, with well-defined complementarity determining regions (CDRs). Some parts of the predicted protein sequences of the light and heavy chains of trans mAbs were also confirmed by Mass Spectrometry.

Examples 2-6 describe the characterization of trans-mAbs 1 through 4 (collectively referred to for the purpose of Example 2-6 as “trans-mAb”). Trans-mAbs 1 through 4 share significant sequence similarity. Each of trans-mAbs 1-4 includes a light chain variable domain selected from SEQ ID NOs: 4 and 12, which differ by only a single amino acid in CDR-L1. Each of trans-mAbs 1-4 also includes a heavy chain variable domain selected from SEQ ID NOs: 8 and 10, which differ only by a single amino acid in CDR-H2.

### Example 2. Development of Monoclonal Antibodies Selectively Against the Nuclear Active Form (Trans-mAb) or the Cytoplasmic Inactive Form (Cis-mAb) of NF-κB

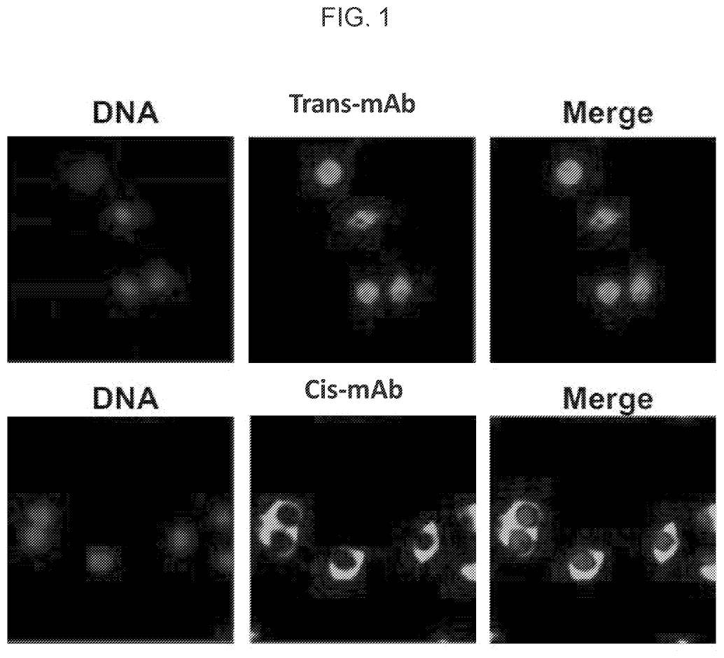

Immune cells, such as macrophages, dendritic cells, or monocytes, were stimulated with Toll-like receptor ligands such as LPS. NF-κB was induced and localized to two different subcellular locations, as detected by our two different antibodies; one was localized in the cytoplasm and the other was localized in the nucleus (FIG. 1). The monoclonal antibody recognizing the nuclear form is an antibody that specifically recognizes the trans conformation of pThr254-Pro of p65 of NF-κB, while the monoclonal antibody recognizing the cytoplasmic form is an antibody that specifically recognizes the cis conformation of pThr254-Pro of p65 of NF-κB.

### Example 3. A Trans-Specific Anti-Nuclear NF-κB Antibody Reduces Cytokine Release in Cell Cultures and Fully Prevents Mortality of LPS-Induced Septic Shock in Animal Models

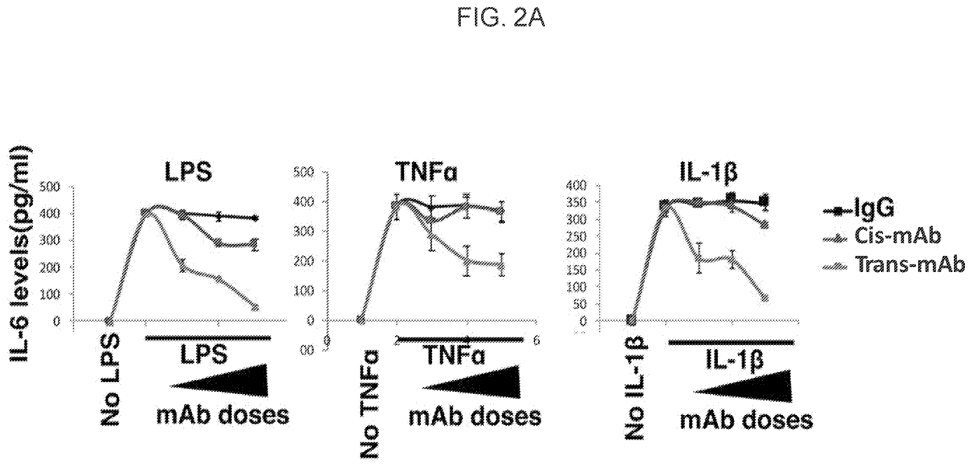

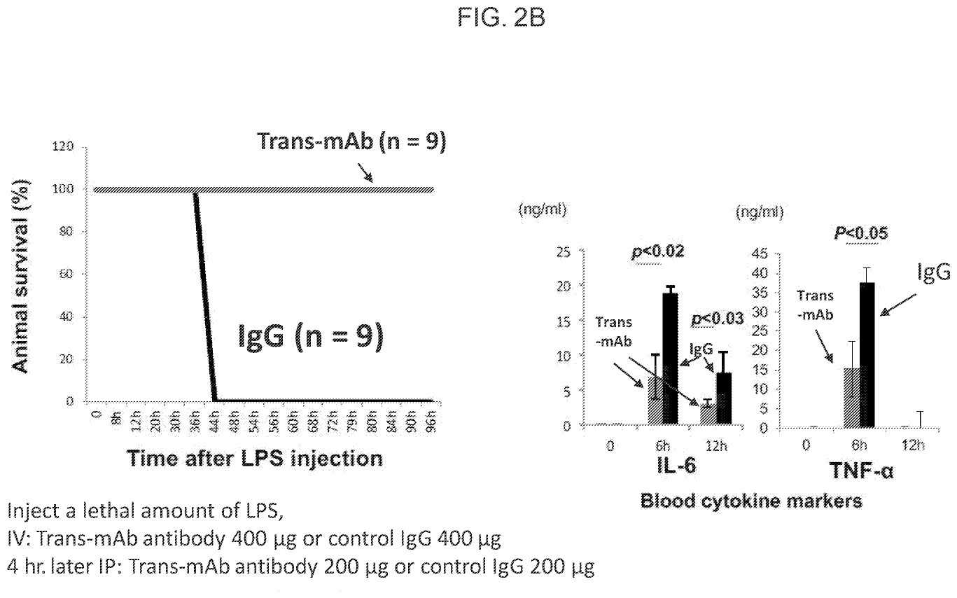

To examine the function of the cytoplasm and nuclear forms of NF-κB, we stimulated bone marrow-derived dendritic cells with LPS, TNFα, or IL-1β, followed by treatments with a trans-mAb, a cis-mAb or control IgG. Although there was little effect for the cis-mAb, the trans-mAb dose-dependently inhibited the ability of LPS, TNFα or IL-1β to induce production of cytokines such as IL-6 from dendritic cells (FIG. 2A). These results imply that the trans-mAb against the nuclear form of NF-κB might effectively prevent toxin-induced septic shock death in animal models. To investigate this possibility, we injected mice with a lethal dose of LPS and then injected the mice twice with the trans-mAb or isotype IgG controls at 0 and 4 hours after LPS injection. In the IgG-treated group, LPS robustly induced proinflammatory cytokines such as IL-6 and TNFα, and all mice died within 44 hours (FIG. 2B). However, the pro-inflammatory cytokines in the blood of mice treated with the trans-mAb were markedly reduced, and none of the mice died. These results indicate that the trans-mAb can effectively prevent death of toxin-induced septic shock in preclinical animal models.

### Example 4. The Nuclear Form (Trans) of NF-κB is Induced in Patients with Bacterial Sepsis and Located in the Nucleus of Peripheral Blood Mononuclear Cells

To confirm whether these two forms of NF-κB were induced in sepsis patients, we examined the expression of these two protein forms in peripheral blood mononuclear cells (PBMCs) in sepsis patients and controls. We observed that neither NF-κB form was detected in normal people but were induced in large quantity in sepsis patients. Importantly, as in vitro LPS-stimulated cells, the cis-mAb recognized NF-κB only in the cytoplasm of the blood immune cells, whereas the trans-mAb recognized NF-κB only in the nucleus of the blood immune cells from septic shock patients (FIG. 3A-3C). Thus, the nuclear form of NF-κB is greatly induced not only in vitro by TLR activation, but also in immune cell in the blood of sepsis patients.

### Example 5. A Trans-Specific Anti-Nuclear NF-κB Antibody Drastically Reduces Proinflammatory Cytokine Storm and Mortality of Bacterial-Induced Septic Shock in Animal Models

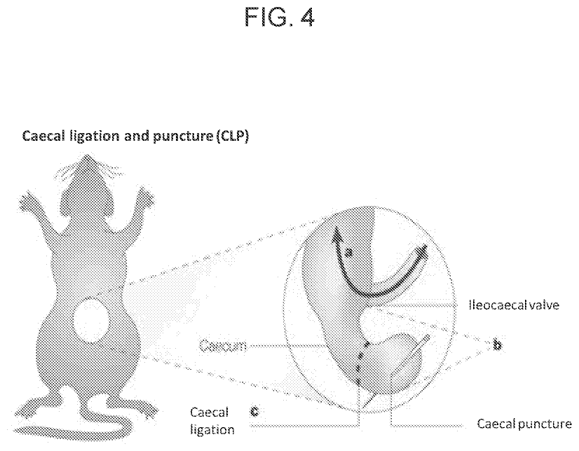

The caecum ligation and puncture (CLP) model is widely recognized as the gold standard for sepsis studies (see, for example, Buras, J. A., Holzmann, B. & Sitkovsky, M. Animal models of sepsis: setting the stage. Nat Rev Drug Discov 4, 854-865 (2005); and Fink, M. P. Animal models of sepsis. Virulence 5, 143-153 (2014)). The CLP model mimics human acute gangrenous perforation of appendicitis (FIG. 4). The technique involves a midline laparotomy to find the cecum, then cecal ligation and cecum puncture, and finally close the abdominal cavity. This process produces perforation of the intestine and fecal content leaks into the peritoneum, which causes infection with mixed bacteria and provides a source of inflammation for necrotic tissue. The severity of the CLP can be adjusted by increasing the size of the needle puncture or the number of punctures, with mortality ranging from a few hours to a few days, or even more slowly than 28 days. Most notably, the CLP model mimics the hemodynamic and metabolic stages of human sepsis. In addition, in patients with clinical sepsis, there is a significant amount of apoptosis in the relevant cells of the congenital and acquired immune system, which provides a clinical relevance for this model. Therefore, the CLP animal model is recognized as the gold standard experimental model of sepsis and septic shock.

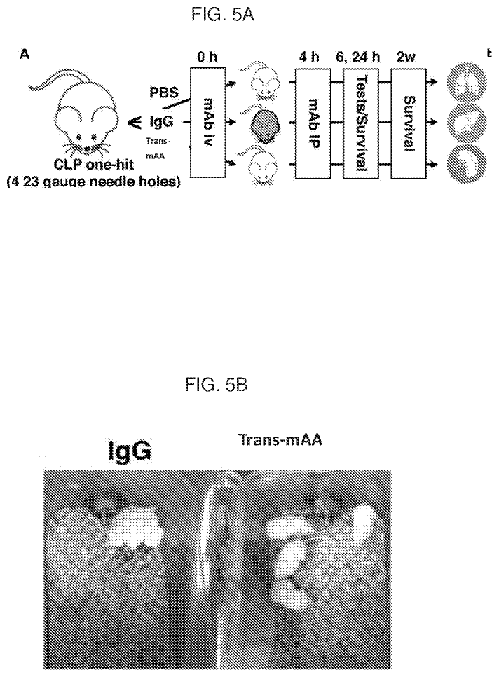

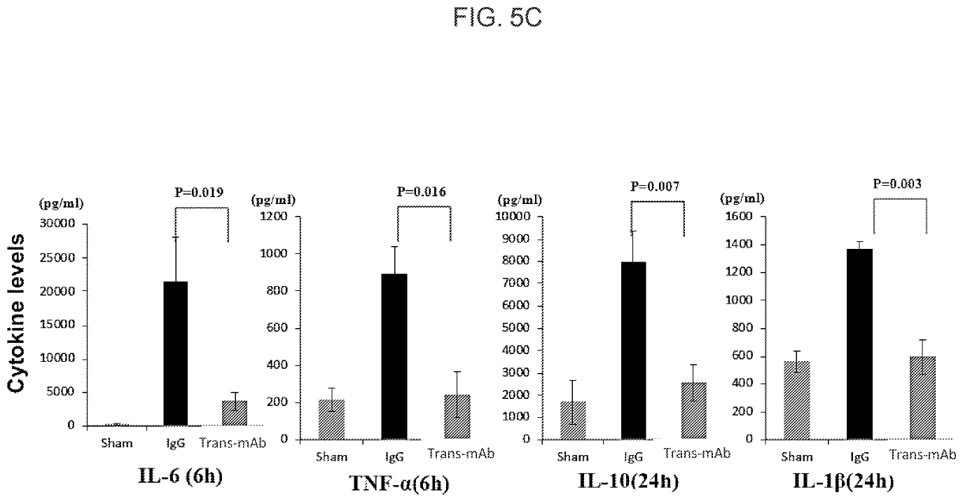

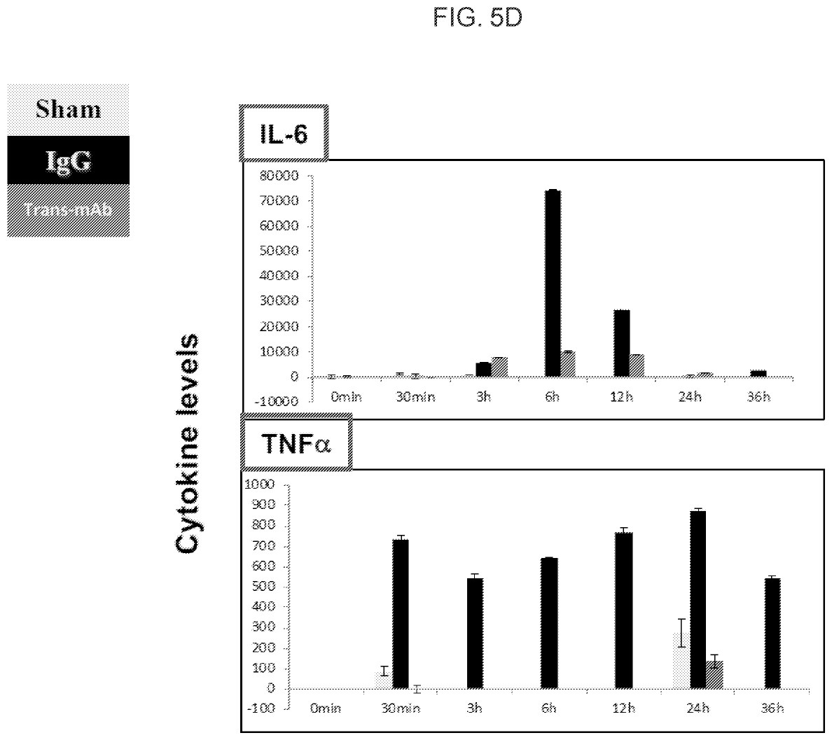

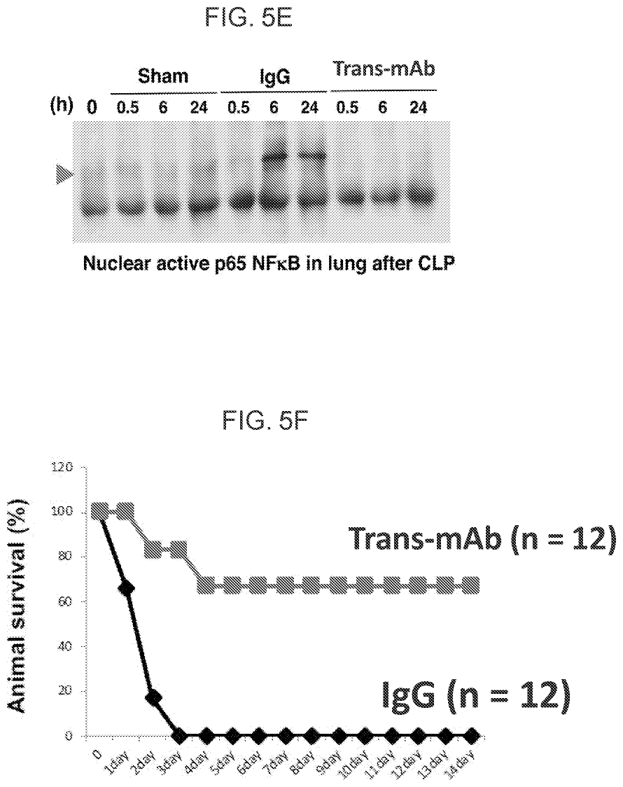

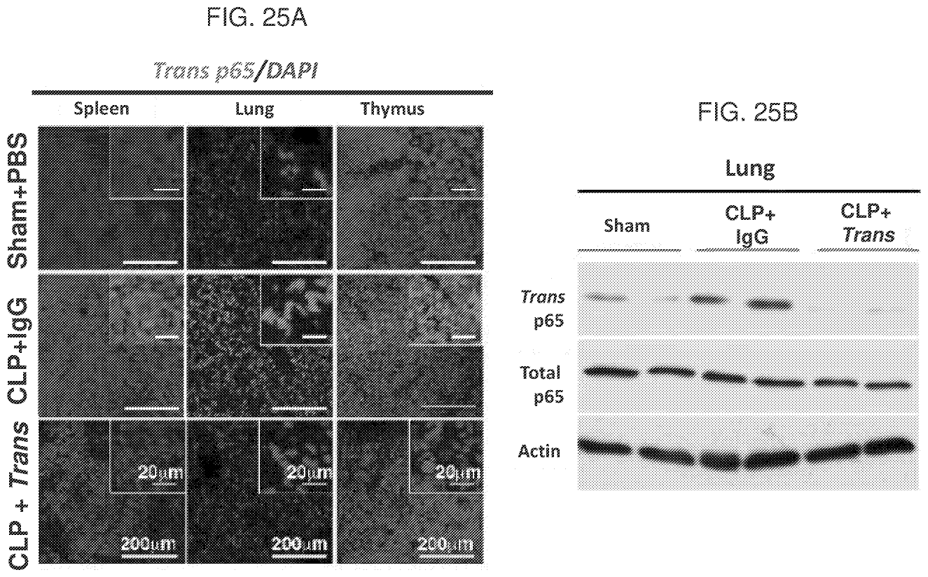

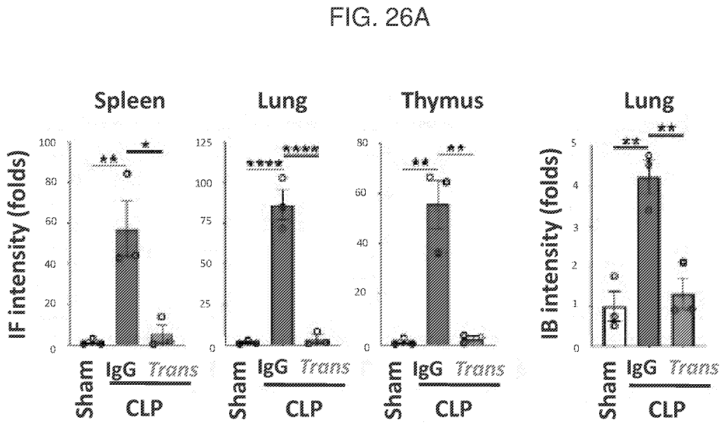

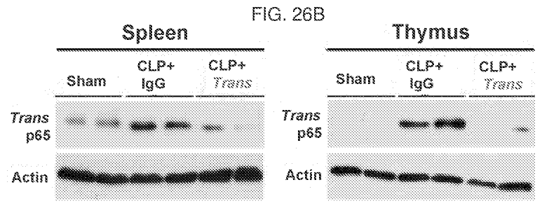

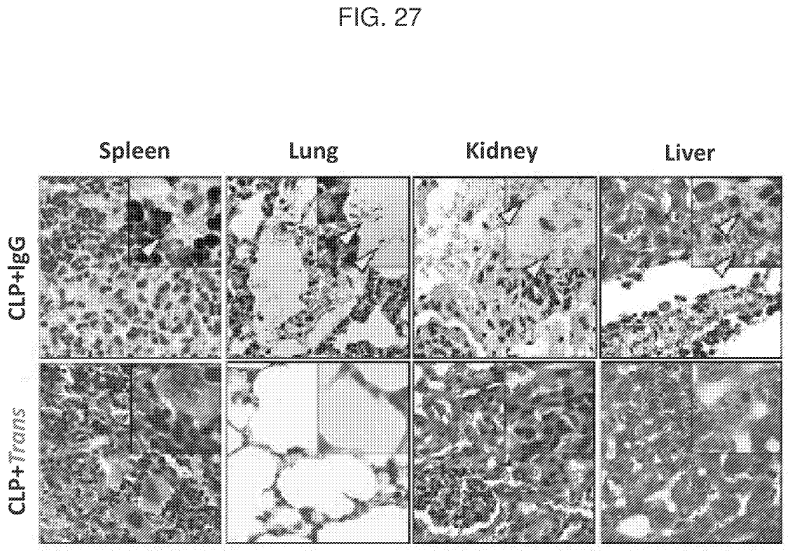

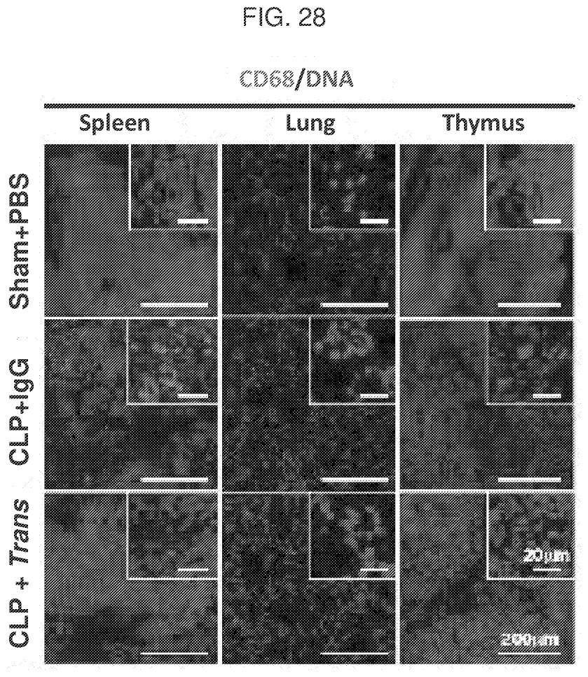

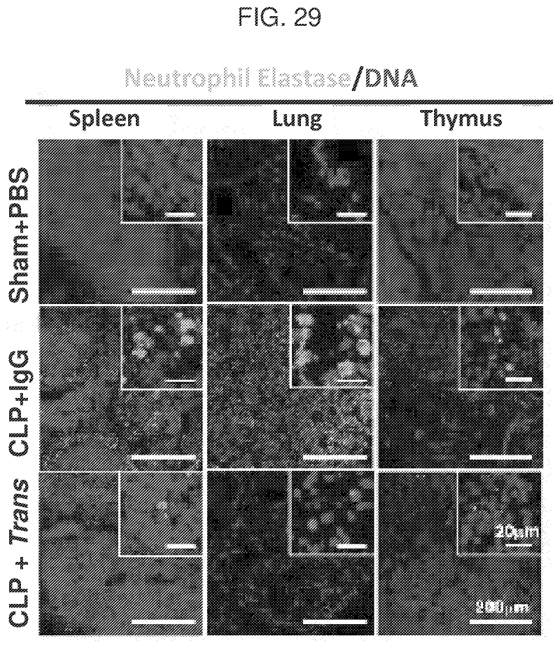

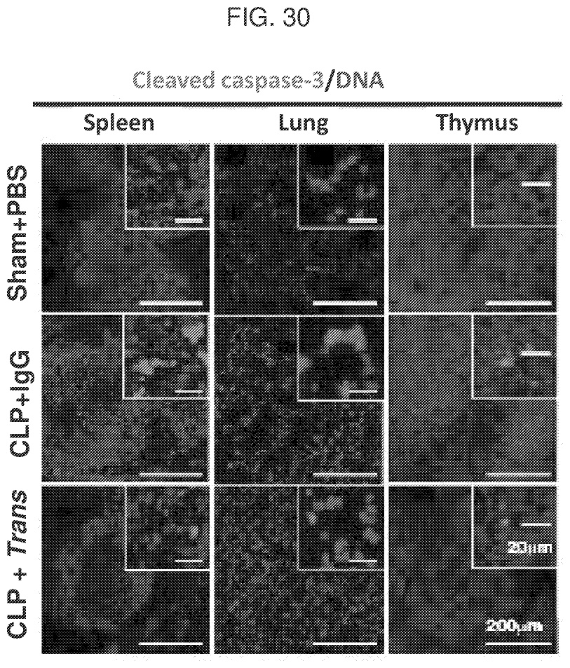

To examine the efficacy of the trans-mAb treatment in sepsis, we performed CLP surgery on mice with a 20 gauge needle, and then injected mice with two trans-mAb or isotype IgG controls (one after the CLP Intravenous injection followed by an intraperitoneal injection 4 hours later (FIG. 5A). Nuclear NF-κB was induced in various body tissues such as lungs and spleen in the IgG control group as expected in the IgG control group within 6 to 24 hours after CLP (FIG. 5E, FIGS. 25A-25B, and FIGS. 26A-26B) accompanied by robust infiltration of bacteria (FIG. 27), macrophages (FIG. 28), and neutrophils (FIG. 29), and by increased apoptosis (FIG. 30). The animals were very sick (FIG. 5B). Various pro-inflammatory cytokines were markedly induced, producing the pro-inflammatory cytokine storm (FIG. 5C). All animals died within 56 hours (FIG. 5F). However, trans-mAb antibody treatments effectively eliminated the induction of nuclear NF-κB in body tissues (FIG. 5E, FIGS. 25A-25B, and FIGS. 26A-26B), significantly reduced pro-inflammatory cytokine storm (FIGS. 5C and 5D), and drastically improved survival of septic mice, with survival rate from 0% in IgG-treated mice to 75% in trans-mAb-treated mice (FIG. 5F). These surviving mice live well after CLP without any obvious phenotype (FIG. 5E). The trans-specific anti-nuclear NF-κB antibody treatment not only effectively eliminates the nuclear NF-κB, but also reduces the proinflammatory cytokine storm and drastically increases the survival of septic shock animals.

### Example 6. A Trans-Specific Anti-Nuclear NF-κB Antibody Also Improves Immunosuppression Following Sepsis, Allowing Mice to Successfully Fight Secondary Bacterial Lung Infections

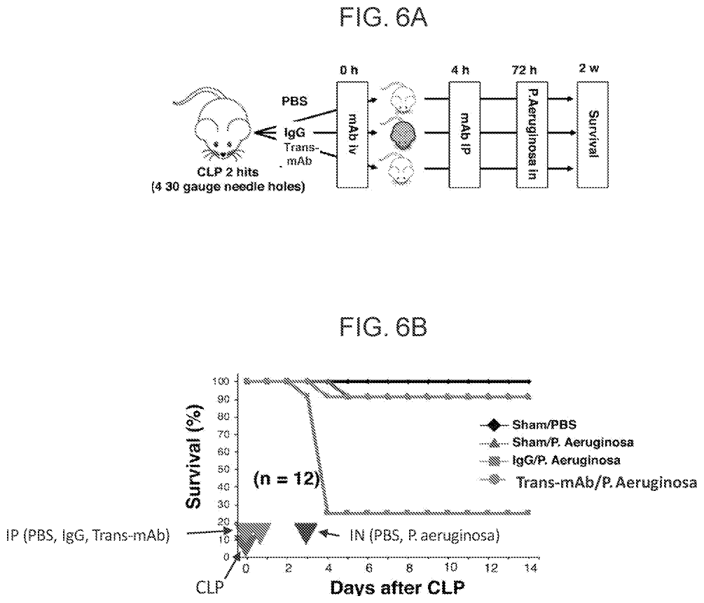

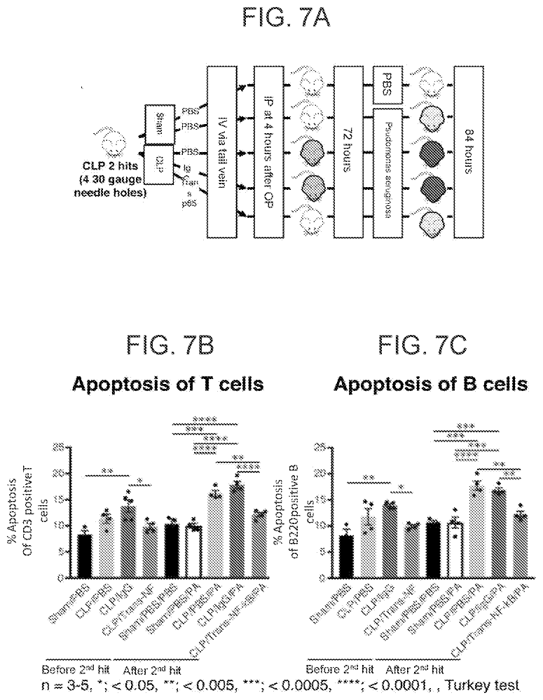

The above results indicate that trans-mAb can effectively prevent the pro-inflammatory cytokines and death caused by septic shock. We further tested trans-mAb for the ability to mitigate immunosuppression in sepsis. We used a 30 gauge needle to perform CLP. Mice were given two injections of trans-mAb or an isotype IgG control (one intravenous injection after CLP and then an intraperitoneal injection 4 hours later) (FIG. 6A). The IgG isotype was used as a control and intranasal administration of Pseudomonas aeruginosa was used to induce lung infections after 3 days after CLP as a second hit. Pseudomonas aeruginosa is one of the most common and refractory iatrogenic bacterial infections. In the control IgG-treated group, 30-needle CLP sepsis mice did not die within 3 days, but 80% of these mice died one day after lung bacterial infection (FIG. 6B). This is likely because sepsis causes immunosuppression so that mice failed to fight secondary lung bacterial infection. Indeed, T cells and B cells underwent apoptosis before and after lung infection in this group of sepsis mice, as compared with sham mice. Trans-mAb, in contrast, effectively prevented lung bacterial infection-induced death in treated mice, with 90% of the mice surviving for at least 2 weeks without any apparent symptoms (FIG. 6B). Moreover, trans-mAb treatment largely prevented apoptosis of T cells and B cells before and after lung infection (FIG. 7A-7C).

The above results demonstrate that administration of monoclonal antibodies against nuclear active form of NF-κB not only dramatically reduces the proinflammatory cytokine storm and early death of septic shock, but also greatly improves immunosuppression, allowing the sepsis mice to successfully fight secondary bacterial infection. This beneficial effect has not been demonstrated using any other previously developed sepsis treatment drug. These unique features of the monoclonal antibodies against nuclear active form of NF-κB confirm their ability to treat sepsis.

### Example 7. A Trans-Specific Anti-Nuclear NF-κB Antibody Eliminates Trans p65 Induction

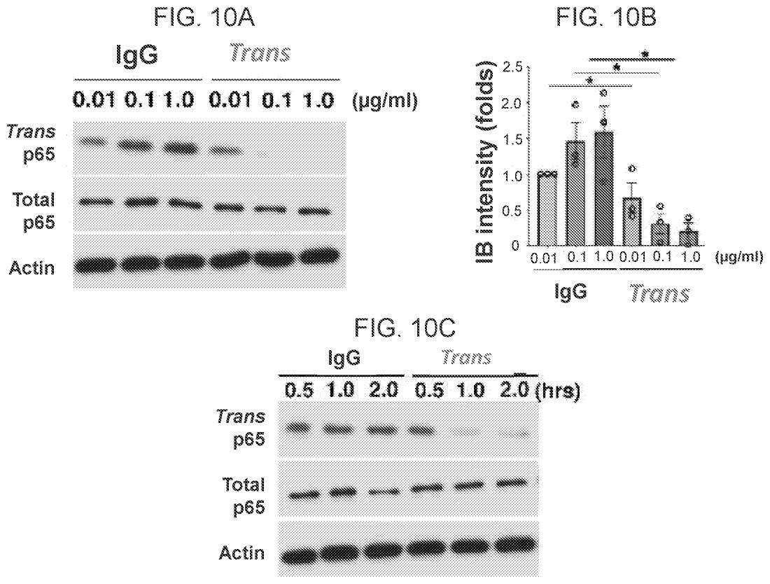

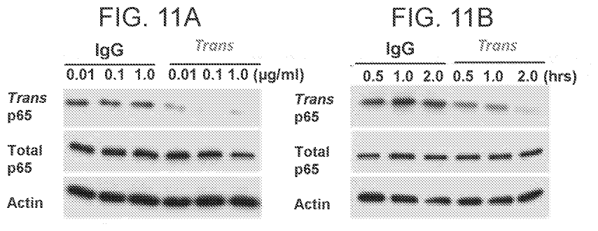

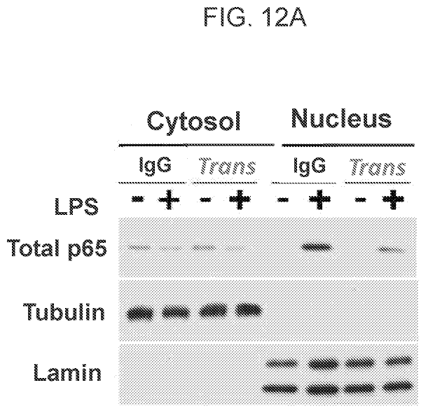

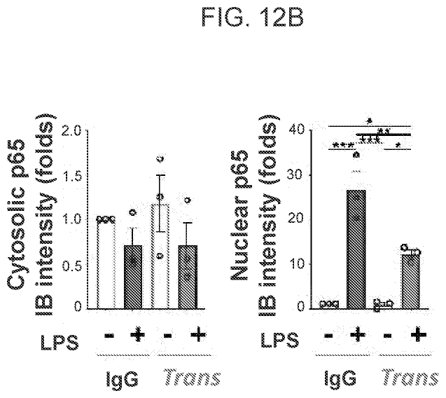

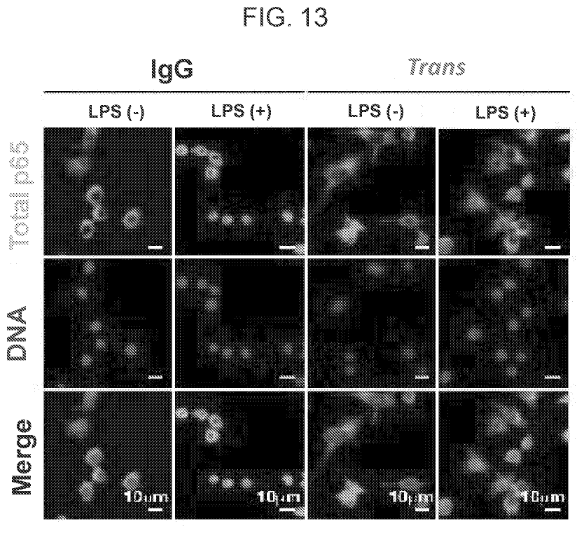

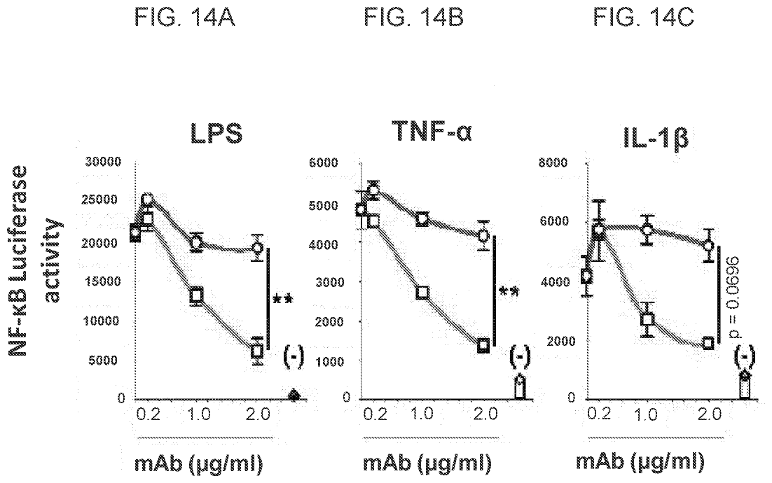

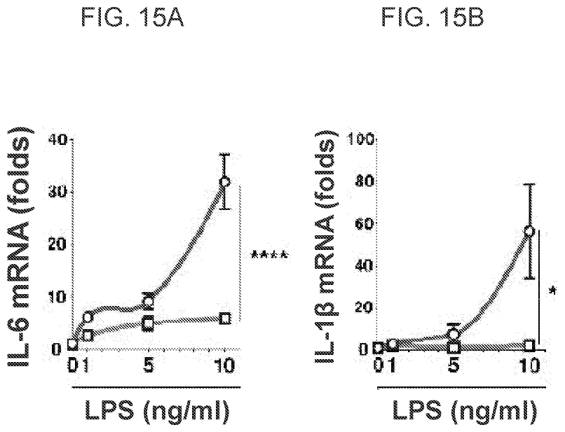

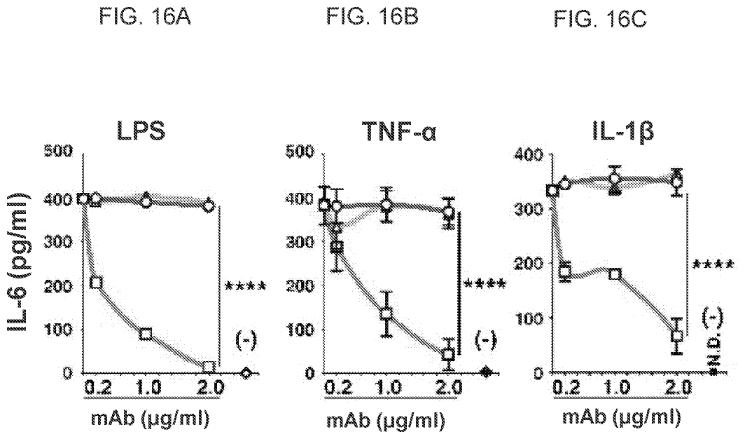

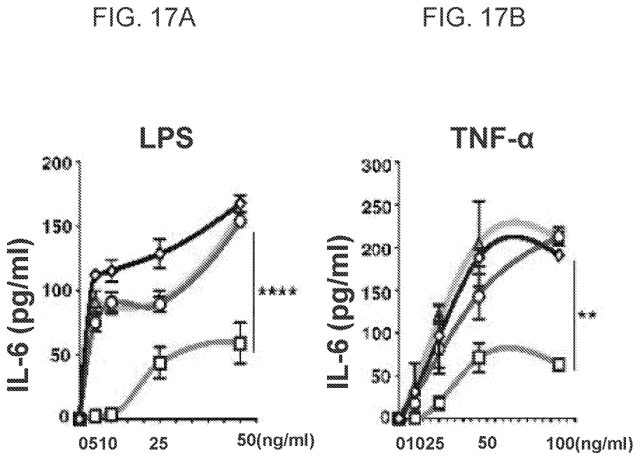

We further tested trans-mAb for the ability to reduce the induction of trans p65 in macrophages (FIGS. 10A-10C) and in dendritic cells (FIGS. 11A-11B). We stimulated macrophages and dendritic cells with LPS in the presence of trans mAb or its IgG isotype control. We observed that trans mAb, but not control IgG, eliminated trans p65 induction, in a dose-dependent (FIGS. 10A-10B) and in a time-dependent manner (FIG. 100) in macrophages. We also observed dose-dependent (FIG. 11A) and time-dependent (FIG. 11B) elimination of trans p65 in dendritic cells. Moreover, using subcellular fractionation (FIGS. 12A-12B) and immunofluorescence analyses (FIG. 13) we found that trans mAb significantly reduced LPS-induced accumulation of total p65 specifically in the nucleus, with little effect on abundant cytoplasmic p65. To confirm these results, we examined the effects of cis and trans mAbs on NF-κB activity since nuclear NF-κB is functionally active. Although cis mAb or control IgG had little effects, trans mAb dose-dependently inhibited NF-κB activation by LPS, TNF-α or IL-1β, as shown by κB promoter reporter activity (FIGS. 14A-140), and cytokine mRNA (FIGS. 15A-15B) and protein levels (FIGS. 16A-16C and FIGS. 17A-17B).

### Example 8. Levels of Trans p65 NFκB are Elevated in Human Sepsis Patients

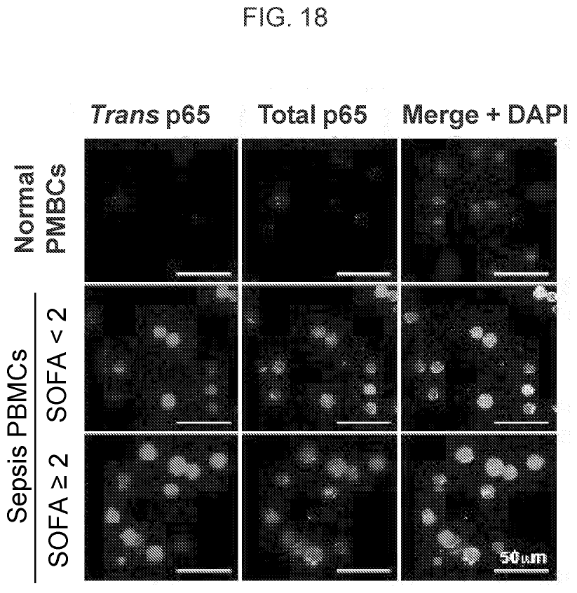

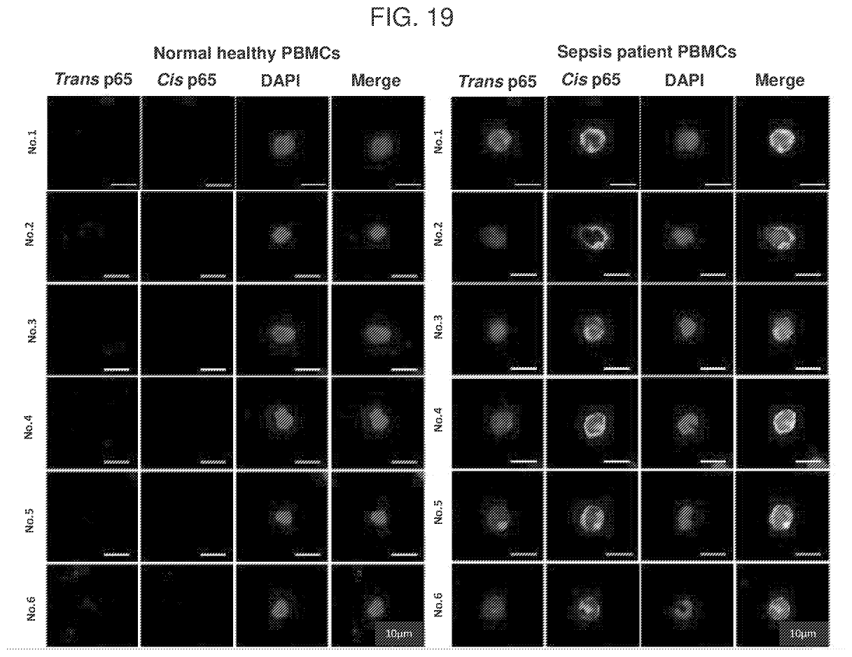

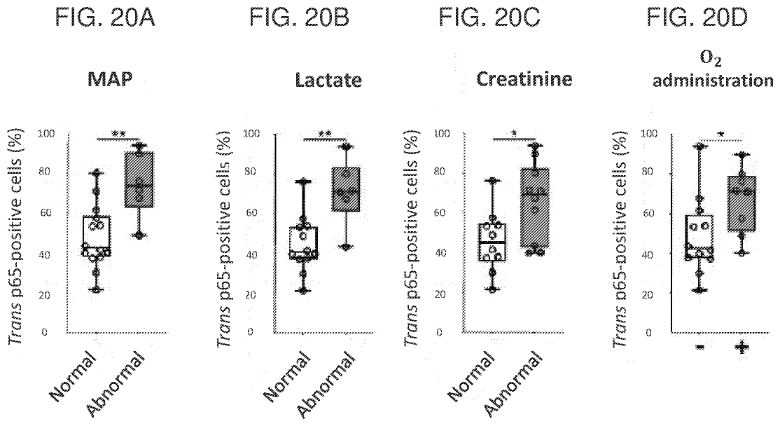

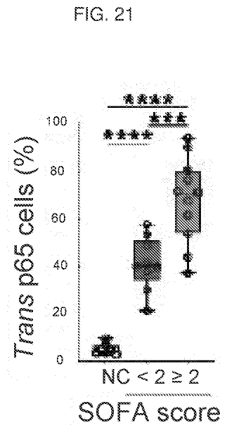

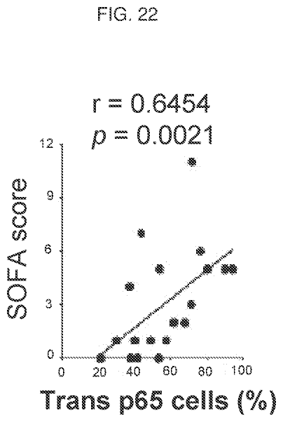

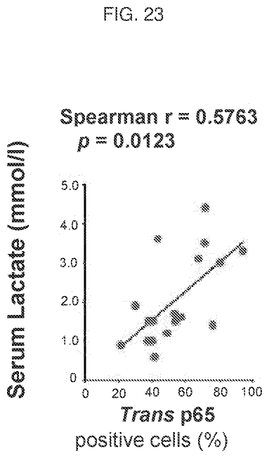

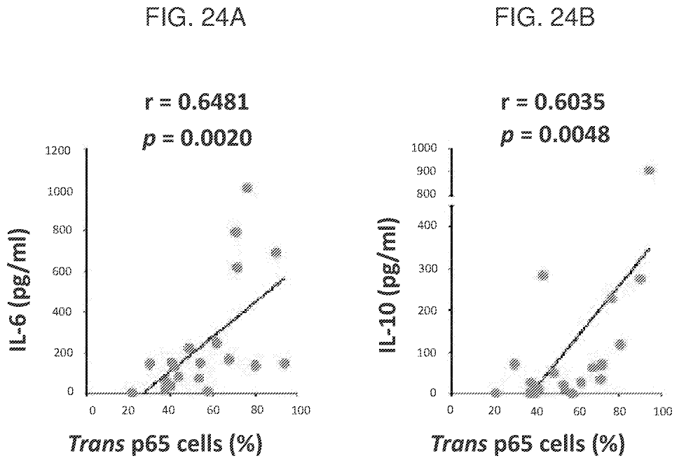

To examine the possibility of therapeutic treatment of NF-κB-mediated inflammation and cytokine storm we first examined the relationship between p65 NF-κB with clinical parameters in sepsis patients. We first isolated peripheral blood mononuclear cells (PBMCs) and plasma from 20 newly admitted patients suspected of sepsis in order to assay p65 conformations and cytokines. Whereas little cis or trans p65 was detected in controls, robust cis and especially trans p65 were detected mainly in the cytoplasm and nucleus, respectively, in PBMCs from all patients examined (FIGS. 18-19). Significantly, high trans p65-positive PBMCs in percentage were significantly correlated with abnormal blood lactate, mean arterial pressure, creatinine level and 02 administration (FIGS. 20A-20D), and with high Sequential Organ Failure Assessment (SOFA) scores (FIGS. 21-22), a disease severity and mortality prediction score based on the dysfunction of six major organs. Trans p65-positive PBMCs in percentage as a continuous variable was also correlated well with SOFA scores, serum lactate levels (FIGS. 22-23), and plasma IL-6 and IL-10 levels (FIGS. 24A-24B). Thus, blood trans p65 NF-κB is robustly induced and significantly correlated with SOFA scores and cytokines, although more patients are needed to validate its clinical significance.

### Example 9. A Trans-Specific Anti-Nuclear NF-κB Antibody Eliminates Trans p65 Induction in Peripheral Blood Mononuclear Cells of Patients with Sepsis

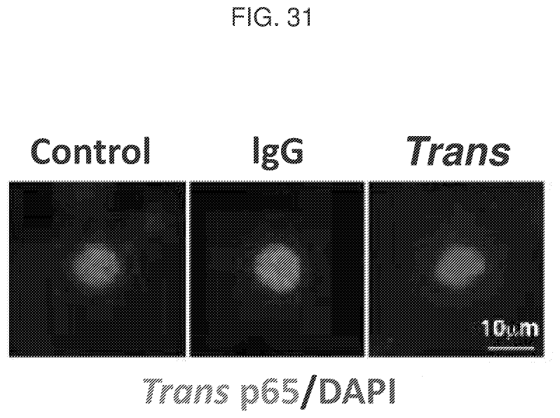

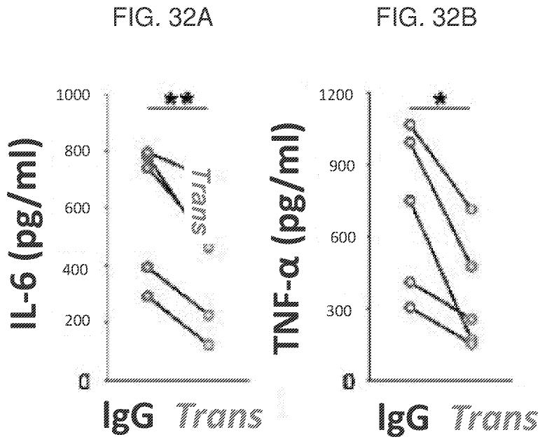

To examine whether trans mAb can attenuate p65 and cytokine production in human sepsis patients, we cultured peripheral blood mononuclear cells (PBMCs) freshly isolated from human sepsis patients and then treated them with trans mAb or controls for 8 hrs. Trans mAb eliminated trans p65 (FIG. 31), and also reduced production of its cytokine targets, IL-6 and TNF-α (FIG. 32A-32B), from all five sepsis patients PBMCs able to produce significant cytokines ex vivo. Despite the limited number of patients analyzed, these data show that trans mAb also eliminates p65 and attenuates cytokine production from human sepsis patient PBMCs ex vivo, corroborating our findings in cells and mice.

### Example 10. A Trans-Specific Anti-Nuclear NF-κB Antibody Eliminates Trans p65 Induction and Attenuated Pancreatitis

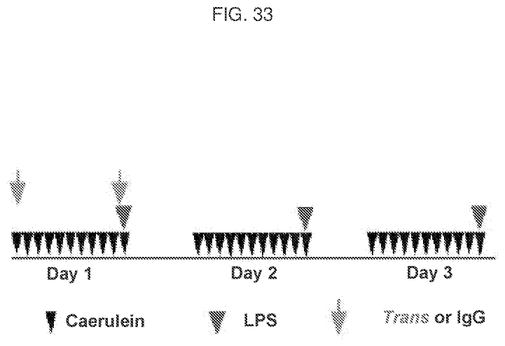

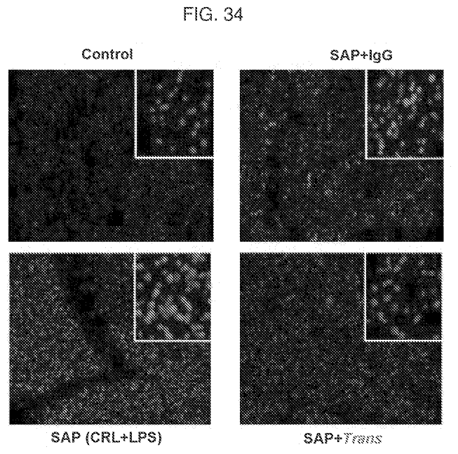

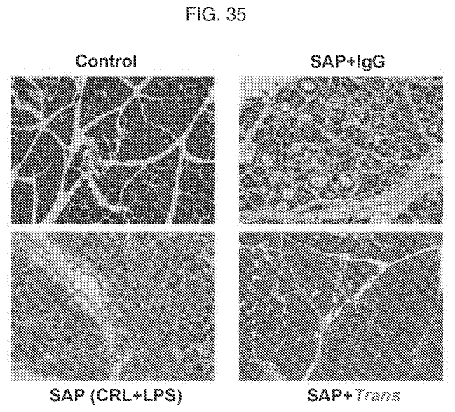

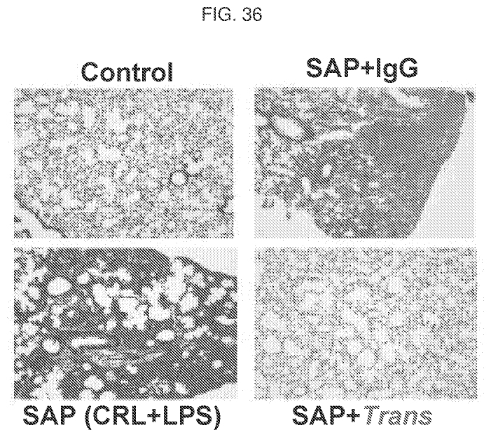

As an independent model for targeting trans p65, we used pancreatitis-associated acute lung injury, a systematic inflammatory disease with high mortality, which is typically seen in sepsis, and is also a major complication in pandemics. Importantly, NF-κB is crucial for linking the initial acinar injury to systemic inflammation and perpetuate the inflammation. Pancreatitis-associated acute lung injury was induced in mice by the CCK analog caerulein and LPS (FIG. 33). Treating these mice with trans p65 mAb not only ablated trans p65 induction (FIG. 34), but also greatly attenuated pancreatitis (FIG. 35), acute lung injury (FIG. 36) and death, further supporting the potency of trans mAb to target trans p65 NF-κB in sepsis and systematic inflammation.

### Example 11. Differentially Expressed Genes are Rescued by Trans-Specific Anti-Nuclear NF-κB Antibody

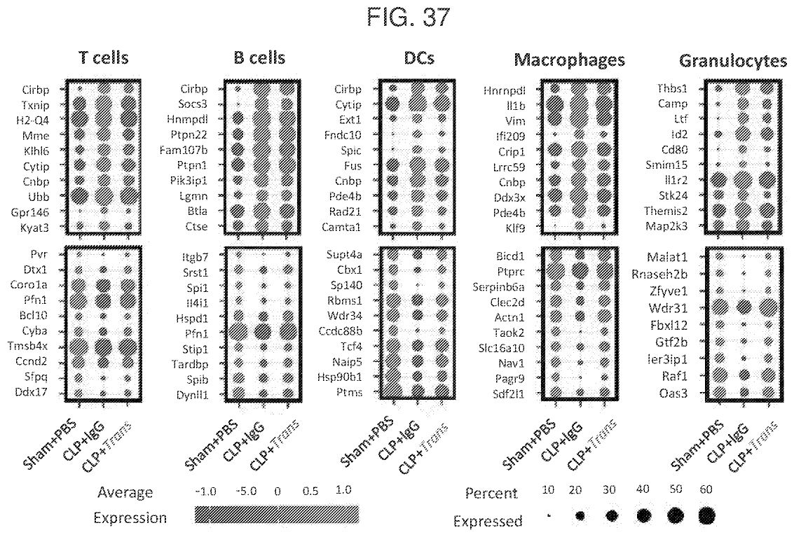

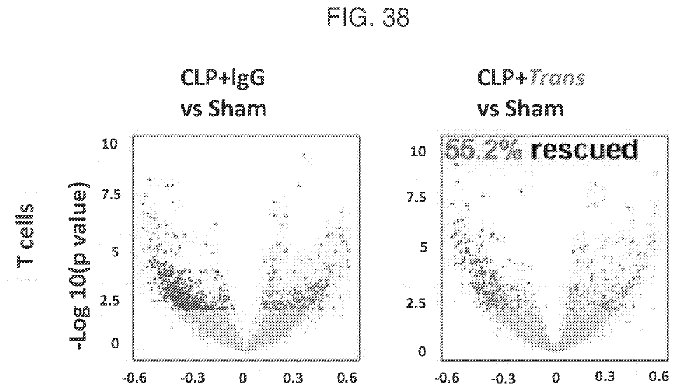

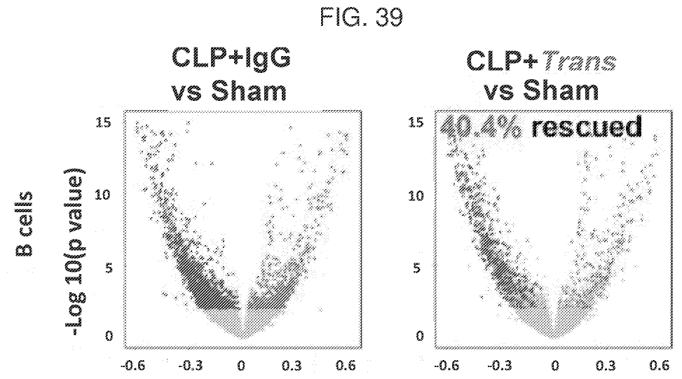

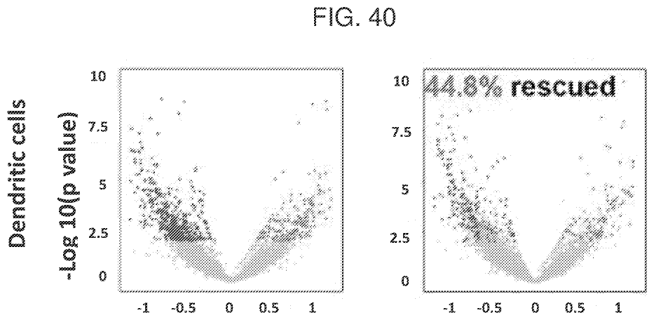

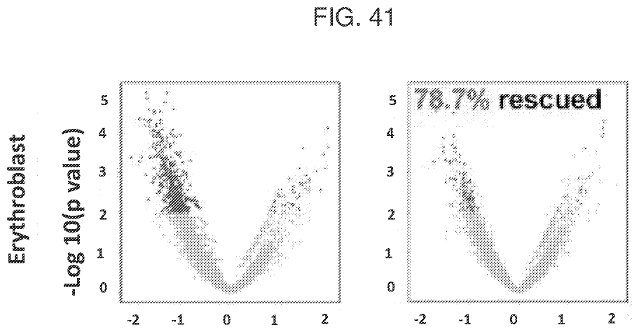

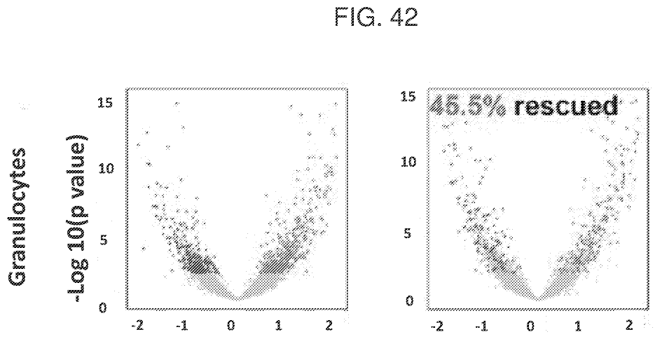

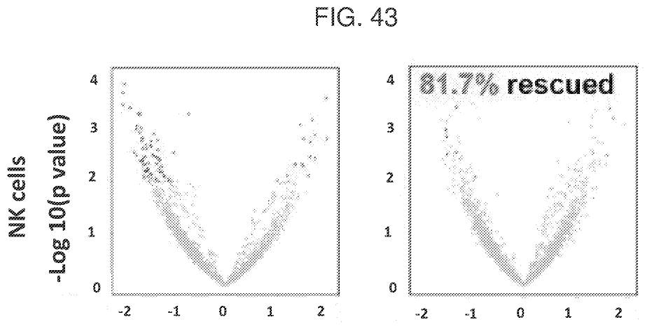

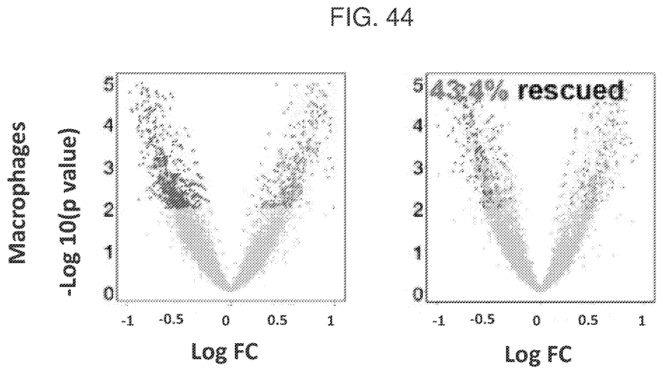

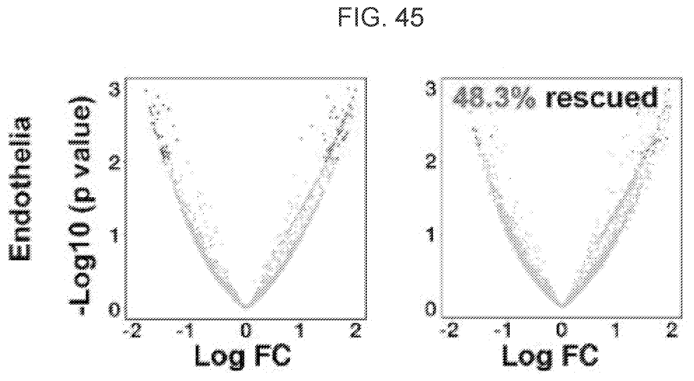

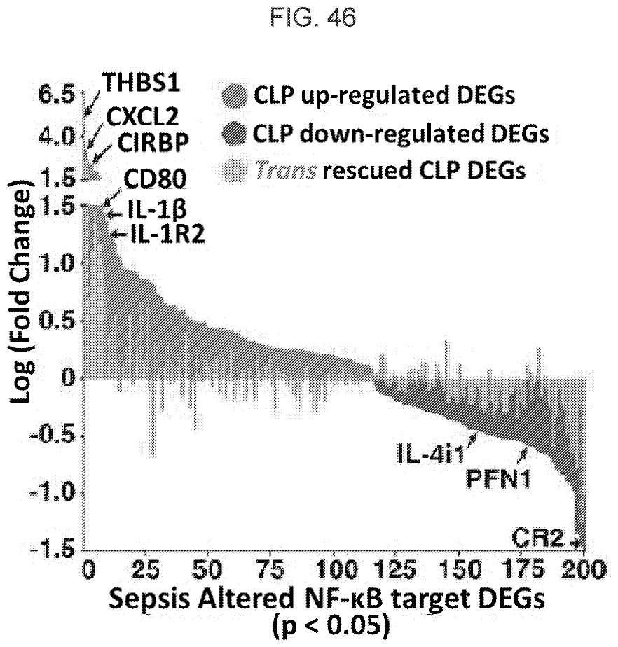

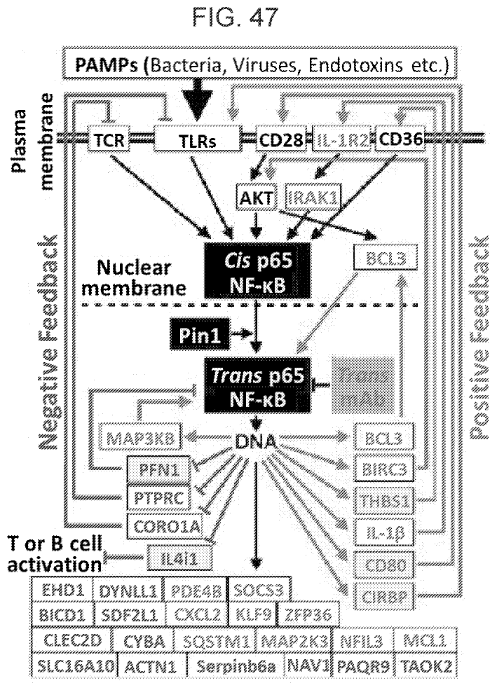

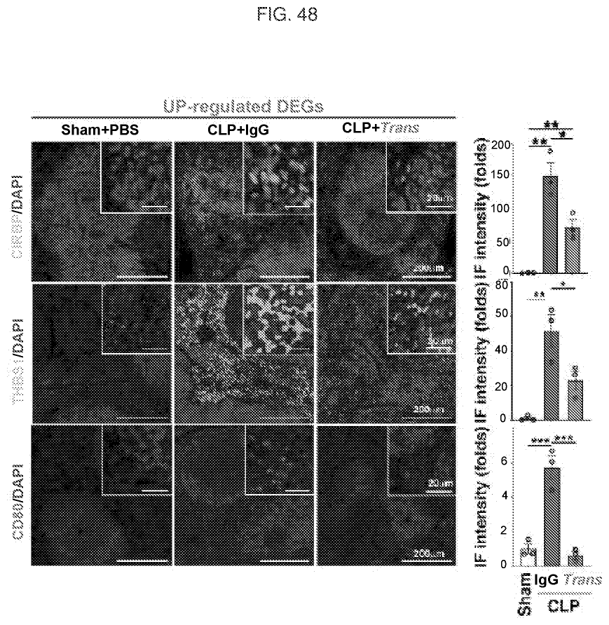

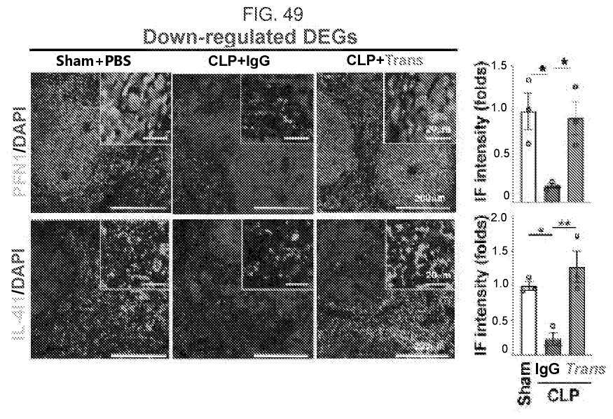

Cell-type specific differentially expressed genes (DEGs) after cecal ligation puncture (CLP) are largely consistent with polymicrobial sepsis, including genes in immune responses to bacteria and LPS, TNF-α signaling via NF-κB and cytokine signaling. Remarkably, 40-80% of these DEGs were significantly rescued by trans mAb treatment depending on cell types (FIG. 37 and FIGS. 38-45). At least 200 trans mAb-rescued DEGs were known NF-κB targets (FIG. 46), which converged onto the NF-κB-centered pathways at different signaling steps (FIG. 47). CLP appeared to induce the immune overreaction by upregulating pro-inflammatory genes towards promotion of positive feedback loops and by downregulating anti-inflammatory genes towards inhibition of negative feedback loops, both of which were significantly reverted, but not completely abolished by trans mAb, towards homeostasis (FIG. 47). We confirmed this mechanism using IF on five NF-κB targets: three upregulated DEGs (CIRBP, CD80 and THBS1) in positive feedback loops and two downregulated DEGs (PFN1 and IL-4i1) in negative feedback loops (FIG. 47) all of which were significantly restored by trans mAb in sepsis mice (FIGS. 48-49). Their altered expression was verified in sepsis patients. Of note, THBS1 is a newly identified human sepsis biomarker.

Notably, the most induced trans mAb-rescuable known DEG in T cells, B cells and DCs in CLP mice was cold-inducible RNA-binding protein (CIRBP, CIRP) (FIG. 37), a stress-response protein and a known DAMP. During sepsis, inflammation triggers CIRBP nuclear-to-cytoplasmic translocation and release to the extracellular space to promote the proinflammatory response via the TLR4/NF-κB pathway, with its blood levels correlating with patient outcomes.

### Example 12. Investigation of the Relationship Between Trans NF-κB and COVID-19

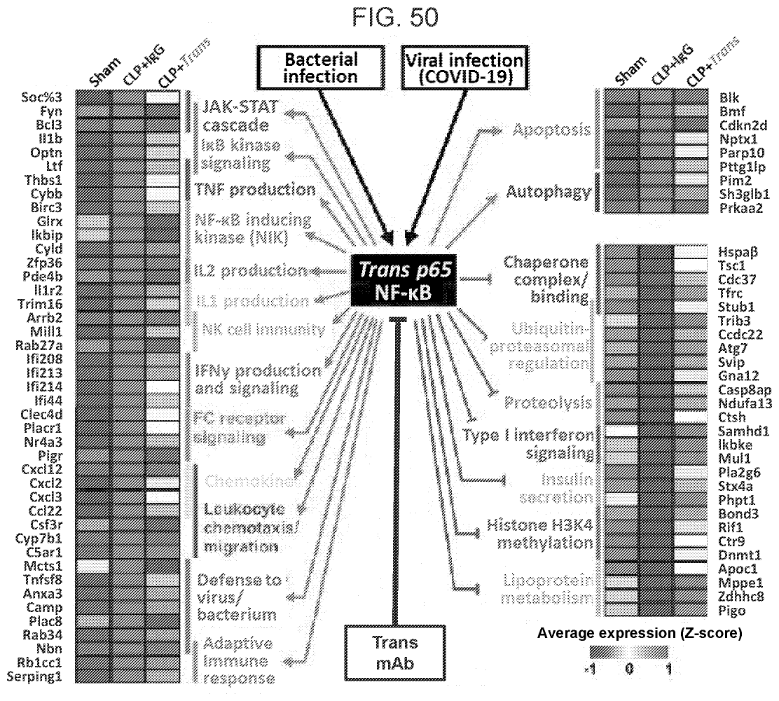

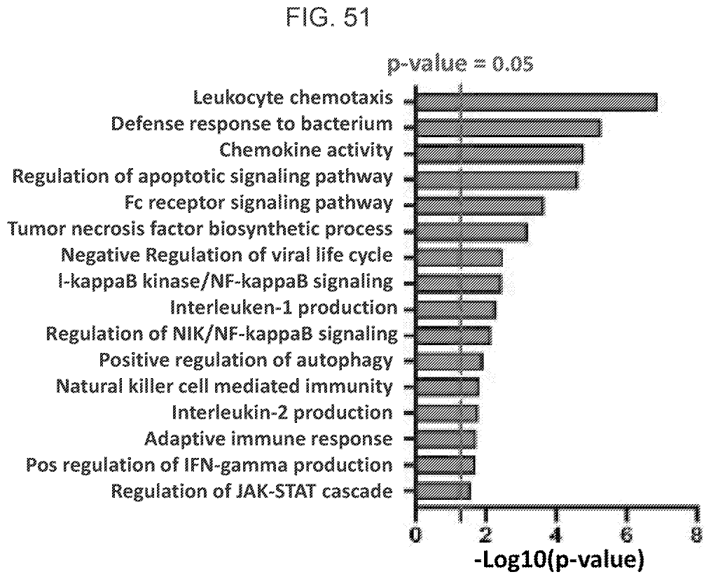

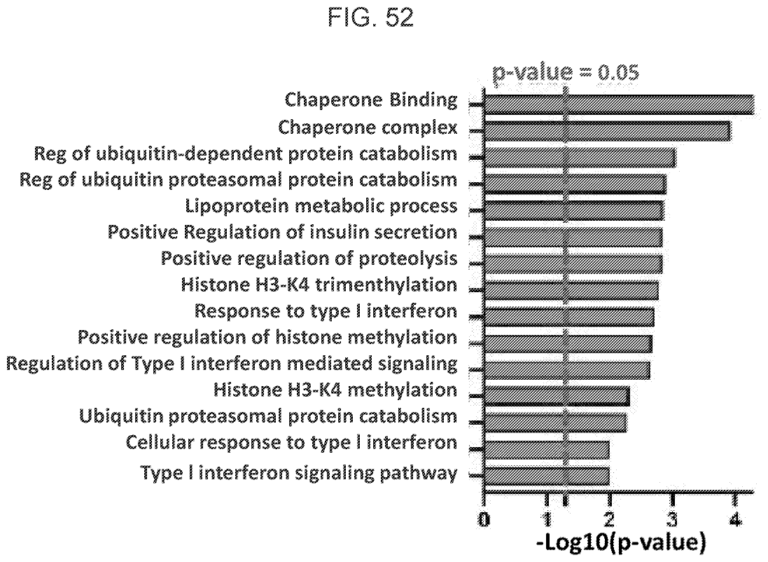

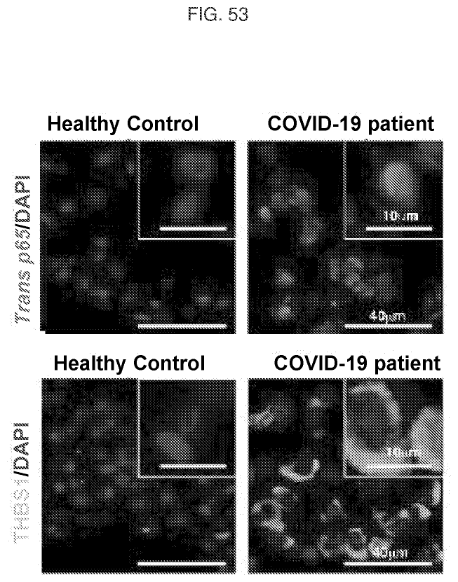

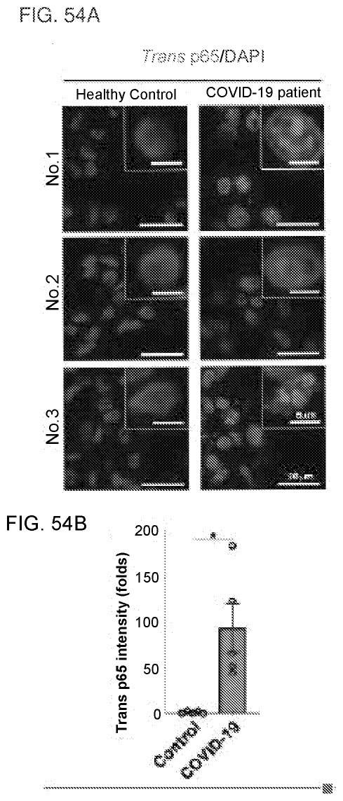

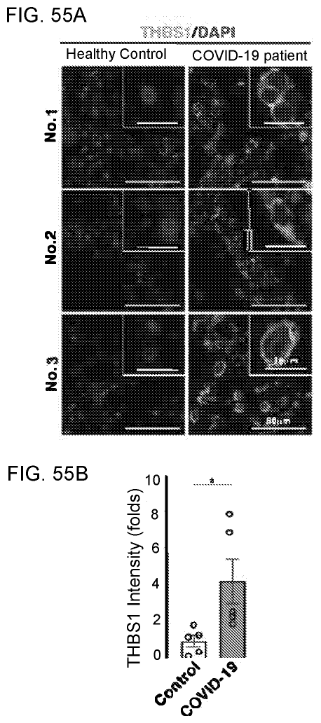

Sepsis is a major cause of COVID-19 deaths, with heightened and imbalanced innate immune response, as exemplified by NF-κB-centered pathways inducing cytokines, chemokines and IFNs. Comparison of our sepsis scRNA-seq data with those in COVID-19 patients revealed conserved hyper- and hypo-activation of a wide range of immune response pathways (FIGS. 50-52). Remarkably, treatment with trans mAb reversed the expression of a total of 439 DEGs back to Sham expression levels, not only inhibiting various pro-inflammatory pathways, but also rescuing many pathways possibly linked to anti-inflammatory response (FIG. 50), suggesting that trans p65 might be hyperactivated in COVID-19. Indeed, trans p65, and the sepsis biomarker THBS1 were induced in all five COVID-19 patients examined (FIGS. 53-55). These data reveal a transcriptomic similarity between sepsis and COVID-19 and suggest targeting trans p65 as a new therapeutic approach.

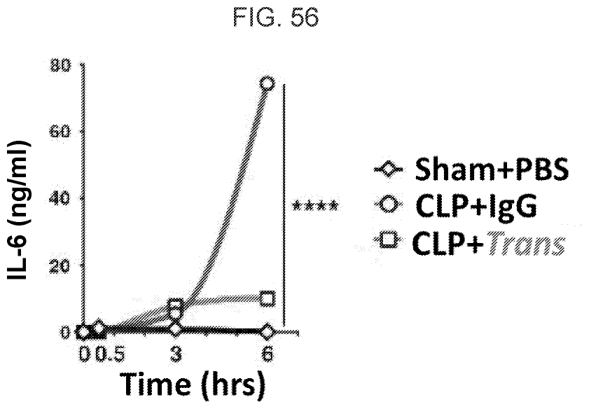

Recent advances have improved early survival in sepsis, but without tangible outcome on overall survival due to exacerbating immune paralysis and ongoing/nosocomial infections. Notably, targeting trans p65 reduced, but did not fully abolish, cytokine response (FIG. 5C and FIG. 56), and also reverted hyperactivation of pro- and anti-inflammatory responses towards homeostasis (FIG. 46), suggesting that such targeted therapy might not compromise the host immune response.

### Example 13. A Trans-Specific Anti-Nuclear NF-κB Antibody Rescues Immune Paralysis

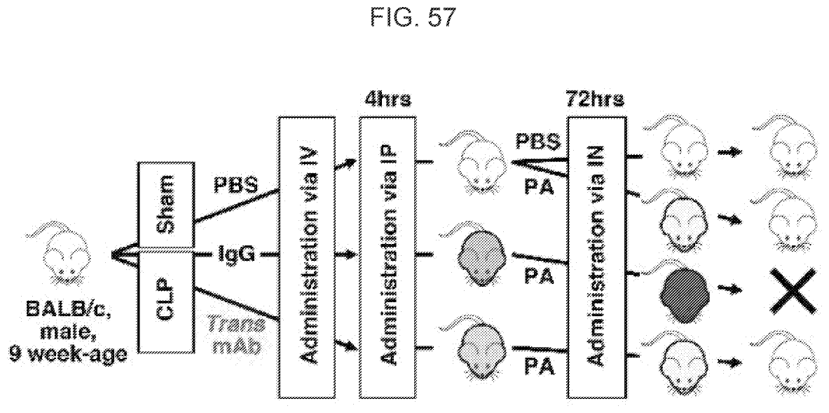

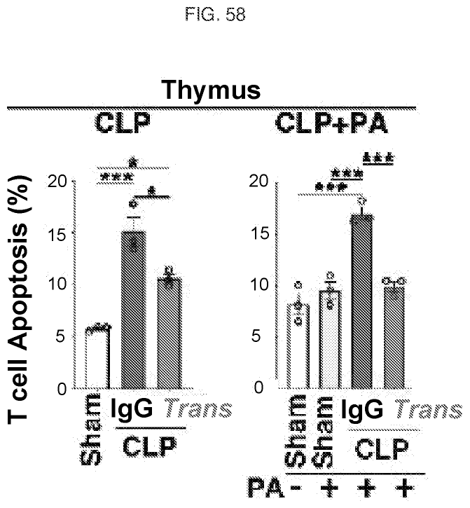

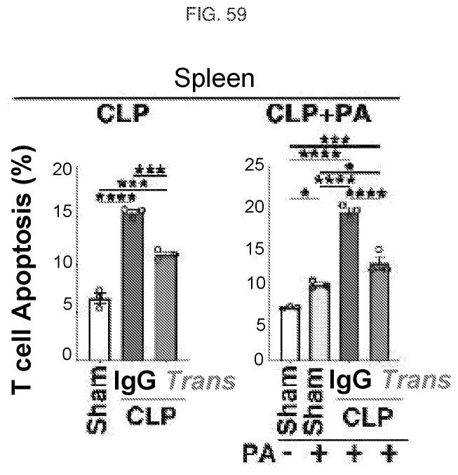

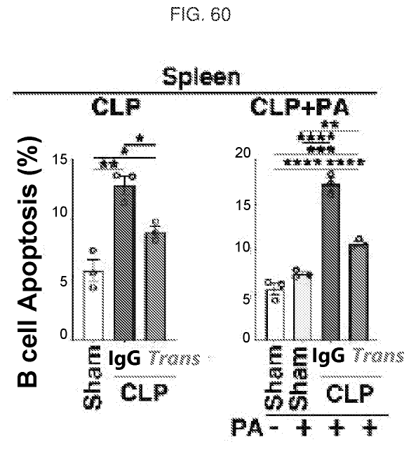

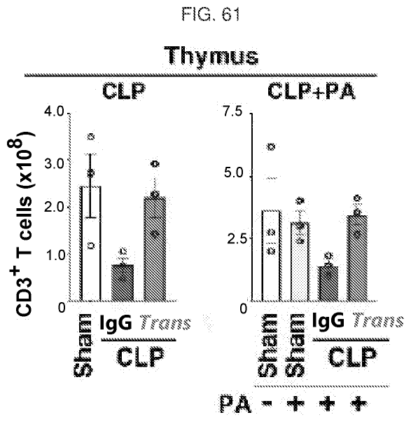

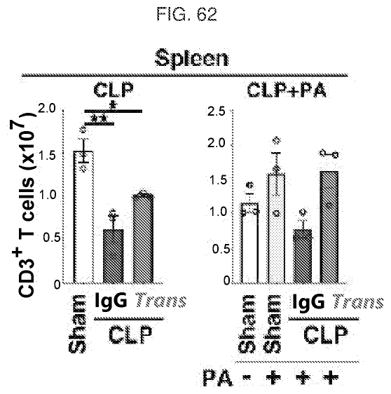

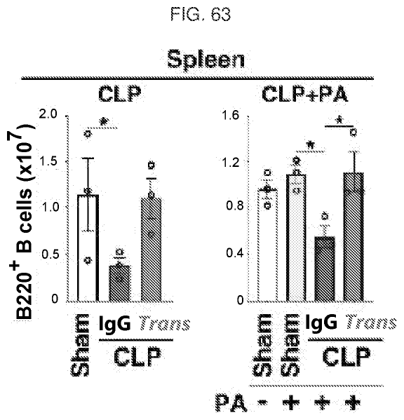

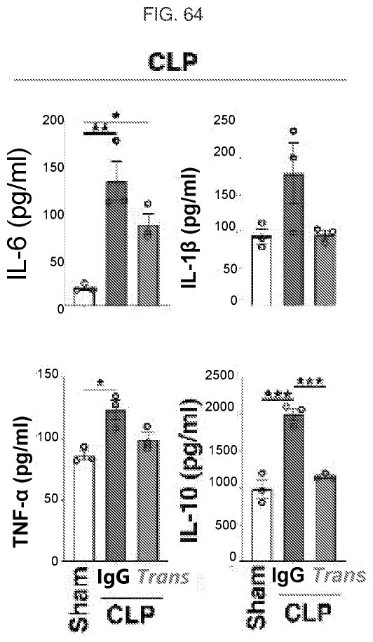

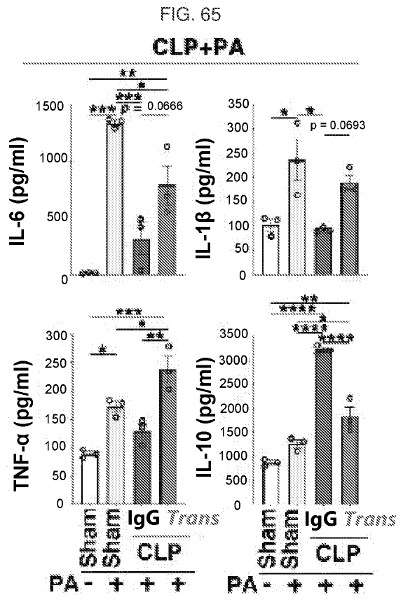

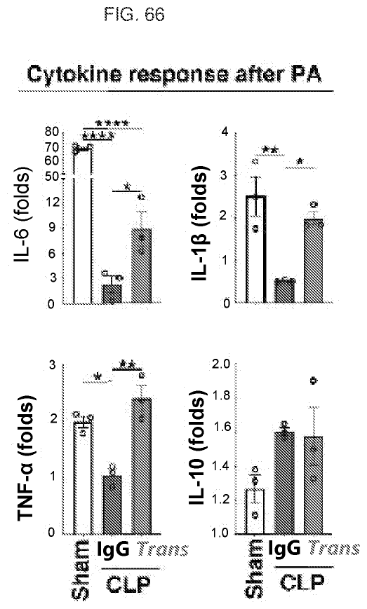

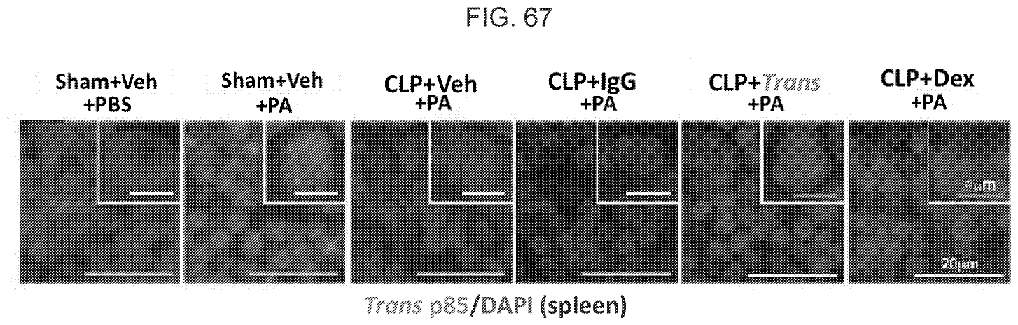

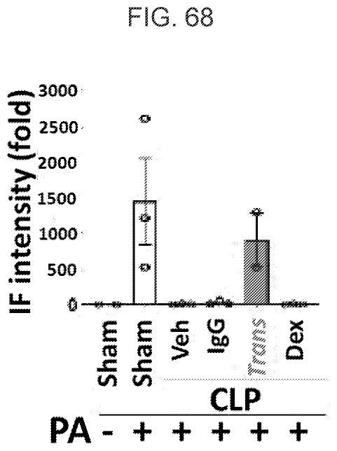

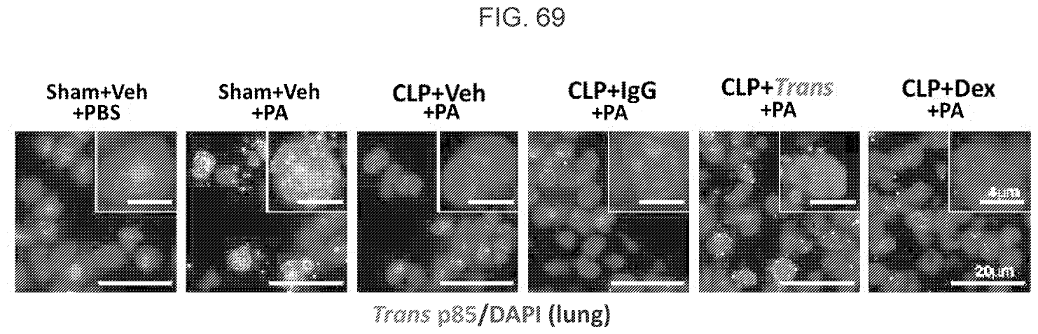

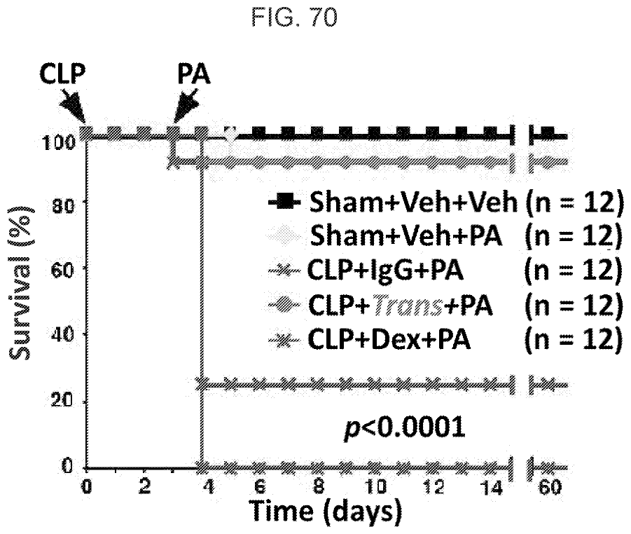

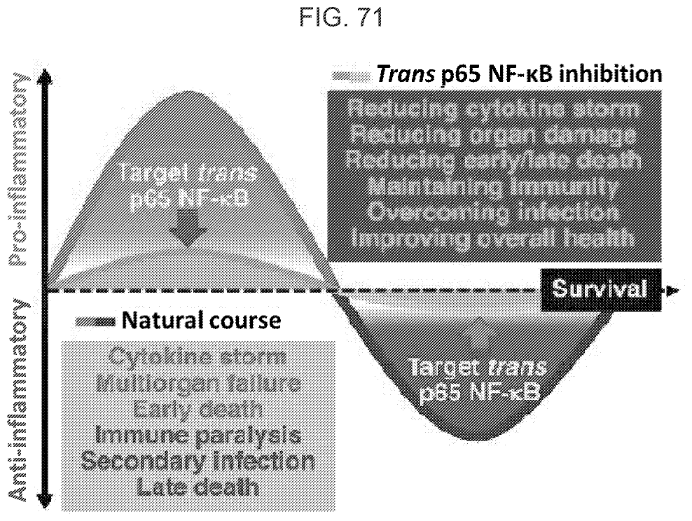

To test whether trans mAb-treated sepsis animals are immune competent to fight secondary infection, we used a mouse model of sepsis followed by pneumonia, a clinically relevant “two-hit” model of sepsis, which would reflect delayed mortality due to secondary nosocomial infection. The first hit was to perform CLP using a shorter ligation and a smaller needle to generate nonlethal sepsis mice, which were treated with trans mAb or IgG, followed by intranasal administration of Pseudomonas aeruginosa (PA) to induce pneumonia 3 days after CLP as a second hit (FIG. 57). We chose PA pneumonia as it is the most common and refractory nosocomial infection especially for patients requiring a ventilator. We assayed T and B cells and their apoptosis, cytokine response and survival to evaluate immunosuppression. Non-lethal CLP increased apoptotic T and/or B cells, with respective cell depletion in the thymus and/or spleen (FIGS. 58-61 and FIGS. 61-63). These changes continued or became exacerbated 24 hrs after the PA infection (FIGS. 58-61 and FIGS. 61-63). However, all these immunosuppressive changes were almost fully rescued by treating sepsis mice with trans mAb, but not IgG control (FIGS. 58-61 and FIGS. 61-63). Consistent with the immunosuppressive changes, IgG-treated mice produced robust cytokines after CLP (FIG. 64) but failed to produce them after the PA challenge (FIG. 65), which was again largely rescued with trans mAb (FIGS. 64-65). Moreover, cytokine response of IgG-treated CLP mice was strongly suppressed after the PA challenge, but largely restored in trans mAb-treated CLP mice, similar to sham mice, except IL-6 (FIG. 66). These host immune responses were also supported by the significant induction of nuclear trans p65 NF-κB in the spleen and lung in trans mAb-treated sepsis mice, but not in PBS- or IgG-treated ones after the PA infection (FIGS. 67-69). Remarkably, whereas ˜80% of IgG-treated CLP mice died one day after the PA infection, over 90% of trans mAb-treated CLP mice successfully overcame the infection (FIG. 70). These effects of trans mAb were specific because when CLP mice were treated with Dexamethasone (DEX) they all died 1 day after the PA infection (FIG. 70), with little nuclear trans p65 NF-κB induction (FIGS. 67-69), consistent with previous findings that DEX inhibits NF-κB, exacerbates immune paralysis and fails to improve overall survival in sepsis. Thus, acute treatment of sepsis mice with trans p65 mAb not only attenuates the cytokine storm and death, but also maintains host immunity to overcome ongoing/nosocomial infections, thereby improving overall survival (FIG. 71).

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations described herein following, in general, the principles described herein and including such departures from the invention that come within known or customary practice within the art to which the invention pertains and may be applied to the essential features hereinbefore set forth, and follows in the scope of the claims. Other embodiments are within the claims.

1. An isolated conformation-specific antibody or antigen-binding fragment thereof, wherein the antibody or antigen-binding fragment thereof specifically binds an epitope comprising the trans conformation of phosphorylated-Threonine254-Proline (pThr254-Pro) of the p65 subunit of nuclear factor kappa-light-chain-enhancer of activated B cells (NF-κB).

2. The antibody or antigen-binding fragment thereof of claim 1, wherein the antibody or antigen-binding fragment thereof comprises a complementarity-determining region (CDR) light chain 1 (CDR-L1) having the amino acid sequence of SEQ ID NO: 1 or a variant thereof; a complementarity-determining region (CDR) light chain 2 (CDR-L2) having the amino acid sequence of SEQ ID NO: 2 or a variant thereof; and/or a complementarity-determining region (CDR) light chain 3 (CDR-L3) having the amino acid sequence of SEQ ID NO: 3 or a variant thereof.

3. The antibody or antigen-binding fragment thereof of claim 2, wherein the antibody or antigen-binding fragment thereof comprises a light chain variable domain having an amino acid sequence that is at least 85% identical to the amino acid sequence of SEQ ID NO: 4.

4. The antibody or antigen-binding fragment thereof of claim 3, wherein the antibody or antigen-binding fragment thereof comprises a light chain variable domain having the amino acid sequence of SEQ ID NO: 4.

5. The antibody or antigen-binding fragment thereof of claim 1, wherein the antibody or antigen-binding fragment thereof comprises a complementarity-determining region (CDR) light chain 1 (CDR-L1) having the amino acid sequence of SEQ ID NO: 11 or a variant thereof; a complementarity-determining region (CDR) light chain 2 (CDR-L2) having the amino acid sequence of SEQ ID NO: 2 or a variant thereof; and/or a complementarity-determining region (CDR) light chain 3 (CDR-L3) having the amino acid sequence of SEQ ID NO: 3 or a variant thereof.

6. The antibody or antigen-binding fragment thereof of claim 5, wherein the antibody or antigen-binding fragment thereof comprises a light chain variable domain having an amino acid sequence that is at least 85% identical to the amino acid sequence of SEQ ID NO: 12.

7. The antibody or antigen-binding fragment thereof of claim 6, wherein the antibody or antigen-binding fragment thereof comprises a light chain variable domain having the amino acid sequence of SEQ ID NO: 12.

8. The antibody or antigen-binding fragment thereof of claim 1, wherein the antibody or antigen-binding fragment thereof comprises a complementarity-determining region (CDR) light chain 1 (CDR-L1) having the amino acid sequence of SEQ ID NO: 13 or a variant thereof; a complementarity-determining region (CDR) light chain 2 (CDR-L2) having the amino acid sequence of SEQ ID NO: 14 or a variant thereof; and/or a complementarity-determining region (CDR) light chain 3 (CDR-L3) having the amino acid sequence of SEQ ID NO: 15 or a variant thereof.

9. The antibody or antigen-binding fragment thereof of claim 8, wherein the antibody or antigen-binding fragment thereof comprises a light chain variable domain having an amino acid sequence that is at least 85% identical to the amino acid sequence of SEQ ID NO: 16.

10. The antibody or antigen-binding fragment thereof of claim 9, wherein the antibody or antigen-binding fragment thereof comprises a light chain variable domain having the amino acid sequence of SEQ ID NO: 16.

11. The antibody or antigen-binding fragment thereof of claim 1, wherein the antibody or antigen-binding fragment thereof comprises a complementarity-determining region (CDR) light chain 1 (CDR-L1) having the amino acid sequence of SEQ ID NO: 17 or a variant thereof; a complementarity-determining region (CDR) light chain 2 (CDR-L2) having the amino acid sequence of SEQ ID NO: 18 or a variant thereof; and/or a complementarity-determining region (CDR) light chain 3 (CDR-L3) having the amino acid sequence of SEQ ID NO: 15 or a variant thereof.

12. The antibody or antigen-binding fragment thereof of claim 11, wherein the antibody or antigen-binding fragment thereof comprises a light chain variable domain having an amino acid sequence that is at least 85% identical to the amino acid sequence of SEQ ID NO: 19.

13. The antibody or antigen-binding fragment thereof of claim 12, wherein the antibody or antigen-binding fragment thereof comprises a light chain variable domain having the amino acid sequence of SEQ ID NO: 19.

14. The antibody or antigen-binding fragment thereof of claim 1, wherein the antibody or antigen-binding fragment thereof comprises a complementarity-determining region (CDR) light chain 1 (CDR-L1) having the amino acid sequence of SEQ ID NO: 20 or a variant thereof; a complementarity-determining region (CDR) light chain 2 (CDR-L2) having the amino acid sequence of SEQ ID NO: 21 or a variant thereof; and/or a complementarity-determining region (CDR) light chain 3 (CDR-L3) having the amino acid sequence of SEQ ID NO: 22 or a variant thereof.

15. The antibody or antigen-binding fragment thereof of claim 14, wherein the antibody or antigen-binding fragment thereof comprises a light chain variable domain having an amino acid sequence that is at least 85% identical to the amino acid sequence of SEQ ID NO: 23.

16. The antibody or antigen-binding fragment thereof of claim 15, wherein the antibody or antigen-binding fragment thereof comprises a light chain variable domain having the amino acid sequence of SEQ ID NO: 23.

17. The antibody or antigen-binding fragment thereof of any one of claim 1-16, wherein the antibody or antigen-binding fragment thereof comprises a complementarity-determining region (CDR) heavy chain 1 (CDR-H1) having the amino acid sequence of SEQ ID NO: 5 or a variant thereof; a complementarity-determining region (CDR) heavy chain 2 (CDR-H2) having the amino acid sequence of SEQ ID NO: 6 or a variant thereof; and/or a complementarity-determining region (CDR) heavy chain 3 (CDR-H3) having the amino acid sequence of SEQ ID NO: 7 or a variant thereof.

18. The antibody or antigen-binding fragment thereof of claim 17, wherein the antibody or antigen-binding fragment thereof comprises a heavy chain variable domain having an amino acid sequence that is at least 85% identical to the amino acid sequence of SEQ ID NO: 8.

19. The antibody or antigen-binding fragment thereof of claim 18, wherein the antibody or antigen-binding fragment thereof comprises a heavy chain variable domain having the amino acid sequence of SEQ ID NO: 8.

20. The antibody or antigen-binding fragment thereof of any one of claims 1-16, wherein the antibody or antigen-binding fragment thereof comprises a complementarity-determining region (CDR) heavy chain 1 (CDR-H1) having the amino acid sequence of SEQ ID NO: 5 or a variant thereof; a complementarity-determining region (CDR) heavy chain 2 (CDR-H2) having the amino acid sequence of SEQ ID NO: 9 or a variant thereof; and/or a complementarity-determining region (CDR) heavy chain 3 (CDR-H3) having the amino acid sequence of SEQ ID NO: 7 or a variant thereof.

21. The antibody or antigen-binding fragment thereof of claim 20, wherein the antibody or antigen-binding fragment thereof comprises a heavy chain variable domain having an amino acid sequence that is at least 85% identical to the amino acid sequence of SEQ ID NO: 10.

22. The antibody or antigen-binding fragment thereof of claim 21, wherein the antibody or antigen-binding fragment thereof comprises a heavy chain variable domain having the amino acid sequence of SEQ ID NO: 10.

23. The antibody or antigen-binding fragment of any one of claims 1-22, wherein the antibody or antigen-binding fragment thereof binds to the trans conformation of pThr254-Pro with at least 10-fold greater affinity than to the cis conformation of pThr254-Pro.

24. The antibody or antigen-binding fragment of claim 23, wherein the antibody or antigen-binding fragment thereof binds to the trans conformation of pThr254-Pro with at least 100-fold greater affinity than to the cis conformation of pThr254-Pro.

25. The antibody or antigen-binding fragment of any one of claims 1-24, wherein the antibody or antigen-binding fragment thereof binds specifically to the active form of NF-κB.

26. The antibody or antigen-binding fragment of claim 25, wherein the antibody or antigen-binding fragment thereof binds to the active form of NF-κB with at least 10-fold greater affinity than to the inactive form of NF-κB.

27. The antibody or antigen-binding fragment of claim 26, wherein the antibody or antigen-binding fragment thereof binds to the active form of NF-κB with at least 100-fold greater affinity than to the inactive form of NF-κB.

28. The antibody or antigen-binding fragment of any one of claims 1-24, wherein the antibody or antigen-binding fragment thereof binds specifically to the nuclear form of NF-κB.

29. The antibody or antigen-binding fragment of claim 28, wherein the antibody or antigen-binding fragment thereof binds to the nuclear form of NF-κB with at least 10-fold greater affinity than to the cytoplasmic form of NF-κB.

30. The antibody or antigen-binding fragment of claim 29, wherein the antibody or antigen-binding fragment thereof binds to the nuclear form of NF-κB with at least 100-fold greater affinity than to the cytoplasmic form of NF-κB.

31. The antibody or antigen-binding fragment thereof of any one of claims 1-30, wherein the antibody or antigen-binding fragment thereof inhibits NF-κB signaling in a cell.

32. The antibody or antigen-binding fragment thereof of any one of claim 31, wherein the cell is an immune cell or a cancer cell.

33. The antibody or antigen-binding fragment thereof of any one of claims 1-32, wherein the antibody or antigen-binding fragment thereof inhibits the expression of one or more genes selected from the group consisting of IGHG4, IGHG3, APOC3, TNFRSF6, CD3G, TNFSF5, CD105, ICAM1, TPMT, IL2RA, SELE, TP53, CRP, IL1A, IL1B, IL1RN, CCR5, IL8, IL2, IL9, TAP1, TNF, LTA, IL6, CD44, NOS2A, SOD2, TNFSF6, IL11, BDKRB1, CSF1, CSF2, CSF3, GSTP1, NQO1, OPRM1, PTAFR, PTGS2, SCNN1A, VCAM1, AGER, ALOX12B, BCL2L1, TNFRSF5, TNFRSF9, IRF7, BLR1, CD48, CD69, CCR7, CR2, F3, HMOX1, TNC, IFNB1, IL13, IL15RA, IRF1, IRF2, LTB, IRF4, MYC, NFKB2, PDGFB, PLAU, LMP2, PTX3, CCL2, CCL5, CCL11, CXCL5, SELP, SLC2A5, STAT5A, VIM, IER3, NFKB1, BM2, BCL2A1, CCL15, CD83, CD74, ELF3, TGM2, DEFB4, MMP9, BCL3, CD80, VEGFC, PLCD1, TNFAIP3, RELB, TFPI2, BCL2, S100A6, TACR1, NFKBIA, CD209, CARD15, CCND1, KLK3, IL15, NR4A2, and HC3.

34. The antibody or antigen-binding fragment thereof of any one of claims 1-33, wherein the antibody or antigen-binding fragment thereof is selected from the group consisting of a monoclonal antibody or antigen-binding fragment thereof, a polyclonal antibody or antigen-binding fragment thereof, a human antibody or antigen-binding fragment thereof, a humanized antibody or antigen-binding fragment thereof, a primatized antibody or antigen-binding fragment thereof, a bispecific antibody or antigen-binding fragment thereof, a multi-specific antibody or antigen-binding fragment thereof, a dual-variable immunoglobulin domain, a monovalent antibody or antigen-binding fragment thereof, a chimeric antibody or antigen-binding fragment thereof, a single-chain Fv molecule (scFv), a diabody, a triabody, a nanobody, an antibody-like protein scaffold, a domain antibody, a Fv fragment, a Fab fragment, a F(ab′)2 molecule, and a tandem scFv (taFv).

35. The antibody or antigen-binding fragment thereof of claim 34, wherein the antibody or antigen-binding fragment thereof is a human, humanized, or chimeric antibody or antigen-binding fragment thereof.

36. The antibody or antigen-binding fragment thereof of any one of claims 1-35, wherein the antibody is conjugated to a therapeutic agent.

37. The antibody or antigen-binding fragment thereof of claim 36, wherein the therapeutic agent is a cytotoxic agent.

38. A polynucleotide encoding the antibody or antigen-binding fragment thereof of any one of claims 1-37.

39. A vector comprising the polynucleotide of claim 38.

40. The vector of claim 39, wherein the vector is an expression vector.

41. The vector of claim 40, wherein the expression vector is a eukaryotic expression vector.

42. The vector of claim 41, wherein the vector is a viral vector.

43. The vector of claim 42, wherein the viral vector is selected from the group consisting of adenovirus (Ad), retrovirus, poxvirus, adeno-associated virus, baculovirus, herpes simplex virus, and a vaccinia virus.

44. A host cell comprising the vector of any one of claims 39-43.

45. The host cell of claim 44, wherein the host cell is a prokaryotic cell.

46. The host cell of claim 44, wherein the host cell is a eukaryotic cell.

47. The host cell of claim 46, wherein the eukaryotic cell is a mammalian cell.

48. The host cell of claim 47, wherein the mammalian cell is a human cell.

49. A pharmaceutical composition comprising the antibody or antigen-binding fragment thereof of any one of claims 1-37, the polynucleotide of claim 38, the vector of any one of claims 39-43, or the host cell of any one of claims 44-48, and a pharmaceutically acceptable carrier or excipient.

50. The pharmaceutical composition of claim 49, wherein the antibody or antigen-binding fragment thereof is present in the pharmaceutical composition in an amount of from about 0.001 mg/ml to about 100 mg/ml.

51. The pharmaceutical composition of claim 49 or 50, wherein the pharmaceutical composition further comprises an additional therapeutic agent.

52. The pharmaceutical composition of claim 51, wherein the additional therapeutic agent is an immunotherapy agent.

53. The pharmaceutical composition of claim 52, wherein the immunotherapy agent is selected from the group consisting of an anti-CTLA-4 agent, an anti-PD-1 agent, an anti-PD-L1 agent, an anti-PD-L2 agent, a TNF-α cross-linking agent, a TRAIL cross-linking agent, an anti-CD27 agent, an anti-CD30 agent, an anti-CD40 agent, an anti-4-1 BB agent, an anti-GITR agent, an anti-OX40 agent, an anti-TRAILR1 agent, an anti-TRAILR2 agent, an anti-TWEAK agent, an anti-TWEAKR agent, an anti-cell surface lymphocyte protein agent, an anti-BRAF agent, an anti-MEK agent, an anti-CD33 agent, an anti-CD20 agent, an anti-HLA-DR agent, an anti-HLA class I agent, an anti-CD52 agent, an anti-A33 agent, an anti-GD3 agent, an anti-PSMA agent, an anti-Ceacan 1 agent, an anti-Galedin 9 agent, an anti-HVEM agent, an anti-VISTA agent, an anti-B7 H4 agent, an anti-HHLA2 agent, an anti-CD155 agent, an anti-CD80 agent, an anti-BTLA agent, an anti-CD160 agent, an anti-CD28 agent, an anti-CD226 agent, an anti-CEACAM1 agent, an anti-TIM3 agent, an anti-TIGIT agent, an anti-CD96 agent, an anti-CD70 agent, an anti-CD27 agent, an anti-LIGHT agent, an anti-CD137 agent, an anti-DR4 agent, an anti-CR5 agent, an anti-TNFRS agent, an anti-TNFR1 agent, an anti-FAS agent, an anti-CD95 agent, an anti-TRAIL agent, an anti-DR6 agent, an anti-EDAR agent, an anti-NGFR agent, an anti-OPG agent, an anti-RANKL agent, an anti-LTβ receptor agent, an anti-BCMA agent, an anti-TACI agent, an anti-BAFFR agent, an anti-EDAR2 agent, an anti-TROY agent, and an anti-RELT agent, optionally wherein the immunotherapy agent is an anti-PD-1 antibody or an anti-PD-L1 antibody.

54. The pharmaceutical composition of claim 52, wherein the immunotherapy agent is selected from the group consisting of an anti-CTLA-4 antibody or antigen-binding fragment thereof, an anti-PD-1 antibody or antigen-binding fragment thereof, an anti-PD-L1 antibody or antigen-binding fragment thereof, an anti-PD-L2 antibody or antigen-binding fragment thereof, a TNF-α cross-linking antibody or antigen-binding fragment thereof, a TRAIL cross-linking antibody or antigen-binding fragment thereof, an anti-CD27 antibody or antigen-binding fragment thereof, an anti-CD30 antibody or antigen-binding fragment thereof, an anti-CD40 antibody or antigen-binding fragment thereof, an anti-4-1 BB antibody or antigen-binding fragment thereof, an anti-GITR antibody or antigen-binding fragment thereof, an anti-OX40 antibody or antigen-binding fragment thereof, an anti-TRAILR1 antibody or antigen-binding fragment thereof, an anti-TRAILR2 antibody or antigen-binding fragment thereof, an anti-TWEAK antibody or antigen-binding fragment thereof, an anti-TWEAKR antibody or antigen-binding fragment thereof, an anti-cell surface lymphocyte protein antibody or antigen-binding fragment thereof, an anti-BRAF antibody or antigen-binding fragment thereof, an anti-MEK antibody or antigen-binding fragment thereof, an anti-CD33 antibody or antigen-binding fragment thereof, an anti-CD20 antibody or antigen-binding fragment thereof, an anti-HLA-DR antibody or antigen-binding fragment thereof, an anti-HLA class I antibody or antigen-binding fragment thereof, an anti-CD52 antibody or antigen-binding fragment thereof, an anti-A33 antibody or antigen-binding fragment thereof, an anti-GD3 antibody or antigen-binding fragment thereof, an anti-PSMA antibody or antigen-binding fragment thereof, an anti-Ceacan 1 antibody or antigen-binding fragment thereof, an anti-Galedin 9 antibody or antigen-binding fragment thereof, an anti-HVEM antibody or antigen-binding fragment thereof, an anti-VISTA antibody or antigen-binding fragment thereof, an anti-B7 H4 antibody or antigen-binding fragment thereof, an anti-HHLA2 antibody or antigen-binding fragment thereof, an anti-CD155 antibody or antigen-binding fragment thereof, an anti-CD80 antibody or antigen-binding fragment thereof, an anti-BTLA antibody or antigen-binding fragment thereof, an anti-CD160 antibody or antigen-binding fragment thereof, an anti-CD28 antibody or antigen-binding fragment thereof, an anti-CD226 antibody or antigen-binding fragment thereof, an anti-CEACAM1 antibody or antigen-binding fragment thereof, an anti-TIM3 antibody or antigen-binding fragment thereof, an anti-TIGIT antibody or antigen-binding fragment thereof, an anti-CD96 antibody or antigen-binding fragment thereof, an anti-CD70 antibody or antigen-binding fragment thereof, an anti-CD27 antibody or antigen-binding fragment thereof, an anti-LIGHT antibody or antigen-binding fragment thereof, an anti-CD137 antibody or antigen-binding fragment thereof, an anti-DR4 antibody or antigen-binding fragment thereof, an anti-CR5 antibody or antigen-binding fragment thereof, an anti-TNFRS antibody or antigen-binding fragment thereof, an anti-TNFR1 antibody or antigen-binding fragment thereof, an anti-FAS antibody or antigen-binding fragment thereof, an anti-CD95 antibody or antigen-binding fragment thereof, an anti-TRAIL antibody or antigen-binding fragment thereof, an anti-DR6 antibody or antigen-binding fragment thereof, an anti-EDAR antibody or antigen-binding fragment thereof, an anti-NGFR antibody or antigen-binding fragment thereof, an anti-OPG antibody or antigen-binding fragment thereof, an anti-RANKL antibody or antigen-binding fragment thereof, an anti-LTβ receptor antibody or antigen-binding fragment thereof, an anti-BCMA antibody or antigen-binding fragment thereof, an anti-TACI antibody or antigen-binding fragment thereof, an anti-BAFFR antibody or antigen-binding fragment thereof, an anti-EDAR2 antibody or antigen-binding fragment thereof, an anti-TROY antibody or antigen-binding fragment thereof, and an anti-RELT antibody or antigen-binding fragment thereof.

55. The pharmaceutical composition of claim 51, wherein the additional therapeutic agent is a chimeric antigen receptor (CAR-T) agent, a chemotherapeutic agent, a small molecule anti-cancer agent, or a cancer vaccine.

56. The pharmaceutical composition of claim 51, wherein the additional therapeutic agent is an antibacterial agent.

57. The pharmaceutical composition of claim 56, wherein the antibacterial agent is selected from the group consisting of Afenide, Amikacin, Amoxicillin, Ampicillin, Arsphenamine, Augmentin, Azithromycin, Azlocillin, Aztreonam, Bacampicillin, Bacitracin, Balofloxacin, Besifloxacin, Capreomycin, Carbacephem (loracarbef), Carbenicillin, Cefacetrile (cephacetrile), Cefaclomezine, Cefaclor, Cefadroxil (cefadroxyl), Cefalexin (cephalexin), Cefaloglycin (cephaloglycin), Cefalonium (cephalonium), Cefaloram, Cefaloridine (cephaloradine), Cefalotin (cephalothin), Cefamandole, Cefaparole, Cefapirin (cephapirin), Cefatrizine, Cefazaflur, Cefazedone, Cefazolin (cephazolin), Cefcanel, Cefcapene, Cefclidine, Cefdaloxime, Cefdinir, Cefditoren, Cefedrolor, Cefempidone, Cefepime, Cefetamet, Cefetrizole, Cefivitril, Cefixime, Cefluprenam, Cefmatilen, Cefmenoxime, Cefmepidium, Cefmetazole, Cefodizime, Cefonicid, Cefoperazone, Cefoselis, Cefotaxime, Cefotetan, Cefovecin, Cefoxazole, Cefoxitin, Cefozopran, Cefpimizole, Cefpirome, Cefpodoxime, Cefprozil (cefproxil), Cefquinome, Cefradine (cephradine), Cefrotil, Cefroxadine, Cefsumide, Ceftaroline, Ceftazidime, Ceftazidime/Avibactam, Cefteram, Ceftezole, Ceftibuten, Ceftiofur, Ceftiolene, Ceftioxide, Ceftizoxime, Ceftobiprole, Ceftriaxone, Cefuracetime, Cefuroxime, Cefuzonam, Cephalexin, Chloramphenicol, Chlorhexidine, Ciprofloxacin, Clarithromycin, Clavulanic Acid, Clinafloxacin, Clindamycin, Cloxacillin, Colimycin, Colistimethate, Colistin, Crysticillin, Cycloserine 2, Demeclocycline, Dicloxacillin, Dirithromycin, Doripenem, Doxycycline, Efprozil, Enoxacin, Ertapenem, Erythromycin, Ethambutol, Flucloxacillin, Flumequine, Fosfomycin, Furazolidone, Gatifloxacin, Geldanamycin, Gemifloxacin, Gentamicin, Glycopeptides, Grepafloxacin, Herbimycin, Imipenem, Isoniazid, Kanamycin, Levofloxacin, Lincomycin, Linezolid, Lipoglycopeptides, Lomefloxacin, Meropenem, Meticillin, Metronidazole, Mezlocillin, Minocycline, Mitomycin, Moxifloxacin, Mupirocin, Nadifloxacin, Nafcillin, Nalidixic Acid, Neomycin, Netilmicin, Nitrofurantoin, Norfloxacin, Ofloxacin, Oxacillin, Oxazolidinones, Oxolinic Acid, Oxytetracycline, Oxytetracycline, Paromomycin, Pazufloxacin, Pefloxacin, Penicillin G, Penicillin V, Pipemidic Acid, Piperacillin, Piromidic Acid, Pivampicillin, Pivmecillinam, Platensimycin, Polymyxin B, Pristinamycin, Prontosil, Prulifloxacin, Pvampicillin, Pyrazinamide, Quinupristin/dalfopristin, Rifabutin, Rifalazil, Rifampin, Rifamycin, Rifapentine, Rosoxacin, Roxithromycin, Rufloxacin, Sitafloxacin, Sparfloxacin, Spectinomycin, Spiramycin, Streptomycin, Sulbactam, Sulfacetamide, Sulfamethizole, Sulfamethoxazole, Sulfanilimide, Sulfisoxazole, Sulphonamides, Sultamicillin, Teicoplanin, Telavancin, Telithromycin, Temafloxacin, Tetracycline, Thiamphenicol, Ticarcillin, Tigecycline, Tinidazole, Tobramycin, Tosufloxacin, Trimethoprim, Trimethoprim-Sulfamethoxazole, Troleandomycin, Trovafloxacin, Tuberactinomycin, Vancomycin, and Viomycin, or a pharmaceutically acceptable salt thereof.

58. The pharmaceutical composition of claim 51, wherein the additional therapeutic agent is an antifungal agent.

59. The pharmaceutical composition of claim 58, wherein the antifungal agent is selected from the group consisting of Abafungin, Albaconazole, Amorolfin, Amphotericin B, Anidulafungin, Bifonazole, Butenafine, Butoconazole, Candicidin, Caspofungin, Ciclopirox, Clotrimazole, Econazole, Fenticonazole, Filipin, Fluconazole, Flucytosine, Griseofulvin, Haloprogin, Hamycin, Isavuconazole, Isoconazole, Itraconazole, Ketoconazole, Micafungin, Miconazole, Naftifine, Natamycin, Nystatin, Omoconazole, Oxiconazole, Polygodial, Posaconazole, Ravuconazole, Rimocidin, Sertaconazole, Sulconazole, Terbinafine, Terconazole, Tioconazole, Tolnaftate, Undecylenic Acid, and Voriconazole, or a pharmaceutically acceptable salt thereof.

60. The pharmaceutical composition of claim 51, wherein the additional therapeutic agent is an antiviral agent.

61. The pharmaceutical composition of claim 60, wherein the antiviral agent is selected from the group consisting of vidarabine, acyclovir, gancyclovir, valgancyclovir, AZT (zidovudine), ddl (didanosine), ddC (zalcitabine), d4T (stavudine), 3TC (lamivudine), nevirapine, delavirdine, saquinavir, ritonavir, indinavir, nelfinavir, ribavirin, and interferon, or a pharmaceutically acceptable salt thereof.

62. A method of producing the antibody or antigen-binding fragment thereof of any one of claims 1-37, the method comprising expressing a polynucleotide encoding the antibody or antigen-binding fragment thereof in a host cell and recovering the antibody or antigen-binding fragment thereof from host cell medium.

63. A method of producing the antibody or antigen-binding fragment thereof of any one of claims 1-37, the method comprising:
(i) administering an antigenic peptide to a non-human host animal, the antigenic peptide comprising a phosphorylated-Threonine-Xaa (pThr-Xaa) motif, where Xaa is any natural or non-natural amino acid;
(ii) isolating antisera containing the antibody or antigen-binding fragment thereof produced in the non-human host animal; and
(iii) purifying the antibody or antigen-binding fragment thereof from the antisera; wherein the antibody or antigen-binding fragment thereof specifically binds to the trans conformation of phosphorylated-Threonine254-Proline (pThr254-Pro) of the p65 subunit of nuclear factor kappa-light-chain-enhancer of activated B cells (NF-κB).

64. The method of claim 63, wherein the host animal is a rabbit, a cow, a horse, a dog, a cat, a goat, a sheep, a chicken, a llama, or a camel.

65. A method of treating a subject having or at risk of developing an immune disorder or an inflammatory disorder, an infection, or a cancer, wherein the method comprises administering to the subject an antigenic peptide, the antigenic peptide including a phosphorylated-Threonine-Xaa (pThr-Xaa), where Xaa is any amino acid, wherein administration of the antigenic peptide produces an antibody or antigen-binding fragment thereof in the subject that specifically binds to the trans conformation of phosphorylated-Threonine254-Proline (pThr254-Pro) of the p65 subunit of nuclear factor kappa-light-chain-enhancer of activated B cells (NF-κB).

66. The method of claim 65, wherein the subject is a human subject.

67. The method of claim any one of claims 63-66, wherein the peptidyl-prolyl bond of the pThr-Xaa motif of the antigenic peptide is preferentially in the trans conformation.

68. The method of claim 67, wherein Xaa is Ala or Gly.

69. The method of any one of claims 63-67, wherein the antigenic peptide is at least 8 amino acid residues in length.

70. The method of any one of claims 63-68, wherein the antigenic peptide is between 8 and 20 amino acid residues in length.

71. A method of treating a subject having or at risk of developing an immune disorder or an inflammatory disorder, an infection, or a cancer, wherein the method comprises administering to the subject a therapeutically effective amount of the antibody or antigen-binding fragment thereof of any one of claims 1-37, the polynucleotide of claim 38, the vector of any one of claims 39-43, or the host cell of any one of claims 44-48.

72. The method of claim 71, wherein the method comprises administering to the subject a therapeutically effective amount of the antibody or antigen-binding fragment thereof of any one of claims 1-37.

73. The method of claim 71 or 72, wherein the immune disorder or inflammatory disorder is selected from acne vulgaris; acute respiratory distress syndrome; Addison's disease; adrenocortical insufficiency; adrenogenital syndrome; allergic conjunctivitis; allergic rhinitis; allergic intraocular inflammatory diseases, ANCA-associated small-vessel vasculitis; angioedema; ankylosing spondylitis; aphthous stomatitis; arthritis, asthma; atherosclerosis; atopic dermatitis; autoimmune disease; autoimmune hemolytic anemia; autoimmune hepatitis; Behcet's disease; Bell's palsy; berylliosis; bronchial asthma; bullous herpetiformis dermatitis; bullous pemphigoid; carditis; celiac disease; cerebral ischaemia; chronic obstructive pulmonary disease; cirrhosis; Cogan's syndrome; contact dermatitis; Crohn's disease; Cushing's syndrome; Cytokine Release Syndrome (CRS); dermatomyositis; diabetes mellitus; discoid lupus erythematosus; eosinophilic fasciitis; epicondylitis; erythema nodosum; exfoliative dermatitis; fibromyalgia; focal glomerulosclerosis; giant cell arteritis; gout; gouty arthritis; graft-versus-host disease; hand eczema; Henoch-Schonlein purpura; herpes gestationis; hirsutism; hypersensitivity drug reactions; idiopathic cerato-scleritis; idiopathic pulmonary fibrosis; idiopathic thrombocytopenic purpura; inflammatory bowel or gastrointestinal disorders, inflammatory dermatoses; juvenile rheumatoid arthritis; laryngeal edema; lichen planus; Loeffler's syndrome; lupus nephritis; lupus vulgaris; lymphomatous tracheobronchitis; macular edema; multiple sclerosis; musculoskeletal and connective tissue disorder; myasthenia gravis; myositis; obstructive pulmonary disease; ocular inflammation; organ transplant rejection; osteoarthritis; pancreatitis; pemphigoid gestationis; pemphigus vulgaris; polyarteritis nodosa; polymyalgia rheumatica; primary adrenocortical insufficiency; primary billiary cirrhosis; pruritus scroti; pruritis/inflammation, psoriasis; psoriatic arthritis; Reiter's disease; relapsing polychondritis; rheumatic carditis; rheumatic fever; rheumatoid arthritis; rosacea caused by sarcoidosis; rosacea caused by scleroderma; rosacea caused by Sweet's syndrome; rosacea caused by systemic lupus erythematosus; rosacea caused by urticaria; rosacea caused by zoster-associated pain; sarcoidosis; scleroderma; segmental glomerulosclerosis; sepsis; serum sickness; shoulder tendinitis or bursitis; Sjogren's syndrome; Still's disease; stroke-induced brain cell death; Sweet's disease; systemic dermatomyositis; systemic inflammatory response syndrome (SIRS); systemic lupus erythematosus; systemic sclerosis; Takayasu's arteritis; temporal arteritis; and thyroiditis; toxic epidermal necrolysis; tuberculosis; type-1 diabetes; ulcerative colitis; uveitis; vasculitis; and Wegener's granulomatosis.

74. The method of claim 73, wherein the immune disorder or inflammatory disorder is sepsis.

75. The method of claim 73, wherein the immune disorder or inflammatory disorder is SIRS.

76. The method of claim 73, wherein the immune or inflammatory disorder is CRS.

77. The method of any one of claims 71-76, wherein the immune or inflammatory disorder is associated with a betacoronavirus infection.

78. A method of treating a subject having or at risk of developing sepsis, wherein the method comprises administering to the subject a therapeutically effective amount of the antibody or antigen-binding fragment thereof of any one of claims 1-37, the polynucleotide of claim 38, the vector of any one of claims 39-43, or the host cell of any one of claims 43-48.

79. The method of claim 78, wherein the method comprises administering to the subject a therapeutically effective amount of the antibody or antigen-binding fragment thereof of any one of claims 1-23.

80. The method of claim 78 or 79, wherein the sepsis is bacterial sepsis.

81. The method of claim 78 or 79, wherein the sepsis is viral sepsis.

82. The method of claim 78 or 79, wherein the sepsis is sterile sepsis.

83. The method of claim 78 or 79, wherein the sepsis is associated with trauma, burns, pancreatitis, or ischaemic reperfusion.

84. The method of claim 78 or 79, wherein the sepsis is associated with a betacoronavirus infection.

85. A method of treating a subject having or at risk of developing systemic inflammatory response syndrome (SIRS), wherein the method comprises administering to the subject a therapeutically effective amount of the antibody or antigen-binding fragment thereof of any one of claims 1-37, the polynucleotide of claim 38, the vector of any one of claims 39-43, or the host cell of any one of claims 44-48.

86. The method of claim 85, wherein the method comprises administering to the subject a therapeutically effective amount of the antibody or antigen-binding fragment thereof of any one of claims 1-37.

87. The method of claim 85 or 86, wherein the SIRS is associated with infection, trauma, burns, pancreatitis, or ischaemic reperfusion.

88. A method of treating a subject having or at risk of developing Cytokine Release Syndrome (CRS), wherein the method comprises administering to the subject a therapeutically effective amount of the antibody or antigen-binding fragment thereof of any one of claims 1-37, the polynucleotide of claim 38, the vector of any one of claims 39-43, or the host cell of any one of claims 44-48.

89. The method of claim 88, wherein the method comprises administering to the subject a therapeutically effective amount of the antibody or antigen-binding fragment thereof of any one of claims 1-37.

90. The method of claim 88 or 89, wherein the CRS is associated with an antibody therapy, a small molecule cancer therapy, stem cell transplantation, graft-versus-host disease, CAR-T, an infection, or a hemophagocytic syndrome.

91. The method of claim 88 or 89, wherein the CRS is associated with a betacoronavirus infection.

92. A method of treating a subject having or at risk of developing an infection, wherein the method comprises administering to the subject a therapeutically effective amount of the antibody or antigen-binding fragment thereof of any one of claims 1-37, the polynucleotide of claim 38, the vector of any one of claims 39-43, or the host cell of any one of claims 44-48.

93. The method of claim 92, wherein the method comprises administering to the subject a therapeutically effective amount of the antibody or antigen-binding fragment thereof of any one of claims 1-37.

94. The method of claim 92 or 93, wherein the infection is a bacterial infection.

95. The method of claim 92 or 93, wherein the infection is a viral infection.

96. The method of claim 95, wherein the infection is a betacoronavirus infection.

97. The method of 96, wherein the betacoronavirus infection is SARS-CoV, MERS-CoV, or SARS-CoV-2.

98. The method of claim 97, wherein the betacoronavirus infection is SARS-CoV-2.

99. The method of any one of claims 95-98, wherein the subject has been diagnosed with COVID-19, is suspected to have COVID-19, has been in contact with someone diagnosed with COVID-19, or has recently traveled to an area experiencing an outbreak of COVID-19.

100. The method of claim 92 or 93, wherein the infection is a fungal infection.

101. The method of claim 92 or 93, wherein the infection is a parasitic infection.

102. A method of treating a subject having or at risk of developing a cancer, wherein the method comprises administering to the subject a therapeutically effective amount of the antibody or antigen-binding fragment thereof of any one of claims 1-37, the polynucleotide of claim 38, the vector of any one of claims 39-43, or the host cell of any one of claims 44-48.

103. The method of claim 102, wherein the method comprises administering to the subject a therapeutically effective amount of the antibody or antigen-binding fragment thereof of any one of claims 1-37.

104. The method of claim 102 or 103, wherein the cancer is selected from leukemia, lymphoma, liver cancer, bone cancer, lung cancer, brain cancer, bladder cancer, gastrointestinal cancer, breast cancer, cardiac cancer, cervical cancer, uterine cancer, ovarian cancer, colon cancer, skin cancer, head and neck cancer, gallbladder cancer, laryngeal cancer, lip and oral cavity cancer, ocular cancer, melanoma, pancreatic cancer, prostate cancer, colorectal cancer, testicular cancer, and throat cancer.

105. The method of any one of claims 71-104, wherein the subject in a human subject.

106. The method of any one of claims 71-105, wherein the method further comprises administering to the subject an additional therapeutic agent.

107. The method of claim 106, wherein the additional therapeutic agent is an immunotherapy agent.

108. The method of claim 107, wherein the immunotherapy agent is selected from the group consisting of an anti-CTLA-4 agent, an anti-PD-1 agent, an anti-PD-L1 agent, an anti-PD-L2 agent, a TNF-α cross-linking agent, a TRAIL cross-linking agent, an anti-CD27 agent, an anti-CD30 agent, an anti-CD40 agent, an anti-4-1 BB agent, an anti-GITR agent, an anti-OX40 agent, an anti-TRAILR1 agent, an anti-TRAILR2 agent, an anti-TWEAK agent, an anti-TWEAKR agent, an anti-cell surface lymphocyte protein agent, an anti-BRAF agent, an anti-MEK agent, an anti-CD33 agent, an anti-CD20 agent, an anti-HLA-DR agent, an anti-HLA class I agent, an anti-CD52 agent, an anti-A33 agent, an anti-GD3 agent, an anti-PSMA agent, an anti-Ceacan 1 agent, an anti-Galedin 9 agent, an anti-HVEM agent, an anti-VISTA agent, an anti-B7 H4 agent, an anti-HHLA2 agent, an anti-CD155 agent, an anti-CD80 agent, an anti-BTLA agent, an anti-CD160 agent, an anti-CD28 agent, an anti-CD226 agent, an anti-CEACAM1 agent, an anti-TIM3 agent, an anti-TIGIT agent, an anti-CD96 agent, an anti-CD70 agent, an anti-CD27 agent, an anti-LIGHT agent, an anti-CD137 agent, an anti-DR4 agent, an anti-CR5 agent, an anti-TNFRS agent, an anti-TNFR1 agent, an anti-FAS agent, an anti-CD95 agent, an anti-TRAIL agent, an anti-DR6 agent, an anti-EDAR agent, an anti-NGFR agent, an anti-OPG agent, an anti-RANKL agent, an anti-LTβ receptor agent, an anti-BCMA agent, an anti-TACI agent, an anti-BAFFR agent, an anti-EDAR2 agent, an anti-TROY agent, and an anti-RELT agent, optionally wherein the immunotherapy agent is an anti-PD-1 antibody or an anti-PD-L1 antibody.

109. The method of claim 107, wherein the immunotherapy agent is selected from the group consisting of an anti-CTLA-4 antibody or antigen-binding fragment thereof, an anti-PD-1 antibody or antigen-binding fragment thereof, an anti-PD-L1 antibody or antigen-binding fragment thereof, an anti-PD-L2 antibody or antigen-binding fragment thereof, a TNF-α cross-linking antibody or antigen-binding fragment thereof, a TRAIL cross-linking antibody or antigen-binding fragment thereof, an anti-CD27 antibody or antigen-binding fragment thereof, an anti-CD30 antibody or antigen-binding fragment thereof, an anti-CD40 antibody or antigen-binding fragment thereof, an anti-4-1 BB antibody or antigen-binding fragment thereof, an anti-GITR antibody or antigen-binding fragment thereof, an anti-OX40 antibody or antigen-binding fragment thereof, an anti-TRAILR1 antibody or antigen-binding fragment thereof, an anti-TRAILR2 antibody or antigen-binding fragment thereof, an anti-TWEAK antibody or antigen-binding fragment thereof, an anti-TWEAKR antibody or antigen-binding fragment thereof, an anti-cell surface lymphocyte protein antibody or antigen-binding fragment thereof, an anti-BRAF antibody or antigen-binding fragment thereof, an anti-MEK antibody or antigen-binding fragment thereof, an anti-CD33 antibody or antigen-binding fragment thereof, an anti-CD20 antibody or antigen-binding fragment thereof, an anti-HLA-DR antibody or antigen-binding fragment thereof, an anti-HLA class I antibody or antigen-binding fragment thereof, an anti-CD52 antibody or antigen-binding fragment thereof, an anti-A33 antibody or antigen-binding fragment thereof, an anti-GD3 antibody or antigen-binding fragment thereof, an anti-PSMA antibody or antigen-binding fragment thereof, an anti-Ceacan 1 antibody or antigen-binding fragment thereof, an anti-Galedin 9 antibody or antigen-binding fragment thereof, an anti-HVEM antibody or antigen-binding fragment thereof, an anti-VISTA antibody or antigen-binding fragment thereof, an anti-B7 H4 antibody or antigen-binding fragment thereof, an anti-HHLA2 antibody or antigen-binding fragment thereof, an anti-CD155 antibody or antigen-binding fragment thereof, an anti-CD80 antibody or antigen-binding fragment thereof, an anti-BTLA antibody or antigen-binding fragment thereof, an anti-CD160 antibody or antigen-binding fragment thereof, an anti-CD28 antibody or antigen-binding fragment thereof, an anti-CD226 antibody or antigen-binding fragment thereof, an anti-CEACAM1 antibody or antigen-binding fragment thereof, an anti-TIM3 antibody or antigen-binding fragment thereof, an anti-TIGIT antibody or antigen-binding fragment thereof, an anti-CD96 antibody or antigen-binding fragment thereof, an anti-CD70 antibody or antigen-binding fragment thereof, an anti-CD27 antibody or antigen-binding fragment thereof, an anti-LIGHT antibody or antigen-binding fragment thereof, an anti-CD137 antibody or antigen-binding fragment thereof, an anti-DR4 antibody or antigen-binding fragment thereof, an anti-CR5 antibody or antigen-binding fragment thereof, an anti-TNFRS antibody or antigen-binding fragment thereof, an anti-TNFR1 antibody or antigen-binding fragment thereof, an anti-FAS antibody or antigen-binding fragment thereof, an anti-CD95 antibody or antigen-binding fragment thereof, an anti-TRAIL antibody or antigen-binding fragment thereof, an anti-DR6 antibody or antigen-binding fragment thereof, an anti-EDAR antibody or antigen-binding fragment thereof, an anti-NGFR antibody or antigen-binding fragment thereof, an anti-OPG antibody or antigen-binding fragment thereof, an anti-RANKL antibody or antigen-binding fragment thereof, an anti-LTβ receptor antibody or antigen-binding fragment thereof, an anti-BCMA antibody or antigen-binding fragment thereof, an anti-TACI antibody or antigen-binding fragment thereof, an anti-BAFFR antibody or antigen-binding fragment thereof, an anti-EDAR2 antibody or antigen-binding fragment thereof, an anti-TROY antibody or antigen-binding fragment thereof, and an anti-RELT antibody or antigen-binding fragment thereof.

110. The method of claim 106, wherein the additional therapeutic agent is a chimeric antigen receptor (CAR-T) agent, a chemotherapeutic agent, a small molecule anti-cancer agent, or a cancer vaccine.

111. The method of claim 106, wherein the additional therapeutic agent is an antibacterial agent.

112. The method of claim 111, wherein the antibacterial agent is selected from the group consisting of Afenide, Amikacin, Amoxicillin, Ampicillin, Arsphenamine, Augmentin, Azithromycin, Azlocillin, Aztreonam, Bacampicillin, Bacitracin, Balofloxacin, Besifloxacin, Capreomycin, Carbacephem (loracarbef), Carbenicillin, Cefacetrile (cephacetrile), Cefaclomezine, Cefaclor, Cefadroxil (cefadroxyl), Cefalexin (cephalexin), Cefaloglycin (cephaloglycin), Cefalonium (cephalonium), Cefaloram, Cefaloridine (cephaloradine), Cefalotin (cephalothin), Cefamandole, Cefaparole, Cefapirin (cephapirin), Cefatrizine, Cefazaflur, Cefazedone, Cefazolin (cephazolin), Cefcanel, Cefcapene, Cefclidine, Cefdaloxime, Cefdinir, Cefditoren, Cefedrolor, Cefempidone, Cefepime, Cefetamet, Cefetrizole, Cefivitril, Cefixime, Cefluprenam, Cefmatilen, Cefmenoxime, Cefmepidium, Cefmetazole, Cefodizime, Cefonicid, Cefoperazone, Cefoselis, Cefotaxime, Cefotetan, Cefovecin, Cefoxazole, Cefoxitin, Cefozopran, Cefpimizole, Cefpirome, Cefpodoxime, Cefprozil (cefproxil), Cefquinome, Cefradine (cephradine), Cefrotil, Cefroxadine, Cefsumide, Ceftaroline, Ceftazidime, Ceftazidime/Avibactam, Cefteram, Ceftezole, Ceftibuten, Ceftiofur, Ceftiolene, Ceftioxide, Ceftizoxime, Ceftobiprole, Ceftriaxone, Cefuracetime, Cefuroxime, Cefuzonam, Cephalexin, Chloramphenicol, Chlorhexidine, Ciprofloxacin, Clarithromycin, Clavulanic Acid, Clinafloxacin, Clindamycin, Cloxacillin, Colimycin, Colistimethate, Colistin, Crysticillin, Cycloserine 2, Demeclocycline, Dicloxacillin, Dirithromycin, Doripenem, Doxycycline, Efprozil, Enoxacin, Ertapenem, Erythromycin, Ethambutol, Flucloxacillin, Flumequine, Fosfomycin, Furazolidone, Gatifloxacin, Geldanamycin, Gemifloxacin, Gentamicin, Glycopeptides, Grepafloxacin, Herbimycin, Imipenem, Isoniazid, Kanamycin, Levofloxacin, Lincomycin, Linezolid, Lipoglycopeptides, Lomefloxacin, Meropenem, Meticillin, Metronidazole, Mezlocillin, Minocycline, Mitomycin, Moxifloxacin, Mupirocin, Nadifloxacin, Nafcillin, Nalidixic Acid, Neomycin, Netilmicin, Nitrofurantoin, Norfloxacin, Ofloxacin, Oxacillin, Oxazolidinones, Oxolinic Acid, Oxytetracycline, Oxytetracycline, Paromomycin, Pazufloxacin, Pefloxacin, Penicillin G, Penicillin V, Pipemidic Acid, Piperacillin, Piromidic Acid, Pivampicillin, Pivmecillinam, Platensimycin, Polymyxin B, Pristinamycin, Prontosil, Prulifloxacin, Pvampicillin, Pyrazinamide, Quinupristin/dalfopristin, Rifabutin, Rifalazil, Rifampin, Rifamycin, Rifapentine, Rosoxacin, Roxithromycin, Rufloxacin, Sitafloxacin, Sparfloxacin, Spectinomycin, Spiramycin, Streptomycin, Sulbactam, Sulfacetamide, Sulfamethizole, Sulfamethoxazole, Sulfanilimide, Sulfisoxazole, Sulphonamides, Sultamicillin, Teicoplanin, Telavancin, Telithromycin, Temafloxacin, Tetracycline, Thiamphenicol, Ticarcillin, Tigecycline, Tinidazole, Tobramycin, Tosufloxacin, Trimethoprim, Trimethoprim-Sulfamethoxazole, Troleandomycin, Trovafloxacin, Tuberactinomycin, Vancomycin, and Viomycin, or a pharmaceutically acceptable salt thereof.

113. The method of claim 106, wherein the additional therapeutic agent is an antifungal agent.

114. The method of claim 113, wherein the antifungal agent is selected from the group consisting of Abafungin, Albaconazole, Amorolfin, Amphotericin B, Anidulafungin, Bifonazole, Butenafine, Butoconazole, Candicidin, Caspofungin, Ciclopirox, Clotrimazole, Econazole, Fenticonazole, Filipin, Fluconazole, Flucytosine, Griseofulvin, Haloprogin, Hamycin, Isavuconazole, Isoconazole, Itraconazole, Ketoconazole, Micafungin, Miconazole, Naftifine, Natamycin, Nystatin, Omoconazole, Oxiconazole, Polygodial, Posaconazole, Ravuconazole, Rimocidin, Sertaconazole, Sulconazole, Terbinafine, Terconazole, Tioconazole, Tolnaftate, Undecylenic Acid, and Voriconazole, or a pharmaceutically acceptable salt thereof.

115. The method of claim 106, wherein the additional therapeutic agent is an antiviral agent.

116. The method of claim 115, wherein the antiviral agent is selected from the group consisting of vidarabine, acyclovir, gancyclovir, valgancyclovir, AZT (zidovudine), ddl (didanosine), ddC (zalcitabine), d4T (stavudine), 3TC (lamivudine), nevirapine, delavirdine, saquinavir, ritonavir, indinavir, nelfinavir, ribavirin, and interferon, or a pharmaceutically acceptable salt thereof.

117. The method of any one of claims 71-116, wherein the antibody or antigen-binding fragment thereof is administered to the subject in an amount of from about 0.001 mg/kg to about 100 mg/kg.

118. A method of determining the level of nuclear NF-κB activity in a sample from subject, the method comprising:
(i) contacting a sample from the subject with the antibody or antigen-binding fragment thereof of any one of claims 1-37; and
(ii) determining the level of the nuclear NF-κB in the sample of (i) by determining the level of the antibody or antigen-binding fragment thereof bound to the nuclear NF-κB.

119. The method of claim 118, wherein the method further comprises:
(iii) comparing the level of the nuclear NF-κB determined in (ii) to a reference value of nuclear NF-κB.

120. The method of claim 118 or 119, wherein the subject has or is at risk of developing an immune disorder or an inflammatory disorder, an infection, or a cancer.

121. The method of claim 119 or 120, wherein the reference value of nuclear NF-κB is the average level of nuclear NF-κB in a population of subjects having an immune disorder or an inflammatory disorder, an infection, or a cancer.

122. The method of claim 119 or 120, wherein the reference value of nuclear NF-κB is the average level of nuclear NF-κB in a population of subjects not having an immune disorder or an inflammatory disorder, an infection, or a cancer.

123. The method of any one of claims 120-122, wherein the immune disorder of inflammatory disorder is sepsis, SIRS, or CRS.

124. The method of any one of claims 119-123, wherein if the level of nuclear NF-κB determined in (ii) is greater than the reference value of nuclear NF-κB, then the subject is treated with a therapeutically effective amount of the antibody or antigen-binding fragment thereof of any one of claims 1-37, the polynucleotide of claim 38, the vector of any one of claims 39-43, or the host cell of any one of claims 44-48.

125. A kit comprising an agent selected from the group consisting of the antibody or antigen-binding fragment thereof of any one of claims 1-37, the polynucleotide of claim 38, the vector of any one of claims 39-43, or the host cell of any one of claims 44-48, or the pharmaceutical composition of any one of claims 49-61.

126. The kit of claim 125, wherein the kit comprises the antibody or antigen-binding fragment thereof any one of claims 1-37.

127. The kit of claim 125, wherein the kit comprises the polynucleotide of claim 28.

128. The kit of claim 125, wherein the kit comprises the vector of any one of claims 39-43.

129. The kit of claim 128, wherein the kit further comprises instructions for transfecting the vector into a host cell.

130. The kit of claim 129, wherein the kit further comprises instructions for expressing the antibody, antigen-binding fragment thereof, or construct in the host cell.

131. The kit of claim 128, wherein the kit comprises the host cell of any one of claims 33-37.

132. The kit of claim 131, wherein the kit further comprises a reagent that can be used to express the antibody, antigen-binding fragment thereof, or construct in the host cell.

133. The kit of claim 131, wherein the kit comprises the pharmaceutical composition of any one of claims 38-50.

134. The kit of claim 131, further comprising instructions for administering the agent to a subject.

135. The kit of claim 125, wherein the subject is a human subject.

136. A polynucleotide encoding the antibody or antigen-binding fragment thereof of any one of claims 1-16.

137. A vector comprising the polynucleotide of claim 136.

138. The vector of claim 137, wherein the vector is an expression vector.

139. The vector of claim 138, wherein the expression vector is a eukaryotic expression vector.

140. The vector of claim 139, wherein the vector is a viral vector.

141. The vector of claim 140, wherein the viral vector is selected from the group consisting of adenovirus (Ad), retrovirus, poxvirus, adeno-associated virus, baculovirus, herpes simplex virus, and a vaccinia virus.

142. A host cell comprising the vector of claim 137.

143. A pharmaceutical composition comprising the antibody or antigen-binding fragment thereof of any one of claims 1-16 and a pharmaceutically acceptable carrier or excipient.

144. A pharmaceutical composition comprising the polynucleotide of claim 136 and a pharmaceutically acceptable carrier or excipient.

145. A pharmaceutical composition comprising the vector of claim 137 and a pharmaceutically acceptable carrier or excipient.

146. A pharmaceutical composition comprising the host cell of claim 142 and a pharmaceutically acceptable carrier or excipient.

147. A method of treating a subject having or at risk of developing an immune disorder, an inflammatory disorder, an infection, a cancer, sepsis, systemic inflammatory response syndrome (SIRS), or Cytokine Release Syndrome (CRS), wherein the method comprises administering to the subject subject a therapeutically effective amount of the antibody or antigen-binding fragment thereof of any one of claims 1-16, a polynucleotide encoding the antibody or antigen-binding fragment thereof, a vector comprising the polynucleotide, or a host cell comprising the vector.

148. The method of claim 147, wherein the subject is a human subject.

149. A kit comprising an agent selected from the group consisting of the antibody or antigen-binding fragment thereof of any one of claims 1-16, a polynucleotide encoding the antibody or antigen-binding fragment thereof, a vector comprising the polynucleotide, or a host cell comprising the vector.
